(19) United States
(12) Patent Application Publication
(10) Pub. No.: US 2024/0131068 A1
(43) Pub. Date: Apr. 25, 2024

(54) CAR-T CONSTRUCTS COMPRISING A NOVEL CD19 BINDER COMBINED WITH IL18 AND METHODS OF USING THE SAME

(71) Applicants: Kite Pharma, Inc., Santa Monica, CA (US); The Trustees of the University of Pennsylvania, Philadelphia, PA (US)

(72) Inventors: Neil C. Sheppard, Philadelphia, PA (US); Yanping Luo, Santa Monica, CA (US); David Barrett, Santa Monica, CA (US); Regina Young, Philadelphia, PA (US)

(73) Assignees: Kite Pharma, Inc., Santa Monica, CA (US); The Trustees of the University of Pennsylvania, Philadelphia, PA (US)

(21) Appl. No.: 18/381,258

(22) Filed: Oct. 17, 2023

Related U.S. Application Data
(60) Provisional application No. 63/417,216, filed on Oct. 18, 2022.
(60) Provisional application No. 63/426,944, filed on Nov. 21, 2022.

(51) Int. Cl. A61K 35/17; A61K 39/00; A61P 35/00; C07K 14/54; C07K 14/705; C12N 15/86

(52) U.S. Cl. CPC A61K 35/17; A61K 39/4631; A61K 39/464411; A61K 39/46444; A61P 35/00; C07K 14/54; C07K 14/70503; C12N 15/86; A61K 2239/15; A61K 2239/21; A61K 2239/22; C12N 2740/15043

(57) ABSTRACT

The disclosure relates to immune cells comprising one or more vectors comprising a nucleic acid sequence encoding a chimeric antigen receptor specific for CD19 and a nucleic acid sequence encoding an enhancer of T cell priming (e.g., IL-18), compositions comprising the T cells, and methods of generating and/or using the T cells to treat diseases associated with the expression of CD19.

### CROSS-REFERENCE

The present application claims priority from U.S. Provisional Application No. 63/417,216, filed on Oct. 18, 2022, and U.S. Provisional Application No. 63/426,944, filed Nov. 21, 2022, the contents of which are hereby incorporated by reference in their entirety for all purposes.

### SEQUENCE LISTING

The present application contains a Sequence Listing which is hereby incorporated by reference in its entirety. Said Sequence Listing XML, file was created on Jan. 3, 2024, is 267 bytes in size, and is named 125400_1744_Sequence_Listing.XML.

### FIELD OF THE INVENTION

The present disclosure relates generally to T cells engineered to express a Chimeric Antigen Receptor (CAR) and Interleukin-18 to treat a disease associated with expression of the Cluster of Differentiation 19 protein (CD19).

### BACKGROUND

Recent developments using chimeric antigen receptor (CAR) modified autologous T cell (CART) therapy, which relies on redirecting T cells to a suitable cell-surface molecule on cancer cells such as B cell malignancies, show promising results in harnessing the power of the immune system to treat B cell malignancies and other cancers. Sadelain et al., Cancer 1907393 1 Discovery 3:388-398 (2013). The clinical results of the murine derived CART19 (i.e., “CTL019”) have shown promise in establishing complete remissions in patients suffering with chronic lymphocytic leukemia (CLL) as well as in childhood acute lymphoid leukemia (ALL). Despite the clinical success of various CD19 CART cell therapies, the therapeutic index of these therapies remains high due to immunogenicity issues, toxicities associated with the infusion of the CAR T cells, and relapse of the tumor.

Accordingly, there is an urgent need in the art for novel approaches that can solve or mitigate the harmful side effects of CAR T cell therapies and allow for more effective, safe, and efficient adoptive immunotherapy. The present disclosure addresses this need.

### SUMMARY OF THE INVENTION

One aspect of the present disclosure provides a vector comprising: (a) a first polynucleotide comprising a constitutive promoter operably linked to a nucleic acid encoding a chimeric antigen receptor (CAR), where the CAR comprises a single chain antibody or a single chain antibody fragment comprising an anti-CD19 binding domain, a transmembrane domain, a costimulatory, and an intracellular signaling domain; and (b) a second polynucleotide comprising a nucleic acid encoding a polypeptide that enhances an immune cell function, or a functional derivative thereof. In some embodiments, the first polynucleotide is operably linked to the second polypeptide via a linker peptide.

In some embodiments, the polypeptide that enhances an immune cell function, or a functional derivative thereof is selected from the group consisting of a cytokine, an interferon, a chemokine, an antibody or antibody fragment, a checkpoint inhibitor antagonist, a dominant negative receptor, a switch receptor, and a combination thereof.

In some embodiments, the polypeptide that enhances an immune cell function, or a functional derivative thereof is: (a) a chemokine, a chemokine receptor, a cytokine, a cytokine receptor, and a combination thereof; (b) a cytokine selected from the group consisting of Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), Interleukin-18 (IL-18), Interleukin-18 receptor (IL-18R), Interleukin-21 (IL-21), granulocyte macrophage colony stimulating factor, alpha, beta or gamma interferon, erythropoietin, and a combination thereof; or (c) a chemokine selected from CCL21, CCL19, or a combination thereof.

In some embodiments, the polypeptide that enhances an immune cell function, or a functional derivative thereof further comprises a leader sequence selected from the group consisting of an IL-2 signal sequence, an IL-12 signal sequence, a kappa leader sequence, a CD8 leader sequence, or any equivalent thereof.

In some embodiments, the polypeptide that enhances an immune cell function, or a functional derivative thereof comprises an IL-18 polypeptide or a polypeptide having the amino acid sequence of SEQ ID NO: 105, SEQ ID NO: 215, SEQ ID NO: 106, SEQ ID NO: 107 or an amino acid sequence having about 90%, about 91%, about 92%, about 93%, about 94%, about 95%, about 96%, about 97%, about 98%, or about 99% identity to SEQ ID NO: 105, SEQ ID NO: 215, SEQ ID NO: 106, or SEQ ID NO: 107.

In some embodiments, the IL-18 polypeptide further comprises a CD8 leader sequence, or the amino acid sequence of SEQ ID NO: 25 or an amino acid sequence having about 90%, about 91%, about 92%, about 93%, about 94%, about 95%, about 96%, about 97%, about 98%, or about 99% identity to SEQ ID NO: 25.

In some embodiments, the IL-18 polypeptide comprises a mutation at a position selected from the group consisting of position 42, 74, 85, 87, 89, 104, 112, 10, 132, 143, 149, 163, and 189 of SEQ ID NO: 107.

In some embodiments, the IL-18 polypeptide comprises E42A; E42K; K89A; E42A and K89A; E42K and K89A; E42A and C74S; E42A, C74S, and K89A; C74S, and K89A; C74S, C112S, and C112S; E42A, C74S, C112S, and C112S; E42A, K89A, C74S, C112S, and C112S in SEQ ID NO: 7.

In some embodiments, the IL-18 polypeptide: (a) exhibits at least an about 2-fold increased activity when compared to WT IL-18; (b) is resistant to IL18BP inhibition when compared to WT IL-18; and/or (c) requires at least an about 4 fold concentration of IL-18BP for neutralization when compared to WT IL-18.

In some embodiments, the vector is selected from the group consisting of a DNA, a RNA, a plasmid, a lentivirus vector, an adenoviral vector, or a retroviral vector. In some embodiments, the vector is an in vitro transcribed vector.

In some embodiments of the vector disclosed herein, the constitutive promoter comprises a promoter selected from the group consisting of an EF-1alpha promoter, a PGK-1 promoter, a truncated PGK-1 promoter, an UBC promoter, a CMV promoter, a CAGG promoter, and an SV40 promoter. In some embodiments, the constitutive promoter: (a) is an EF-1 promoter; or (b) comprises the sequence of SEQ ID NO: 101.

In some embodiments, the vector further comprises a rev response element (RRE), a poly(A) tail, a 3′ UTR, a woodchuck hepatitis virus posttranscriptional regulatory element (WPRE), and/or a cPPT sequence. In that embodiment, the WPRE comprises the sequence of SEQ ID NO: 100.

In some embodiments, the anti-CD19 binding domain comprises: (a) a light chain variable domain including a light chain complementary determining region 1 (LC CDR1) of SEQ ID NO: 1, a light chain complementary determining region 2 (LC CDR2) of SEQ ID NO: 2, and a light chain complementary determining region 3 (LC CDR3) of SEQ ID NO: 3; and a heavy chain variable domain including a heavy chain complementary determining region 1 (HC CDR1) of SEQ ID NO: 4, a heavy chain complementary determining region 2 (HC CDR2) of SEQ ID NO: 5, and a heavy chain complementary determining region 3 (HC CDR3) of SEQ ID NO: 6; or (b) a light chain variable domain including a light chain complementary determining region 1 (LC CDR1) of SEQ ID NO: 193, a light chain complementary determining region 2 (LC CDR2) of SEQ ID NO: 194, and a light chain complementary determining region 3 (LC CDR3) of SEQ ID NO: 195; and a heavy chain variable domain including a heavy chain complementary determining region 1 (HC CDR1) of SEQ ID NO: 196, a heavy chain complementary determining region 2 (HC CDR2) of SEQ ID NO: 197, and a heavy chain complementary determining region 3 (HC CDR3) of SEQ ID NO: 198; or (c) a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

In some embodiments, the light chain variable region comprises the amino acid sequence of SEQ ID NO: 7 or 199, or an amino acid sequence having at least about 90%, about 91%, about 92%, about 93%, about 94%, about 95%, about 96%, about 97%, about 98%, or about 99% identity to SEQ ID NO: 7 or 199. In some embodiments, the heavy chain variable region comprises the amino acid sequence of SEQ ID NO: 8 or 200, or an amino acid sequence having at least about 90%, about 91%, about 92%, about 93%, about 94%, about 95%, about 96%, about 97%, about 98%, or about 99% identity to SEQ ID NO: 8 or 200. In some embodiments, the light chain variable region comprises the amino acid sequence of SEQ ID NO: 7 and the heavy chain variable region comprises the amino acid sequence of SEQ ID NO: 8. In some embodiments, the light chain variable region comprises the amino acid sequence of SEQ ID NO: 199 and the heavy chain variable region comprises the amino acid sequence of SEQ ID NO: 200.

In some embodiments, the CD19 binding domain is an scFv. In some embodiments, the anti-CD19 binding domain comprises an amino acid sequence selected from the group consisting of SEQ ID NO: 9, 18, 64, 75, 86, 190, 157, 212, 201, 226, 179, 168, and 146, or a sequence having 90%, about 91%, about 92%, about 93%, about 94%, about 95%, about 96%, about 97%, about 98%, or about 99% identity to SEQ ID NO: 9, 18, 64, 75, 86, 190, 157, 212, 201, 226, 179, 168, or 146.

In some embodiments, the anti-CD19 binding domain comprises: (a) a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; or (b) a sequence having 90%, about 91%, about 92%, about 93%, about 94%, about 95%, about 96%, about 97%, about 98%, or about 99% identity to SEQ ID NO: 21, SEQ ID NO: 24 SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

In some embodiments, the anti-CD19 binding domain comprises a nucleic acid sequence selected from the group consisting of SEQ ID NO: 19, SEQ ID NO: 20, SEQ ID NO: 21, SEQ ID NO: 22, SEQ ID NO: 23, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216, or (b) a sequence having 90%, about 91%, about 92%, about 93%, about 94%, about 95%, about 96%, about 97%, about 98%, or about 99% identity to SEQ ID NO: 19-24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

In some embodiments, the transmembrane domain comprises a transmembrane domain of a protein selected from the group consisting of the alpha, beta or zeta chain of the T-cell receptor, CD2, CD28, CD3 epsilon, CD45, CD4, CD5, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9.

In some embodiments, the transmembrane domain comprises an amino acid sequence selected from SEQ ID NO: 29, 31, or 33, or an amino acid sequence having 90%, about 91%, about 92%, about 93%, about 94%, about 95%, about 96%, about 97%, about 98%, or about 99% identity to SEQ ID NO: 29, 31, or 33. In some embodiments, the transmembrane domain comprises a nucleic acid sequence selected from SEQ ID NO: 30, SEQ ID NO: 32, or SEQ ID NO: 34, or a nucleic acid sequence having 90%, about 91%, about 92%, about 93%, about 94%, about 95%, about 96%, about 97%, about 98%, or about 99% identity to SEQ ID NO: 30, 32, or 34. In some embodiments, the transmembrane domain comprises a CD8 transmembrane domain, and/or an amino acid sequence of SEQ ID NO: 29, or an amino acid sequence having about 90%, about 91%, about 92%, about 93%, about 94%, about 95%, about 96%, about 97%, about 98%, or about 99% identity to SEQ ID NO: 29.

In some embodiments, the transmembrane domain comprises a nucleic acid sequence of SEQ ID NO: 30, or a nucleic acid sequence having 90%, about 91%, about 92%, about 93%, about 94%, about 95%, about 96%, about 97%, about 98%, or about 99% identity to SEQ ID NO: 30.

In some embodiments, the encoded anti-CD19 binding domain is connected to the transmembrane domain by a hinge region. In some embodiments, the hinge region: (a) is from a protein selected from the group consisting of an Fc fragment of an antibody, a hinge region of an antibody, a CH2 region of an antibody, a CH3 region of an antibody, an artificial spacer sequence, an IgG hinge, a CD8 hinge, and any combination thereof; or (b) comprises the amino acid sequence of SEQ ID NO: 27 or SEQ ID NO: 35, or an amino acid sequence having about 90%, about 91%, about 92%, about 93%, about 94%, about 95%, about 96%, about 97%, about 98%, or about 99% identity to SEQ ID NO: 27 or 35.

In some embodiments, the hinge region comprises a CD8 hinge region and/or the amino acid sequence of SEQ ID NO: 27, or an amino acid sequence having about 90%, about 91%, about 92%, about 93%, about 94%, about 95%, about 96%, about 97%, about 98%, or about 99% identity to SEQ ID NO: 27. In some embodiments, the hinge region comprises a nucleic acid sequence of selected from SEQ ID NO: 28, or SEQ ID NO: 36, or a nucleic acid sequence having 90%, about 91%, about 92%, about 93%, about 94%, about 95%, about 96%, about 97%, about 98%, or about 99% identity to SEQ ID NO: 28 or 36.

In some embodiments of the vector disclosed herein, the costimulatory domain of the CAR is a functional signaling domain of a protein selected from the group consisting of a TNFR superfamily member, OX40 (CD134), CD2, CD5, CD7, CD27, CD28, CD30, CD40, PD-1, CD8, ICAM-1, lymphocyte function-associated antigen-1 (LFA-1), CD11a, CD18, ICOS (CD278), LIGHT, NKG2C, B7-H3, a ligand that specifically binds to CD83, DAP10, DAP12, Lck, Fas and 4-1BB (CD137). In some embodiments, the costimulatory domain comprises an amino acid sequence selected from SEQ ID NO: 37, SEQ ID NO: 39, SEQ ID NO: 41, SEQ ID NO: 43, SEQ ID NO: 46, SEQ ID NO: 48, or SEQ ID NO: 50, or an amino acid sequence having about 90%, about 91%, about 92%, about 93%, about 94%, about 95%, about 96%, about 97%, about 98%, or about 99% identity to SEQ ID NO: 37, 39, 41, 43, 46, 48, or 50. In some embodiments, the costimulatory domain comprises a nucleic acid sequence selected from SEQ ID NO: 38, SEQ ID NO: 40, SEQ ID NO: 42, SEQ ID NO: 44, SEQ ID NO: 45, SEQ ID NO: 47, or SEQ ID NO: 49, or a nucleic acid sequence having 90%, about 91%, about 92%, about 93%, about 94%, about 95%, about 96%, about 97%, about 98%, or about 99% identity to SEQ ID NO: 38, 40, 42, 44, 45, 47, or 49.

In some embodiments of the vector disclosed herein, the intracellular signaling domain of the CAR comprises a signaling domain of a protein selected from the group consisting of CD3 zeta, FcγRIII, FcεRI, a cytoplasmic tail of an Fc receptor, an immunoreceptor tyrosine-based activation motif (ITAM) bearing cytoplasmic receptor, TCR zeta, FcR gamma, FcR beta, CD3 gamma, CD3 delta, CD3 epsilon, CD5, CD22, CD79a, CD79b, and CD66d.

In some embodiments, the intracellular signaling domain comprises the intracellular signaling domain of CD3 zeta, the amino acid sequence of SEQ ID NO: 52 or 54, or an amino acid sequence having about 90%, about 91%, about 92%, about 93%, about 94%, about 95%, about 96%, about 97%, about 98%, or about 99% identity to SEQ ID NO: 52 or 54. In some embodiments, the intracellular signaling domain comprises the nucleic acid sequence of SEQ ID NO: 53 or 55, or a nucleic acid sequence having about 90%, about 91%, about 92%, about 93%, about 94%, about 95%, about 96%, about 97%, about 98%, or about 99% identity to SEQ ID NO: 53 or 55. In some embodiments, the CAR comprises a functional signaling 4-1BB costimulatory domain and a functional CD3 zeta intracellular signaling domain.

In some embodiments, the CAR comprises the amino acid sequence of SEQ ID NO: 37, SEQ ID NO: 52, or SEQ ID NO:54, or an amino acid sequence having about 90%, about 91%, about 92%, about 93%, about 94%, about 95%, about 96%, about 97%, about 98%, or about 99% identity to SEQ ID NO: 37, SEQ ID NO: 52 or SEQ ID NO: 54. In some embodiments, the intracellular signaling domain comprises the amino acid sequence of SEQ ID NO: 37 and the amino acid sequence of SEQ ID NO: 52 or SEQ ID NO:54, or an amino acid sequence having about 90%, about 91%, about 92%, about 93%, about 94%, about 95%, about 96%, about 97%, about 98%, or about 99% identity to SEQ ID NO: 37, SEQ ID NO: 52 or SEQ ID NO:54. In that embodiment, the sequences are expressed in the same frame and as a single polypeptide chain.

In some embodiments of the vector disclosed herein, (a) the nucleic acid sequence comprises a sequence of SEQ ID NO: 38, or a nucleic acid sequence having 90%, about 91%, about 92%, about 93%, about 94%, about 95%, about 96%, about 97%, about 98%, or about 99% identity to SEQ ID NO: 38, and/or (b) the nucleic acid sequence comprises a sequence of SEQ ID NO: 53 or SEQ ID NO:55, or a nucleic acid sequence having about 90%, about 91%, about 92%, about 93%, about 94%, about 95%, about 96%, about 97%, about 98%, or about 99% identity to SEQ ID NO: 53 or 55.

In some embodiments, the CAR further comprises a leader sequence. In some embodiments, the leader sequence comprises SEQ ID NO: 25. In some embodiments, the linker peptide: (a) is selected from F2A, E2A, P2A, T2A, or Furin-(G4S)2-T2A (F-GS2-T2A); and/or (b) comprises the amino acid sequence of SEQ ID NO: 92, SEQ ID NO: 94, SEQ ID NO: 96, or SEQ ID NO: 99; and/or (c) comprises the nucleic acid sequence of SEQ ID NO: 93, SEQ ID NO: 95, SEQ ID NO: 97, or SEQ ID NO: 98.

One aspect of the present disclosure provides a vector comprising: (a) a first polynucleotide comprising a constitutive promoter operably linked to a nucleic acid encoding an anti-CD19 chimeric antigen receptor (CAR), where the CAR comprises: (i) an anti-CD19 binding domain comprising: (1) LC CDR1 of SEQ ID NO: 1, LC CDR2 of SEQ ID NO: 2, and LC CDR3, HC CDR1 of SEQ ID NO: 4, HC CDR2 of SEQ ID NO: 5, and HC CDR3 of SEQ ID NO: 6; or (2) LC CDR1 of SEQ ID NO: 193, LC CDR2 of SEQ ID NO: 194, LC CDR3 of SEQ ID NO: 195; HC CDR1 of SEQ ID NO: 196, HC CDR2 of SEQ ID NO: 197, and HC CDR3 of SEQ ID NO: 198; or (c) a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2; (ii) a transmembrane domain selected from CD28 or CD8 transmembrane domain; (iii) a costimulatory domain comprising an intracellular signaling domain of a protein selected from the group consisting of OX40, CD27, CD2, CD28, ICOS, and 4-1BB; and (iv) an intracellular signaling domain comprising of CD3-zeta; (b) a second polynucleotide comprising a nucleic acid encoding Interleukin-18 (IL-18), and/or Interleukin-18 receptor (IL-18R). In some embodiments, the first polynucleotide is operably linked to the second polypeptide via a linker peptide selected from the group consisting of F2A, E2A, P2A, T2A, and Furin-(G4S)2-T2A (F-GS2-T2A).

One aspect of the present disclosure provides a vector comprising: (a) a first polynucleotide comprising a constitutive promoter operably linked to a nucleic acid encoding an anti-CD19 chimeric antigen receptor (CAR), where the CAR comprises: (i) an anti-CD19 binding domain comprising the amino acid sequence of SEQ ID NO: 9, 18, 64, 75, 86, 190, 157, 212, 201, 226, 179, 168, or 146; (ii) a transmembrane domain selected from CD28 or CD8 transmembrane domain; (iii) a costimulatory domain comprising an intracellular signaling domain of a protein selected from the group consisting of OX40, CD27, CD2, CD28, ICOS, and 4-1BB; and (iv) an intracellular signaling domain comprising of CD3-zeta; and (b) a second polynucleotide comprising a nucleic acid encoding Interleukin-18 (IL-18) and/or Interleukin-18 receptor (IL-18R). In that embodiment, the first polynucleotide is operably linked to the second polypeptide via a linker peptide selected from the group consisting of F2A, E2A, P2A, T2A, or Furin-(G4S)2-T2A (F-GS2-T2A).

One aspect of the present disclosure provides a vector comprising: (a) a first polynucleotide comprising a constitutive promoter operably linked to a nucleic acid encoding an anti-CD19 chimeric antigen receptor (CAR), wherein the CAR comprises: (i) an anti-CD19 binding domain comprising the amino acid sequence of SEQ ID NO: 9, 18, 64, 75, 86, 190, 157, 212, 201, 226, 179, 168, or 146; (ii) a transmembrane domain comprising the amino acid sequence selected from the group consisting of SEQ ID NO: 29, 31, and 33; (iii) a costimulatory domain comprising an amino acid sequence selected from the group consisting of SEQ ID NO: 37, SEQ ID NO: 39, SEQ ID NO: 41, SEQ ID NO: 43, SEQ ID NO: 46, SEQ ID NO: 48, and SEQ ID NO: 50; and (iv) an intracellular signaling domain comprising the amino acid sequence of SEQ ID NO: 52 or SEQ ID NO: 54; and (b) a second polynucleotide comprising: (i) a nucleic acid encoding the amino acid of SEQ ID NO: 105, 215, 106, or 107, and/or (ii) an IL-18 polypeptide comprising E42A; E42K; K89A; E42A and K89A; E42K and K89A; E42A and C74S; E42A, C74S, and K89A; C74S, and K89A; C74S, C112S, and C112S; E42A, C74S, C112S, and C112S; E42A, K89A, C74S, C112S, and C112S in SEQ ID NO: 107. In that embodiment, the first polynucleotide is operably linked to the second polypeptide via a linker peptide selected from the group consisting of F2A, E2A, P2A, T2A, and Furin-(G4S)2-T2A (F-GS2-T2A).

One aspect of the present disclosure provides a vector comprising: a vector comprising: (a) a first polynucleotide comprising a constitutive promoter operably linked to a nucleic acid encoding an anti-CD19 chimeric antigen receptor (CAR), wherein the CAR comprises: (i) an anti-CD19 binding domain comprising the amino acid sequence of SEQ ID NO: 9, 18, 64, 75, 86, 190, 157, 212, 201, 226, 179, 168, or 146; (ii) a transmembrane domain comprising the amino acid sequence of SEQ ID NO: 29; (iii) a costimulatory domain comprising the amino acid sequence of SEQ ID NO: 37; and (iv) an intracellular signaling domain of SEQ ID NO: 52 or SEQ ID NO: 54; and (b) a second polynucleotide comprising: (i) a nucleic acid encoding the amino acid sequence of SEQ ID NO: 105, 215, 106, or 107, and/or (ii) an IL-18 polypeptide comprising E42A; E42K; K89A; E42A and K89A; E42K and K89A; E42A and C74S; E42A, C74S, and K89A; C74S, and K89A; C74S, C112S, and C112S; E42A, C74S, C112S, and C112S; E42A, K89A, C74S, C112S, and C112S in SEQ ID NO: 107. In that embodiments, the first polynucleotide is operably linked to the second polypeptide via a linker peptide selected from the group consisting of F2A, E2A, P2A, T2A, and Furin-(G4S)2-T2A (F-GS2-T2A).

In some embodiments, the first polynucleotide comprises: (a) an amino acid sequence encoded by a nucleic acid sequence selected from the group consisting of SEQ ID NO: 66, 77, 88, 148, 170, 181, 203, 214, 159, 192, 23, and 20; and/or (b) an amino acid sequence encoded by a nucleic acid sequence selected from the group consisting of SEQ ID NO: 65, 76, 87, 147, 169, 180, 202, 213, 158, 191, 22, and 19.

Another aspect of the present disclosure provides a modified cell comprising a vector described herein. In some embodiments, the modified cell is an immune cell or precursor cell thereof.

In some embodiments, the modified cell is selected from the group consisting of a T cell, a Natural Killer (NK) cell, a cytotoxic T lymphocyte (CTL), a regulatory T cell, a Natural Killer T (NKT) cell, a dendritic cell, a macrophage, a human embryonic stem cell, and a pluripotent stem cell from which lymphoid cells may be differentiated. In some embodiments, the modified cell is an autologous cell, a heterologous cell, or an allogeneic cell. In some embodiments, the cell is a modified T cell or a modified human T cell. In some embodiments, the modified T cell is a CD8⁺ T cell.

In some embodiments, the modified cell is a CD8⁺ T cell with a central memory phenotype (CD44⁻; Ly6C⁺), a macrophage with an M1 phenotype (MHC-II⁺), or a dendritic cell with a mature and activated phenotype (CD86⁺; MCH-II⁺).

In some embodiments, the modified cell further comprises: (a) a switch receptor comprising a first polypeptide that comprises at least a portion of an inhibitory molecule selected from the group consisting of PD1, TGFβR, TIM-2 and BTLA, conjugated to a second polypeptide that comprises an intracellular signaling domain of a molecule selected from the group consisting of OX40, CD27, CD28, IL-12R, ICOS, and 4-1BB; (b) a dominant negative receptor comprising a truncated variant of a receptor selected from the group consisting of PD1, TGFβR, TIM-2 and BTLA; and/or (c) a polypeptide that enhances an immune cell function, or a functional derivative thereof selected from the group consisting of a chemokine, a chemokine receptor, a cytokine, a cytokine receptor, Interleukin-7 (IL-7), Interleukin-7 receptor (IL-7R), Interleukin-15 (IL-15), Interleukin-15 receptor (IL-15R), Interleukin-21 (IL-21), CCL21, CCL19, and a combination thereof.

Another aspect of the present disclosure provides a composition comprising a modified cell or a population of modified cells described herein.

Another aspect of the present disclosure provides a method of making a modified cell comprising transfecting a cell with the vector described herein.

Another aspect of the present disclosure provides a method of providing an anti-tumor immunity to a mammal comprising administering to the mammal an effective amount of: (a) a composition comprising the modified cells described herein or a modified cell made by the methods described herein; (b) the modified cells described herein; or a modified cell made by the methods described herein; or (c) the composition described herein.

Another aspect of the present disclosure provides a method of treating a mammal having a disease associated with expression of CD19 comprising administering to the mammal an effective amount of: (a) a composition comprising the modified cells described herein or a modified cell made by the methods described herein; (b) the modified cells described herein; or a modified cell made by the methods described herein; or (c) the composition described herein.

In some embodiments, the modified cell is an autologous modified T cell. In some embodiments, the modified cell is an allogeneic modified T cell. In some embodiments, the mammal is a human.

In some embodiments, the disease associated with CD19 expression is selected from: (a) a proliferative disease, a malignancy, a precancerous condition, or a non-cancer related indication associated with expression of CD19; or (b) a cancer, an atypical and/or a non-classical cancer, a myelodysplasia, a myelodysplastic syndrome, or a preleukemia.

In some embodiments, the disease is a hematologic cancer selected from the group consisting of: (a) an acute leukemia, a chronic leukemia, a hematologic condition and combinations thereof; or (b) B-cell acute lymphoid leukemia (“BALL”), T-cell acute lymphoid leukemia (“TALL”), acute lymphoid leukemia (ALL), chronic myelogenous leukemia (CIVIL), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, ineffective production (or dysplasia) of myeloid blood cells, and combinations thereof.

In some embodiments, the modified cells or the composition are administered in combination with: (a) an agent that increases the efficacy of a modified cell comprising the vector described herein, a modified cell described herein, or a modified cell made by the methods described herein; (b) an agent that ameliorates one or more side effects associated with administration of a modified cell comprising the vector described herein, a modified cell described herein, or a modified cell made by the methods described herein; or (c) an agent that treats the disease associated with CD19 overexpression.

### BRIEF DESCRIPTION OF THE DRAWINGS

FIG. 1 shows a schematic outlining the identification of unique CD19-specific antibody clones from phage display libraries followed by biotinylated baculovirus binding, SIGLEC binding, and/or NALM6 tumor cells binding selections.

FIGS. 2A-E show an alignment of the nucleic acid sequences of the novel CD19 binders of the present disclosure.

FIG. 2F shows a percent identity matrix illustrating the similarity of the novel binders at the nucleic acid level.

FIGS. 3A-B show graphs demonstrating the effects of armoring CD19 CAR T cells expressing CD19 CARs comprising original and optimized CD19 binders 42 and 52 (42og, 42op, 52og, and 52op) with an interleukin-18 (IL-18) on CAR T cells expansion based on cell doublings (FIG. 3A) and size (FIG. 3B). CD19 CART cells expressing CD19-52 op, CD19-52op-IL18, CD19-52og-IL-18, and CD19-42op-IL-18 continued to expand after 12 days in culture without stimulation while the expansion of CD19 CAR T cells expressing CD19-52og, CD19-42og, and CD19-42og-IL-18 decreased steadily over time. Among the armored constructs tested, CD19-42og-IL-18 CAR T cells showed the least continuous expansion after activation. The expansion size appeared relatively similar between all tested groups (FIG. 3B).

FIGS. 4A-B show histograms demonstrating that armoring the CD19 CAR T cells with the IL-18 did not change the expression of the CD19 CARs on the surface of ND609 CAR T cells. In particular, the mean fluorescence intensity (mfi) of CD19 CARs on ND609 CAR T cells was not lost with the co-expression of 2A-IL-18. However, CD19 42og-CARs and CD19-42op CARs (FIG. 4A) appeared to have more distinctive expression profiles when compared to CD19-52og CARs and CD19-52op CARs (FIG. 4B). The IL-18 co-expression may have enhanced the surface expression of CD19-42og CARs.

FIG. 5 shows a schematic illustrating the timeline used to evaluate the in vivo cytotoxic effectiveness (e.g., killing) of the IL-18 armored CD19 CAR T cells. ND609 CAR T cells were transduced with original and optimized CD19 CAR (binders 42 and 52)-2A-IL18 constructs, and the CD19 CAR T cells were evaluated in vivo using the Jeko NSG mouse model. Animals were bled during the 2^{nd}, 4^{th}, and later weeks to assess the peripheral blood levels of huCD45 and serum for IL-18. Health, weight, and BLI were also evaluated at the indicated times (downward arrows). In addition, during weeks 2, 4 and later, depending on tumor clearance, cells were harvested from the femur and spleen in mixed group to evaluate their competitiveness. Table 4 shows experimental set up.

FIGS. 6A-K show graphs illustrating anti-tumor activity (tumor control) of IL-18 armored CD19 CAR T cells in Jekol NSG mice. Mice were administered with CD19 CAR T cells armored with an IL-18 (FIGS. 6A-G) or without an IL-18 armor (FIGS. 6H-K) and assessed as shown in FIG. 5. When used at a concentration of 1×10⁵ CAR+/mouse, all tested IL-18 armored CD19 CART cells cleared tumors without relapse during the duration of the experiment (about 80 days) based on tumor Bioluminescence Imaging (BLI). These figures show that CAR T cells comprising original CD19 binders 42 CAR armored with IL-18 (CD19-42og-IL-18) cleared tumor faster than CAR T cells comprising a CD19-42op-IL-18, a control CD19-IL-18, CD19-52op-IL-18 and CD19-52og-IL-18. Anti-tumor activity of CD19 binders in Jekol NSG mouse model were tested using primary T cells from donor ND609 (FIGS. 6A-C), ND585 (FIGS. 6D-G), and ND608 (FIGS. 6H-K).

FIGS. 7A-D show graphs demonstrating the percentage of human CD45 positive (huCD45±) cells in the peripheral blood of animals administered IL-18 armored ND609 CAR T cells expressing CD19-42 original or optimized or CD19-52 original overtime and illustrating that most tested IL-18 armored CD19 CAR T cells contracted in number after tumor clearance. A good general contraction of peripheral huCD45 blood levels following tumor clearance was observed in all tested animals. A mouse in FIG. 7A showed continued expansion of peripheral huCD45 blood levels following tumor clearance probably due to GVHD response periodically observed in model.

FIGS. 8A-F show graphs demonstrating the percentage of CAR positive and human CD45 positive (CD45⁺CAR T cells; FIGS. 8A-C) and the percentage of human CD45 positive and CD4 positive (huCD45⁺CD4⁺; FIGS. 8D-F)) cells in the peripheral blood of animals administered armored CAR T cells comprising the original or optimized CD19-42 CARs; and original CD19-52 CARs overtime. IL-18 co-expression enhanced or maintained a high percentage of CD4⁺CAR T cells in treated animals. In addition, the Figures show mouse #534 with continued expansion of peripheral huCD45 blood levels following tumor clearance in FIG. 7A most likely experienced GvHD T cell response because the zero response drove a CAR independent T cell expansion (FIG. 8D) as the percent of positive T cells decreased (FIG. 8A). Each line represents an individual animal.

FIGS. 9A-F show graphs characterizing the human CD45 positive (huCD45±) cells in the peripheral blood of animals administered IL-18 armored ND585 CART cells expressing CD19-42 original or optimized CARs overtime. The levels of IL-18 armored CD19 CAR T cells in peripheral blood (FIGS. 9A-B); the percentage of huCD45⁺ that expressed a CD19 CAR (FIGS. 9C-D) and the percent of CD4⁺ cells in the CD45⁺ populations (FIGS. 9E-F). Most tested IL-18 armored CD19 CAR T cells contracted in number after tumor clearance. No human CD45 was detected in blood on D14 in any group tested. Each line represents an individual animal.

FIGS. 10A-F show graphs characterizing the human CD45 positive (huCD45⁺) cells in the peripheral blood of animals administered IL-18 armored ND585 CART cells expressing CD19-52 original or optimized CARs overtime. The levels of IL-18 armored CD19 CAR T cells in peripheral blood (FIGS. 10A-B); the percentage of huCD45⁺ that expressed a CD19 CAR (FIGS. 10C-D) and the percent of CD4⁺ cells in the CD45⁺ populations (FIGS. 10E-F). Most tested IL-18 armored CD19 CAR T cells contracted in number after tumor clearance. No human CD45 was detected in blood on D14 in any group tested. Each line represents an individual animal.

FIGS. 11A-F show weight loss associated with Jekol tumor clearance in animals injected with IL-18 armored CD19 CAR T cells as described in FIGS. 6A-K. Animals shown in FIGS. 11A-D correlate with animals shown in FIGS. 6D-G.

FIG. 12 shows a Kaplan-Meier survival curve of animals administered with 1×10⁵ IL-18 armored ND585 CD19 CAR T cells expressing original or optimized CD19 binders 42 and 52. 100% of mice administered with CD19-42OP-IL18 and CD19-52OP-IL18 CAR T cells remained alive for the duration of the study (70 days). 80% of mice administered with CD19-42og-IL18 CAR T cells remained alive at the end of the study and one mouse died on Day 22 due to endpoint weight loss during tumor clearance. 60% of mice administered with CD19-52og-IL18 CAR T cells remained alive at the end of the study; and one mouse died on Day 22 due to endpoint weight loss during tumor clearance; and a second mouse died on day 66 from spontaneous death. Mice expressing positive control CD19 CAR T cells died at day 28 and mice administered with untransduced CAR T cells died at day 29 of excessive weigh loss.

FIGS. 13A-B show graphs demonstrating the effects of IL-18 armoring on CD19 CAR T cells expressing CAR comprising original or optimized 42 and 52 binders (CD19 binders-IL18) on expansion or growth curve growth curve (FIG. 13A) and the mean cell size or contraction (FIG. 13B) in human primary cells from donor ND585. All CART cells tested showed similar robust expansion and contraction. However, IL-18 armored CD19 CAR T cells comprising the original 42 CD19 binder (CD19-42og-IL18) expanded and contracted faster than armored CAR T cells comprising 42 optimized (CD19-42op-IL18), 52 original (CD19-52og-IL18), and 52 optimized (CD19-52op-IL18).

FIGS. 14A-B show bar graphs quantifying raw mean fluorescence intensity from flow cytometry analyses demonstrating the expression of CD19 CARs on primary human T cells from ND585 donor (FIG. 14A) and ND307 donor (FIG. 14B). In particular, CD19 CAR+IL-18 constructs were transduced in ND585 T cells and assessed by flow cytometry Expression of the CD19 CARs was stable over the time period tested in all groups tested. Cells were gated on CD4⁺ T cells. The expression of the CD19CAR based on mfi showed that Control CD19-IL18 expression>CD19-42-IL18 expression>CD19-52-IL18 expression.

FIGS. 15A-F show bar graphs quantifying IL-2 and TNF-α production (IL-2 alone, TNF α alone, and combination of IL-2 and TNF-α) from ND585 donor (FIGS. 15A-C) or ND307 donor (FIGS. 15D-F) CD4⁺CAR T cells expressing CD19-42og-IL-18, CD19-42op-IL-18, CD19-52og-IL-18, and CD19-52op-IL-18, upon Nalm6 (FIGS. 15B and E) and Jeko-1 (FIGS. 15A and D) stimulation on Day 9, or in the absence of any stimulation (FIGS. 15C and F). The figures show that IL-2 and TNF-α production was substantially similar in all group tested (CD19 CAR comprising original and optimized CD19 binders 42 and 52). Gated on CD4⁺ T cells. The mean fluorescence intensity (mfi) of CD19 surface expression in Nalm6 cells was about 13606 mfi and 6851 mfi in Jeko cells. Table 8 shows the production of INF-y and TNF-α gated on CD8⁺ T cells.

FIGS. 16A-F show bar graphs quantifying cytokine production (IL-2, TNF-α, and IFN-γ) by ND307 CD4⁺ and CD8⁺CAR T cells expressing CD19-42og-IL-18, CD19-42op-IL-18, CD19-52og-IL-18, and CD19-52op-IL-18 upon stimulation with recombinant K562 cells transfected with different amount of RNA encoding a truncated CD19 antigen (CD19Ag RNA). K562 cells were transfected with no CD19 antigen, low CD19 antigen (about 3% CD19 antigen expression), medium CD19 antigen (about 49% CD19 antigen expression); or high CD19 antigen (about 71% CD19 antigen expression). CD19 antigen expression was determined by flow cytometry. TNF-α production in ND307 CD4⁺CAR T cells was higher from CAR T cells expressing CD19-42og-IL-18.

### DETAILED DESCRIPTION

### I. Overview

The present disclosure provides novel CD19 CAR constructs operably linked to a recombinant interleukin-18 construct (CD19 CAR-IL-18), CAR T cells comprising the CD19 CAR-IL-18 constructs, and method of using the IL-18 armored CD19 CAR T cells. Armoring the novel CD19 CAR T cells with IL-18: (1) enhanced the expression of the CD19 CAR; (2) reduced their expansion in the absence of stimulation; (3) enhanced their tumor clearance efficacy; (4) stimulated the CD19 CAR T cells contraction after tumor clearance; (5) enhanced or maintained a high percentage of CD4⁺CD19 CAR T cells in treated animals; and (6) reduced side effects (e.g., weight loss). Generally, co-expressing the novel CD19 CARs with IL-18 reduced systemic CAR-induced toxicity (e.g., weight loss) and tumor relapse or remission. As shown herein, 80-100% of animals administered with the IL-18 armored CD19 CART cells described herein survived during the duration of the test, while animals administered with a “positive control” CD19 CAR T cells (e.g., a clinically approved CD19 binder) died at day 28 and animals administered with untransduced CAR T cells died at day 29.

A. Identification of Novel CD19 Binders

The present disclosure provides novel CD19 chimeric antigen receptors with low affinity and fast off-rate when compared to CD19 CARs known in the prior art or clinically approved CD19 binders-based CARs (e.g., FMC63). FMC63 is an IgG2a mouse monoclonal antibody specific for CD19, which is a target for the immunotherapy of B lineage leukemias and lymphomas. The complete characterizations of these novel CD19 binders are described in the co-pending PCT application and U.S. application, which claim priority to U.S. Provisional Application No. 63/417,220, filed on Oct. 18, 2022, and U.S. Provisional Application No. 63/426,967, filed on Nov. 21, 2022, the contents of which are hereby incorporated by reference in their entirety for all purposes.

Chimeric antigen receptor-modified T cells (CAR T cells) directed against CD19 have shown promise as a novel therapy for hematological malignancies. Remarkable antitumor responses have been achieved from anti-CD19 CAR-T therapies against B-cell acute lymphoblastic leukemia (B-ALL) and other refractory B-cell malignancies. Complete remission (CR) has been achieved in as many as 70-90% of cases of relapsed/refractory acute lymphoblastic leukemia (R/R B-ALL). In light of these outstanding experimental results, the U.S. Food and Drug Administration (FDA) and the European Medicines Agency (EMA) approved several CD19-directed CAR T cell products, including tisagenlecleucel (KYMRIAH®, Novartis), axicabtagene ciloleucel (YESCARTA®, Kite Pharma-Gilead), and lisocabtagene maraleucel (BREYANZI®, Juno Therapeutics-Celgene-BMS) for treating large B-cell lymphoma. In addition, brexucabtagene autoleucel (TECARTUS®, Kite Pharma-Gilead) was approved for treating relapsed/refractory mantle cell lymphoma. Despite the range of validated CAR T cell products, the success of these approved CAR T cell products has been limited. This is because about 40-50% of patients responding to CD19 CART cell therapy relapse within 1 year, and nearly half of these relapses included CD19-positive leukemic cells. Recent evidence suggests that resistance to CD19 chimeric antigen receptor (CAR)-modified T cell therapy may be due to the presence of CD19 isoforms that lose binding to the single-chain variable fragment (scFv) in current use. Additional resistance mechanisms that limit current CAR T cell therapies include T-cell exhaustion, immunosuppression, antigen loss, cytokine-release syndrome (CRS), immune effector cell-associated neurotoxicity syndrome, and/or neurotoxicity.

To resolve these issues, the present disclosure provides improved CD19 CAR constructs operably linked to interleukin-18 (IL-18) and IL-18 armored CD19 CAR T cells comprising a novel CD19 binder that targets distinct and non-overlapping epitopes on the CD19 protein.

The novel CD19 binders (e.g., antibody, antibody fragment, or scFv) were specifically screened to have desired characteristics. In particular, the novel CD19 binders were screened to have low affinity and fast off-rate. While low affinity binding can be determined by either on-rate (Kₒₙ) or an off rate (K_{off}), the anti-CD19 binders (e.g., scFv) disclosed herein were selected for a fast off rate. This fast off-rate allows the CD19 CAR to rapidly dissociate from CD19, thereby resulting in a shorter CAR T cell-tumor interaction. This shorter interaction time can then reduce cytokine release and thereby reducing toxicity. In one aspect, the CD19 binders disclosed herein have a K_{D} value of about 1 nM to about 50 nM. In another aspect, the CD19 binders disclosed herein have a K_{off} value of about 1.0×10⁻³ s⁻¹ to about 5.0×10⁻³ s⁻¹.

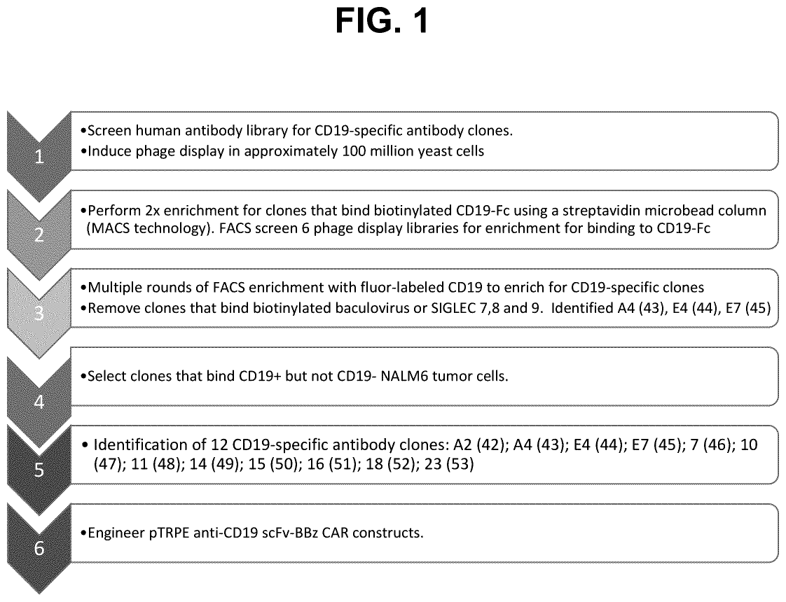

In addition, the short interaction time reduced T-cell exhaustion, which enhanced CAR T-cell persistence. As such, the novel binders were identified by specifically screening a human antibody library for CD19-specific antibodies or antibody fragments for low binding affinity (e.g., a K_{D} of 1 nM to about 50 nM) and a fast off-rate (e.g., K_{off} about 1.0×10⁻³ s⁻¹ to about 5.0×10⁻³ s⁻¹). FIG. 1 shows a schematic outlining the general steps used to identify the 12 unique CD19 binders from phage display libraries and yeast display screening to selection by biotinylated baculovirus binding, SIGLEC binding, and/or NALM6 tumor cells binding.

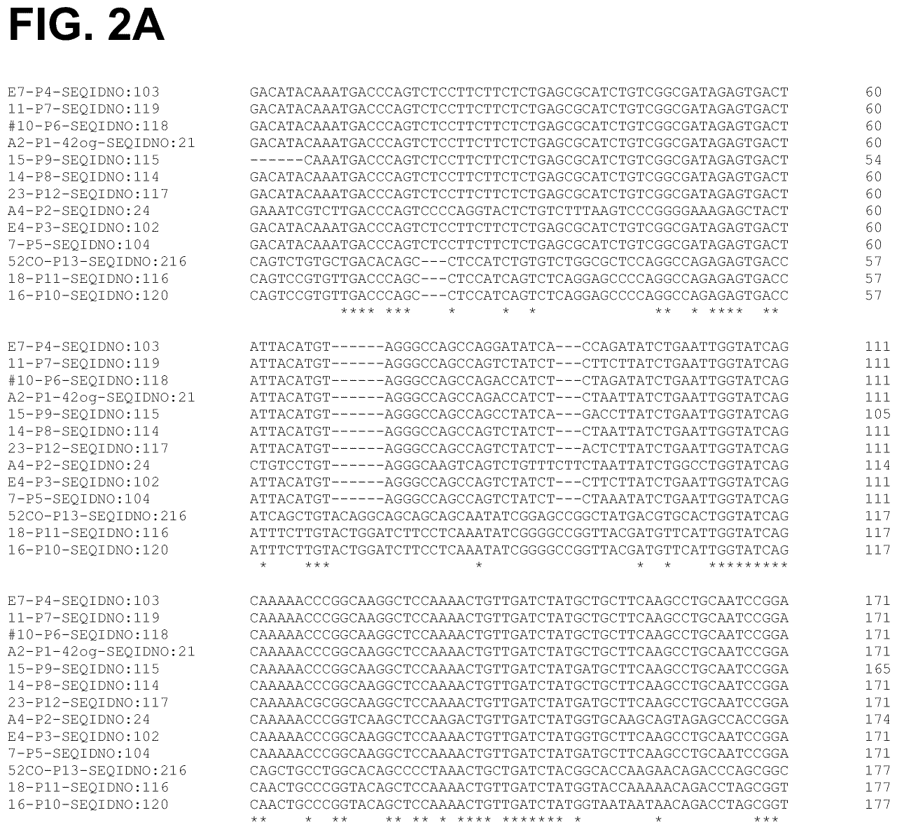

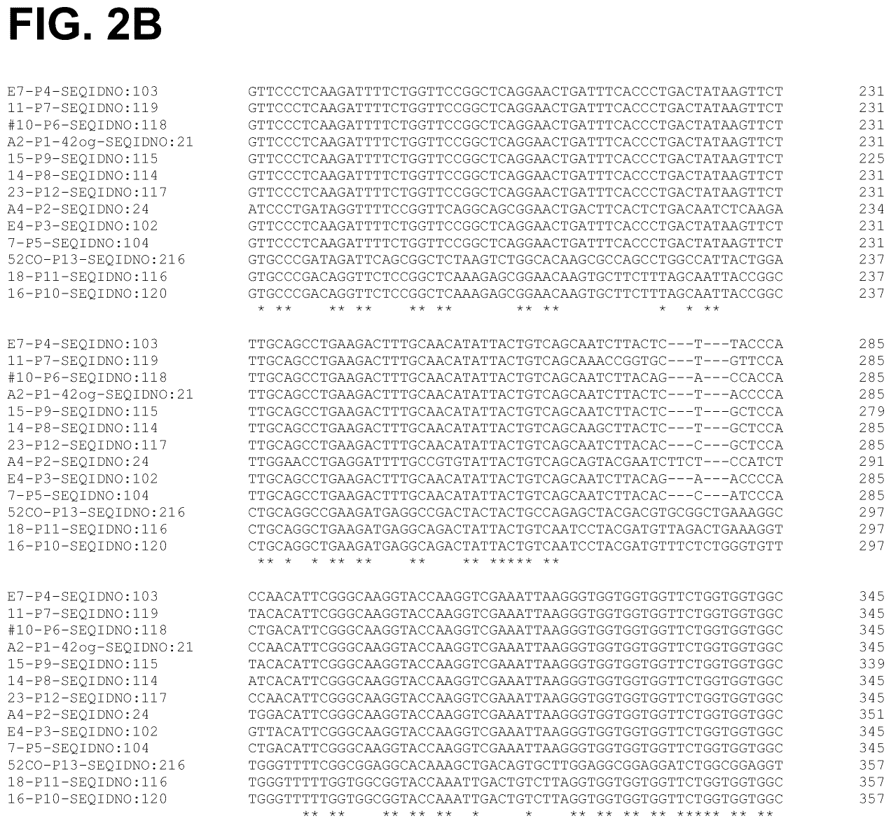

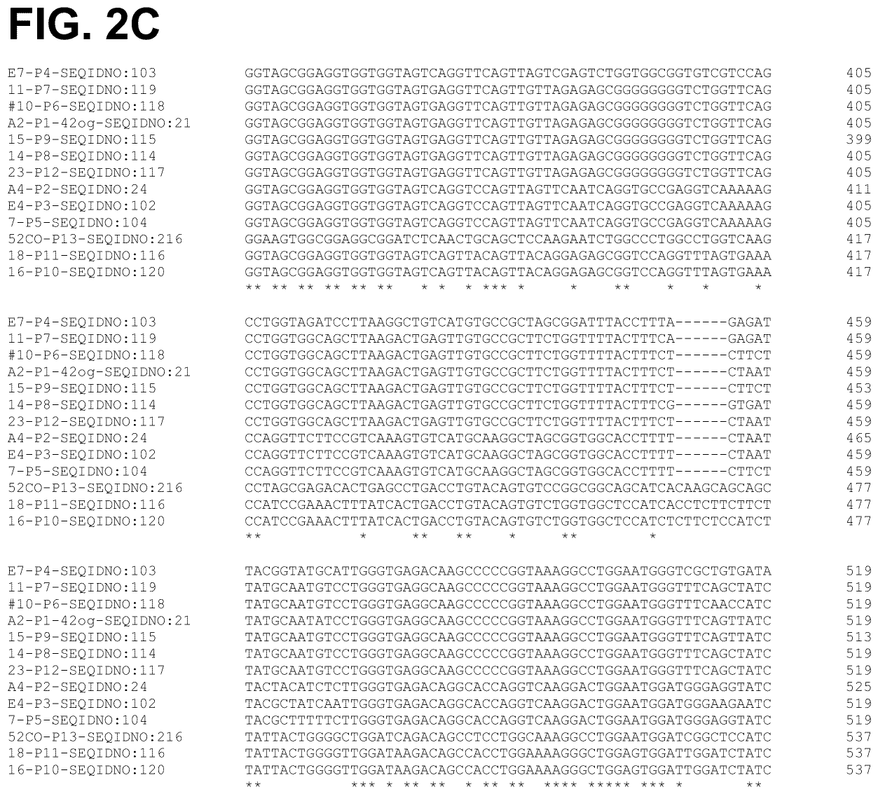

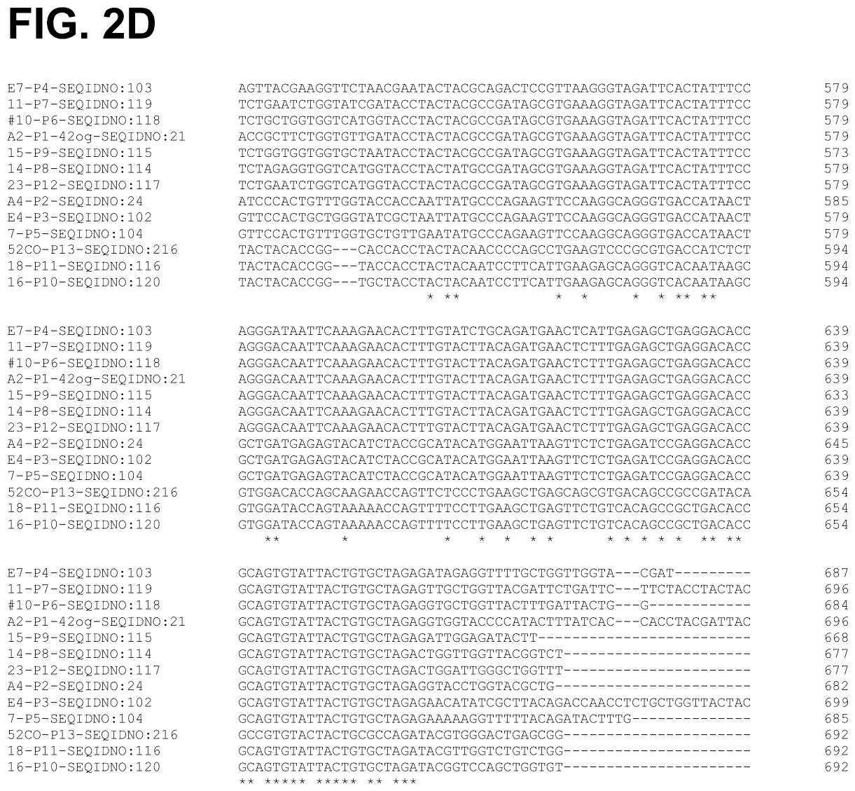

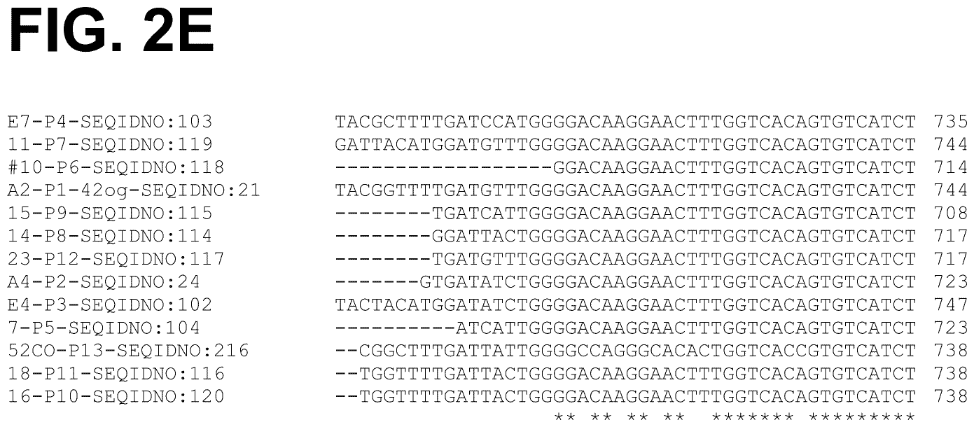

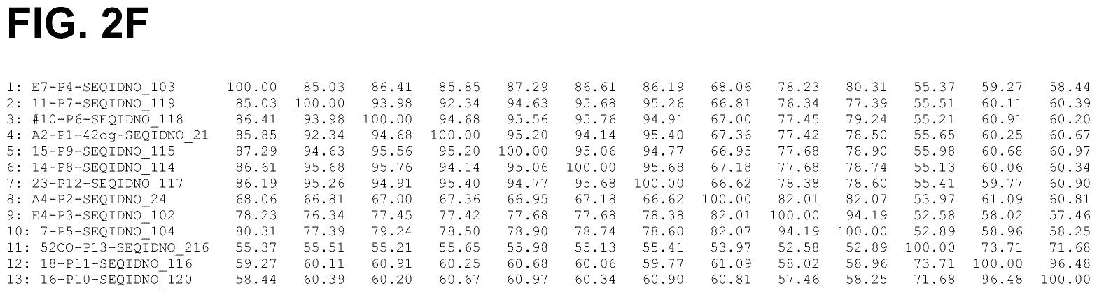

This yeast display screen yielded about 13 novel binders shown in Table 3 and FIGS. 2A-2E. The nucleic acid sequences of the novel CD19 binders disclosed herein are about 58% to about 97% identical to each other as shown in FIG. 2F.

T cells expressing CD19 CARs comprising the novel binders of the present disclosure exhibited higher efficacy, enhanced in vivo persistence, and low toxicity when compared to T cells expressing known CD19 CARs (e.g., FMC63-based CARs). However, T cells expressing the low affinity CD19 CAR of the present disclosure killed tumor cells as well as T cells expressing a high affinity CD19 CAR. Furthermore, T cells expressing the low affinity CD19 CARs of the present disclosure can show similar cytokine production (e.g., interferon γ or IL-2 production) and proliferation as T cells expressing a high affinity CD19 CAR (e.g., FMC63-based CAR).

Selection of the top of the 12 novel CD19 binder candidates was ultimately based on the following functional characteristics in view of known CD19 binders: (1) low tonic signal; (2) strong activation rate; (3) healthy expansion profiles; (4) robust stable surface expression; and (5) cytokine production. Based on these criteria, CD19 binders 42 (P1) and 52 (P11 and P13) appeared to be exemplary candidates. Preliminary analyses showed that the 12 novel CD19 binders produced similar transcriptional profiles. As described herein, these 12 novel CD19 binders exhibited unusual and unique functional characteristics, signaling, pharmacology, and tumor suppression properties. The new properties described herein will addressed current CD19 CAR issues, such as e.g., T-cell exhaustion, immunosuppression, antigen loss, cytokine-release syndrome (CRS), immune effector cell-associated neurotoxicity syndrome, and/or neurotoxicity.

Furthermore, CAR T cells expressing CARs comprising original or optimized CD19 binders 42 and 52 effectively controlled tumor growth in Jeko NSG mouse model. Tumor growth was suppressed at the greatest level by CD19 binder 42 original CAR T cells. CD19 binder 42opt CAR T cells, CD19 binder 52 original CAR T cells, and CD19 binder 52op CAR T cells also suppressed tumor growth. The complete characterizations of these novel CD19 binders are described in the co-pending PCT application and U.S. application, which claim priority to U.S. Provisional Application No. 63/417,220, filed on Oct. 18, 2022, and U.S. Provisional Application No. 63/426,967, filed on Nov. 21, 2022, the contents of which are hereby incorporated by reference in their entirety for all purposes.

B. Epitope Mapping of the Novel CD19 Binders

The present disclosure provides improved CD19 CAR T cells comprising novel CD19 binders that target distinct and non-overlapping epitopes on the CD19 protein and armored with (i.e., co-expressing) interleukin-18 (IL-18).

An initial evaluation of epitope binding region of CD19 binders was conducted using binding assays to determine if the novel CD19 binders bound to the anti-FMC63 antibody and if they shared the same binding site (e.g., epitope), or if they bound to the same region. These data showed that the anti-FMC63 antibody was not an idiotype antibody for the novel CD19 binders. For example, the anti-FMC63 antibody did not bind to any cells expressing a CAR comprising a the novel CD19 binder described herein. In addition, the anti-FMC63 antibody did not block the interaction between any of the novel CD19 binders tested and a recombinant CD19 protein.

In addition, a high-throughput shotgun mutagenesis analysis was performed to map the epitope of the novel CD19 binders on the extracellular domain of the full-length CD19 protein (SEQ ID NO: 217). The high-throughput shotgun mutagenesis analysis of the CD19 42original (42og) showed that CD19 42og bound to a distinct epitope on the extracellular domain of CD19 (Table 10). CD19 42og scFv bound to a completely region of the extracellular domain of CD19 that did not overlap with regions bound by well characterized CD19 antibodies, such as FMC63, 4G7, or 3B10.

TABLE 10
Critical Residues for 42og scFv binding to CD19 extracellular domain
Binding Reactivity (% WT)

| | | | |
|---|---|---|---|
| | 42og | anti-CD19 | |
| Mutation | scFv-Fc HS | control MAb | Clone types |

| | | | | | |
|---|---|---|---|---|---|
| Q98A | 5.1 | (8) | 84.2 | (12) | Primary |
| E104A | 19.2 | (1) | 101.8 | (35) | Primary |
| K105A | 1.1 | (1) | 88.0 | (23) | Primary |
| A106S | 31.7 | (18) | 107.7 | (17) | Secondary |
| V207A | 29.9 | (27) | 82.3 | (21) | Secondary |

Klesmith et al. (Biochemistry 58:4869-4881 (2019)) characterized the conformational epitopes of FMC63, 4G7, and 3B10 (e.g., anti-CD19 clinical antibodies) using high-throughput screening strategies to comprehensively map the binding sequences of these antibodies to the extracellular domain of CD19 variant CD19.1. These extensive analyses of conformational epitope maps of FMC63, 4G7 and 3B10 showed that all three antibodies have partially overlapping epitopes near the published epitope of antibody B43 co-crystallized with CD19. Two main epitope regions were identified. The first region comprises amino acid sequence WAKDRPEIWEGEP (SEQ ID NO: 219) located at positions 159-171 of the full-length CD19 protein (SEQ ID NO:217). The second region comprises the amino acid sequence of PKGPKSLLSLE (SEQ ID NO: 220) and was located at positions 219-229 of SEQ ID NO: 217.

In contrast, CD19 42og scFv bound primarily to amino acid sequence of QPGPPSEKAWQP (SEQ ID NO: 221) located at positions 98-109 of SEQ ID NO: 217. CD19 42og scFv also interacted with another region comprising the amino acid sequence VPPDSVSRGPL (SEQ ID NO: 222) located at positions 202-212 of SEQ ID NO: 217 (Full-length CD19). Accordingly, CD19 42og does not bind to the same epitope as FMC63, 4G7, 3B10, or B43 (e.g., anti-CD19 clinical antibodies).

These results further demonstrate the unique functional characteristics of the novel CD19 binders described herein, in particular CD19 42og. The novel binders disclosed herein have uncovered new clinically relevant CD19 epitopes that do not overlap with epitopes from at least three well-characterized clinically relevant antibodies, namely, the FMC63, 4G7, and 3B10 (Table 10).

Lastly, the specificity and selectivity of the novel CD19 disclosed herein was assessed using a high-throughput membrane proteome array (Integral Molecular). These experiments demonstrated that CD19 42og selectively bound to CD19 when assessed for cross-reactivity against an array of 5,220 human membrane proteins, which represented over 94% of the human membrane proteome.

C. IL-18 Armored Enhanced CD19-42 and CD19-52 CAR T Cells Effectiveness

Among the 12 novel CD19 CAR T cells tested, CAR T cells expressing a CAR comprising either original (og) or optimized (op) CD19 binder 42, or 52 had the best attributes of low tonic signaling, strong activation rate (e.g., NFAT), similar doublings during manufacturing expansion phase (e.g., expansion profiles), robust and stable surface expression (e.g., with good maintenance of mfi), enhanced killing, and long term persistence in therapeutic activity. To further enhance the effectiveness of the novel CD19 CAR T cells, the effect of various immune modulators (e.g., enhancers, payloads, or armors) on original (og) or optimized (op) CD19-42, and CD19-52 CAR T cells were determined.

Several immune modulators increase the efficacy of engineered CAR T cells. These immune modulators can enhance the efficacy of CAR T cells using different mechanisms. For example, the immune modulator can increase the recruitment of endogenous immune cells to the tumor site (e.g., NK cell infiltration), increase persistence, reduce T cell exhaustion; and/or enable resistance to checkpoint inhibitors. In addition, immune modulators, such as cytokines (e.g., IL-2, IL-7, IL-12, IL-15, IL-15/IL-15 sushi, IL-15/IL-15 sushi anchor, IL-15/IL-15RA, IL-18, IL-21, IL-21) can enhance T cell priming, antigen presentation, and T cell infiltration in a solid tumor. However, not all known immune modulators can enhance the effectiveness (enhanced tumor clearance and low toxicity) and persistence of CAR T cells in vivo. The present disclosure shows that IL-18 enhanced the efficacy of CART cells expressing a CAR comprising a novel CD19 binder 42 or 52 in their original or optimized version.

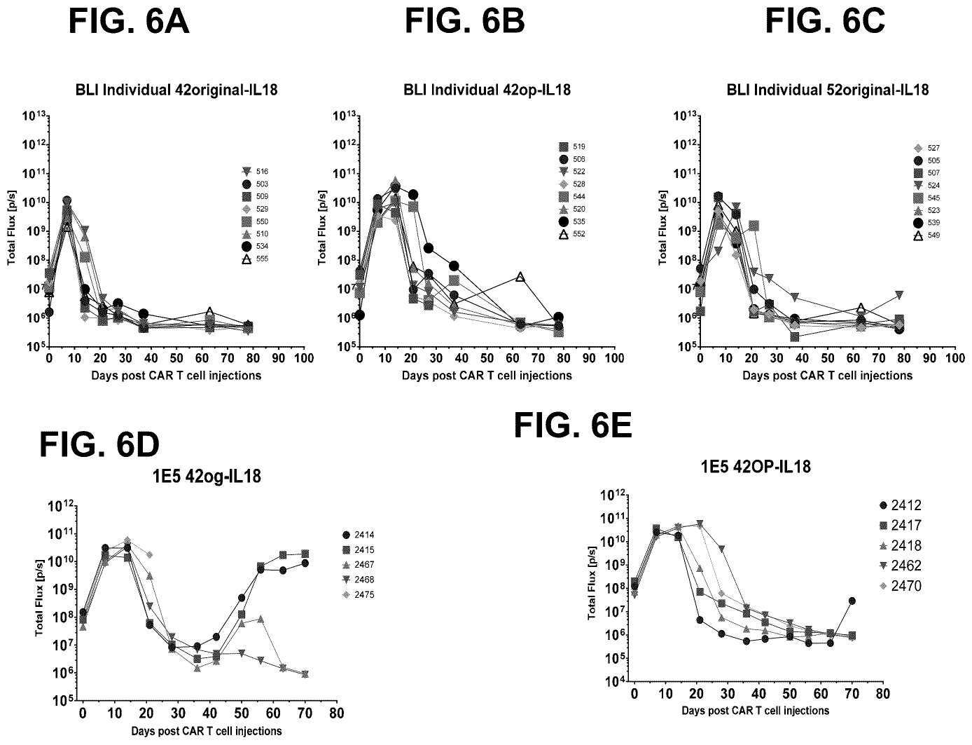

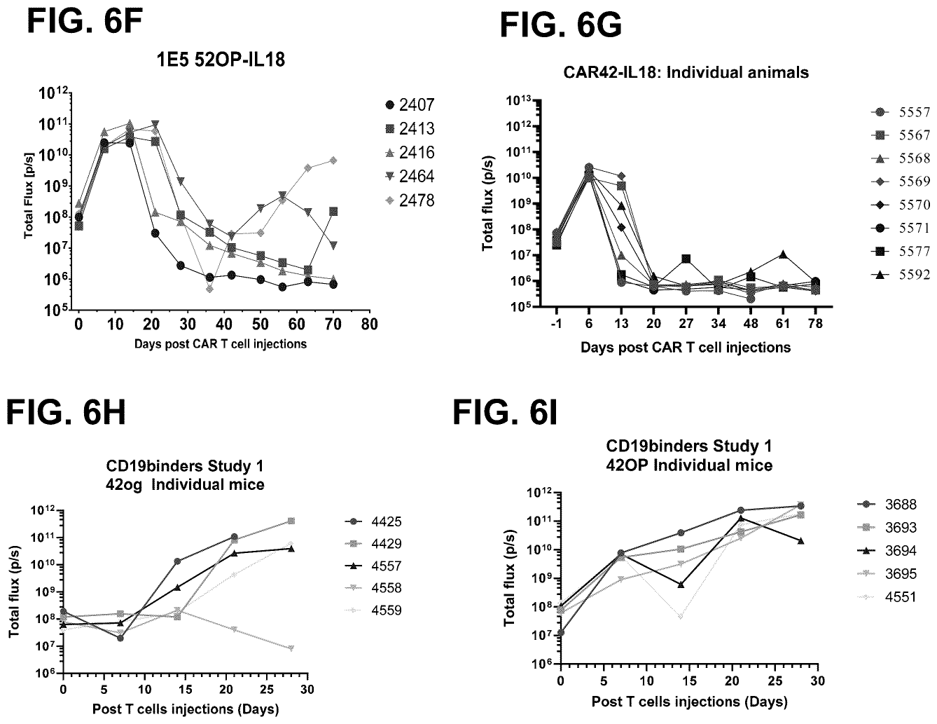

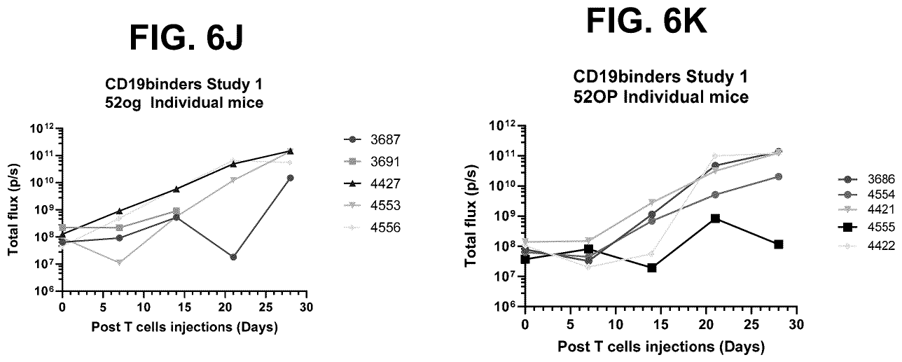

Each of the CD19-42-IL18 and CD19-52-IL18 CAR T cells showed an ability to clear tumor in mice. However, the CD19-42(original)-IL18 CAR T cells showed little more overall tightness in their time to initially clear tumor when compared to CD19-42(optimized)-IL18 CAR T cells. Similar tumor clearance efficacy was observed in mice injected with CD19-52 (original)-IL18 CAR T cells and CD19-52 (optimized)-IL18 CART cells. In all tested CAR T cells, tumor relapsed in at least one treated animal. However, CD19-42og-IL-18 CAR T cells had the ability to regain control of the relapsed tumors (FIGS. 6D-G). In contrast, CD19-42 CAR T cells that were not armored with IL-18 were not able to regain control of the relapsed tumors (FIGS. 6H-K). These data suggest that armoring CD19-42 and 52 original or optimized CAR T cells enhanced the efficacy of CD19 CAR T cells.

Mice injected with a well-known CD19 CAR relapsed. This was unexpected because these mice were positive control. In addition, the positive control CD19 CAR had been described to consistently clear tumor. Yet, the positive control CD19 CAR T cells did not have the expected tumor control. Even though they showed signs of controlling tumor, the mice were euthanized because of excessive weight loss. The novel CD19 binders disclosed herein showed a stronger tumor clearance efficacy and less side effects when armored with IL-18 when compared to the positive control CAR. Thus, the positive control weakened response in this assay indicated that the novel CD19 binders disclosed herein are better, more efficient/effective and less toxic than known CD19 binders when armored with IL-18.

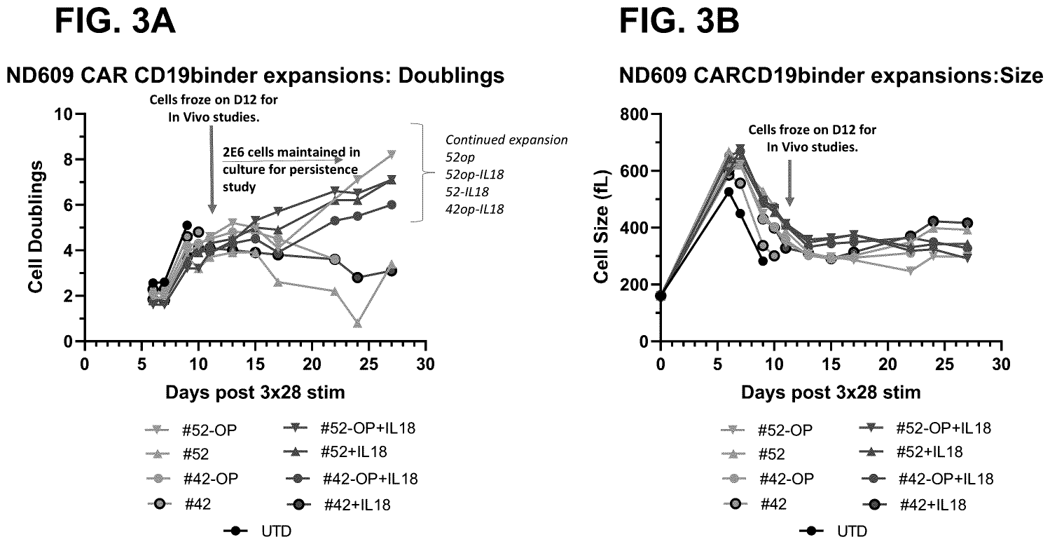

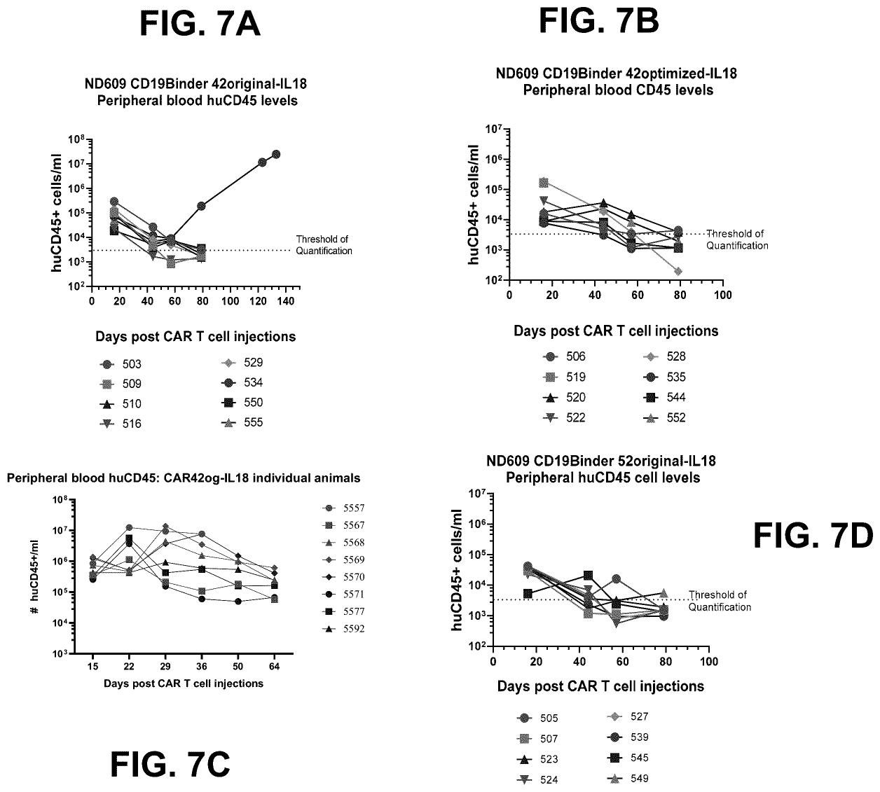

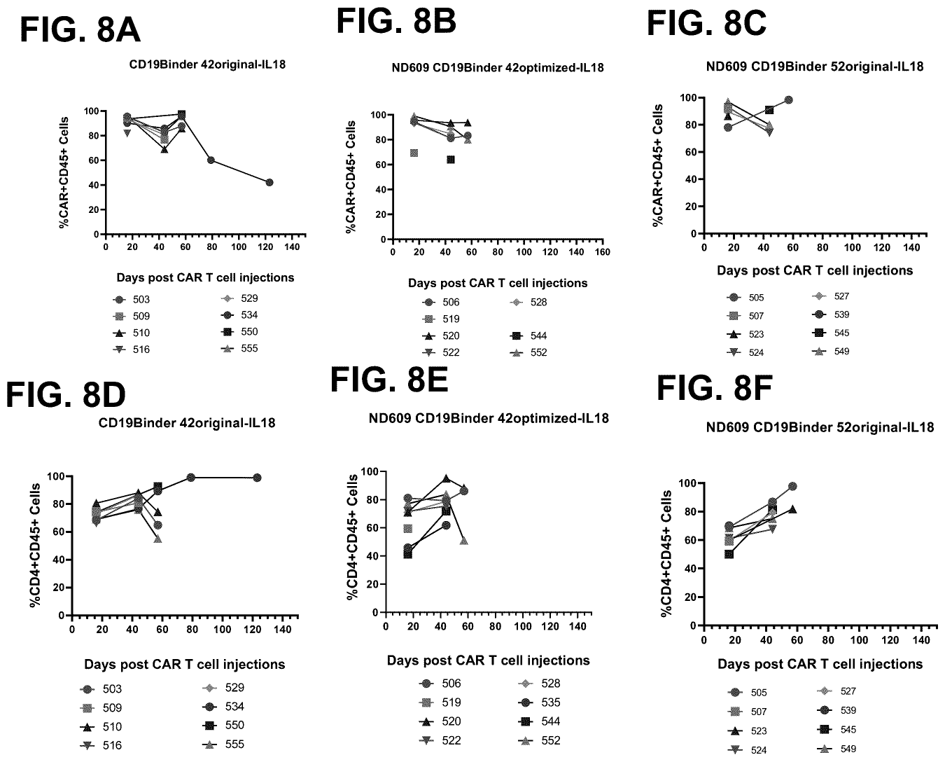

FIGS. 8A-F, 9C-F, and 10C-F show that IL-18 co-expression enhanced or maintained a high percentage of CD4⁺CD19 CART cells in treated animals. The concentration of huCD45⁺ T cells in the blood of administered animals decreased over time. Indeed, the huCD45⁺ T cells in murine peripheral blood linearly decreased started at day 20 (FIGS. 7A-D). This decreased correlated with the timing of tumor clearance shown in FIGS. 6A-C and G. These results further showed that CD19-42(og)-IL18 CAR T cells, CD19-42(op)-IL18 CAR T cells, CD19-52(og)-IL18 CAR T cells, and CD19-52(op)-IL18 CAR T cells contracted in numbers after tumor clearance. See FIG. 3 (compare IL-18 vs no IL-18).

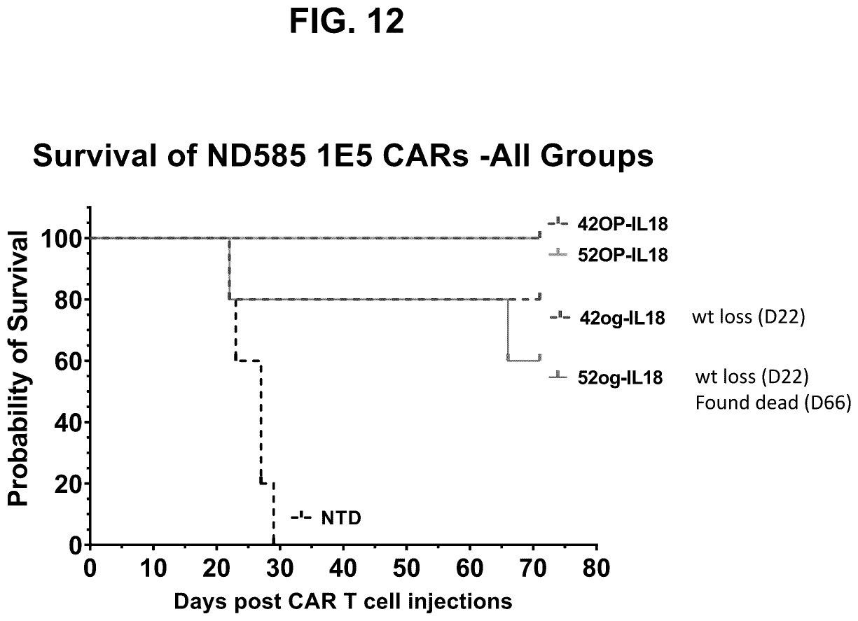

In FIG. 12, the Kaplan-Meier survival curve shows the survival of animals administered with 1×10⁵ IL-18 armored ND585 CD19 CAR T cells expressing original or optimized CD19 binders 42 and 52. 100% of mice administered with CD19-42OP-Th18 CAR T cells and CD19-52OP-IL18 CAR T cells remained alive for the duration of the study (70 days). 80% of mice administered with CD19-42og-IL18 CART cells remained alive at the end of the study and one mouse died on Day 22 due to endpoint weight loss during tumor clearance. 60% of mice administered with CD19-52og-IL18 CAR T cells remained alive at the end of the study. In one mouse administered CD19-52og-IL18 CAR T cells died on Day 22 due to endpoint weight loss during tumor clearance; and a second mouse died on day 66 from spontaneous death. Mice expressing a positive control CD19 CAR T cells died at day 28 and mice administered with untransduced CAR T cells died at day 29 of excessive weigh loss. The results from the positive control CD19 CAR were unexpected, but consistent with the observations made with the tumor clearance assay above. These data showed that enhanced efficacy and tolerability of the novel CD19 binders described herein over existing CD19 binders.

TABLE 3
P1-P13 Novel CD19 binders

| | | | scFv SEQ ID NO | | V_{H} SEQ IDNO | | V_{L} SEQ ID NO | |
|---|---|---|---|---|---|---|---|---|
| | | Other | Amino | Nucleic | Amino | Nucleic | Amino | Nucleic |
| Name | Description | names | acid | acid | acid | acid | acid | acid |
| P1 | CD19 42og | 42 or A2 | 9 | 21 | 8 | 20 | 7 | 19 |
| P2 | CD19 A4 | 43 | 18 | 24 | 17 | 23 | 16 | 22 |
| P3 | CD19 E4 | 44 | 64 | 102 | 63 | 66 | 62 | 65 |
| P4 | CD19 E7 | 45 | 75 | 103 | 74 | 77 | 73 | 76 |
| P5 | CD19 7 | 46 | 86 | 104 | 85 | 88 | 84 | 87 |
| P6 | CD19 10 | 47 | 146 | 118 | 145 | 148 | 144 | 147 |
| P7 | CD19 11 | 48 | 157 | 119 | 156 | 159 | 155 | 158 |
| P8 | CD19 14 | 49 | 168 | 114 | 167 | 170 | 166 | 169 |
| P9 | CD19 15 | 50 | 179 | 115 | 178 | 181 | 177 | 180 |
| P10 | CD19 16 | 51 | 190 | 120 | 189 | 192 | 188 | 191 |
| P11 | CD19 18 | 52 | 201 | 116 | 200 | 203 | 199 | 202 |
| P12 | CD19 23 | 53 | 212 | 117 | 21 | | 214 | 210 |
| P13 | CD19 52 opt | Optimized | 201 | 216 | 200 | | 199 | |
| | or OP | 52 | | | | | | |
| P14 | CD19 42 opt | Optimized | 226 | 225 | | | | |
| | or OP | 42 | | | | | | |

Accordingly, one aspect of the present disclosure provides isolated nucleic acid molecules encoding a chimeric antigen receptor (CAR) comprising a CD19 binder disclosed herein. In some embodiments, the CAR comprises an anti-CD19 binding domain selected from P1, P2, P3, P4, P5, P6, P7, P8, P9, P10, P11, P12, or P13. In some embodiments, the anti-CD19 binding domain comprises a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

Another aspect of the present disclosure provides an isolated polypeptide molecule encoded by the nucleic acid molecule disclosed in Table 3 or Table 1.

Another aspect of the present disclosure provides a vector and/or a T cell comprising a nucleic acid molecule encoding any one of the novel CD19 binders (e.g., P1-P13) described herein and a nucleic acid encoding a polypeptide that enhances an immune cell function, or a functional derivative thereof. The inventors of the present disclosure further discovered that arming CAR T cells comprising a low affinity CD19 CAR of the present disclosure with one or more molecules that enhance T cell priming significantly enhanced the anti-tumor activities of the low affinity CD19 CAR T cells while reducing side effects associated with CD19 CAR as disclosed herein. Indeed, the armor (e.g., T cell priming molecule) endowed the low affinity CAR T cells with an antigen presenting function (“APC). This new property allows the low affinity CD19 CAR T cells to kill CD19⁺ cells, while inducing endogenous naive T cells to differentiate into an effector cytotoxic T cell after being stimulated with an antigen.

### II. Chimeric Antigen Receptors (Cars)

One aspect of the present disclosure provides compositions of matter and methods of use for the treatment of a disease such as cancer using anti-CD19 chimeric antigen receptors (CAR). In particular, the present disclosure provides a number of chimeric antigen receptors (CAR) comprising an antibody or antibody fragment engineered for enhanced binding to a CD19 protein. In some embodiments, the CAR comprises an amino acid sequence of any one of SEQ ID NO: 63, SEQ ID NO: 74, SEQ ID NO: 85, SEQ ID NO: 145, SEQ ID NO: 167, SEQ ID NO: 178, SEQ ID NO: 200, SEQ ID NO: 211, SEQ ID NO: 156, SEQ ID NO: 189, SEQ ID NO: 17, SEQ ID NO: 8, SEQ ID NO: 62, SEQ ID NO: 73, SEQ ID NO: 84, SEQ ID NO: 144, SEQ ID NO: 166, SEQ ID NO: 177, SEQ ID NO: 199, SEQ ID NO: 210, SEQ ID NO: 155, SEQ ID NO: 188, SEQ ID NO: 16, and SEQ ID NO: 7. In some embodiments, the CAR comprises a polypeptide encoded by the nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; or a nucleic sequence having 90%, about 91%, about 92%, about 93%, about 94%, about 95%, about 96%, about 97%, about 98%, or about 99% identity to SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 21, SEQ ID NO:116, SEQ ID NO: 117, SEQ ID NO: 118, SEQ ID NO: 119, SEQ ID NO: 120, SEQ ID NO: 225, or SEQ ID NO: 216.

In some embodiments, the CARs of the present disclosure, comprising an anti-CD19 antigen binding domain described herein, have a low affinity and a fast Off-rate when compared to CARs comprising anti-CD19 antigen binding domain known in the art.

Accordingly, the present disclosure provides a cell (e.g., T cell) engineered to express a CAR, wherein the CAR T cell (“CART”) exhibits an antitumor property. The cell is transformed with the CAR and the CAR is expressed on the cell surface. The cell (e.g., T cell) is transduced with a viral vector encoding a CAR. The viral vector is a retroviral vector. In some embodiments, the viral vector is a lentiviral vector. The cell may stably express the CAR. The cell (e.g., T cell) may be transfected with a nucleic acid (e.g., mRNA, cDNA, DNA, encoding a CAR). In some embodiments, the cell may transiently express the CAR.

In some embodiments, the anti-CD19 protein binding portion of the CAR is a scFv antibody fragment. Such antibody fragments may be functional in that they retain the equivalent binding affinity. For example, they bind the same antigen with comparable efficacy as the IgG antibody from which they were derived. Such antibody fragments may be functional in that they provide a biological response that can include, but is not limited to, activation of an immune response, inhibition of signal-transduction origination from its target antigen, inhibition of kinase activity, and the like, as will be understood by a skilled artisan. In some embodiments, the anti-CD 19 antigen binding domain of the CAR is a scFv antibody fragment that is human derived.

The novel CD19 antigen binding domains were engineered to have low affinity and a fast off-rate. The CD19 antigen binding domains were identified based on binding to CD19 on HEK 293 cells followed by binding to NALM6 expressing or lacking CD19 expression. In some embodiments, the novel anti-CD19 antigen binding domain described herein may have a binding affinity for the human CD19 (hCD19) antigen. For example, the anti-CD19 antigen binding domain described herein may have an association rate constant or Kₒₙ rate (the association rate constant (M⁻¹ min⁻¹); antibody (Ab)+antigen (Ag)→Ab-Ag) of at least about 2×10⁵ M⁻¹ s⁻¹, at least about 5×10⁵ M⁻¹ s⁻¹, at least about 10⁶ M⁻¹ s⁻¹, at least about 5×10⁶ M⁻¹ s⁻¹, at least about 10⁷ M⁻¹ s⁻¹ at least about 5×10⁷ M⁻¹ s⁻¹, or at least about 10⁸ M⁻¹ s⁻¹.

A. Chimeric Antigen Receptor

The present disclosure provides engineered immune effector cells (for example, T cells or NK cells) comprising one or more CARs that direct the immune effector cells to cancer. In some embodiments, the CAR comprises an antigen-binding domain, a transmembrane domain, a co-stimulatory domain, and an intracellular domain. The CAR may comprise any antigen binding domain, any hinge, any transmembrane domain, any costimulatory domain, and any intracellular signaling domain described herein.

The antigen binding domain may be operably linked to another domain of the CAR, such as the transmembrane domain or the intracellular domain, both described herein, for expression in any immune cell described herein. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present disclosure. A subject CAR of the present disclosure may also include a spacer domain as described herein. In some embodiments, each of the antigen binding domain, transmembrane domain, and intracellular domain is separated by a linker.

One aspect of the present disclosure provides a chimeric antigen receptor (CAR) comprising a single chain antibody or a single chain antibody fragment comprising an anti-CD19 binding domain, a transmembrane domain, a costimulatory, and an intracellular signaling domain. The anti-CD19 binding domain comprises a light chain variable domain comprising a light chain complementary determining region 1 (LC CDR1) of SEQ ID NO: 1, a light chain complementary determining region 2 (LC CDR2) of SEQ ID NO: 2, and a light chain complementary determining region 3 (LC CDR3) of SEQ ID NO: 3; and a heavy chain variable domain comprising a heavy chain complementary determining region 1 (HC CDR1) of SEQ ID NO: 4, a heavy chain complementary determining region 2 (HC CDR2) of SEQ ID NO: 5, and a heavy chain complementary determining region 3 (HC CDR3) of SEQ ID NO: 6.

Alternatively, the anti-CD19 binding domain can comprise a light chain variable domain comprising a light chain complementary determining region 1 (LC CDR1) of SEQ ID NO: 193, a light chain complementary determining region 2 (LC CDR2) of SEQ ID NO: 194, and a light chain complementary determining region 3 (LC CDR3) of SEQ ID NO: 195; and a heavy chain variable domain comprising a heavy chain complementary determining region 1 (HC CDR1) of SEQ ID NO: 196, a heavy chain complementary determining region 2 (HC CDR2) of SEQ ID NO: 197, and a heavy chain complementary determining region 3 (HC CDR3) of SEQ ID NO: 198.

In another embodiment, the anti-CD19 binding domain comprises a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

TABLE 2
P1-P12 CDR Amino Acid sequences

| | SEQ | | |
|---|---|---|---|
| | ID | | |
| Name | NO | Description | Sequences |
| P1 | 1 | 42og CDR-L1 | RASQTISNYLN |
| | 2 | 42og CDR-L2 | AASSLQS |
| | 3 | 42og CDR-L3 | QQSYSTPPT |
| | 4 | 42og CDR-H1 | AASGFTFSNYAIS |
| | 5 | 42og CDR-H2 | VSVITASGVDTYYADSV |
| | 6 | 42og CDR-H3 | GGTPYFITTYDYYGFDV |
| P2 | 10 | A4 CDR-L1 | RASQSVSSNYLA |
| | 11 | A4 CDR-L2 | GASSRAT |
| | 12 | A4 CDR-L3 | QQYESSPSWT |
| | 13 | A4 CDR-H1 | KASGGTFSNYYIS |
| | 14 | A4 CDR-H2 | MGGIIPLFGTTNYAQ |
| | 15 | A4 CDR-H3 | GTWYAGDI |
| P3 | 56 | E4 CDR-L1 | RASQSISSYLN |
| | 57 | E4 CDR-L2 | GASSLQS |
| | 58 | E4 CDR-L3 | QQSYRTPVT |
| | 59 | E4 CDR-H1 | KASGGTFSNYAIN |
| | 60 | E4 CDR-H2 | MGRIVPLLGIANYAQ |
| | 61 | E4 CDR-H3 | EHIAYRPTSAGYYYYMDI |
| P4 | 67 | E7 CDR-L1 | RASQDITRYLN |
| | 68 | E7 CDR-L2 | AASSLQS |
| | 69 | E7 CDR-L3 | QQSYSYPPT |
| | 70 | E7 CDR-H1 | AASGFTFRDYGMH |
| | 71 | E7 CDR-H2 | VAVISYEGSNEYYADSV |
| | 72 | E7 CDR-H3 | DRGFAGWYDYAFDP |
| P5 | 78 | 7 CDR-L1 | RASQSISKYLN |
| | 79 | 7 CDR-L2 | DASSLQS |
| | 80 | 7 CDR-L3 | QQSYTIPLT |
| | 81 | 7 CDR-H1 | KASGGTFSSYAFS |
| | 82 | 7 CDR-H2 | MGGIVPLFGAVEYAQ |
| | 83 | 7 CDR-H3 | EKGFYRYFDH |
| P6 | 89 | 10 CDR-L1 | RASQTISRYLN |
| | 90 | 10 CDR-L2 | AASSLQS |
| | 91 | 10 CDR-L3 | QQSYRPPLT |
| | 141 | 10 CDR-H1 | AASGFTFSSYAMS |
| | 142 | 10 CDR-H2 | VSTISAGGHGTYYADSV |
| | 143 | 10 CDR-H3 | GAGYFDY |
| P7 | 149 | 11 CDR-L1 | RASQSISSYLN |
| | 150 | 11 CDR-L2 | AASSLQS |
| | 151 | 11 CDR-L3 | QQTGAVPYTF |
| | 152 | 11 CDR-H1 | AASGFTFRDYAMS |
| | 153 | 11 CDR-H2 | VSAISESGIDTYYADSV |
| | 154 | 11 CDR-H3 | VAGYDSDSSTYYDYMDV |
| P8 | 160 | 14 CDR-L1 | RASQSISNYLN |
| | 161 | 14 CDR-L2 | AASSLQS |
| | 162 | 14 CDR-L3 | QQAYSAPIT |
| | 163 | 14 CDR-H1 | AASGFTFGDYAMS |
| | 164 | 14 CDR-H2 | VSAISRGGHGTYYADSV |
| | 165 | 14 CDR-H3 | LVGYGLDY |
| P9 | 171 | 15 CDR-L1 | RASQPIRPYLN |
| | 172 | 15 CDR-L2 | DASSLQS |
| | 173 | 15 CDR-L3 | QQSYSAPYT |
| | 174 | 15 CDR-H1 | AASGFTFSSYAMS |
| | 175 | 15 CDR-H2 | VSVISGGGANTYYADSVK |
| | 176 | 15 CDR-H3 | DWRYFDH |
| P10 | 182 | 16 CDR-L1 | TGSSSNIGAGYDVH |
| | 183 | 16 CDR-L2 | GNNNRPS |
| | 184 | 16 CDR-L3 | QSYDVSLGVWV |
| | 185 | 16 CDR-H1 | TVSGGSISSPSYYWG |
| | 186 | 16 CDR-H2 | IGSIYYTGATYYNPSL |
| | 187 | 16 CDR-H3 | YGPAGVGFDY |
| P11 | 193 | 18 CDR-L1 | TGSSSNIGAGYDVH |
| | 194 | 18 CDR-L2 | GTKNRPS |
| | 195 | 18 CDR-L3 | QSYDVRLKGWV |
| | 196 | 18 CDR-H1 | TVSGGSITSSSYYWG |
| | 197 | 18 CDR-H2 | IGSIYYTGTTYYNPSL |
| | 198 | 18 CDR-H3 | YVGLSGGFDY |
| P12 | 204 | 23 CDR-L1 | RASQSIYSYLN |
| | 205 | 23 CDR-L2 | DASSLQS |
| | 206 | 23 CDR-L3 | QQSYTAPPT |
| | 207 | 23 CDR-H1 | AASGFTFSNYAMS |
| | 208 | 23 CDR-H2 | VSAISESGHGTYYADSV |
| | 209 | 23 CDR-H3 | LDWAGFDV |

In some embodiments, the light chain variable region comprises the amino acid sequence of SEQ ID NO: 7 or 199; or an amino acid sequence having at least about 90%, about 91%, about 92%, about 93%, about 94%, about 95%, about 96%, about 97%, about 98%, or about 99% identity to SEQ ID NO: 7 or 199. In some embodiments, the heavy chain variable region comprises the amino acid sequence of SEQ ID NO: 8 or 200, or an amino acid sequence having at least about 90%, about 91%, about 92%, about 93%, about 94%, about 95%, about 96%, about 97%, about 98%, or about 99% identity to SEQ ID NO: 8 or 200.

In some embodiments, the light chain variable region comprises the amino acid sequence of SEQ ID NO: 7 and the heavy chain variable region comprises the amino acid sequence of SEQ ID NO: 8. In some embodiments, the light chain variable region comprises the amino acid sequence of SEQ ID NO: 199 and the heavy chain variable region comprises the amino acid sequence of SEQ ID NO: 200. In some embodiments, the CD19 binding domain is a scFv.

In some embodiments, the anti-CD19 binding domain comprises an amino acid sequence selected from the group consisting of SEQ ID NO: 9, 18, 64, 75, 86, 190, 157, 212, 201, 226, 179, 168, and 146, or a sequence having 90%, about 91%, about 92%, about 93%, about 94%, about 95%, about 96%, about 97%, about 98%, or about 99% identity to SEQ ID NO: 9, 18, 64, 75, 86, 190, 157, 212, 201, 226, 179, 168, or 146.

In some embodiments, the anti-CD19 binding domain comprises a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; or a sequence having 90%, about 91%, about 92%, about 93%, about 94%, about 95%, about 96%, about 97%, about 98%, or about 99% identity to SEQ ID NO: 21, 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

In some embodiments, the anti-CD19 binding domain comprises a light chain variable region or a heavy chain variable region encoded by (a) a nucleic acid sequence selected from the group consisting of SEQ ID NO: 19, SEQ ID NO: 20, SEQ ID NO:21, SEQ ID NO:22, SEQ ID NO: 23, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; or (b) a sequence having 90%, about 91%, about 92%, about 93%, about 94%, about 95%, about 96%, about 97%, about 98%, or about 99% identity to SEQ ID NO: 19-24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

1. Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g., a tumor associated antigen) on a target cell (e.g., a cancer cell). The target antigen may include any type of protein, or epitope thereof, associated with the target cell. For example, the CAR may comprise affinity to a target antigen on a target cell that indicates a particular status of the target cell.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In some embodiments, the antigen binding domain comprises a full-length antibody. In some embodiments, the antigen binding domain comprises an antigen binding fragment (Fab), e.g., Fab, Fab′, F(ab′)2, a monospecific Fab2, a bispecific Fab2, a trispecific Fab2, a single-chain variable fragment (scFv), dAb, tandem scFv, VhH, V-NAR, camelid, diabody, minibody, triabody, or tetrabody. In some embodiments, the antigen-binding domain is selected from the group consisting of (a) a full-length antibody or antigen-binding fragment thereof, (b) a Fab, (c) a single-chain variable fragment (scFv), and (d) a single-domain antibody.

In some embodiments, a CAR of the present disclosure may have affinity for one or more target antigens on one or more target cells. In some embodiments, a CAR may have affinity for one or more target antigens on a single target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through a polypeptide linker, an Fc hinge region, or a membrane hinge region.

In some instances, the antigen binding domain may be derived from the same species in which the CAR will ultimately be used. For example, for use in humans, the antigen binding domain of the CAR may comprise a human antibody as described elsewhere herein, or a fragment thereof.

Accordingly, a CAR encoded by a lentiviral vector or retroviral vector of the present disclosure may target one of the following cancer associated antigens (tumor antigens): CD19; CD20; CD22 (Siglec 2); CD37; CD 123; CD22; CD30; CD 171; CS-1 (also referred to as CD2 subset 1, CRACC, SLAMF7, CD319, and 19A24); C-type lectin-like molecule-1 (CLL-1 or CLECL1); CD33; CD133; epidermal growth factor receptor (EGFR); epidermal growth factor receptor variant III (EGFRvIII); human epidermal growth factor receptor (HER1); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); Folate receptor alpha; Receptor tyro sine-protein kinase ERBB2 (Her2/neu); Mucin 1, cell surface associated (MUC 1); GalNAca1-O-Ser/Thr (Tn) MUC 1 (TnMUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); tyrosine-protein kinase Met (c-Met); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-2 (GPC2); Glypican-3 (GPC3); NKG2D; KRAS; GDNF family receptor alpha-4 (GFRa4); IL13Ra2; Fc receptor-like 5 (FCRL5); and immunoglobulin lambda-like polypeptide 1 (IGLL1).

In some embodiments, the CAR targets CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1. In some embodiments, the antigen-binding domain specifically binds a target antigen selected from the group consisting of CD4, CD19, CD20, CD22, BCMA, CD123, CD133, EGFR, EGFRvIII, mesothelin, Her2, PSMA, CEA, GD2, IL-13Ra2, glypican-3, GPC2, TnMuc1, CIAX, LI-CAM, CA 125, CTAG1B, Mucin 1, and Folate receptor-alpha. In some embodiments, the CAR targets CD19.

Accordingly, one aspect of the present disclosure provides an anti-CD19 binding domain comprising a light chain variable domain comprising a light chain complementary determining region 1 (LC CDR1) of SEQ ID NO: 1, a light chain complementary determining region 2 (LC CDR2) of SEQ ID NO: 2, and a light chain complementary determining region 3 (LC CDR3) of SEQ ID NO: 3; and a heavy chain variable domain comprising a heavy chain complementary determining region 1 (HC CDR1) of SEQ ID NO: 4, a heavy chain complementary determining region 2 (HC CDR2) of SEQ ID NO: 5, and a heavy chain complementary determining region 3 (HC CDR3) of SEQ ID NO: 6. Alternatively, the anti-CD19 binding domain comprises a light chain variable domain comprising a light chain complementary determining region 1 (LC CDR1) of SEQ ID NO: 193, a light chain complementary determining region 2 (LC CDR2) of SEQ ID NO: 194, and a light chain complementary determining region 3 (LC CDR3) of SEQ ID NO: 195; and a heavy chain variable domain comprising a heavy chain complementary determining region 1 (HC CDR1) of SEQ ID NO: 196, a heavy chain complementary determining region 2 (HC CDR2) of SEQ ID NO: 197, and a heavy chain complementary determining region 3 (HC CDR3) of SEQ ID NO: 198. In another embodiment, the anti-CD19 binding domain comprises a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

In some embodiments, the anti-CD19 binding domain is a scFv comprising a light chain variable region comprising the amino acid sequence of SEQ ID NO: 7 or 199, or a sequence having about 90%, about 91%, about 92%, about 93%, about 94%, about 95%, about 96%, about 97%, about 98%, or about 99% identity to SEQ ID NO: 7 or 199; and/or a heavy chain variable region comprising the amino acid sequence of SEQ ID NO: 8, or 200, or a sequence having about 90%, about 91%, about 92%, about 93%, about 94%, about 95%, about 96%, about 97%, about 98%, or about 99% identity to SEQ ID NO: 8 or 200.

One aspect of the present disclosure provides an anti-CD19 binding domain (e.g., scFv) comprising a light chain variable domain or a heavy variable domain encoded by the nucleic acid sequence selected from SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216. In some embodiments, the nucleic acid sequence of the light chain variable domain or the heavy variable domain of the anti-CD19 binding domain (e.g., scFv) is encoded by a nucleic acid sequence having 90%, about 91%, about 92%, about 93%, about 94%, about 95%, about 96%, about 97%, about 98%, or about 99% identity to SEQ ID NO: 21, SEQ ID NO: 24 SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

The anti-CD19 antigen binding domain of the present disclosure may have a K_{off} rate (the dissociation rate constant (min⁻¹); (Ab-Ag)→antibody (Ab)+antigen (Ag)) of less than about 5×10⁻¹ s⁻¹, less than about 10⁻¹ s⁻¹, less than about 5×10⁻¹ s⁻¹, less than about 10⁻¹ s⁻¹ less than about 5×10⁻¹ s⁻¹, less than about 10⁻¹ s⁻¹, less than about 5×10⁻¹ s⁻¹, or less than about 10⁻¹ s⁻¹. In an another embodiment, an antibody of the disclosure has a K_{off} of less than about 5×10⁻¹ s⁻¹, less than about 10⁻¹ s⁻¹, less than about 5×10⁻¹ s⁻¹, less than about 10⁻¹ s⁻¹, less than about 5×10⁻¹ s⁻¹, less than about 10⁻¹ s⁻¹, less than about 5×10⁻¹ s⁻¹, less than about 10⁻¹ s⁻¹, less than about 5×10⁻¹s⁻¹, less than about 10⁻¹ s⁻¹, or less than about 10⁻¹s⁻¹.

The anti-CD19 antigen binding domain of the present disclosure may have an affinity constant or Kₐ (Kₒₙ/K_{off}) of at least about 10² M⁻¹, at least about 5×10² M⁻¹, at least about 10³ M⁻¹, at least about 5×10³ M⁻¹, at least about 10⁴ M⁻¹, at least about 5×10⁴ M⁻¹, at least about 10⁵ M⁻¹, at least about 5×10⁵ M⁻¹, at least about 10⁶ M⁻¹, at least about 5×10⁶ M⁻¹, at least about 10⁷ M⁻¹, at least about 5×10⁷ M⁻¹, at least about 10⁸ M⁻¹, at least about 5×10⁸ M⁻¹, at least about 10⁹ M⁻¹, at least about 5×10⁹ M⁻¹, at least about 10¹⁰ M⁻¹, at least about 5×10¹⁰ M⁻¹, at about least 10¹¹ M⁻¹, at least about 5×10¹¹ M⁻¹, at least about 10¹² M⁻¹, at least about 5×10¹² M⁻¹, at least about 10¹³ M⁻¹ at least about 5×10¹³ M⁻¹ at least about 10¹⁴ M⁻¹ at least about 5×10¹⁴ M⁻¹, at least about 10¹⁵ M⁻¹, or at least about 5×10¹⁵ M⁻¹.

The anti-CD19 antigen binding domain of the present disclosure may have a dissociation constant or K_{D} (K_{off}/Kₒₙ) of less than about 5×10⁻² M, less than about 10⁻² M, less than about 5×10⁻³ M, less than about 10⁻³ M, less than 5×10⁻⁴ M, less than about 10⁻⁴ M, less than about 5×10⁻⁵ M, less than about 10⁻⁵ M, less than 5×10⁻⁶ M, less than about 10⁻⁶ M, less than about 5×10⁻⁷ M, less than about 10⁻⁷ M, less than about 5×10⁻⁸ M, less than about 10⁻⁸ M, less than about 5×10⁻⁹ M, less than about 10⁻⁹ M, less than about 5×10⁻¹⁰ M, less than about 10⁻¹⁰ M, less than about 5×10⁻¹¹ M, less than about 10⁻¹¹ M, less than about 5×10⁻¹² M, less than about 10⁻¹² M, less than about 5×10⁻¹³ M, less than about 10⁻¹³ M, less than about 5×10⁻¹⁴ M, less than about 10⁻¹⁴ M, less than about 5×10⁻¹⁵ M, or less than about 10⁻¹⁵ M.

When used with the method described herein, the anti-CD19 antigen binding domain of the present disclosure may specifically bind to human CD19 with a dissociation constant (Kₐ) of less than about 3000 nM, less than about 2500 nM, less than about 2000 nM, less than about 1500 nM, less than about 1000 nM, less than about 750 nM, less than about 500 nM, less than about 250 nM, less than about 200 nM, less than about 150 nM, less than about 100 nM, or less than about 75 nM as assessed using a method described herein or known to one of skill in the art (e.g., a BIAcore assay, ELISA) (Biacore International AB, Uppsala, Sweden).

In some embodiments, the anti-CD19 antigen binding domain of the present disclosure may specifically bind to a human CD19 antigen with a dissociation constant (K_{d}) of between about 25 to about 3400 nM, about 25 to about 3000 nM, about 25 to about 2500 nM, about 25 to about 2000 nM, about 25 to about 1500 nM, about 25 to about 1000 nM, about 25 to about 750 nM, about 25 to about 500 nM, about 25 to about 250 nM, about 25 to about 100 nM, about 25 to about 75 nM, about 25 to about 50 nM as assessed using a method described herein or known to one of skill in the art (e.g., a BIAcore assay, ELISA). In another embodiment, the anti-CD19 antigen binding domain may specifically bind to hCD19 with a dissociation constant (K_{d}) of at least about 500 nM, at least about 100 nM, at least about 75 nM or at least about 50 nM as assessed using a method described herein or known to one of skill in the art (e.g., a BIAcore assay, ELISA).

2. Transmembrane Domain

A CAR of the present disclosure can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen-binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

In some embodiments, the transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. In some embodiments, the transmembrane domain of particular use in this disclosure includes, without limitation, a transmembrane domain derived from (the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR).

In some embodiments, the transmembrane domain comprises at least a transmembrane region of a protein selected from the group consisting of the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR).

In some embodiments, the transmembrane domain may be synthetic. In some embodiments, the synthetic transmembrane domain comprises predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In one embodiment, the transmembrane domain comprises a CD8α transmembrane domain. In some embodiments, the transmembrane domain comprises a CD8α transmembrane domain comprising the amino acid sequence set forth in SEQ ID NO: 29. In some embodiments, the transmembrane domain comprises the nucleotide sequence set forth in SEQ ID NO: 30.

In some embodiments, the transmembrane domain comprises a CD28 transmembrane domain. In some embodiments, the CAR comprises a CD28 transmembrane domain comprising the amino acid sequence set forth in SEQ ID NO: 31. In some embodiments, the CD28 transmembrane domain comprises the nucleotide sequence set forth in SEQ ID NO: 32.

In some embodiments, the transmembrane domain comprises a CD28 transmembrane domain. In some embodiments, the CAR comprises a ICOS transmembrane domain comprising the amino acid sequence set forth in SEQ ID NO: 33. In some embodiments, the ICOS transmembrane domain comprises the nucleotide sequence set forth in SEQ ID NO: 34.

Tolerable variations of the transmembrane and/or hinge domain will be known to those of skill in the art, while maintaining its intended function. In some embodiments, the transmembrane domain comprises an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the amino acid sequences set forth in SEQ ID NOs: 29, 31, and/or 33. In some embodiments the transmembrane domain is encoded by a nucleic acid sequence comprising the nucleotide sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the nucleotide sequences set forth in SEQ ID NOs:, 30, 32, and/or 34. The transmembrane domain may be combined with any hinge domain and/or may comprise one or more transmembrane domains described herein.

In some embodiments, the CAR comprises: any transmembrane domain selected from the group consisting of the transmembrane domain of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR); any costimulatory signaling domains, and any intracellular domains or cytoplasmic domains described herein, or any of the other domains described herein that may be included in the CAR, and optionally a hinge domain.

In some embodiments, the CAR further comprises a spacer domain between the extracellular domain and the transmembrane domain of the CAR, or between the intracellular domain and the transmembrane domain of the CAR. In some embodiments, the spacer domain may be a short oligo- or polypeptide linker, e.g., between about 2 and about 10 amino acids in length. For example, glycine-serine doublet provides a particularly suitable linker between the transmembrane domain and the intracellular signaling domain of the subject CAR. Accordingly, the CAR of the present disclosure may comprise any of the transmembrane domains, hinge domains, or spacer domains described herein.

3. Hinge Domain

In some embodiments, a CAR of the present disclosure further comprises a hinge region. The hinge region of the CAR is a hydrophilic region which is located between the antigen binding domain and the transmembrane domain. In some embodiments, the hinge domain facilitates proper protein folding for the CAR. In some embodiments, the hinge domain is an optional component for the CAR. In some embodiments, the hinge domain comprises a domain selected from Fc fragments of antibodies, hinge regions of antibodies, CH2 regions of antibodies, CH3 regions of antibodies, artificial hinge sequences or combinations thereof. In some embodiments, the hinge domain is selected from but not limited to, a CD8α hinge, artificial hinges made of polypeptides that may be as small as, three glycines (Gly). In some embodiments, the hinge region is a hinge region polypeptide derived from a receptor. In some embodiments, the hinge region is a CD8-derived hinge region). In one embodiment, the hinge domain comprises an amino acid sequence derived from human CD8, or a variant thereof. In some embodiments, a subject CAR comprises a CD8α hinge domain and a CD8α transmembrane domain. In some embodiment, the CD8α hinge domain comprises the amino acid sequence set forth in SEQ ID NO: 27 or 35. In some embodiments, the CD8α hinge domain comprises the nucleotide sequence set forth in SEQ ID NO: 28 or 36.

In some embodiments the hinge domain comprises an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the amino acid sequences set forth in SEQ ID NO: 27 or 35.

In some embodiments the hinge domain is encoded by a nucleic acid sequence comprising the nucleotide sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the nucleotide sequences set forth in SEQ ID NO: 28 or 36.

In some embodiments, the hinge domain connects the antigen-binding domain to the transmembrane domain, which, is linked to the intracellular domain. In exemplary embodiments, the hinge region is capable of supporting the antigen binding domain to recognize and bind to the target antigen on the target cells. In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge domain has a length selected from about 4 to about 50, from about 4 to about 10, from about 10 to about 15, from about 15 to about 20, from about 20 to about 25, from about 25 to about 30, from about 30 to about 40, or from about 40 to about 50 amino acids. Suitable hinge regions can be readily selected and can be of any of a number of suitable lengths, such as from about 1 amino acid (e.g., Glycine (Gly) to about 20 amino acids, from about 2 to about 15, from about 3 to about 12 amino acids, including about 4 to about 10, about 5 to about 9, about 6 to about 8, or about 7 to about 8 amino acids, and can be about 1, about 2, about 3, about 4, about 5, about 6, or about 7 amino acids.

In some embodiments, the amino acid is a glycine (Gly). Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. In some embodiment, the hinge regions comprise glycine polymers (G)n, glycine-serine polymers. In some embodiments, the hinge region comprises glycine-serine polymers selected from the group consisting of (GS)n, (GSGGS)n and (GGGS)n, where n is an integer of at least one). In some embodiments, the hinge domain comprises an amino acid sequence of including, but not limited to, GGSG (SEQ ID NO: 121), GGSGG (SEQ ID NO: 122), GSGSG (SEQ ID NO: 123), GSGGG (SEQ ID NO: 124), GGGSG (SEQ ID NO: 125), GSSSG (SEQ ID NO: 126). In some embodiment, the hinge region comprises glycine-alanine polymers, alanine-serine polymers, or other flexible linkers known in the art.

In some embodiments, the hinge region is an immunoglobulin heavy chain hinge region. Immunoglobulin hinge region amino acid sequences are known in the art. In some embodiments, an immunoglobulin hinge domain comprises an amino acid sequence selected from the group consisting of DKTHT (SEQ ID NO: 130); CPPC (SEQ ID NO: 131); CPEPKSCDTPPPCPR (SEQ ID NO: 132) (see, e.g., Glaser et al., J. Biol. Chem. (2005) 280:41494-41503); ELKTPLGDTTHT (SEQ ID NO: 133); KSCDKTHTCP (SEQ ID NO: 134); KCCVDCP (SEQ ID NO:135); KYGPPCP (SEQ ID NO: 136); EPKSCDKTHTCPPCP (SEQ ID NO: 137) (human IgG1 hinge); ERKCCVECPPCP (SEQ ID NO: 138) (human IgG2 hinge); ELKTPLGDTTHTCPRCP (SEQ ID NO: 139) (human IgG3 hinge); SPNMVPHAHHAQ (SEQ ID NO: 49) (human IgG4 hinge); and the like.

In some embodiments, the hinge region is an immunoglobulin heavy chain hinge region. In some embodiments, the hinge is selected from CH1 and CH3 domains of IgGs (such as human IgG4). In some embodiments, the hinge domain comprises an amino acid sequence of a human IgG1, IgG2, IgG3, or IgG4 hinge domain. In some embodiments, the hinge region can include one or more amino acid substitutions and/or insertions and/or deletions compared to a wild-type (naturally-occurring) hinge region. In some embodiment, histidine at position 229 (His229) of human IgG1 hinge is substituted with tyrosine (Tyr). In some embodiments, the hinge domain comprises the amino acid sequence EPKSCDKTYTCPPCP (SEQ ID NO: 137).

4. Intracellular Domain

A CAR of the present disclosure also comprises an intracellular domain. The intracellular domain or otherwise the cytoplasmic domain of the CAR is responsible for activation of the cell in which the CAR is expressed. The term “intracellular domain” is thus meant to include any portion of the intracellular domain sufficient to transduce the activation signal. In one embodiment, the intracellular domain includes a domain responsible for an effector function. The term “effector function” refers to a specialized function of a cell. Effector function of a T cell, for example, may be cytolytic activity or helper activity including the secretion of cytokines. In one embodiment, the intracellular domain of the CAR includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

Examples of an intracellular domain for use in the invention include, but are not limited to, the cytoplasmic portion of a T cell receptor (TCR), and any co-stimulatory molecule, or any molecule that acts in concert with the TCR to initiate signal transduction in the T cell, following antigen receptor engagement, as well as any derivative or variant of these elements and any synthetic sequence that has the same functional capability.

In certain embodiments, the intracellular domain comprises an intracellular signaling domain. Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD2, CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD lib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, syk family tyrosine kinases (Syk, ZAP 70, etc.), src family tyrosine kinases (Lck, Fyn, Lyn, etc.), other co-stimulatory molecules described herein, any derivative, variant, or fragment thereof, any synthetic sequence of a co-stimulatory molecule that has the same functional capability, and any combination thereof.

In some embodiments, the intracellular signaling domain comprises an intracellular domain selected from the group consisting of cytoplasmic signaling domains of a human CD2, CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof. In some embodiments, the intracellular signaling domain comprises CD3 zeta intracellular signaling domain.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors. Additionally, intracellular signaling domains may include signaling domains used by NK and NKT cells such as signaling domains of NKp30 (B7-H6), and DAP 12, NKG2D, NKp44, NKp46, DAP10, and CD3z.

Intracellular signaling domains suitable for use in the CAR of the present disclosure include any desired signaling domain that transduces a signal in response to the activation of the CAR (i.e., activated by antigen and dimerizing agent). In some embodiments, a distinct and detectable signal e.g., comprises increased production of one or more cytokines by the cell; change in transcription of a target gene; change in activity of a protein; change in cell behavior (e.g., cell death); cellular proliferation; cellular differentiation; cell survival; and/or modulation of cellular signaling responses. e.g., In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present disclosure include immunoreceptor tyrosine-based activation motif (ITAM)-containing intracellular signaling polypeptides. In some embodiments, the intracellular signaling domain includes at least one at least two, at least three, at least four, at least five, or at least six ITAM motifs as described below. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcR gamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-zeta, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in the CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR. In some embodiment, the intracellular domain of the CAR comprises dual signaling domains. The dual signaling domains may include a fragment or domain from any of the molecules described herein. In some embodiments, the intracellular domain comprises 4-1BB costimulatory domain and CD3 zeta signaling domain; CD28 costimulatory domain and CD3 zeta signaling domain; CD2 costimulatory domain and CD3 zeta signaling domain. In some embodiments, the intracellular domain of the CAR includes any portion of a co-stimulatory molecule, such as at least one signaling domain from CD3, CD27, CD28, ICOS, 4-1BB, PD-1, T cell receptor (TCR), any derivative or variant thereof, any synthetic sequence thereof that has the same functional capability, and any combination thereof.

Further, variant intracellular signaling domains suitable for use in a subject CAR are known in the art. The YMFM motif is found in ICOS and is a SH2 binding motif that recruits both p85 and p50alpha subunits of PI3K, resulting in enhanced AKT signaling. In one embodiment, a CD28 intracellular domain variant may be generated to comprise a YMFM motif.

In one embodiment, the intracellular domain of a subject CAR comprises a CD3 zeta intracellular signaling domain comprising the amino acid sequence set forth in SEQ ID NO: 52 or SEQ ID NO: 54, which may be encoded by a nucleic acid sequence comprising the nucleotide sequence set forth in SEQ ID NO: 53 or SEQ ID NO: 55, respectively.

Tolerable variations of the intracellular domain will be known to those of skill in the art, while maintaining specific activity. In some embodiments, the intracellular domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 52 or 54. In some embodiments, the intracellular domain is encoded by a nucleic acid sequence comprising a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NO: 53 or 55.

5. Costimulatory Domain

In some embodiments, the intracellular domain comprises a costimulatory signaling domain and an intracellular signaling. In certain embodiments, the intracellular domain comprises a costimulatory signaling domain. In one embodiment, the intracellular domain of the CAR comprises a costimulatory signaling domain selected from the group consisting of a portion of a signaling domain from proteins in the TNFR superfamily, CD27, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR, any derivative or variant thereof, any synthetic sequence thereof that has the same functional capability, and any combination thereof.

In some embodiments, the costimulatory domain comprises one or more of a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof. In some embodiments, the costimulatory domain comprises one or more of a costimulatory domain of a protein selected from the group consisting of proteins in the CD28, 4-1BB (CD137), OX40 (CD134), CD27, CD2, or a combination thereof. In some embodiments, the costimulatory signaling domain comprises 4-1BB costimulatory domain. In some embodiments, the costimulatory signaling domain comprises CD2 costimulatory domain. In some embodiments, the costimulatory signaling domain comprises CD28 costimulatory domain.

In some embodiments, the costimulatory domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 37, 39, 41, 43, 46, 48, or 50. In some embodiments, the intracellular domain is encoded by a nucleic acid sequence comprising a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NO: 38, 40, 42, 44, 45, 47, 49, or 51.

In one embodiment, the intracellular domain of a subject CAR comprises an ICOS costimulatory domain and a CD3 zeta intracellular signaling domain. In one embodiment, the intracellular domain of a subject CAR comprises a CD28 costimulatory domain and a CD3 zeta intracellular signaling domain. In one embodiment, the intracellular domain of a subject CAR comprises a CD28 YMFM variant costimulatory domain and a CD3 zeta intracellular signaling domain. In one embodiment, the intracellular domain of a subject CAR comprises a CD27 costimulatory domain and a CD3 zeta intracellular signaling domain. In one embodiment, the intracellular domain of a subject CAR comprises a OX40 costimulatory domain and a CD3 zeta intracellular signaling domain. In one exemplary embodiment, the intracellular domain of a subject CAR comprises a 4-1BB costimulatory domain and a CD3 zeta intracellular signaling domain. In one exemplary embodiment, the intracellular domain of a subject CAR comprises a CD2 costimulatory domain and a CD3 zeta intracellular signaling domain.

B. Additional Antigen-Binding Polypeptides

In some embodiments, the modified T cell expresses an antigen-binding polypeptide, a cell surface receptor ligand, or a polypeptide that binds to a tumor antigen. In some instances, the antigen-binding domain comprises an antibody that recognizes a cell surface protein or a receptor expressed on a tumor cell. In some instances, the antigen-binding domain comprises an antibody that recognizes a tumor antigen. In some instances, the antigen-binding domain comprises a full length antibody or an antigen-binding fragment thereof, a Fab, a F(ab)2, a monospecific Fab2, a bispecific Fab2, a trispecific Fab2, a single-chain variable fragment (scFv), a diabody, a triabody, a minibody, a V-NAR, or a VhH.

C. Cell Surface Receptor Ligands

In some embodiments, a lentiviral vector or retroviral vector of the present disclosure further comprises a nucleic acid encoding a cell surface receptor ligand. In some instances, the ligand binds to a cell surface receptor expressed on a tumor cell. In some cases, the ligand comprises a wild-type protein or a variant thereof that binds to the cell surface receptor. In some instances, the ligand comprises a full-length protein or a functional fragment thereof that binds to the cell surface receptor. In some cases, the functional fragment comprises about 90%, about 80%, about 70%, about 60%, about 50%, or about 40% in length as compared to the full length version of the protein but retains binding to the cell surface receptor. In some cases, the ligand is a de novo engineered protein that binds to the cell surface receptor. Exemplary ligands include, but are not limited to, epidermal growth factor (EGF), platelet-derived growth factor (PDGF), or Wnt3A.

D. Tumor Antigens

In some embodiments, a lentiviral vector or retroviral vector of the present disclosure further comprises a nucleic acid encoding a polypeptide that binds to a tumor antigen. In some embodiments, the tumor antigen is associated with a hematologic malignancy. Exemplary tumor antigens include, but are not limited to, CD19, CD20, CD22, CD33/IL3Ra, ROR1, mesothelin, c-Met, PSMA, PSCA, Folate receptor alpha, Folate receptor beta, EGFRvIII, GPC2, Tn-MUC1, GDNF family receptor alpha-4 (GFRa4), fibroblast activation protein (FAP), and IL13Ra2. In some instances, the tumor antigen comprises CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1. In some instances, the polypeptide is a ligand of the tumor antigen, e.g., a full-length protein that binds to the tumor antigen, a functional fragment thereof, or a de novo engineered ligand that binds to the tumor antigen. In some instances, the polypeptide is an antibody that binds to the tumor antigen.

E. Engineered T Cell Receptors

In some embodiments, the antigen binding domain of a CAR described herein can be grafted to one or more constant domains of a T cell receptor (“TCR”) chain (e.g., a TCR alpha or TCR beta chain), to create a chimeric TCR. Chimeric TCRs can signal through the TCR complex upon antigen binding. For example, an scFv as disclosed herein, can be grafted to the constant domain, or at least a portion of the extracellular constant domain, the transmembrane domain of a TCR chain. As another example, an antibody fragment, for example a VL domain as described herein, can be grafted to the constant domain of a TCR alpha chain. Such chimeric TCRs may be produced, for example, by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

F. Switch Receptors and Dominant Negative Receptors

In one aspect, a lentiviral vector or retroviral vector of the present disclosure further comprises a nucleic acid encoding a dominant negative receptor, a switch receptor, or a combination thereof. In some embodiments, the lentiviral vector or retroviral vector described herein comprises a chimeric antigen receptor (CAR), and/or a dominant negative receptor. In some embodiments, the lentiviral vector or retroviral vector comprises a CAR, and/or a switch receptor. In some embodiments, the lentiviral vector or retroviral vector described herein comprises an engineered TCR, and a switch receptor. In some embodiments, the lentiviral vector or retroviral vector described herein comprises an engineered TCR, and a dominant negative receptor. In some embodiments, the lentiviral vector or retroviral vector described herein comprises a KIR, and a switch receptor. In some embodiments, the lentiviral vector or retroviral vector described herein further comprises a KIR, and a dominant negative receptor.

1. Switch Receptors

The present disclosure provides quick and efficient manufacturing processes for engineering modified immune cells comprising a CAR, or an exogenous TCR and/or a switch receptor. In some embodiments, the CAR, the TCR and/or the switch receptor are encoded by one or more nucleic acids. In some embodiments, the lentiviral vector or retroviral vector disclosed herein comprises one or more nucleic acid sequence encoding the CAR, the TCR and/or the switch receptor. In some embodiments, the nucleic acid sequence encoding the CAR is operably linked to a nucleic acid sequence encoding the switch receptor. In some embodiments, the switch receptor can enhance the efficiency of the CAR or the CAR expressing cell.

Tumor cells generate an immunosuppressive microenvironment that serves to protect them from immune recognition and elimination. This immunosuppressive microenvironment can limit the effectiveness of immunosuppressive therapies such as CAR-T or TCR-T cell therapy. For example, the secreted cytokine Transforming Growth Factor β (TGF β) directly inhibits the function of cytotoxic T cells and additionally induces regulatory T cell formation to further suppress immune responses. T cell immunosuppression due to TGFβ in the context of prostate cancers has been previously demonstrated. To reduce the immunosuppressive effects of TGF on the immune cells can be modified to express an engineered TGFβR comprising the extracellular ligand-binding domain of the TGFβR fused to the intracellular signaling domain of, for example, Interleukin-12 receptor (IL12R; TGFβR-IL12R). Therefore, a modified immune cell comprising a switch receptor may bind a negative signal transduction molecule in the microenvironment of the modified immune cell, and convert the negative signal transduction signal of an inhibitory molecule may have on the modified immune cell into a positive signal that stimulate the modified immune cell. A switch receptor of the present disclosure may be designed to reduce the effects of a negative signal transduction molecule, or to convert the negative signal into a positive signal, by virtue of comprising an intracellular domain associated with the positive signal.

As used herein, the term “switch receptor” refers to a molecule designed to reduce the effect of a negative signal transduction molecule on a modified immune cell of the present disclosure. The switch receptor comprises: a first domain that is derived from a first polypeptide that is associated with a negative signal (a signal transduction that suppresses or inhibits a cell or T cell activation); and a second domain that is derived from a second polypeptide that is associated with a positive signal (a signal transduction signal that stimulate a cell or a T cell). In some embodiments, the protein associated with the negative signal is selected from the group consisting of CTLA4, PD-1, TGFβRII, BTLA, VSIG3, VSIG8, and TIM-3. In some embodiments, the protein associated with the positive signal is selected from the group consisting of CD28, 4-1BB, IL12Rβ1, IL12Rβ2, CD2, ICOS, and CD27.

In one embodiment, the first domain comprises at least a portion of the extracellular domain of the first polypeptide that is associated with a negative signal, and the second domain comprises at least a portion of the intracellular domain of the second polypeptide that is associated with a positive signal. As such, a switch receptor comprises an extracellular domain associated with a negative signal fused to an intracellular domain associated with a positive signal. In some embodiments, the switch receptor comprises an extracellular domain of a signaling protein associated with a negative signal, a transmembrane domain, and an intracellular domain of a signaling protein associated with a positive signal. In some embodiments, the transmembrane domain of the switch receptor is selected from the transmembrane of the protein associated with a negative signal or the transmembrane domain of the protein associated with the negative signal. In some embodiments, the transmembrane domain of the switch receptor is selected from a transmembrane domain of a protein selected from the group consisting of CTLA4, PD-1, VSIG3, VSIG8, TGFβRII, BTLA, TIM-3, CD28, 4-1BB, IL12Rβ1, IL12Rβ2, CD2, ICOS, and CD27.

In some embodiments, the switch receptor is selected from the group consisting of PD-1-CD28, PD-1A132L-CD28, PD-1-CD27, PD-1A132L-CD27, PD-1-4-1BB, PD-1A132L-4-1BB, PD-1-ICOS, PD-1A132L-ICOS, PD-1-IL12Rβ1, PD-1A132L-IL12Rβ1, PD-1-IL12Rβ2, PD-1A132L-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

2. Dominant Negative Receptors

The present disclosure provides a quick and efficient manufacturing process for engineering modified immune cells comprising a CAR, or an exogenous TCR and a dominant negative receptor. In some embodiments, the CAR, the TCR and/or the switch receptor are encoded by one or more nucleic acid. In some embodiments, the lentiviral vector or retroviral vector disclosed herein comprises one or more nucleic acid sequence encoding the CAR, the TCR and/or the dominant negative receptor. In some embodiments, the nucleic acid sequence encoding the CAR is operably linked to a nucleic acid sequence encoding the dominant negative receptor. In some embodiments, the dominant negative receptor enhances the efficiency of the CAR or the CAR expressing cell.

As used herein, the term “dominant negative receptor” refers to a molecule designed to reduce the effect of a negative signal transduction molecule (e.g., the effect of a negative signal transduction molecule on a modified immune cell of the present disclosure). A dominant negative receptor is a truncated variant of a wild-type protein associated with a negative signal. In some embodiments, the protein associated with a negative signal the protein associated with the negative signal is selected from the group consisting of CTLA4, PD-1, BTLA, TGFβRII, VSIG3, VSIG8, and TIM-3.

A dominant negative receptor of the present disclosure may bind a negative signal transduction molecule (e.g., CTLA4, PD-1, BTLA, TGFβRII, VSIG3, VSIG8, and TIM-3) by virtue of an extracellular domain associated with the negative signal, may reduce the effect of the negative signal transduction molecule. For example, a modified immune cell comprising a dominant negative receptor may bind a negative signal transduction molecule in the microenvironment of the modified immune cell, but this binding will not transduce this signal inside the cell to modify the activity of the modified T cell. Rather, the binding sequesters the negative signal transduction molecule and prevents its binding to endogenous receptor/ligand, thereby reducing the effect of the negative signal transduction molecule may have on the modified immune cell. As such, to reduce the immunosuppressive effects of certain molecule, immune cells can be modified to express a dominant negative receptor that is a dominant negative receptor.

In some embodiments, the dominant negative receptor comprises a truncated variant of a wild-type protein associated with a negative signal. In some embodiments, the dominant negative receptor comprises a variant of a wild-type protein associated with a negative signal comprising an extracellular domain, a transmembrane domain, and substantially lacking an intracellular signaling domain. In some embodiments, the dominant negative receptor comprises an extracellular domain of a signaling protein associated with a negative signal, and a transmembrane domain. In some embodiments, the dominant negative receptor is PD-1, CTLA4, BTLA, TGFβRII, VSIG3, VSIG8, or TIM-3 dominant negative receptor. In some embodiments, the dominant negative receptor is PD-1, or TGFβRII dominant negative receptor. Tolerable variations of the dominant negative receptor will be known to those of skill in the art, while maintaining its intended biological activity (e.g., blocking a negative signal and/or sequestering a molecule having a negative signal when expressed in a cell).

G. Chemokine and Cytokine as Immune Enhancing Factors for Improved Fitness

The present disclosure provides quick and efficient manufacturing processes for engineering modified immune cells comprising a CAR, or an exogenous TCR and/or an immune enhancing factor that improves the fitness of the engineered immune cells. In some embodiments, the immune enhancing factor or a functional derivative thereof is a polypeptide that enhances the immune cell function.

In some embodiments, a polypeptide that enhances the immune cell function, or a functional derivative thereof is selected from a chemokine, a chemokine receptor, a cytokine, a cytokine receptor, Interleukin-7 (IL-7), Interleukin-7 receptor (IL-7R), Interleukin-15 (IL-15), Interleukin-15 receptor (IL-15R), Interleukin-21 (IL-21), Interleukin-18 (IL-18), Interleukin-18 receptor (IL-18R),CCL21, CCL19, or a combination thereof. In some embodiments, a chemokine, a chemokine receptor, a cytokine, a cytokine receptor, IL-7, IL-7R, IL-15, IL-15R, IL-21, IL-18, IL-18R, C-C Motif Chemokine Ligand 21 (CCL21), or C-C Motif Chemokine Ligand 19 (CCL19) is an immune function-enhancing factor that improves the fitness of the claimed modified immune cell. Without wishing to be bound by theory, the addition of a nucleic acid encoding a chemokine, a chemokine receptor, a cytokine, a cytokine receptor, IL-7, IL-7R, IL-15, IL-15R, IL-21, IL-18, IL-18R, CCL21, or CCL19 to the modified immune cell of the present disclosure enhances the immunity-inducing effect and antitumor activity of the modified immune cell.

1. T Cell Infiltration

Without wishing to be bound by theory, interleukins and chemokines, may promote increase T cell priming and/or T cell infiltration in a solid tumor. For instance, in microsatellite stable colorectal cancers (CRCs) with low T cell infiltration, IL-15 promotes T cell priming. In some embodiments, the combination of a CAR and chemokine/interleukine receptor complex promotes T cell priming. Furthermore, IL-15 may induce NK cell infiltration. In some embodiments, response to an IL-15/IL-15RA complex can result in NK cell infiltration. In certain embodiments, the modified immune cell described herein further comprises an IL-15/IL-15Ra complex. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. In some embodiments, IL-15 stimulates Natural Killer cells to eliminate (e.g., kill) pancreatic cancer cells. In some embodiments, therapeutic response to a modified immune cell described herein further comprising IL-15/IL15Ra is associated with Natural Killer cell infiltration in an animal model of colorectal cancer. In some embodiments, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra.

The ineffectiveness of CAR T cell therapy against solid tumors is partially caused by the limited recruitment and accumulation of immune cells and CAR T cells in solid tumors. One approach to solve this problem is to engineer CAR T cells that mimic the function of T-zone fibroblastic reticular cells (FRC). The lymph node is responsible for detecting pathogens and immunogens. The T-zone contains three types of cells: (1) innate immunity cells such as dendritic cells, monocytes, macrophages, and granulocytes; (2) adaptive immunity cells, such as CD4 and CD8 lymphocytes, and (3) stromal cells (FRCs). These cells cooperate to mount an effective immune response against a pathogen by facilitating the activation, differentiation and maturation of CD4 T cells. FRCs are particularly important because they form a network that allows dendritic cells and T cells to travel throughout the lymph node, and attracts B cells. In particular, FRCs provide a network for: (i) the recruitment of naive T cells, B cells and dendritic cells to the lymph node by releasing two chemokines (CCL21 and CCL19); (ii) T cell survival by secreting IL-7, which is a survival factor particularly for naive T cells; and (iii) trafficking of CD4 T cells toward the germinal center (GC; a different part of the lymph node). Accordingly, a CAR armored with exogenous CCL21, or CCL19 and IL-7, will enhance the recruitment of T cells, B cells and dendritic cells to solid tumors. In some embodiments, the modified immune cells engineered by the method disclosed herein comprises a lentiviral vector or retroviral vector comprising a nucleic acid encoding an immune function-enhancing factor, and a CAR. In that embodiment, the nucleic acid encoding the immune function-enhancing factor is a nucleic acid encoding interleukin-7 and a nucleic acid encoding CCL19 or CCL21.

In some embodiments, the nucleic acid of the immune function-enhancing factor (i.e., chemokine, the chemokine receptor, the cytokine, the cytokine receptor, IL-7, IL-7R, IL-15, IL-15R, IL-21, IL-18, CCL21, or CCL19) is fused to a CAR. In some embodiments, the chemokine, the chemokine receptor, the cytokine, the cytokine receptor, IL-7, IL-7R, IL-15, IL-15R, IL-21, IL-18, CCL21, or CCL19 is fused to a CAR via a self-cleaving peptide, such as a P2A, a T2A, an E2A, or an F2A.

2. T Cell Priming (IL-18)

The present disclosure provides quick and efficient manufacturing processes for engineering modified immune cells comprising a CAR, or an exogenous TCR and/or polypeptide which enhances T cell priming (i.e., T cell priming polypeptide). In some embodiments, the polypeptide that enhances T cell priming (ETP) is selected from the group consisting of a costimulatory molecule, a soluble cytokine, a polypeptide involved in antigen presentation, a polypeptide involved in trafficking and/or migration, or a polypeptide involved in dendritic cell targeting, or a functional fragment or variant thereof. In an embodiment, the T cell priming costimulatory molecule is selected from the group consisting of CD70, CD83, CD80, CD86, CD40, CD154, CD137L (4-1BBL), CD252 (OX40L), CD275 (ICOS-L), CD54 (ICAM-1), CD49a, CD43, CD48, CD112 (PVRL2), CD150 (SLAM), CD155 (PVR), CD265 (RANK), CD270 (HVEM), TL1A, CD127, IL-4R, GITR-L, CD160, CD258, TIM-4, CD153 (CD30L), CD200R (OX2R), CD44, ligands thereof, and functional fragments and variants thereof. In an embodiment, the soluble cytokine is selected from the group consisting of: IL-2, IL-12, IL-6, IL-7, IL-15, IL-18, IL-21, GM-CSF, IL-18, IL-21, IL-27, and functional fragments and variants thereof. In an embodiment, the polypeptide involved in antigen presentation is selected from the group consisting of CD64, MHC I, MHC II, and functional fragments and variants thereof. In an embodiment, the polypeptide involved in trafficking and/or migration is selected from the group consisting of CD183, CCR2, CCR6, CD50, CD197, CD58, CD62L, and functional fragments and variants thereof. In an embodiment, the polypeptide involved in DC targeting is selected from the group consisting of TLR ligands, anti-DEC-205 antibody, an anti-DC-SIGN antibody, and functional fragments and variants thereof.

In some embodiments, the T cell priming polypeptide comprises an amino acid sequence of interleukin 2 (IL-2) (e.g., GenBank Acc. No. AAB46833.1), or a nucleic acid sequence of IL-2 (e.g., GenBank Acc. No. S82692.1). In some embodiments, the T cell priming polypeptide comprises an amino acid sequence of interleukin 12 (IL-12) (e.g., GenBank Acc. No. AAD16432.1), or a nucleic acid sequence of IL-12 (e.g., GenBank Acc. No. AF101062.1). In some embodiments, the T cell priming polypeptide comprises an amino acid sequence of interleukin 6 (IL-6) (e.g., GenBank Acc. No. AAD13886.1 or NP_000591.1), or a nucleic acid sequence of IL-6 (e.g., GenBank Acc. No. 556892.1 or NM_000600.3). In some embodiments, the T cell priming polypeptide comprises an amino acid sequence of interleukin 7 (IL-7) (e.g., GenBank Acc. No. AAH47698.1 or NP_000871.1), or a nucleic acid sequence of IL-7 (e.g., GenBank Acc. No. BC047698.1 or NM_000880.3). In some embodiments, the T cell priming polypeptide comprises an amino acid sequence of interleukin 15 (IL-15) (e.g., GenBank Acc. No. AAU21241.1), or a nucleic acid sequence of IL-15 (e.g., GenBank Acc. No. AY720442.1). In some embodiments, the T cell priming polypeptide comprises an amino acid sequence of interleukin 18 (IL-18) (e.g., GenBank Acc. No. AAK95950.1), or a nucleic acid sequence of IL-18 (e.g., GenBank Acc. No. AY044641.1). In some embodiments, the T cell priming polypeptide comprises an amino acid sequence of interleukin 21 (IL-21) (e.g., GenBank Acc. No. AAG29348.1), or a nucleic acid sequence of IL-21 (e.g., GenBank Acc. No. AF254069.1). In some embodiments, the T cell priming polypeptide comprises an amino acid sequence of GM-CSF (e.g., GenBank Acc. No. AAA52578.1), or a nucleic acid sequence of GM-CSF (e.g., GenBank Acc. No. Mil 1220.1). In some embodiments, the T cell priming polypeptide is an IL-18.

In some embodiments, the expression of the CAR or CARs does not substantially affect the level of expression of the T cell priming polypeptide in the armored CAR T cell. In some embodiments, the CAR comprises an antigen binding domain that binds the antigen, and the expression of the T cell priming polypeptide does not substantially affect the level of expression or cell-killing function of the CAR or CARs in the armored CART cell.

In some embodiments, the lentiviral vector or retroviral vector disclosed herein comprises and delivers more than one T cell priming polypeptides. In an embodiment, the lentiviral vector or retroviral vector comprises 2, 3, 4, 5, 6 or more nucleic acids encoding one or more T cell priming polypeptides; and further comprises a nucleic acid sequence encoding a CAR. In some embodiments, the co-delivery of one or more T cell priming polypeptides does not affect (e.g., substantially decrease or substantially inhibit), the expression or activity of the co-expressed CAR in the armored CAR T cell or armored CAR-expressing immune cell. In some embodiments, the CAR does not affect (e.g., substantially decrease or substantially inhibit), the expression or activity of the co-expressed T cell priming polypeptide.

### III. Nucleic Acids and Expression Vectors

A. Nucleic Acid Encoding a CAR

The present disclosure provides nucleic acid molecules encoding one or more CAR constructs described herein. The nucleic acid molecule can be a messenger RNA transcript. The nucleic acid molecule can also be a DNA construct.

In one aspect, the present disclosure provides an isolated nucleic acid molecule encoding a chimeric antigen receptor (CAR), which may comprise a single chain antibody or a single chain antibody fragment comprising an anti-CD19 binding domain, a transmembrane domain, a costimulatory, and an intracellular signaling domain. In some embodiments, the anti-CD19 binding domain is encoded by a nucleic acid sequence selected from SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216. In some embodiments, the anti-CD19 binding domain is encoded by a nucleic acid isolated nucleic acid molecule having 90%, about 91%, about 92%, about 93%, about 94%, about 95%, about 96%, about 97%, about 98%, or about 99% identity to SEQ ID NO: 21, SEQ ID NO:24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216. In some embodiments, the anti-CD19 binding domain comprise a nucleotide sequence of SEQ ID NO: 21. In some embodiments, the anti-CD19 binding domain comprise a nucleotide sequence of SEQ ID NO: 24. In some embodiments, the anti-CD19 binding domain comprise a nucleotide sequence of SEQ ID NO: 102. In some embodiments, the anti-CD19 binding domain comprise a nucleotide sequence of SEQ ID NO: 103. In some embodiments, the anti-CD19 binding domain comprise a nucleotide sequence of SEQ ID NO: 104. In some embodiments, the anti-CD19 binding domain comprise a nucleotide sequence of SEQ ID NO: 114. In some embodiments, the anti-CD19 binding domain comprise a nucleotide sequence of SEQ ID NO: 115. In some embodiments, the anti-CD19 binding domain comprise a nucleotide sequence of SEQ ID NO: 116. In some embodiments, the anti-CD19 binding domain comprise a nucleotide sequence of SEQ ID NO: 117. In some embodiments, the anti-CD19 binding domain comprise a nucleotide sequence of SEQ ID NO: 118. In some embodiments, the anti-CD19 binding domain comprise a nucleotide sequence of SEQ ID NO: 119. In some embodiments, the anti-CD19 binding domain comprise a nucleotide sequence of SEQ ID NO: 120. In some embodiments, the anti-CD19 binding domain comprise a nucleotide sequence of SEQ ID NO: 216. In some embodiments, the anti-CD19 binding domain comprise a nucleotide sequence of SEQ ID NO: 225.

In some embodiments, the CAR comprises an anti-CD19 binding domain comprising a light chain variable domain comprising a light chain complementary determining region 1 (LC CDR1) of SEQ ID NO: 1, a light chain complementary determining region 2 (LC CDR2) of SEQ ID NO: 2, and a light chain complementary determining region 3 (LC CDR3) of SEQ ID NO: 3; and a heavy chain variable domain comprising a heavy chain complementary determining region 1 (HC CDR1) of SEQ ID NO: 4, a heavy chain complementary determining region 2 (HC CDR2) of SEQ ID NO: 5, and a heavy chain complementary determining region 3 (HC CDR3) of SEQ ID NO: 6.

In some embodiments, the CAR comprises an anti-CD19 binding domain comprising a light chain variable domain comprising a light chain complementary determining region 1 (LC CDR1) of SEQ ID NO: 193, a light chain complementary determining region 2 (LC CDR2) of SEQ ID NO: 194, and a light chain complementary determining region 3 (LC CDR3) of SEQ ID NO: 195; and a heavy chain variable domain comprising a heavy chain complementary determining region 1 (HC CDR1) of SEQ ID NO: 196, a heavy chain complementary determining region 2 (HC CDR2) of SEQ ID NO: 197, and a heavy chain complementary determining region 3 (HC CDR3) of SEQ ID NO: 198. In another embodiment, the anti-CD19 binding domain comprises a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

The light chain variable region may comprise the amino acid sequence of SEQ ID NO: 7 or 199; or an amino acid sequence having at least about 90%, about 91%, about 92%, about 93%, about 94%, about 95%, about 96%, about 97%, about 98%, or about 99% identity to the amino acid sequence of SEQ ID NO: 7 or 199. Alternatively, the heavy chain variable region may comprise the amino acid sequence of SEQ ID NO: 8 or 200, or an amino acid sequence having at least about 90%, about 91%, about 92%, about 93%, about 94%, about 95%, about 96%, about 97%, about 98%, or about 99% identity to the amino acid sequence of SEQ ID NO: 8 or 200.

In some embodiments, the anti-CD19 binding domain comprises the light chain variable region comprises the amino acid sequence of SEQ ID NO: 7 and the heavy chain variable region comprises the amino acid sequence of SEQ ID NO: 8.

In some embodiments, the light chain variable region comprises the amino acid sequence of SEQ ID NO: 199 and the heavy chain variable region comprises the amino acid sequence of SEQ ID NO: 200. In some embodiments, the CD19 binding domain may be a scFv.

In some embodiments, the anti-CD19 binding domain comprises an amino acid sequence selected from the group consisting of SEQ ID NO: 9, 18, 64, 75, 86, 190, 157, 212, 201, 226, 179, 168, and 146, or a sequence having 90%, about 91%, about 92%, about 93%, about 94%, about 95%, about 96%, about 97%, about 98%, or about 99% identity to SEQ ID NO: 9, 18, 64, 75, 86, 190, 157, 212, 201, 226, 179, 168, or 146.

In some embodiments, the anti-CD19 binding domain comprises a light chain variable region or a heavy chain variable region encoded by a nucleic acid sequence selected from a group consisting of SEQ ID NO: 19, SEQ ID NO: 20, SEQ ID NO: 21, SEQ ID NO: 22, SEQ ID NO: 23, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216. The anti-CD19 binding domain may comprise a light chain variable region or a heavy chain variable region encoded by a nucleic acid sequence having 90%, about 91%, about 92%, about 93%, about 94%, about 95%, about 96%, about 97%, about 98%, or about 99% identity to SEQ ID NO: 19-24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

In some embodiments of the isolated nucleic acid molecule described herein, the transmembrane domain of the CAR may comprise a transmembrane domain of a protein selected from the group consisting of the alpha, beta or zeta chain of the T-cell receptor, CD2, CD28, CD3 epsilon, CD45, CD4, CD5, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain comprises an amino acid sequence selected from SEQ ID NO: 29, 31, or 33, or an amino acid sequence having 90%, about 91%, about 92%, about 93%, about 94%, about 95%, about 96%, about 97%, about 98%, or about 99% identity to SEQ ID NO: 29, 31, or 33. In Some embodiments, the transmembrane domain comprises a nucleic acid sequence selected from SEQ ID NO: 30, SEQ ID NO: 32, or SEQ ID NO: 34 or a sequence having 90%, about 91%, about 92%, about 93%, about 94%, about 95%, about 96%, about 97%, about 98%, or about 99% identity to SEQ ID NO: 30, 32, or 34. In some embodiments, the transmembrane domain comprises a CD8 transmembrane domain, and/or an amino acid sequence of SEQ ID NO: 29; or an amino acid sequence having about 90%, about 91%, about 92%, about 93%, about 94%, about 95%, about 96%, about 97%, about 98%, or about 99% identity to SEQ ID NO: 29. In some embodiments, the transmembrane domain comprises a nucleic acid sequence of SEQ ID NO: 30, or a sequence having 90%, about 91%, about 92%, about 93%, about 94%, about 95%, about 96%, about 97%, about 98%, or about 99% identity to SEQ ID NO: 30.

In some embodiments of the isolated nucleic acid molecule described herein, the CAR further comprises a hinge domain. In some embodiments, the anti-CD19 binding domain is connected to the transmembrane domain by a hinge region as described herein. In some embodiments, the hinge region may be from a protein selected from the group consisting of an Fc fragment of an antibody, a hinge region of an antibody, a CH2 region of an antibody, a CH3 region of an antibody, an artificial spacer sequence, an IgG hinge, a CD8 hinge, and any combination thereof.

In some embodiments of the isolated nucleic acid molecule described herein, the CAR comprises a costimulatory domain, which may be a functional signaling domain of a protein selected from the group consisting of a TNFR superfamily member, OX40 (CD134), CD2, CD5, CD7, CD27, CD28, CD30, CD40, PD-1, CD8, ICAM-1, lymphocyte function-associated antigen-1 (LFA-1), CD11a, CD18, ICOS (CD278), LIGHT, NKG2C, B7-H3, a ligand that specifically binds to CD83, DAP10, DAP12, Lck, Fas and 4-1BB (CD137). In some embodiments, the costimulatory domain comprises an amino acid sequence selected from SEQ ID NO: 37, SEQ ID NO: 39, SEQ ID NO: 41, SEQ ID NO: 43, SEQ ID NO: 46, SEQ ID NO: 48, or SEQ ID NO: 50, or a sequence having about 90%, about 91%, about 92%, about 93%, about 94%, about 95%, about 96%, about 97%, about 98%, or about 99% identity to SEQ ID NO: 37, 39, 41, 43, 46, 48, or 50. In some embodiments, the costimulatory domain comprises a nucleic acid sequence selected from SEQ ID NO: 38, SEQ ID NO:40, SEQ ID NO:42, SEQ ID NO:44, SEQ ID NO: 45, SEQ ID NO:47, or SEQ ID NO:49, or a nucleic acid sequence having 90%, about 91%, about 92%, about 93%, about 94%, about 95%, about 96%, about 97%, about 98%, or about 99% identity to SEQ ID NO: 38, 40, 42, 44, 45, 47, or 49.

In some embodiments of the isolated nucleic acid molecule described herein, the CAR may comprise an intracellular signaling domain. The signaling domain may be from a protein selected from the group consisting of CD3 zeta, FcγRIII, FcsRI, a cytoplasmic tail of an Fc receptor, an immunoreceptor tyrosine-based activation motif (ITAM) bearing cytoplasmic receptor, TCR zeta, FcR gamma, FcR beta, CD3 gamma, CD3 delta, CD3 epsilon, CD5, CD22, CD79a, CD79b, and CD66d. In that embodiments, the intracellular signaling domain comprises the intracellular signaling domain of CD3 zeta, the amino acid sequence of SEQ ID NO: 52 or 54, or a sequence having about 90%, about 91%, about 92%, about 93%, about 94%, about 95%, about 96%, about 97%, about 98%, or about 99% identity to SEQ ID NO: 52 or 54. Alternatively, the intracellular signaling domain comprises the nucleic acid sequence of SEQ ID NO: 53 or 55, or a sequence having about 90%, about 91%, about 92%, about 93%, about 94%, about 95%, about 96%, about 97%, about 98%, or about 99% identity to SEQ ID NO: 53 or 55.

In some embodiments, the CAR comprises a functional 4-1BB costimulatory domain and a functional CD3 zeta intracellular signaling domain. In some embodiments, the CAR comprises the amino acid sequence of SEQ ID NO: 37, SEQ ID NO: 52, or SEQ ID NO:54 or a sequence having about 90%, about 91%, about 92%, about 93%, about 94%, about 95%, about 96%, about 97%, about 98%, or about 99% identity to an amino acid sequence of SEQ ID NO: 37, SEQ ID NO: 52 or SEQ ID NO:54.

The intracellular signaling domain may comprise the sequence of SEQ ID NO: 37 and the sequence of SEQ ID NO: 52 or SEQ ID NO: 54, or a sequence having about 90%, about 91%, about 92%, about 93%, about 94%, about 95%, about 96%, about 97%, about 98%, or about 99% identity to SEQ ID NO: 37, SEQ ID NO: 52 or SEQ ID NO: 54. These sequences may be expressed in the same frame and as a single polypeptide chain. In some embodiments, the nucleic acid sequence comprises a sequence of SEQ ID NO: 38, or a sequence having 90%, about 91%, about 92%, about 93%, about 94%, about 95%, about 96%, about 97%, about 98%, or about 99% identity to SEQ ID NO: 38. In some embodiments, the nucleic acid sequence comprises a sequence of SEQ ID NO: 53 or SEQ ID NO: 55, or a sequence having about 90%, about 91%, about 92%, about 93%, about 94%, about 95%, about 96%, about 97%, about 98%, or about 99% identity to SEQ ID NO: 53 or 55.

In some embodiments of the isolated nucleic acid molecule described herein, the CAR further comprises a leader sequence. The leader sequence may comprise the amino acid of SEQ ID NO: 25.

One aspect of the present disclosure provides an isolated nucleic acid molecule comprising an scFv comprising an anti-CD19 binding domain described herein. One aspect of the present disclosure provides an isolated nucleic acid molecule comprising a CAR comprising an anti-CD19 binding domain described herein, a transmembrane domain, a costimulatory domain and an intracellular domain. In some embodiments, the anti-CD19 binding domain may comprise LC CDR1 of SEQ ID NO: 1, LC CDR2 of SEQ ID NO: 2, and LC CDR3, HC CDR1 of SEQ ID NO: 4, HC CDR2 of SEQ ID NO: 5, and HC CDR3 of SEQ ID NO: 6; or LC CDR1 of SEQ ID NO: 193, LC CDR2 of SEQ ID NO: 194, LC CDR3 of SEQ ID NO: 195; HC CDR1 of SEQ ID NO: 196, HC CDR2 of SEQ ID NO: 197, and HC CDR3 of SEQ ID NO: 198; or any LC CDR1, LC CDR2, LC CDR3, HC CDR1, HC CDR2, and HC CDR3 disclosed in Table 2, the transmembrane domain is selected from CD28 or CD8 transmembrane domain, the costimulatory domain comprises an intracellular signaling domain of a protein selected from the group consisting of OX40, CD27, CD2, CD28, ICOS, and 4-1BB; and the intracellular signaling domain comprises CD3-zeta or FcR gamma.

In another embodiment, the anti-CD19 binding domain comprises a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

One aspect of the present disclosure provides an isolated nucleic acid molecule comprising a CAR comprising an anti-CD19 binding domain, a transmembrane domain, a costimulatory domain, and an intracellular domain. The anti-CD19 binding domain comprises the amino acid sequence of SEQ ID NO: 9, 18, 64, 75, 86, 190, 157, 212, 201, 226, 179, 168, or 146. The transmembrane domain is selected from CD28 or CD8 transmembrane domain, the costimulatory domain may comprise an intracellular signaling domain of a protein selected from the group consisting of OX40, CD27, CD2, CD28, ICOS, and 4-1BB; and an intracellular signaling domain comprising CD3-zeta or FcR gamma.

One aspect of the present disclosure provides an isolated nucleic acid molecule comprising an scFv comprising an anti-CD19 binding domain. One aspect of the present disclosure provides an isolated nucleic acid molecule comprising a CAR comprising an anti-CD19 binding domain (e.g., scFv), a transmembrane domain, a costimulatory domain, and an intracellular domain. The anti-CD19 binding domain comprises the amino acid sequence of SEQ ID NO: 9, 18, 64, 75, 86, 190, 157, 212, 201, 226, 179, 168, or 146; the transmembrane domain comprising the amino acid sequence of selected from the group consisting of SEQ ID NO: 29, 31, and 33; the costimulatory domain comprising an amino acid sequence selected from the group consisting of SEQ ID NO: 37, SEQ ID NO: 39, SEQ ID NO: 41, SEQ ID NO: 43, SEQ ID NO: 46, SEQ ID NO: 48, and SEQ ID NO: 50; and the intracellular signaling domain comprising the amino acid sequence of SEQ ID NO: 52 or SEQ ID NO: 54.

One aspect of the present disclosure provides an isolated nucleic acid molecule comprising an anti-CD19 binding domain comprising the amino acid sequence of SEQ ID NO: 9, 18, 64, 75, 86, 190, 157, 212, 201, 226, 179, 168, or 146; a transmembrane domain comprising the amino acid sequence of SEQ ID NO: 29; a costimulatory domain comprising the amino acid sequence of SEQ ID NO: 37; and an intracellular signaling domain comprising of SEQ ID NO: 52 or 54.

One aspect of the present disclosure provides an isolated nucleic acid comprising an amino acid sequence encoded by a nucleic acid sequence selected from the group consisting of SEQ ID NO: 66, 77, 88, 148, 170, 181, 203, 214, 159, 192, 23, and 20; and/or an amino acid sequence encoded by a nucleic acid sequence selected from the group consisting of SEQ ID NO: 65, 76, 87, 147, 169, 180, 202, 213, 158, 191, 22, and 19.

One aspect of the present disclosure provides an isolated nucleic acid comprising a sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216.

One aspect of the present disclosure provides an isolated polypeptide molecule encoded by the nucleic acid molecule described herein. The isolated polypeptide may comprise a sequence selected from the group consisting of SEQ ID NO: 63, 74, 85, 145, 167, 178, 200, 211, 156, 189, 17, 8, 62, 73, 84, 144, 166, 177, 199, 210, 155, 188, 16, and 7.

B. Expression Vectors

One aspect of the present disclosure provides a vector (e.g., expression vector) comprising the isolated nucleic acid described. Another aspect of the present disclosure provides A vector comprising a first polynucleotide comprising a constitutive promoter operably linked to a nucleic acid encoding a chimeric antigen receptor (CAR) described herein; and (b) a second polynucleotide comprising a nucleic acid encoding a polypeptide that enhances an immune cell function, or a functional derivative thereof.

In some embodiments, the CAR comprises a single chain antibody or a single chain antibody fragment comprising an anti-CD19 binding domain (e.g., P1-P13), a transmembrane domain, a costimulatory, and an intracellular signaling domain. In some embodiments, the first polynucleotide is operably linked to the second polypeptide via a linker peptide.

In some embodiments, the polypeptide that enhances an immune cell function, or a functional derivative thereof can be selected from the group consisting of a cytokine, an interferon, a chemokine, an antibody or antibody fragment, a checkpoint inhibitor antagonist, a dominant negative receptor, a switch receptor, and a combination thereof.

In some embodiments, the polypeptide that enhances an immune cell function, or a functional derivative thereof can be a chemokine, a chemokine receptor, a cytokine, a cytokine receptor, and a combination thereof. Alternatively, the polypeptide that enhances an immune cell function, or a functional derivative thereof can be a cytokine selected from the group consisting of Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), Interleukin-18 (IL-18), Interleukin-18 receptor (IL-18R), Interleukin-21 (IL-21), granulocyte macrophage colony stimulating factor, alpha, beta or gamma interferon, erythropoietin, and a combination thereof. The polypeptide that enhances an immune cell function, or a functional derivative thereof can also be a chemokine selected from CCL21, CCL19, or a combination thereof. In some embodiments, the polypeptide that enhances an immune cell function, or a functional derivative thereof is IL-18.

In some embodiments of the methods disclosed herein, the vector encodes a CAR comprising an anti-CD19 binding domain that comprise a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2. In some embodiments, the anti-CD19 binding domain comprises an amino acid sequence selected from the group consisting of SEQ ID NO: 9, 18, 64, 75, 86, 190, 157, 212, 201, 226, 179, 168, and 146, or a sequence having 90%, about 91%, about 92%, about 93%, about 94%, about 95%, about 96%, about 97%, about 98%, or about 99% identity to SEQ ID NO: 9, 18, 64, 75, 86, 190, 157, 212, 201, 226, 179, 168, and 14.

In another embodiment, the anti-CD19 binding domain comprises: (a) a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; or (b) a sequence having 90%, about 91%, about 92%, about 93%, about 94%, about 95%, about 96%, about 97%, about 98%, or about 99% identity to SEQ ID NO: 21, SEQ ID NO: 24 SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

One aspect of the present disclosure provides a vector comprising: (a) a first polynucleotide comprising a constitutive promoter operably linked to a nucleic acid encoding an anti-CD19 chimeric antigen receptor (CAR), where the CAR comprises: (i) an anti-CD19 binding domain comprising: a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2; (ii) a transmembrane domain selected from CD28 or CD8 transmembrane domain; (iii) a costimulatory domain comprising an intracellular signaling domain of a protein selected from the group consisting of OX40, CD27, CD2, CD28, ICOS, and 4-1BB; and (iv) an intracellular signaling domain comprising of CD3-zeta; and (b) a second polynucleotide comprising a nucleic acid encoding Interleukin-18 (IL-18), and/or Interleukin-18 receptor (IL-18R).

In some embodiments, the first polynucleotide is operably linked to the second polypeptide via a linker peptide selected from the group consisting of F2A, E2A, P2A, T2A, and Furin-(G4S)2-T2A (F-GS2-T2A).

In some embodiments, the anti-CD19 binding domain comprises LC CDR1 of SEQ ID NO: 1, LC CDR2 of SEQ ID NO: 2, and LC CDR3, HC CDR1 of SEQ ID NO: 4, HC CDR2 of SEQ ID NO: 5, and HC CDR3 of SEQ ID NO: 6; or (2) LC CDR1 of SEQ ID NO: 193, LC CDR2 of SEQ ID NO: 194, LC CDR3 of SEQ ID NO: 195; HC CDR1 of SEQ ID NO: 196, HC CDR2 of SEQ ID NO: 197, and HC CDR3 of SEQ ID NO: 198.

The vector may be selected from the group consisting of a DNA, a RNA, a plasmid, a lentivirus vector, an adenoviral vector, or a retroviral vector.

The lentiviral vector may be based on a virus selected from the group consisting of a retrovirus, an alpha retrovirus, a beta retrovirus, a gamma retrovirus, a delta retrovirus, and an epsilon retrovirus. For example, the lentiviral vector may be based on a Human immunodeficiency virus (HIV), an Equine infectious anemia virus (EIAV), a visna-maedi virus (VMV) virus, a caprine arthritis-encephalitis virus (CAEV), a feline immunodeficiency virus (Hy), a bovine immune deficiency virus (BIV), a VISNA virus, and a simian immunodeficiency virus (SIV). In some embodiments, the lentiviral vector may be pseudotyped with an envelope glycoprotein (Env) from a virus selected from the group consisting of a murine leukemia virus (MLV), a vesicular stomatitis virus (VSV) Indiana strain, VSV New Jersey strain, Cocal virus, Chandipura virus, Piry virus, spring viremia of carp virus (SVCV), Sigma virus, infectious hematopoietic necrosis virus (IHNV), Mokola virus, rabies virus CVS virus, Isfahan virus, Alagoas virus, Calchaqui virus, Jurona vrus, La Joya virus, Maraba virus, Feline Endogenous Retrovirus (RD114) Envelope Protein, Perinet virus, Yug Bugdanovac virus, a prototypic foamy virus (PFV), and gibbon ape leukemia virus (GaLV). In some embodiments, the lentiviral vector may be pseudotyped with an envelope glycoprotein (Env) selected from the group consisting of vesicular stomatitis virus (VSV) Indiana strain, VSV New Jersey strain, and Cocal virus.

In some embodiments of the lentiviral vector described herein, the viral envelope protein (Env) comprises a VSV-G glycoprotein selected from the group consisting of VSV-G of the Indiana strain, VSV-G of the New Jersey strain, the Cocal virus envelope protein, the Isfahan virus envelope protein, Chandipura virus envelope protein, Pyri virus envelope protein, a murine leukemia virus (MLV) envelope glycoprotein, a SVCV virus envelope protein, and a variant thereof. The lentiviral vector may also comprise a nucleotide sequence encoding a heterologous VSV-G envelope protein.

The heterologous VSV G envelope protein may be codon-optimized for human expression. Alternatively, the heterologous VSV G envelope protein may be a VSV G protein variant. In some embodiments, the lentiviral vector comprises a nucleotide sequence encoding the VSV-G envelope protein, or a VSV G protein variant.

In some embodiments of the lentiviral vector described herein, the heterologous envelope protein may be under the control of a transcriptional regulatory element. The transcriptional regulatory element maybe a promoter selected from a eukaryotic promoter or a constitutive promoter.

The lentiviral vector described herein can further comprise a transcriptional regulatory element and the transcriptional regulatory element may be upstream of the heterologous envelope glycoprotein (i.e., in the 5′ direction of the nucleotide sequence encoding the heterologous envelope glycoprotein). For example, the transcriptional regulatory element may control the expression (i.e., transcription and, accordingly, but optionally, translation) of the nucleic acid encoding the heterologous envelope glycoprotein. In some embodiments, the transcriptional regulatory element is constitutively active or is a constitutive promoter. In exemplary embodiments, the constitutively active transcriptional regulatory element or the constitutive promoter may be a cytomegalovirus (CMV) promoter, such as the CMV major immediate early promoter (CMV IE1), a murine stem cell virus promoter, Elongation Factor-1 alpha promoter (EF-1 alpha), a viral simian virus 40 (SV40) (e.g., early or late), a Moloney murine leukemia virus (MoMLV), an ubiquitin C promoter, a phosphoglycerokinase (PGK) promoter, a Rous sarcoma virus (RSV), or herpes simplex virus (HSV) (thymidine kinase) promoter.

In other embodiments, the activity of the transcriptional regulatory element may be inducible or the promoter may be an inducible promoter. In some embodiments, the transcriptional regulatory element may be a eukaryotic promoter, such as phosphoglycerate kinase promoter. Other transcriptional regulatory elements, including prokaryotic and eukaryotic, constitutive and inducible promoters, and origins of replication are known in the art.

In some embodiments, the lentiviral vector described herein can be structured and arranged so that the expression of the proteins, enzymes, and viral elements necessary for producing retroviral particles (i.e., cis-acting and trans-acting genes) are under the control of a transcriptional regulatory element. In a preferred embodiment, the lentiviral vector can further comprise a transcriptional regulatory element and the transcriptional regulatory element is upstream (i.e. in the 5′ direction) of the proteins, enzymes, and viral elements necessary for producing retroviral particles (i.e. cis-acting and trans-acting genes) and, optionally, the transcriptional regulatory element controls the expression (i.e. transcription or translation) of the nucleic acid encoding proteins, enzymes, and viral elements necessary for producing retroviral particles (i.e. cis-acting and trans-acting genes). In some embodiments, the transcriptional regulatory element may be constitutively active or may be a constitutive promoter.

In some embodiments, the lentiviral vectors described herein and nucleic acids encoding the heterologous envelope protein may be amplified or produced prior to the introduction into producer cells and, accordingly, prior to the production of viral particles. In some embodiment, the lentiviral vectors and nucleic acids encoding the other proteins, enzymes, and elements necessary for retroviral particle production may be amplified or produced prior to the introduction into producer cells, and, accordingly, the production of the retroviral proteins.

In some embodiments, the lentiviral vectors and nucleic acids encoding the heterologous envelope protein may be structured and arranged such that a transcriptional control element drives the transcription, and therefore translation, of the heterologous envelope protein in a producer cell to facilitate the production the lentiviral particles. In some embodiments, the lentiviral vectors and nucleic acids encoding the proteins, enzymes, viral elements (i.e. cis- and trans-acting genes, including rev and gag/pol) necessary for the production of the retroviral particles may be structured and arranged so that a transcriptional control element may drive the transcription, and therefore translation, of the proteins, enzymes, viral elements (i.e. cis- and trans-acting genes, including rev and gag/pol) in a producer cell so that the producer cell produces the retroviral particles.

In some embodiment, the retroviral or lentiviral vector described herein comprises a transcriptional regulatory elements. In some embodiments, the transcriptional regulatory element is a promoter selected from an eukaryotic promoter or a constitutive promoter. Physiologic promoters (e.g., an EF-1α promoter) can be less likely to induce integration mediated genotoxicity, and can abrogate the ability of the retroviral vector to transform stem cells. Other physiological promoters suitable for use in a retroviral or lentiviral vector are known to those of skill in the art and can be incorporated into exemplary embodiments of the nucleic acid vector. In some embodiment, the promoter is an elongation-factor-1-alpha promoter (EF-1α promoter). Use of an EF-1α promoter can increase the efficiency in expression of downstream transgenes (e.g., a TCR and/or CAR encoding nucleic acid sequence).

In some embodiments, the lentiviral or retroviral vector further comprises a non-requisite cis-acting sequence that can improve titers and gene expression. One non-limiting example of a non-requisite cis-acting sequence is the central polypurine tract and central termination sequence (cPPT/CTS) which is important for efficient reverse transcription and nuclear import. Other non-requisite cis-acting sequences are known to those of skill in the art and can be incorporated into the lentiviral or retroviral vector particle.

In some embodiments, the lentiviral or retroviral vector disclosed herein further comprises a posttranscriptional regulatory element. Posttranscriptional regulatory elements can improve RNA translation, improve transgene expression and stabilize RNA transcripts. One example of a posttranscriptional regulatory element is the woodchuck hepatitis virus posttranscriptional regulatory element (WPRE). Accordingly, in some embodiments a nucleic acid vector further comprises a WPRE sequence. Various posttranscriptional regulator elements are known to those of skill in the art and can be incorporated into the lentiviral or retroviral vector.

The lentiviral or retroviral vector disclosed herein can further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, the lentiviral or retroviral vector comprises a 3′ U3 deleted LTR, a non-functional LTR and/or lacks a functional 3′ or 5′ LTR. Accordingly, the lentiviral or retroviral vector disclosed herein can comprise any combination of the elements described herein to enhance the efficiency of functional expression of transgenes. For example, a lentiviral or retroviral vector can comprise a WPRE sequence, cPPT sequence, RRE sequence, 5′LTR, 3′ U3 deleted LTR′ in addition to a nucleic acid encoding for a TCR or CAR.

In some embodiments, the further comprising a promoter, a rev response element (RRE), a poly(A) tail, a 3′ UTR, a woodchuck hepatitis virus posttranscriptional regulatory element (WPRE); and/or a cPPT sequence. The promoter may be a constitutive promoter. In some embodiments, the promoter is selected from the group consisting of an EF-1alpha promoter, a PGK-1 promoter, a truncated PGK-1 promoter, an UBC promoter, a CMV promoter, a CAGG promoter, and an SV40 promoter. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, the promoter is an EF-1 promoter. The promoter may comprise the sequence of SEQ ID NO: 101.

In some embodiments, the vector comprises the isolated nucleic acid molecule comprising a CAR described herein operably linked via a linker peptide to a nucleic acid sequence encoding a switch receptor, a dominant negative receptor, or a polypeptide that can enhance an immune cell function, or a functional derivative.

In some embodiments, the linker peptide is selected from F2A, E2A, P2A, T2A, or Furin-(G4S)2-T2A (F-GS2-T2A). Alternatively, the linker may comprise the amino acid sequence of SEQ ID NO: 92, SEQ ID NO:94, SEQ ID NO:96, or SEQ ID NO: 99. The linker may comprise the nucleic acid sequence of SEQ ID NO: 93, 95, 97, or 98.

C. Methods of Introducing Nucleic Acids into a Cell

Methods of introducing nucleic acids into a cell include physical, biological, chemical methods, and combination thereof. Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics (e.g., gene gun), transfection, lipofection (e.g., cationic liposome), polymer encapsulation, or the like. The host cell (e.g., immune cell or CD4⁺ and CD8⁺ cell) may be grown and expanded in culture before introduction of the expression vectors, followed by the appropriate treatment for introduction and integration of the vectors. The host cells (e.g., immune cells) may then be expanded and may be screened by virtue of a marker present in the vectors. Methods for producing cells including vectors and/or exogenous nucleic acids are well-known in the art.

In some embodiments, the host cell (e.g., immune cell, CD4⁺ and CD8⁺ cell) or population of host cells (e.g., population of immune cells, or CD4⁺ and CD8⁺ cells) can be modified using any method known in the art, such as activation, expansion, induction of apoptosis, genetic manipulation, induction of antigen-specificity. In some embodiments, the host cell (e.g., immune cell, CD4⁺ and CD8⁺ cell) or population of host cells (e.g., population of immune cells, or CD4⁺ and CD8⁺ cells) can be modified by the addition of cytokines, cross-linking specific receptors, addition of antigens, introduction of nucleic acid molecules (DNA, RNA, and/or modified versions thereof), protein agents, addition of drugs or small molecules, or any combination thereof. In some embodiments, the introduction of exogenous nucleic acid molecules comprises viral transfection (transduction), non-viral transfection, electroporation, lipofection, cationic liposome mediated transfection using lipofection, polymer encapsulation, peptide mediated transfection, or biolistic particle delivery systems such as “gene guns”.

Regardless of the method used to introduce isolated nucleic acids molecule described herein into a host cell or otherwise expose a cell to the CD19 CAR of the present invention, to confirm the presence of the nucleic acids in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular peptide (e.g., immunological means (ELISAs and Western blots) or by assays described herein to identify agents falling within the scope of the invention.

Moreover, the nucleic acids may be introduced by any means, such as transducing the expanded host cells (e.g., immune cells), transfecting the expanded host cells (e.g., immune cells), and electroporating the expanded host cells (e.g., immune cells). One isolated nucleic acid molecule may be introduced by one method and another nucleic acid may be introduced into the host cell (e.g., immune cells) by a different method.

1. Biological Methods

Biological methods for introducing a polynucleotide of interest into a host cell (e.g., immune cell) include the use of DNA and RNA vectors. Viral vectors, and especially retroviral vectors (viral transfection), have become the most widely used method for inserting genes into mammalian (e.g., human cells). Viral vectors can be derived from lentivirus, poxviruses, herpes simplex virus I, adenoviruses and adeno-associated viruses, and the like.

In some embodiments, a nucleic acid encoding a subject CAR, a subject engineered TCR, a subject KIR, a subject antigen-binding polypeptide, a subject cell surface receptor ligand, a subject tumor antigen, a subject switch receptor, a subject dominant negative receptor, and/or a subject polypeptide that enhances immune function (e.g., T cell priming or T cell infiltration) can be introduced into a cell with an expression vector (viral transfection). Expression vectors (e.g., lentiviral vector or retroviral vector) comprising a nucleic acid encoding a subject CAR, a subject engineered TCR, a subject KIR, a subject antigen-binding polypeptide, a subject cell surface receptor ligand, a subject tumor antigen, a subject switch receptor, a subject dominant negative receptor, and/or a subject polypeptide that enhances immune function (e.g., T cell priming or T cell infiltration) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

In some embodiments, the nucleic acids, encoding a subject CAR (e.g., a CD-19 CAR), a subject engineered TCR, a subject KIR, a subject antigen-binding polypeptide, a subject cell surface receptor ligand, a subject tumor antigen, a subject switch receptor, a subject dominant negative receptor, and/or a subject polypeptide that enhances immune function (e.g., T cell priming or T cell infiltration), are introduced into the immune cell by viral transduction. In some embodiments, the viral vector is selected from the group consisting of a retroviral vector, sendai viral vectors, adenoviral vectors, adeno-associated virus vectors, and lentiviral vectors. Various markers that may be used are known in the art, and may include hprt, neomycin resistance, thymidine kinase, hygromycin resistance, etc.

The modified immune cell, CD4⁺ and CD8⁺ cell or population of immune cells, or CD4⁺ and CD8⁺ cells of the present disclosure (e.g., comprising a nucleic acid encoding a subject CAR, a subject engineered TCR, a subject KIR, a subject antigen-binding polypeptide, a subject cell surface receptor ligand, a subject tumor antigen, a subject switch receptor, subject dominant negative receptor, and/or a subject polypeptide that enhances immune function (e.g., T cell priming or T cell infiltration)) may be produced by stably transfecting host cells (e.g. immune cells) with an expression vector including a nucleic acid of the present disclosure.

Transfected cells (i.e. immune cells) expressing a nucleic acid encoding a CAR, a KIR, a TCR, a KIR, an antigen-binding polypeptide, a cell surface receptor ligand, a tumor antigen, a subject switch receptor, a subject dominant negative receptor, and/or a subject polypeptide that enhances immune function (e.g., T cell priming or T cell infiltration) of the present disclosure may be expanded ex vivo. In some embodiments, transfected cells (i.e. immune cells) expressing a nucleic acid encoding a CAR, a KIR, a TCR, a KIR, an antigen-binding polypeptide, a cell surface receptor ligand, a tumor antigen, a subject switch receptor, subject dominant negative receptor, and/or a subject polypeptide that enhances immune function (e.g., T cell priming or T cell infiltration) of the present disclosure are not expanded ex vivo.

Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection).

2. Physical Methods

Physical methods for introducing a polynucleotide (RNA, or DNA) or an expression vector into a host cell (e.g., an immune cell) include lipofection, particle bombardment, microinjection, electroporation, and the like. The expression vector or polynucleotide can be introduced into target cells using commercially available methods which include electroporation (Amaxa Nucleofector-II (Amaxa Biosystems, Cologne, Germany)), (ECM 830 (BTX) (Harvard Instruments, Boston, MA) or the Gene Pulser II (BioRad, Denver, CO), Multiporator (Eppendorf, Hamburg Germany).

Regardless of the method used to introduce isolated nucleic acids molecule described herein into a host cell or otherwise expose a cell to the CD19 CAR of the present invention, to confirm the presence of the nucleic acids in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular peptide (e.g., immunological means (ELISAs and Western blots) or by assays described herein to identify agents falling within the scope of the invention.

Moreover, the nucleic acids may be introduced by any means, such as transducing the expanded host cells (e.g., immune cells), transfecting the expanded host cells (e.g., immune cells), and electroporating the expanded host cells (e.g., immune cells). One isolated nucleic acid molecule may be introduced by one method and another nucleic acid may be introduced into the host cell (e.g., immune cells) by a different method.

### IV. Car T Cells

One aspect of the present disclosure provides a modified cell, a modified immune cell, or a modified CD4⁺ and CD8⁺ cell engineered by the methods described herein. The modified cell is a modified immune cell, a modified natural killer (NK) cell, a modified natural killer T (NKT) cell, or a modified T cell. The modified cell is a modified T cell or a modified human T cell. The modified T cell can be a CD8⁺ T cell. The modified cell contemplated herein can be an autologous cell, heterologous cell, or an allogeneic cell.

In some embodiments, the modified cell (e.g., modified immune cell, or a modified CD4⁺ and CD8⁺ cell) comprises a chimeric antigen receptor (CAR) comprising a single chain antibody or a single chain antibody fragment comprising an anti-CD19 binding domain, a transmembrane domain, a costimulatory, and an intracellular signaling domain

In some embodiments, the modified cell (e.g., modified immune cell, or a modified CD4⁺ and CD8⁺ cell) comprises the isolated nucleic acid molecule described herein. In some embodiments, the nucleic acid molecule comprises a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216. In some embodiments, the nucleic acid molecule comprises a nucleic acid sequence having 90%, about 91%, about 92%, about 93%, about 94%, about 95%, about 96%, about 97%, about 98%, or about 99% identity to SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

In some embodiments, the modified cell (e.g., modified immune cell, or a modified CD4⁺ and CD8⁺ cell) comprises an isolated polypeptide encoded by the nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216.

In some embodiments, the modified cell (e.g., modified immune cell, or a modified CD4⁺ and CD8⁺ cell) comprises a CAR comprising a single chain antibody or a single chain antibody fragment comprising an anti-CD19 binding domain, a transmembrane domain, a costimulatory, and an intracellular signaling domain. In that embodiment, the anti-CD19 binding domain comprises a light chain variable domain comprising a light chain complementary determining region 1 (LC CDR1) of SEQ ID NO: 1, a light chain complementary determining region 2 (LC CDR2) of SEQ ID NO: 2, and a light chain complementary determining region 3 (LC CDR3) of SEQ ID NO: 3; and a heavy chain variable domain comprising a heavy chain complementary determining region 1 (HC CDR1) of SEQ ID NO: 4, a heavy chain complementary determining region 2 (HC CDR2) of SEQ ID NO: 5, and a heavy chain complementary determining region 3 (HC CDR3) of SEQ ID NO: 6. In another embodiment, the anti-CD19 binding domain comprises a light chain variable domain comprising a light chain variable domain comprising a light chain complementary determining region 1 (LC CDR1) of SEQ ID NO: 193, a light chain complementary determining region 2 (LC CDR2) of SEQ ID NO: 194, and a light chain complementary determining region 3 (LC CDR3) of SEQ ID NO: 195; and a heavy chain variable domain comprising a heavy chain complementary determining region 1 (HC CDR1) of SEQ ID NO: 196, a heavy chain complementary determining region 2 (HC CDR2) of SEQ ID NO: 197, and a heavy chain complementary determining region 3 (HC CDR3) of SEQ ID NO: 198. In another embodiment, the anti-CD19 binding domain comprises a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

In some embodiments, the anti-CD19 binding domain comprises the light chain variable region comprising the amino acid sequence of SEQ ID NO: 7 or 199; or an amino acid sequence having at least about 90%, about 91%, about 92%, about 93%, about 94%, about 95%, about 96%, about 97%, about 98%, or about 99% identity to SEQ ID NO: 7 or 199; and/or the heavy chain variable region comprises the amino acid sequence of SEQ ID NO: 8 or 200, an amino acid sequence having at least about 90%, about 91%, about 92%, about 93%, about 94%, about 95%, about 96%, about 97%, about 98%, or about 99% identity to SEQ ID NO: 8 or 200.

In some embodiments, the modified cell (e.g., modified immune cell, or a modified CD4⁺ and CD8⁺ cell) comprises a CAR comprising the light chain variable region comprises the amino acid sequence of SEQ ID NO: 7 and the heavy chain variable region comprises the amino acid sequence of SEQ ID NO: 8. Alternatively, the light chain variable region comprises the amino acid sequence of SEQ ID NO: 199 and the heavy chain variable region comprises the amino acid sequence of SEQ ID NO: 200. In some embodiments, the modified cell (e.g., modified immune cell, or a modified CD4⁺ and CD8⁺ cell) comprises a CAR comprising a CD-19 scFv. The anti-CD19 binding domain comprises an amino acid sequence selected from the group consisting of SEQ ID NO: 9, 18, 64, 75, 86, 190, 157, 212, 201, 226, 179, 168, and 146, or a sequence having 90%, about 91%, about 92%, about 93%, about 94%, about 95%, about 96%, about 97%, about 98%, or about 99% identity to SEQ ID NO: 9, 18, 64, 75, 86, 190, 157, 212, 201, 226, 179, 168, or 146.

In some embodiments, the modified cell (e.g., modified immune cell, or a modified CD4⁺ and CD8⁺ cell) comprises a CAR comprising the anti-CD19 binding domain encoded by a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216. In some embodiments, the anti-CD19 binding domain is encoded by a nucleic acid sequence having 90%, about 91%, about 92%, about 93%, about 94%, about 95%, about 96%, about 97%, about 98%, or about 99% identity to SEQ ID NO: 21, or 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

In some embodiments, the modified cell (e.g., modified immune cell, or a modified CD4⁺ and CD8⁺ cell) comprises a CAR comprising the anti-CD19 binding domain comprising a light chain variable region or a heavy chain variable region encoded by a nucleic acid sequence selected from the group consisting of SEQ ID NO: 19, SEQ ID NO: 20, SEQ ID NO:21, SEQ ID NO:22, SEQ ID NO: 23, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216. Alternatively, the anti-CD19 binding domain comprising a light chain variable region or a heavy chain variable region encoded by a nucleic acid sequence having 90%, about 91%, about 92%, about 93%, about 94%, about 95%, about 96%, about 97%, about 98%, or about 99% identity to SEQ ID NO: 19-24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

In some embodiments, the modified cell (e.g., modified immune cell, or a modified CD4⁺ and CD8⁺ cell) comprises a CAR comprising the anti-CD19 binding domain comprising a light chain variable region or a heavy chain variable region encoded by a nucleic acid sequence selected from the group consisting of SEQ ID NO: 19, SEQ ID NO: 20, SEQ ID NO:21, SEQ ID NO:22, SEQ ID NO: 23, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216 and transmembrane domain comprising a transmembrane domain of a protein selected from the group consisting of the alpha, beta or zeta chain of the T-cell receptor, CD2, CD28, CD3 epsilon, CD45, CD4, CD5, CD8, CD9, CD16, CD22, CD33, CD37, CD64, CD80, CD86, CD134 (OX-40), CD137 (4-1BB), CD 154 (CD40L), CD278 (ICOS), CD357 (GITR), Toll-like receptor 1 (TLR1), TLR2, TLR3, TLR4, TLR5, TLR6, TLR7, TLR8, and TLR9. In that embodiment, the transmembrane domain may comprise an amino acid sequence selected from SEQ ID NO: 29, 31, or 33, or an amino acid sequence 90%, about 91%, about 92%, about 93%, about 94%, about 95%, about 96%, about 97%, about 98%, or about 99% identity to SEQ ID NO: 29, 31, or 33. Alternatively, the transmembrane domain may comprise a nucleic acid sequence selected from SEQ ID NO: 30, SEQ ID NO: 32, or SEQ ID NO: 34 or a sequence having 90%, about 91%, about 92%, about 93%, about 94%, about 95%, about 96%, about 97%, about 98%, or about 99% identity to SEQ ID NO: 30, 32, or 34. The transmembrane domain may comprise a CD8 transmembrane domain, and/or an amino acid sequence of SEQ ID NO: 29; or an amino acid sequence having about 90%, about 91%, about 92%, about 93%, about 94%, about 95%, about 96%, about 97%, about 98%, or about 99% identity to SEQ ID NO: 29. The transmembrane domain may comprise a nucleic acid sequence of SEQ ID NO: 30, or a sequence having 90%, about 91%, about 92%, about 93%, about 94%, about 95%, about 96%, about 97%, about 98%, or about 99% identity to SEQ ID NO: 30.

In some embodiments, the modified cell (e.g., modified immune cell, or a modified CD4⁺ and CD8⁺ cell) comprises a CAR comprising the anti-CD19 binding domain comprising a light chain variable region or a heavy chain variable region encoded by a nucleic acid sequence selected from the group consisting of SEQ ID NO: 19, SEQ ID NO: 20, SEQ ID NO:21, SEQ ID NO:22, SEQ ID NO: 23, and SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; and the anti-CD19 binding domain may be connected to the transmembrane domain by a hinge region. The hinge region may be from a protein selected from the group consisting of an Fc fragment of an antibody, a hinge region of an antibody, a CH2 region of an antibody, a CH3 region of an antibody, an artificial spacer sequence, an IgG hinge region, a CD8 hinge, and any combination thereof. The hinge may comprises the amino acid sequence of SEQ ID NO: 27 or SEQ ID NO: 35, or a sequence having about 90%, about 91%, about 92%, about 93%, about 94%, about 95%, about 96%, about 97%, about 98%, or about 99% identity to SEQ ID NO: 27 or 35. The hinge region may comprise a CD8 hinge region and/or the amino acid sequence of SEQ ID NO: 27, or a sequence having about 90%, about 91%, about 92%, about 93%, about 94%, about 95%, about 96%, about 97%, about 98%, or about 99% identity to SEQ ID NO: 27. In that embodiment, the CAR further comprises a functional signaling domain (e.g., the costimulatory domain) of a protein selected from the group consisting of a TNFR superfamily member, OX40 (CD134), CD2, CD5, CD7, CD27, CD28, CD30, CD40, PD-1, CD8, ICAM-1, lymphocyte function-associated antigen-1 (LFA-1), CD11a, CD18, ICOS (CD278), LIGHT, NKG2C, B7-H3, a ligand that specifically binds to CD83, DAP10, DAP12, Lck, Fas and 4-1BB (CD137).

In some embodiments, the modified cell (e.g., modified immune cell, or a modified CD4⁺ and CD8⁺ cell) comprises a CD19-4BBz CAR, a CD19CD2z, a CD19CD2z CAR, a CD19CD27z CAR, a CD190×40z CAR, a CD1928z YMFM, a CD19ICOSz, a CD19ICOS-1z.

One aspect of the present disclosure provides a modified cell, a modified immune cell, or a modified CD4⁺ and CD8⁺ cell comprising a chimeric antigen receptor (CAR) comprising an anti-CD19 binding domain, a switch receptor, a dominant negative receptor, and/or a polypeptide that enhances an immune cell function. In some embodiments, the switch receptor comprises a first polypeptide that comprises at least a portion of an inhibitory molecule selected from the group consisting of PD1, TGFβR, TIM-2 and BTLA, conjugated to a second polypeptide that comprises a positive signal from an intracellular signaling domain selected from the group consisting of OX40, CD27, CD28, IL-12R, ICOS, and 4-1BB. In some embodiments, the switch receptor is selected from the group consisting of PD-1-CD28, PD-1A132L-CD28, PD-1-CD27, PD-1A132L-CD27, PD-1-4-1BB, PD-1A132L-4-1BB, PD-1-ICOS, PD-1A132L-ICOS, PD-1-IL12Rβ1, PD-1A132L-Th12Rβ1, PD-1-IL12Rβ2, PD-1A 132L-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

In some embodiments, the dominant negative receptor comprises a truncated variant of a receptor selected from the group consisting of PD1, TGFβR, TIM-2 and BTLA. In some embodiments, the dominant negative receptor is PD-1, CTLA4, BTLA, TGFβRII, VSIG3, VSIG8, or TIM-3 dominant negative receptor.

In some embodiments, a polypeptide that enhances the immune cell function, or a functional derivative thereof is selected from a chemokine, a chemokine receptor, a cytokine, a cytokine receptor, Interleukin-7 (IL-7), Interleukin-7 receptor (IL-7R), Interleukin-15 (IL-15), Interleukin-15 receptor (IL-15R), Interleukin-21 (IL-21), Interleukin-18 (IL-18), Interleukin-18 receptor (IL-18R),CCL21, CCL19, or a combination thereof. In some embodiments, a chemokine, a chemokine receptor, a cytokine, a cytokine receptor, IL-7, IL-7R, IL-15, IL-15R, IL-21, IL-18, IL-18R, C-C Motif Chemokine Ligand 21 (CCL21), or C-C Motif Chemokine Ligand 19 (CCL19) is an immune function-enhancing factor that improves the fitness of the claimed modified immune cell. Without wishing to be bound by theory, the addition of a nucleic acid encoding a chemokine, a chemokine receptor, a cytokine, a cytokine receptor, IL-7, IL-7R, IL-15, IL-15R, IL-21, IL-18, IL-18R, CCL21, or CCL19 to the modified immune cell of the present disclosure enhances the immunity-inducing effect and antitumor activity of the modified immune cell.

In some embodiments, the polypeptide that enhances an immune cell function, or a functional derivative thereof selected from the group consisting of a chemokine, a chemokine receptor, a cytokine, a cytokine receptor, Interleukin-7 (IL-7), Interleukin-7 receptor (IL-7R), Interleukin-15 (IL-15), Interleukin-15 receptor (IL-15R), Interleukin-21 (IL-21), Interleukin-18 (IL-18), Interleukin-18 receptor (IL-18R), CCL21, CCL19, and a combination thereof.

In some embodiments, the modified cell, the modified immune cell, or the modified CD4⁺ and CD8⁺ cell comprises a CAR (e.g., CD19 CAR), an engineered TCR (e.g., a CD19 TCR), a KIR (a CD19 KIR), an antigen-binding polypeptide, a cell surface receptor ligand, a tumor antigen, a switch receptor, a dominant negative receptor, and/or a polypeptide that enhances immune function (e.g., T cell priming or T cell infiltration).

In some embodiments, a polypeptide that enhances immune function is selected from the group consisting of a chemokine, a chemokine receptor, a cytokine, a cytokine receptor, IL-7, IL-7R, IL-15, IL-15R, IL-21, IL-18, IL-18R, CCL21, CCL19, or a combination thereof.

Another aspect of the present disclosure provides a population of modified cells, a population of modified immune cells, or a population of modified CD4⁺ and CD8⁺ cells comprising a lentiviral vector described herein. In some embodiments, the modified CD4⁺ and CD8⁺ cell engineered described herein is for use in the production of a protein of interest (e.g., a CD19 CAR).

In some embodiments of the modified CD4⁺ and CD8⁺ cell engineered described herein, the protein of interest may be selected from the group consisting of an industrial protein, or a therapeutic protein. In some embodiments, the protein of interest may be selected from the group consisting of enzymes, regulatory proteins, receptors, peptides, peptide hormones, cytokines, membrane or transport proteins, vaccine antigens, antigen-binding proteins, immune stimulatory proteins, allergens, full-length antibodies or antibody fragments or derivatives; single chain antibodies, (scFv), Fab fragments, Fv fragments, single domain antibodies (VH or VL fragment), domain antibodies, camelid single domain antibodies (VHH), nanobodies and a combination thereof.

One aspect of the present disclosure provides a method of making a modified cell comprising transfecting a cell with the isolated nucleic acid molecule described herein. In some embodiments, the isolated nucleic acid molecule encoded a CAR described herein. In some embodiments, the isolated nucleic acid molecule comprises a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216.

In some embodiments, the isolated nucleic acid molecule comprises a nucleic acid sequence having 90%, about 91%, about 92%, about 93%, about 94%, about 95%, about 96%, about 97%, about 98%, or about 99% identity to SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

One aspect of the present disclosure provides a method of making a modified cell comprising transfecting a cell with a nucleic acid encoding the anti-CD19 binding domain described herein; or a vector comprising the isolated nucleic acid described herein.

### V. Methods of Generating a Modified T Cell

One aspect of the present disclosure provides a method for manufacturing a population of engineered immune cells comprising the novel CD19 binders disclosed herein. Another aspect of the present disclosure provides a method of making a modified cell comprising transfecting a cell with any of the vector described herein. In some embodiments, the vector comprises a first polynucleotide comprising a constitutive promoter operably linked to a nucleic acid encoding a chimeric antigen receptor (CAR); and (b) a second polynucleotide comprising a nucleic acid encoding a polypeptide that enhances an immune cell function, or a functional derivative thereof. In that embodiment, the CAR comprises a single chain antibody or a single chain antibody fragment comprising an anti-CD19 binding domain, a transmembrane domain, a costimulatory, and an intracellular signaling domain. In some embodiments, the first polynucleotide is operably linked to the second polypeptide via a linker peptide.

In some embodiments, the polypeptide that enhances an immune cell function, or a functional derivative thereof can be selected from the group consisting of a cytokine, an interferon, a chemokine, an antibody or antibody fragment, a checkpoint inhibitor antagonist, a dominant negative receptor, a switch receptor, and a combination thereof.

In some embodiments, the polypeptide that enhances an immune cell function, or a functional derivative thereof can be a chemokine, a chemokine receptor, a cytokine, a cytokine receptor, and a combination thereof. Alternatively, the polypeptide that enhances an immune cell function, or a functional derivative thereof can be a cytokine selected from the group consisting of Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), Interleukin-18 (IL-18), Interleukin-18 receptor (IL-18R), Interleukin-21 (IL-21), granulocyte macrophage colony stimulating factor, alpha, beta or gamma interferon, erythropoietin, and a combination thereof. The polypeptide that enhances an immune cell function, or a functional derivative thereof can also be a chemokine selected from CCL21, CCL19, or a combination thereof. In some embodiments, the polypeptide that enhances an immune cell function, or a functional derivative thereof is IL-18.

In some embodiments of the methods disclosed herein, the vector encodes a CAR comprising an anti-CD19 binding domain that comprise a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2. In some embodiments, the anti-CD19 binding domain comprises an amino acid sequence selected from the group consisting of SEQ ID NO: 9, 18, 64, 75, 86, 190, 157, 212, 201, 226, 179, 168, and 146, or a sequence having 90%, about 91%, about 92%, about 93%, about 94%, about 95%, about 96%, about 97%, about 98%, or about 99% identity to SEQ ID NO: 9, 18, 64, 75, 86, 190, 157, 212, 201, 226, 179, 168, and 14. In another embodiment, the anti-CD19 binding domain comprises: (a) a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; or (b) a sequence having 90%, about 91%, about 92%, about 93%, about 94%, about 95%, about 96%, about 97%, about 98%, or about 99% identity to SEQ ID NO: 21, SEQ ID NO: 24 SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

### VI. Compositions

One aspect of the present disclosure provides a composition comprising a modified cell, modified lymphocyte, a modified immune cell, or a modified CD4⁺ and CD8⁺ cell produced by the methods described herein. Another aspect of the present disclosure provides a composition comprising a population of modified lymphocytes, a population of modified cells, a population of modified immune cells, or a population of modified CD4⁺ and CD8⁺ cells generated by the methods described herein. Another aspect of the present disclosure provides a composition comprising a lentiviral vector described herein. In some embodiments, the composition further comprises one or more pharmaceutically or physiologically acceptable carriers, diluents, or excipients.

In some embodiments, the compositions described herein are used in medicaments for use in treating a disease or a described herein (e.g., cancer, any malignancy, autoimmune diseases involving cells or tissues which express a tumor antigen as described herein). In some embodiments, the compositions described herein is used in methods for treating, treating a disease or a described herein (e.g., cancer, any malignancy, autoimmune diseases involving cells or tissues which express a tumor antigen as described herein). In some embodiments, provided herein are pharmaceutical compositions comprising a CAR-expressing cell, for example, a plurality of CAR-expressing cells, made by a manufacturing process described herein (for example, the cytokine process, or the activation process described herein).

### VII. Method of Treatment

In one aspect, the present disclosure provides a method for adoptive cell transfer therapy comprising administering to a subject in need thereof a modified immune cell engineered by the methods described herein. In some embodiments, disclosed herein is a method of treating a disease or a condition in a subject, which comprises administering to the subject a population of modified T cells described herein, e.g., a population of modified unstimulated T cells or a population of modified stimulated T cells described herein. In some embodiments, the disclosure includes a method of treating a disease or condition in a subject comprising administering to a subject in need thereof a composition comprising the modified immune cells described herein.

One aspect of the present disclosure provides a method of treating a disease or condition in a subject, the method comprising administering to the subject in need thereof a therapeutically effective amount of the modified cell, the modified immune cell, or the modified CD4⁺ and CD8⁺ cell, thereby treating the disease or condition in the subject. The method of treating a disease or condition in a subject may also comprise administering to the subject in need thereof a therapeutically effective amount of the population of modified cells, the population of modified immune cells, or the population of modified CD4⁺ and CD8⁺ cells made by the methods described herein. The method of treating a disease or condition in a subject may also comprise administering to the subject in need thereof a therapeutically effective amount of the composition described herein.

In some embodiments, the modified immune cell, or the modified CD4⁺ and CD8⁺ cell. In some embodiments, the modified immune cell, or the modified CD4⁺ and CD8⁺ cell is allogeneic to the subject. In some embodiments, the modified immune cell, or the modified CD4⁺ and CD8⁺ cell is a xenogeneic to the subject. In some embodiments, the subject is a human.

A. Diseases and Conditions

One aspect of the present disclosure provides a method of providing an anti-tumor immunity in a mammal comprising administering to the mammal an effective amount of a composition, or a modified cell described herein. In some embodiments, the composition comprises a modified cell expressing a CAR as described herein. The composition may also comprise a modified cell or a population of modified cells.

Another aspect of the present disclosure provides a method of treating a mammal having a disease associated with expression of CD19 comprising administering to the mammal an effective amount of a composition or a modified cell described herein. In some embodiments, the composition comprises a modified cell expressing a CAR described herein.

The modified cell may be an autologous modified T cell or an allogeneic modified T cell. In some embodiments, the mammal is a human.

In some embodiments, the disease associated with CD19 expression is selected from a proliferative disease, a malignancy, a precancerous condition, or a non-cancer related indication associated with expression of CD19. In some embodiments, the disease associated with CD19 expression is a cancer, an atypical and/or a non-classical cancer, a myelodysplasia, a myelodysplastic syndrome, or a preleukemia.

In some embodiments, the disease is a hematologic cancer selected from the group consisting of an acute leukemia, a chronic leukemia, a hematologic condition, and combinations thereof. The disease may also be a B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, ineffective production (or dysplasia) of myeloid blood cells, and combinations thereof.

In some embodiments, the modified cells or the composition are administered in combination with an agent that increases the efficacy of a cell expressing a CAR molecule. In some embodiments, the modified cells or the composition are administered in combination with an agent that ameliorates one or more side effects associated with administration of a cell expressing a CAR molecule. In some embodiments, the modified cells or the composition are administered in combination with an agent that treats the disease associated with CD19.

One aspect of the present disclosure provides an adoptive cell transfer therapy method for a disease or condition. In some embodiments, the disease or condition may be selected from the group consisting of cancer, an autoimmune disease, Lupus, a neurodegenerative disease or condition, Alzheimer disease, multiple sclerosis, an infectious disease, a fibrotic condition, liver fibrosis, lung fibrosis, post-ischemic fibrosis, a genetic disorder, sickle cell anemia, hemophilia, and/or beta-thalassemia. In some embodiments, the disease or condition selected from a cancer, any malignancy, autoimmune diseases involving cells or tissues which express a tumor antigen as described herein.

B. Combination Therapy

In some embodiments, the method of treating a disease further comprises administering to the subject an additional therapeutic agent or an additional therapy. In some cases, an additional therapeutic agent disclosed herein comprises a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone. In some cases, the additional therapeutic agent comprises a first-line therapy. As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers. In some cases, the additional therapeutic agent comprises an immune checkpoint inhibitor. In some instances, the immune checkpoint inhibitor comprises an inhibitors such as an antibody or fragments (e.g., a monoclonal antibody, a human, humanized, or chimeric antibody) thereof, RNAi molecules, or small molecules to PD-1, PD-L1, CTLA4, PD-L2, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. Exemplary checkpoint inhibitors include pembrolizumab, nivolumab, tremelimumab, or ipilimumab. In some embodiments, the additional therapy comprises radiation therapy.

In some embodiments, the additional therapy comprises surgery.

### VIII. Kits

One aspect of the present disclosure provides a kit comprising a population of modified immune cells or a population of modified CD4⁺ and CD8⁺ cells, or a population of engineered by the methods described herein. Another aspect of the present disclosure provides a kit comprising a lentiviral vector comprising a CAR described herein.

### IX. Definitions

Unless defined otherwise, all technical and scientific terms used herein have the same meaning as commonly understood by one of ordinary skill in the art to which the disclosure pertains.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein. It is also to be understood that the terminology used herein is for the purpose of describing particular embodiments only, and is not intended to be limiting.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); Antibodies, A Laboratory Manual; Greenfield ed. (2014) Antibodies, A Laboratory Manual; Freshney (2010); Lundblad and Macdonald eds. (2010) Handbook of Biochemistry and Molecular Biology, 4th edition.

As used herein, the singular forms “a”, “an,” and “the” include plural referents unless the context clearly indicates otherwise. For example, the term “a cell” includes a plurality of cells, including mixtures thereof, and means one cell or more than one cell.

As used herein, the term “About” refers to a value includes the standard deviation of error for the device or method being employed to determine the value. The term “about” when used before a numerical designation, e.g., temperature, time, amount, and concentration, including range, indicates approximations which may vary by (+) or (—) (±) 20%, 15%, 10%, 5%, 3%, 2%, or 1%. Preferably ±5%, more preferably ±1%, and still more preferably ±0.1% from the specified value, as such variations are appropriate to perform the disclosed methods.

As used herein, the term “Activation” refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, the term “Affinity” means a measure of the binding strength between antibody and a simple hapten or antigen determinant. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. In the case of an antibody (Ab) binding to an antigen (Ag), the affinity constant is used (expressed as inverted dissociation constant).

Ab+Ag=AbAg Ka=i[AbAg][Ab][Ag]=1 Kₐ

The chemical equilibrium of antibody binding is also the ratio of the on-rate (k_{forward}) and off-rate (k_{back}) constants. Two antibodies can have the same affinity, but one may have both a high on- and off-rate constant, while the other may have both a low on- and off-rate constant.

Antibody activity in functional assays (e.g., cell lysis assay) may also be reflective of antibody affinity. In some embodiments, the antigen recognizing receptor has low affinity. Low affinity includes micromolar and nanomolar affinities. A low affinity may comprise 10⁻³, 10⁻⁴, 10⁻⁵, 5×10⁻⁵, 5×10⁻⁶, 10⁻⁶, 5×10⁻⁷, 10⁻⁷, 5×10⁻⁸, 10⁻⁸, 5×10⁻⁹, or 10⁻⁹ M.

Antibody and affinities can be phenotypically characterized and compared using functional assay (e.g., cell lysis assay). A wide variety of methods for determining binding affinity are known in the art. An exemplary method for determining binding affinity employs surface plasmon resonance. Surface plasmon resonance is an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.).

As used herein, the term “Allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

As used herein, the term “Analogue”, in relation to polypeptides or polynucleotides includes any mimetic, that is, a chemical compound that possesses at least one of the endogenous functions of the polypeptides or polynucleotides which it mimics. Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the present disclosure may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine. Conservative substitutions may be made.

As used herein, the term “Antibody” refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)2, as well as single chain antibodies (scFv) and humanized antibodies. In some embodiments, antibody refers to such assemblies (e.g., intact antibody molecules, immunoadhesins, or variants thereof) which have significant known specific immunoreactive activity to an antigen of interest (e.g., a tumor associated antigen). Antibodies and immunoglobulins comprise light and heavy chains, with or without an interchain covalent linkage between them. Basic immunoglobulin structures in vertebrate systems are relatively well understood.

The term “Antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

In some embodiments the term antibody fragment refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The portion of the CAR composition of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv), a human derived antibody, and a humanized antibody. In one aspect, the antigen binding domain of a CAR composition of the disclosure comprises an antibody fragment. In a further aspect, the CAR comprises an antibody fragment that comprises a scFv.

As used herein, the term “Antibody heavy chain,” as used herein, refers to the larger of the two types of polypeptide chains present in all antibody molecules in their naturally occurring conformations.

As used herein, an “Antibody light chain,” refers to the smaller of the two types of polypeptide chains present in all antibody molecules in their naturally occurring conformations. α and β light chains refer to the two major antibody light chain isotypes. The antigen binding domain of (e.g., a chimeric antigen receptor) includes antibody variants. As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein, the term “Antigen” or “Ag” is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit a desired immune response. Moreover, the skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “Antigen presenting cell” or “APC” refers to an immune system cell such as an accessory cell (e.g., a B-cell, a dendritic cell, and the like) that displays a foreign antigen complexed with major histocompatibility complexes (WIC's) on its surface. T-cells may recognize these complexes using their T-cell receptors (TCRs). APCs process antigens and present them to T-cells.

As used herein, the term “Anti-tumor effect” refers to a biological effect which can be manifested by a decrease in tumor volume, a decrease in the number of tumor cells, a decrease in the number of metastases, an increase in life expectancy, or amelioration of various physiological symptoms associated with the cancerous condition. In some embodiments, an “anti-tumor effect” can also be manifested by the ability of the peptides, polynucleotides, cells and antibodies of the disclosure in prevention of the occurrence of tumor in the first place.

As used herein, the term “Autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia areata, ankylosing spondylitis, autoimmune hepatitis, autoimmune parotitis, cancer, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “Autologous” is meant to refer to any material derived from the same individual into whom the material may later be re-introduced.

As used herein, the term “Cancer” refers to a disease characterized by the rapid and uncontrolled growth of aberrant cells. Cancer cells can spread locally or through the bloodstream and lymphatic system to other parts of the body. As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, metastatic castrate-resistant prostate cancer, melanoma, synovial sarcoma, advanced TnMuc1 positive solid tumors, neuroblastoma, neuroendocrine tumors, and the like. In certain embodiments, CD19-positive tumor, the cancer is medullary thyroid carcinoma. In certain embodiments, the cancer is prostate cancer. In certain embodiments, the cancer is mesothelioma or a mesothelin expressing cancer. In some embodiments, the cancer is metastatic castrate-resistant prostate cancer. The terms “cancer” and “tumor” are used interchangeably herein, and both terms encompass solid and liquid tumors, diffuse or circulating tumors. In some embodiments, the cancer or tumor includes premalignant, as well as malignant cancers and tumors.

As used herein, the term “Cancer associated antigen” or “Tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells (e.g., a lineage marker such as CD19 on B cells). In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present disclosure includes CARs comprising an antigen binding domain (e.g., antibody or antibody fragment) that binds to an MHC presented peptide. Normally, peptides derived from endogenous proteins fill the pockets of Major histocompatibility complex (MHC) class I molecules and are recognized by T cell receptors (TCRs) on CD8⁺ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described. For example, TCR-like antibody can be identified from screening a library, such as a human scFv phage displayed library.

As used herein, the term “Cancer-supporting antigen” or “tumor-supporting antigen” interchangeably refers to a molecule (typically a protein, carbohydrate or lipid) that is expressed on the surface of a cell that is, itself, not cancerous, but supports the cancer cells by promoting their growth or survival (e.g., resistance to immune cells). Exemplary cells of this type include stromal cells and myeloid-derived suppressor cells (MDSCs). The tumor-supporting antigen itself need not play a role in supporting the tumor cells so long as the antigen is present on a cell that supports cancer cells.

As used herein, a “Cell-surface marker” refers to any molecule that is expressed on the surface of a cell. Cell-surface expression usually requires that a molecule possesses a transmembrane domain. Many naturally occurring cell-surface markers are termed “CD” or “cluster of differentiation” molecules. Cell-surface markers often provide antigenic determinants to which antibodies can bind.

As used herein, the term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below.

In some embodiments, CAR refers to an artificial T cell receptor that is engineered to be expressed on an immune effector cell or precursor cell thereof and specifically bind an antigen. CARs may be used in adoptive cell therapy with adoptive cell transfer. In some embodiments, adoptive cell transfer (or therapy) comprises removal of T cells from a patient, and modifying the T cells to express the receptors specific to a particular antigen. In some embodiments, the CAR has specificity to a selected target, for example, CD19, ROR1, mesothelin, c-Met, PSMA, PSCA, Folate receptor alpha, Folate receptor beta, EGFR, EGFRvIII, GPC2, GPC2, Mucin 1 (MUC1), Tn antigen ((Tn Ag) or (GalNAca-Ser/Thr)), TnMUC1, GDNF family receptor alpha-4 (GFRa4), fibroblast activation protein (FAP), or Interleukin-13 receptor subunit alpha-2 (IL-13Ra2 or CD213A2).

In some embodiments, the stimulatory molecule is the zeta chain associated with the T cell receptor complex. I In some embodiments, the cytoplasmic signaling domain further comprises one or more functional signaling domains derived from at least one costimulatory molecule as defined below. In some embodiments, the costimulatory molecule is chosen from 4-1BB (i.e., CD137), CD27 and/or CD28. In some embodiments, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule.

As used herein, the term “Signaling domain” refers to the functional portion of a protein, which acts by transmitting information within the cell to regulate cellular activity via defined signaling pathways by generating second messengers or functioning as effectors by responding to such messengers.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD 19 can be found as UniProt/Swiss-Prot Accession No. P15391 and the nucleotide sequence encoding of the human CD19 can be found at Accession No. NM_001178098. CD19 is expressed on most B lineage cancers, including, e.g., acute lymphoblastic leukemia, chronic lymphocyte leukaemia and non-Hodgkin's lymphoma. Other cells with express CD 19 are provided below in the definition of “disease associated with expression of CD19.” It is also an early marker of B cell progenitors. See, e.g., Nicholson et al. Mol. Immun. 34 (16-17): 1157-1165 (1997). In some embodiments, the antigen-binding portion of the CART recognizes and binds an antigen within the extracellular domain of the CD19 protein. In some embodiments, the CD19 protein is expressed on a cancer cell.

As used herein, the term “Conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

As used herein, the term “Co-stimulatory ligand,” includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MEW molecule loaded with peptide, mediates a T cell response, including, but not limited to, proliferation, activation, differentiation, and the like. A co-stimulatory ligand can include, but is not limited to, CD2, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

As used herein, a “Co-stimulatory molecule” refers to the cognate binding partner on a T cell that specifically binds with a co-stimulatory ligand, thereby mediating a co-stimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA, a Toll ligand receptor, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR). In some embodiments, a co-stimulatory molecule includes OX40, CD27, CD2, CD28, ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

As used herein, the term “Co-stimulatory signal” refers to a signal, which in combination with a primary signal, such as TCR/CD3 ligation, leads to T cell proliferation and/or upregulation or downregulation of key molecules. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like.

As used herein, the term “Derived from” refers to a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, for example, it does not mean that, to provide the intracellular signaling domain, one must start with a CD3zeta sequence and delete unwanted sequence, or impose mutations, to arrive at the intracellular signaling domain.

As used herein, the term “Disease” refers to a state of health of an animal wherein the animal cannot maintain homeostasis, and wherein if the disease is not ameliorated then the animal's health continues to deteriorate. In contrast, the term “disorder” in an animal refers to a state of health in which the animal is able to maintain homeostasis, but in which the animal's state of health is less favorable than it would be in the absence of the disorder. Left untreated, a disorder does not necessarily cause a further decrease in the animal's state of health.

As used herein, “Disease associated with expression of a tumor antigen” includes, but is not limited to, a disease associated with expression of a tumor antigen or condition associated with cells which express a tumor antigen including, but not limited to proliferative diseases such as a cancer or malignancy or a precancerous condition such as a myelodysplasia, a myelodysplastic syndrome or a preleukemia; or a noncancer related indication associated with cells, which express a tumor antigen. In some embodiments, a cancer associated with expression of a tumor antigen is a hematological cancer. In some embodiments, a cancer associated with expression of a tumor antigen is a solid cancer. Further diseases associated with expression of a tumor antigen include, but not limited to, atypical and/or non-classical cancers, malignancies, precancerous conditions or proliferative diseases associated with expression of a tumor antigen. Non-cancer related indications associated with expression of a tumor antigen include, but are not limited to, autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiment, the tumor antigen-expressing cells produced detectable levels of a tumor antigen protein at one point, and subsequently produced substantially no detectable tumor antigen protein.

As used herein, the term “Disease associated with expression of CD19” includes, but is not limited to, a disease associated with expression of CD19 or condition associated with cells which express CD19 including, proliferative diseases such as a cancer or malignancy or a precancerous condition such as a myelodysplasia, a myelodysplasia syndrome or a preleukemia; or a noncancer related indication associated with cells which express CD19. In some embodiments, a cancer associated with expression of CD 19 is a hematolical cancer. In one aspect, the hematolical cancer is a leukemia or a lymphoma. In one aspect, a cancer associated with expression of CD19 includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of CD 19 comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplasia syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further diseases associated with expression of CD19 expression include, but not limited to, e.g., atypical and/or non-classical cancers, malignancies, precancerous conditions or proliferative diseases associated with expression of CD19. Non-cancer related indications associated with expression of CD19 include, but are not limited to, e.g., autoimmune disease, (e.g., lupus), inflammatory disorders (allergy and asthma) and transplantation.

As used herein, the term “Downregulation” refers to the decrease or elimination of gene expression of one or more genes.

As used herein, the term “Encoding” refers to the inherent property of specific sequences of nucleotides in a polynucleotide (such as a gene, a cDNA, or an mRNA), to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “Effective amount” and “Therapeutically effective amount” are used interchangeably herein, refer to an amount of a compound, formulation, material, pharmaceutical agent, or composition, as described herein effective to achieve a desired physiological, therapeutic, or prophylactic outcome in a subject in need thereof. Such results may include, but are not limited to an amount that when administered to a mammal, causes a detectable level of immune response compared to the immune response detected in the absence of the composition of the disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

As used herein, the term “Endogenous” refers to any material from or produced inside an organism, cell, tissue or system.

As used herein, the term “Expression” refers to the transcription and/or translation of a particular nucleotide sequence driven by a promoter.

As used herein, the term “Exogenous” refers to any material introduced from or produced outside an organism, cell, tissue or system.

As used herein, the term “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “Extended packaging signal” or “Extended packaging sequence” refers to the use of sequences around the psi sequence with further extension into the gag gene. The inclusion of these additional packaging sequences may increase the efficiency of insertion of vector RNA into viral particles. As an example, for the Murine Leukemia Virus (MoMLV) the minimum core packaging signal is encoded by the sequence (counting from the 5′ LTR cap site) from approximately nucleotide 144, up through the Pst I site (nucleotide 567). The extended packaging signal of MoMLV includes the sequence beyond nucleotide 567 up through the start of the gag/pol gene (nucleotide 621), and beyond nucleotide 1040. These sequences include about a third of the gag gene sequence.

As used herein, the term “ex vivo,” refers to cells that have been removed from a living organism, (e.g., a human) and propagated outside the organism (e.g., in a culture dish, test tube, or bioreactor).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, the term “Flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3. n=4, n=5 and n=6, n=7, n=8, n=9 and n=10. Exemplary linkers are shown in Table 1.

As used herein, a “Fragment” is also a variant and the term typically refers to a selected region of a polypeptide or polynucleotide that is of interest either functionally or, for example, in an assay. “Fragment” thus refers to an amino acid or nucleic acid sequence that is a portion of a full-length polypeptide or polynucleotide.

As used herein, “Functional variant” refers to a polypeptide that has a substantially identical amino acid sequence to a reference amino acid sequence, or is encoded by a substantially identical nucleotide sequence, and is capable of having one or more activities of the reference amino acid sequence.

As used herein, the term “Host cell” includes cells transfected, infected, or transduced in vivo, ex vivo, or in vitro with a recombinant vector or a polynucleotide of the disclosure. Host cells may include packaging cells, producer cells, and cells infected with viral vectors. In some embodiments, host cells infected with the lentiviral vector of the disclosure are administered to a subject in need of therapy. In some embodiments, the term “target cell” is used interchangeably with host cell and refers to transfected, infected, or transduced cells of a desired cell type. In preferred embodiments, the target cell is a T cell.

As used herein, the term “Homologous” refers to the subunit sequence identity between two polymeric molecules (e.g., between two nucleic acid molecules, such as, two DNA molecules or two RNA molecules), or between two polypeptide molecules. When a subunit position in both of the two molecules is occupied by the same monomeric subunit, then they are homologous at that position. For example, if a position in each of two DNA molecules is occupied by adenine, then the two DNA molecules are homologous. The homology between two sequences is a direct function of the number of matching or homologous positions. For example, if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

As used herein, the term “Homologue” means an entity having a certain homology with the wild type amino acid sequence and the wild type nucleotide sequence. The term “homology” can be equated with “identity”. In the present context, a homologous sequence is taken to include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e., amino acid residues having similar chemical properties/functions), in the context of the present disclosure it is preferred to express homology in terms of sequence identity.

A homologous sequence is taken to include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present disclosure it is preferred to express homology in terms of sequence identity. Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology.

However, these more complex methods assign “Gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (FASTA and the GENEWORKS suite of comparison tools. Both BLAST and FASTA are available for offline and online searching. However, for some applications, it is preferred to use the GCG Bestfit program. Another tool, called BLAST 2 Sequences is also available for comparing protein and nucleotide sequences.

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62. Once the software has produced an optimal alignment, it is possible to calculate percentage homology, preferably percentage sequence identity. The software typically does this as part of the sequence comparison and generates a numerical result.

As used herein, the term “Hybrid vector” refers to a vector, LTR or other nucleic acid containing both retroviral sequences (e.g., lentiviral), and non-retroviral sequences (e.g., lentiviral viral sequences). In one embodiment, a hybrid vector refers to a vector or transfer plasmid comprising retroviral (e.g., lentiviral) sequences for reverse transcription, replication, integration and/or packaging.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the disclosure to make the encoded protein. These methods are only illustrative of the numerous standard techniques known in the art for manipulation of DNA sequences and other known techniques may also be used.

As used herein, the term “Identity” refers to the subunit sequence identity between two polymeric molecules particularly between two amino acid molecules, such as, between two polypeptide molecules. When two amino acid sequences have the same residues at the same positions, then they are identical at that position. For example, if a position in each of two polypeptide molecules is occupied by an Arginine, then the two polypeptides are identical. The identity or extent to which two amino acid sequences have the same residues at the same positions in an alignment is often expressed as a percentage. The identity between two amino acid sequences is a direct function of the number of matching or identical positions. For example, if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the term “Immunoglobulin” or “Ig,” defines a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “Immune response” as used herein is defined as a cellular response to an antigen that occurs when lymphocytes identify antigenic molecules as foreign and induce the formation of antibodies and/or activate lymphocytes to remove the antigen.

As used herein, the term “Immune effector cell,” refers to a cell that is involved in an immune response, e.g., in the promotion of an immune effector response. Examples of immune effector cells include T cells (e.g., alpha/eta T cells and gamma/delta T cells), B cells, natural killer (NK) cells, natural killer T (NKT) cells, mast cells, and myeloic-derived phagocytes.

As used herein, the term “Immune effector function or immune effector response,” refers to a function or response that enhances or promotes an immune attack of a target cell. In some embodiment, an immune effector function or response refers to a property of a T or NK cell that promotes the killing or the inhibition of growth or proliferation, of a target cell. In the case of a T cell, primary stimulation and co-stimulation are examples of immune effector function or response.

As used herein, the term “Inhibitory molecule” refers to a molecule, which when activated, causes or contributes to an inhibition of cell survival, activation, proliferation and/or function; and the gene encoding said molecule and its associated regulatory elements (e.g., promoters). In some embodiments, an inhibitory molecule is a molecule expressed on an immune effector cell (e.g., on a T cell). Non-limiting examples of inhibitory molecules are PD-1, PD-L1, PD-L2, CTLA4, TIN/13, LAG3, CEACAM (e.g., CEACAM-1, CEACAM-3 and/or CEACAM-5), VISTA, TGFβIIR, VSIG3, VSIG 8, BTLA, TIGIT, LAIR1, CD160, 2B4, CD80, CD86, B7-H3 (CD276), B7-H4 (VTCN1), HVEM (TNFRSF14 or CD107), KIR, A2aR, MHC class I, MHC class II, GALS, adenosine, and TGF beta. It will be understood that the term inhibitory molecule refers to the gene (and its associated regulatory elements) encoding an inhibitory molecule protein when it is used in connection with a target sequence or gRNA molecule. In some embodiments, gene encoding the inhibitory molecule is BTLA, PD-1, TIM-3, VSIG3, VSIG8, CTLA4, or TGFβIIR. In some embodiments, the gene encoding the inhibitory molecule is VSIG3. In some embodiments, the gene encoding the inhibitory molecule is PD-1. In some embodiments, the gene encoding the inhibitory molecule is TGFβIIR.

As used herein, the term “Induced pluripotent stem cell” or “iPS cell” refers to a pluripotent stem cell that is generated from adult cells, such immune cells (i.e., T cells). The expression of reprogramming factors, such as Klf4, Oct3/4 and Sox2, in adult cells convert the cells into pluripotent cells capable of propagation and differentiation into multiple cell types.

As used herein, the term “Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, “In vitro transcribed RNA” refers to RNA that has been synthesized in vitro. In some embodiments the RNA is mRNA. Generally, the in vitro transcribed RNA is generated from an in vitro transcription vector. The in vitro transcription vector comprises a template that is used to generate the in vitro transcribed RNA.

As used herein, the term “Knockout” refers to the ablation of gene expression of one or more genes.

As used herein, the term “K_{D}” refers to the equilibrium dissociation constant between an antibody and its antigen. In particular, K_{D} is the equilibrium dissociation constant, a ratio of K_{off}/Kₒₙ, between the antibody and its antigen. K_{D} and affinity are inversely related. The K_{D} value relates to the concentration of antibody (the amount of antibody needed for a particular experiment) and so the lower the K_{D} value (lower concentration) and thus the higher the affinity of the antibody. Most antibodies have K_{D} values in the low micromolar (10⁻⁶) to nanomolar (10⁻⁷ to 10⁻⁹) range. High affinity antibodies generally considered to be in the low nanomolar range (10⁻⁹) with very high affinity antibodies being in the picomolar (10⁻¹²) range.

The term “Kₒₙ”, or “association reaction,” is the “on-rate,” which is a constant a constant used to characterize how quickly an antibody binds to its target.

The term “K_{off}”, or “disassociation reaction,” is the “off-rate,” which is a constant used to characterize how quickly an antibody dissociates from its target. The ratio of experimentally measured off- and on-rates (K_{off}/Kₒₙ) is used to calculate the K_{D} value.

As used herein, the term “Lentiviral vector” refers to a viral vector or plasmid containing structural and functional genetic elements, or portions thereof, including LTRs that are primarily derived from a lentivirus. In some embodiments, the terms “Lentiviral vector,” and “Lentiviral expression vector” may be used to refer to lentiviral transfer plasmids and/or infectious lentiviral particles. Where reference is made herein to elements such as cloning sites, promoters, regulatory elements, heterologous nucleic acids, etc. In some embodiments, the sequences of these elements are present in RNA form in the lentiviral particles of the disclosure and are present in DNA form in the DNA plasmids of the disclosure.

A lentiviral or lentivirus vector, as used herein, is a vector which comprises at least one component part derivable from a lentivirus. Preferably, that component part is involved in the biological mechanisms by which the vector infects cells, expresses genes or is replicated. The lentiviral vector may be a “non-primate” vector, i.e., derived from a virus which does not primarily infect primates, especially humans. The non-primate lentivirus may be any member of the family of lentiviridae, which does not naturally infect a primate and may include a feline immunodeficiency virus (Hy), a bovine immunodeficiency virus (BIV), a caprine arthritis encephalitis virus (CAEV), a Maedi visna virus (MVV) or an equine infectious anaemia virus (EIAV).

As used herein, the term “Lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

As used herein, the term “Modified” means a changed state or structure of a molecule or cell of the disclosure. Molecules may be modified in many ways, including chemically, structurally, and functionally. Cells may be modified through the introduction of nucleic acids.

As used herein, the term “Modulating,” means mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

As used herein, a “Naive T cell” refers to a T cell that is antigen-inexperienced. In some embodiments, an antigen-inexperienced T cell has encountered its cognate antigen in the thymus but not in the periphery. In some embodiments, naive T cells are precursors of memory cells. In some embodiments, naive T cells express both CD45RA and CCR7, but do not express CD45RO. In some embodiments, naive T cells may be characterized by expression of CD62L, CD27, CCR7, CD45RA, CD28, and CD127, and the absence of CD95 or CD45RO isoform. In some embodiments, naive T cells express CD62L, IL-7 receptor-a, IL-6 receptor, and CD132, but do not express CD25, CD44, CD69, or CD45RO. In some embodiments, naive T cells express CD45RA, CCR7, and CD62L and do not express CD95 or IL-2 receptor f3. In some embodiments, surface expression levels of markers are assessed using flow cytometry.

Unless otherwise specified, a “Nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may contain an intron(s).

As used herein, the term “Operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “Overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

As used herein, the term “Parenteral” administration of an immunogenic composition includes, e.g., subcutaneous (s.c.), intravenous (i.v.), intramuscular (i.m.), or intrasternal injection, or infusion techniques.

As used herein, the terms “Peptide,” “Polypeptide,” and “Protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, a “poly(A)” is a series of adenosines attached by polyadenylation to the mRNA. In some embodiments of a construct for transient expression, the poly(A) is between 50 and 5000. In some embodiments the poly (A) is greater than 64. In some embodiments the poly(A) is greater than 100. In some embodiments the poly(A) is greater than 300. In some embodiments the poly(A) is greater than 400. poly(A) sequences can be modified chemically or enzymatically to modulate mRNA functionality such as localization, stability or efficiency of translation.

As used herein, “Polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, the term “Transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the term “Polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the term “Promoter” is defined as a DNA sequence recognized by the synthetic machinery of the cell, or introduced synthetic machinery, required to initiate the specific transcription of a polynucleotide sequence.

As used herein, the term “Promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “Constitutive promoter” is a nucleotide sequence which, when operably linked with a polynucleotide which encodes or specifies a gene product, causes the gene product to be produced in a cell under most or all physiological conditions of the cell.

As used herein, the term “Inducible promoter” is a nucleotide sequence which, when operably linked with a polynucleotide which encodes or specifies a gene product, causes the gene product to be produced in a cell substantially only when an inducer which corresponds to the promoter is present in the cell.

As used herein, the term “Tissue-specific promoter” is a nucleotide sequence which, when operably linked with a polynucleotide encodes or specified by a gene, causes the gene product to be produced in a cell substantially only if the cell is a cell of the tissue type corresponding to the promoter.

As used herein, the term “Pseudotype” or “Pseudotyping” refers to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells. In a preferred embodiment of the disclosure, lentiviral envelope proteins are pseudotyped with VSV-G. In one embodiment, the disclosure provides packaging cells, which produce recombinant retrovirus, e.g., lentivirus, pseudotyped with the VSV-G envelope glycoprotein.

As used herein, the term “Recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

As used herein, the term “Recombinant viral vector” (RRV) refers to a vector with sufficient viral genetic information to allow packaging of an RNA genome, in the presence of packaging components, into a viral particle capable of infecting a target cell. The RRV carries non-viral coding sequences which are to be delivered by the vector to the target cell. A RRV is incapable of independent replication to produce infectious viral particles within the final target cell. Usually the RRV lacks a functional gag-pol and/or env gene and/or other genes essential for replication. The vector of the present disclosure may be configured as a split-intron vector. Preferably the RRV vector of the present disclosure has a minimal viral genome.

As used herein, the term “Retroviral vector” refers to a viral vector or plasmid containing structural and functional genetic elements, or portions thereof, that are primarily derived from a retrovirus.

In some embodiments, an additional safety enhancement is provided by replacing the U3 region of the 5′ LTR with a heterologous promoter to drive transcription of the viral genome during production of viral particles. The heterologous promoters may be selected from the group consisting of viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In some embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

As used herein, the term “Signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

As used herein, the term “Single chain antibodies” refer to antibodies formed by recombinant DNA techniques in which immunoglobulin heavy and light chain fragments are linked to the Fv region via an engineered span of amino acids. Various methods of generating single chain antibodies are known in the art.

As used herein, the term “Single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., Tn-MUC1 binding domain, PSMA binding domain, or mesothelin binding domain) comprises an scFv having the configuration from N-terminus to C-terminus, VH-linker-VL. In some embodiments, the antigen binding domain (e.g., a Tn-MUC1 binding domain, a PSMA binding domain, or a mesothelin binding domain) comprises an scFv having the configuration from N-terminus to C-terminus, VL-linker-VH. Those of skill in the art would be able to select the appropriate configuration for use in the present disclosure.

The linker is typically rich in glycine for flexibility, as well as serine or threonine for solubility. The linker can link the heavy chain variable region and the light chain variable region of the extracellular antigen-binding domain. Various linker sequences are known in the art, including, without limitation, glycine serine (GS) linkers such as (GS)n, (GSGGS)n, (GGGS)n, and (GGGGS)n, where n represents an integer of at least 1. Exemplary linker sequences can comprise amino acid sequences including, without limitation, GGSG (SEQ ID NO: 121), GGSGG (SEQ ID NO:122), GSGSG (SEQ ID NO: 123), GSGGG (SEQ ID NO: 124), GGGSG (SEQ ID NO: 125), GSSSG (SEQ ID NO: 126), GGGGS (SEQ ID NO: 127), or GGGGSGGGGSGGGGS (SEQ ID NO: 128), and the like. Those of skill in the art would be able to select the appropriate linker sequence for use in the present disclosure. In one embodiment, an antigen binding domain (e.g., a CD19 binding domain) of the present disclosure comprises a heavy chain variable region (VH) and a light chain variable region (VL). In some embodiments, the VH and VL is separated by the linker sequence having the amino acid sequence GGGGSGGGGSGGGGS (SEQ ID NO:128). In some embodiments, the linker nucleic acid sequence comprises the nucleotide sequence GGTGGCGGTGGCTCGGGCGGTGGTGGGTCGGGTGGCGGCGGATCT (SEQ ID NO: 129).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences. Antagonistic scFvs having inhibitory activity have been described.

As used herein, the term “Specificity” refers to the ability to specifically bind (e.g., immunoreact with) a given target antigen (e.g., a human target antigen). A chimeric antigen receptor may be monospecific and contain one or more binding sites, which specifically bind a target or a chimeric antigen receptor may be multi-specific and contain two or more binding sites which specifically bind the same or different targets. In certain embodiments, a chimeric antigen receptor is specific for two different (e.g., non-overlapping) portions of the same target. In certain embodiments, a chimeric antigen receptor is specific for more than one target.

As used herein, the term “Spacer domain” generally means any oligo- or polypeptide that functions to link the transmembrane domain to, either the extracellular domain or, the intracellular domain in the polypeptide chain. A spacer domain may comprise up to about 300 amino acids, e.g., about 10 to about 100 amino acids, or about 25 to about 50 amino acids.

As used herein, the term “Specifically binds,” with respect to an antibody, means an antibody or binding fragment thereof (e.g., scFv) which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “Specific binding” or “Specifically binding,” can be used in reference to the interaction of an antibody, a protein, a chimeric antigen receptor, or a peptide with a second chemical species, to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, the term “Stimulation,” means a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, clonal expansion, and differentiation into distinct subsets.

As used herein, the term “Stimulatory molecule” means a molecule on a T cell that specifically binds with a cognate stimulatory ligand present on an antigen presenting cell. Stimulatory molecule may be expressed by a T cell that provides the primary cytoplasmic signaling sequence(s) that regulate primary activation of the TCR complex in a stimulatory way for at least some aspect of the T cell signaling pathway. For example, the primary signal is initiated by, for instance, binding of a TCR/CD3 complex with an MEW molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or IT AM. Examples of an IT AM containing primary cytoplasmic signaling sequence that is of particular use in the disclosure includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d. In a specific CAR of the disclosure, the intracellular signaling domain in any one or more CARS of the disclosure comprises an intracellular signaling sequence, e.g., a primary signaling sequence of CD3-zeta. In a specific CAR of the disclosure, the primary signaling sequence of CD3-zeta is the sequence provided as SEQ ID NO: 52, or the equivalent residues from a non-human species, e.g., mouse, rodent, monkey, ape and the like. In a specific CAR of the disclosure, the primary signaling sequence of CD3-zeta is the sequence as provided in SEQ ID NO:54, or the equivalent residues from a non-human species, e.g., mouse, rodent, monkey, ape and the like.

As used herein, the term “Stimulatory ligand” means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, the terms “Subject” refers to a vertebrate. A vertebrate can be a mammal, such as a non-primate (e.g., cows, pigs, horses, cats, dogs, rats, etc.) or a primate (e.g., monkey and human). Mammals can include, without limitation, humans, non-human primates, wild animals, feral animals, farm animals, sport animals, and pets. In some embodiments, “Subject” and “Patient” are used interchangeably. Any living organism in which an immune response can be elicited may be a subject or patient. In certain exemplary embodiments, a subject is a human.

As used herein, the term “Substantially identical”, in the context of a nucleotide sequence, refers to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 75%, 80%, 85%, 90%, 91%, 92%, 93%, 94%, 95%, 96%, 97%, 98% or 99% identity to a reference sequence, for example, a sequence provided herein.

In some embodiments, the context of an amino acid sequence, the term “Substantially identical” refers to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%. 91%, 92%, 93%, 94%, 95%, 96%, 97%, 98% or 99% identity to a reference sequence, for example, a sequence provided herein.

As used herein, the term “Substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

As used herein, “Substantially Complementary” refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

As used herein, the term “T cell priming” refers to the process by which a naive T cell undergoes clonal expansion and differentiation into an effector cytotoxic T cell (CTL) after being stimulated by contact with an antigen. Several molecules can enhance T cell priming and/or cell's antigen presentation when they are introduced into a cell. These molecules include but are not limited to, costimulatory molecules (e.g., CD70, CD83, CD80, CD86, CD40, CD154, CD137L (4-1BBL), CD252 (OX40L), CD275 (ICOS-L), CD54 (ICAM-1), CD49a, CD43, CD48, CD112 (PVRL2), CD150 (SLAM), CD155 (PVR), CD265 (RANK), CD270 (HVEM), TL1A, CD127, IL-4R, GITR-L, CD160, CD258, TIM-4, CD153 (CD30L), CD200R (OX2R), CD44, and ligands thereof), soluble cytokines (e.g., IL-2, IL-12, IL-6, IL-7, IL-15, IL-18, IL-21, GM-CSF, IL-18, IL-21, IL-27), polypeptides involved in antigen presentation (e.g., CD64, MHC I, MHC II), polypeptides involved in trafficking and/or migration (e.g., CD183, CCR2, CCR6, CD50, CD197, CD58, CD62L), and polypeptides involved in dendritic cell targeting (e.g., TLR ligands, anti-DEC-205 antibody, anti-DC-SIGN antibody).

As used herein, the term “Target site” or “Target sequence” refers to a genomic nucleic acid sequence that defines a portion of a nucleic acid to which a binding molecule may specifically bind under conditions sufficient for binding to occur.

As used herein, the term “Targeting domain” used in connection with a gRNA, refers to a portion of the gRNA molecule that recognizes, or is complementary to, a target sequence. For example, a target sequence within the nucleic acid of a cell (e.g., within a gene).

As used herein, the term “Target sequence” refers to a sequence of nucleic acids complimentary, for example fully complementary, to a gRNA targeting domain. In some embodiments, the target sequence is disposed on genomic DNA. In some embodiment the target sequence is adjacent to (either on the same strand or on the complementary strand of DNA) a protospacer adjacent motif (PAM) sequence recognized by a protein having nuclease or other effector activity, e.g., a PAM sequence recognized by Cas9. In some embodiments, the target sequence is a target sequence of an allogeneic T cell target. In some embodiments, the target sequence is a target sequence of an inhibitory molecule. In some embodiments, the target sequence is a target sequence of a downstream effector of an inhibitory molecule.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, coupled to three dimeric modules CD3δ/CD3ε, CD3γ/CD3ε, and CD3ζ/CD3ζ. In some cells the TCR consists of gamma and delta (γ/δ) chains (CD3γ/CD3ε). In some embodiments, TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “Therapeutic” as used herein means a treatment and/or prophylaxis. A therapeutic effect is obtained by suppression, remission, or eradication of a disease state.

As used herein, the term “Therapy” refers to any protocol, method and/or agent (e.g., a CAR-T) that can be used in the prevention, management, treatment and/or amelioration of a disease or a symptom related thereto. In some embodiments, the terms “therapies” and “therapy” refer to a biological therapy (e.g., adoptive cell therapy), supportive therapy (e.g., lymphodepleting therapy), and/or other therapies useful in the prevention, management, treatment and/or amelioration of a disease or a symptom related thereto, known to one of skill in the art such as medical personnel.

As used herein, the term “Tumor antigen” or “Hyperproliferative disorder antigen” or “Antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, lung cancer, liver cancer, non-Hodgkin's lymphoma, non-Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

As used herein, the term “Transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny. The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

As used herein the term “Transduction” refers to the delivery of a gene(s) or other polynucleotide sequence using a retroviral or lentiviral vector by means of viral infection rather than by transfection. In some embodiments, the lentiviral vectors of the present disclosure are transduced into a cell through infection and provirus integration. In some embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

As used herein, the terms “Treat,” “Treatment” and “Treating” refer to the reduction or amelioration of the progression, severity, frequency and/or duration of a disease or a symptom related thereto, resulting from the administration of one or more therapies (including, but not limited to, a CAR-T therapy directed to the treatment of solid tumors). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

As used herein, the term a “Variant of any given sequence” is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein. In some embodiments, a “Variant” refers to a polypeptide that has a substantially identical amino acid sequence to a reference amino acid sequence, or is encoded by a substantially identical nucleotide sequence. In some embodiments, the variant is a functional variant. As used herein, the term “Functional variant” refers to a polypeptide that has a substantially identical amino acid sequence to a reference amino acid sequence, or is encoded by a substantially identical nucleotide sequence, and is capable of having one or more activities of the reference amino acid sequence.

As used herein, the term “Vector” refers to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In some embodiments, a vector is a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, viral vectors, Sendai viral vectors, adenoviral vectors, adeno-associated virus vectors, retroviral vectors, lentiviral vectors, plasmids (e.g., DNA plasmids or RNA plasmids), transposons, cosmids, bacterial artificial chromosomes, and variant viral vectors.

As used herein, the term “Xenogeneic” refers to a graft derived from an animal of a different species.”

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or IT AM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

As used herein, a 5′ cap (also termed an RNA cap, an RNA 7-methylguanosine cap or an RNA m G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples specifically point out various aspects of the present disclosure and are not to be construed as limiting in any way the remainder of the disclosure.

### Example 1: Materials and Methods

Primary cells and Cells lines. Purified CD4⁺ and CD8⁺ T cells obtained from de-identified healthy human donors by Human Immunology Core at the University of Pennsylvania. Leukemic cell line (Nalm6.CBG.GFP), CRISPER knocked out leukemic cell line (Nalm6-CD19KO, CD19-Ve Nalm6.CBG.GFP), Jeko-1 (CBG-GFP) and were established available from ATCC authenticated cell lines. These leukemic cell lines were maintained in culture with RPMI-1640 (Life Technologies) supplemented with 10% FBS (Seradigm), 50 UI/ml penicillin/streptomycin (Life Technologies), 1% of 2 mM GlutaMAX™ (Life Technologies) and 1% of 25 mM of HEPES (Life Technologies). Fresh primary cells (CD4⁺ and CD8⁺ T cells) from HIC were used for all studies. Jurkat NFAT-GFP reporter cell line was obtained from Saar Gill.

Cell culture. Human T cells were purified by negative selection by using RosetteSep™ Human CD3⁺ T cells Enrichment Cocktails (Stem-Cell Technologies) according to the manufacturer protocol. T cells were cultured at 1×10⁶ cells per ml in either complete RPMI: RPMI 1640 (Life Technologies) supplemented with 10% fetal calf serum (Seradigm), 1% Penicillin-Streptomycin (Pen Strep) (Life Technologies), 2 mM GlutaMAX™ (Life Technologies), and 25 mM HEPES buffer (Life Technologies) or in CTS™ OpTmizer™ T-Cell Expansion SFM (Gibco) added with T-cell expansion supplement provided with Media, 1% Penicillin-Streptomycin, 2 mM GlutaMAX™ and 25 mM HEPES buffer. T cells were stimulated either with anti-CD3/CD28 Dynabeads™ (Life Technologies) at 1:3 (cell/bead) ratio or with irradiated K562.OKT3.64.86 at 2:1 (T cells/K562) ratio with 100-300 IU/mL of recombinant human interleukin-2 (Proleukin® from Clinigen). 20 hrs after stimulation, medium was reduced by and replaced with 200 μl of the appropriate lentivirus supernatant. Alternatively, 24 hrs after stimulation, titered virus was added at a 3:1 ratio (infectious particles:T cell). On day 3 of T cell activation, volume was doubled with fresh media. Expansions were de-beaded on day 5, counted every other day adjusting cell counts to 0.5×10⁶ cells per ml with fresh media. When irradiated K562 were used for T cells activation, after day 2 of T cells activation, the medium was quadruplet and medium was changed every second day up to resting stage of T cells (day 10-11).

Flow cytometry. BD LSRFortessa™ instruments using BD FACSDiva™ Software v.8.0.1 (BD Biosciences) was used to acquire samples for different assays. Data were analyzed using FlowJo™ v.10 software. Anti-human antibodies were purchased from BD and BioLegend. CD19 binder CAR expression was evaluated on transduced T cells using FITC-Labeled human CD19 protein (ACROBiosystem, Cat-CD9-HF251).

Intracellular cytokine assay and protein binding to binders. Functionality of CART cells were evaluated following co-cultures of 2×10⁵ CAR or NTD T cells with 4×10⁵ Nalm6.CBG.GFP, Nalm6-CD19KO, or Jeko-1. One hour after start of co-culture, 1× Brefeldin A and Monensin Solution (BioLegend). After 6 hrs co-culture at 37° C., intracellular cytokine production was measured by flow cytometry staining with anti-human antibodies specific for IFN-γ, TNF-α and IL-2.

In another assay, killing of target cells (GFP) was assayed by intracellular staining with active caspase3 (564096, BD). In another assays, to see the binding specificity of CD19 protein towards all CD19 binders, FITC-labeled human CD19 protein was incubated with anti-CD19Ab (clone-FMC63) for 15 min at 1:1 and 1:3 ratio. All original binders were stained with this cocktail to determine if detection of CAR CD19 binders was blocked.

Lentivirus production and transfection. Lentiviral packaging mix containing Rev, Gag/Pol and Cocal-G glycoprotein along with the appropriate pTRPE transfer vector were transfected into HEK293T cells using Lipofectamine™ 2000 (Life Technologies). At 24 hrs and 48 hrs after transfection, the HEK293T cell supernatant was collected, filtered through a 0.45-μm syringe-driven filter and then concentrated the lentivirus by ultracentrifugation at 25,000 r.p.m. for 2.5 hrs at 4° C. The supernatant was discarded and the lentivirus pellet was resuspended in 1000 μl of complete RPMI and stored at −80° C.

RNA Electroporation of truncated CD19 antigen (CD19Ag) in K562 cell line. K562-wt cells were transfected by electroporation with varying amounts of truncated CD19 antigen (CD19Ag) RNA (20 ug, 5 ug or 0.5 ug) for 500 us at 300V using BTX. After electroporation, cells were incubated overnight in 37° C. incubator. Next day, K562 expressing different level of human CD19 (huCD19) were stained for expression and co-cultured with CD19 Binder's CART cells at 1:2 ratio to measure stimulated intracellular cytokine staining.

Western blot. CART cells were lysed in a 70111 of RIPA lysis buffer (1× protease and phosphatase inhibitor cocktail (Thermo Scientific™ Halt™ Protease and Phosphatase Inhibitor Cocktail), incubated at 4° C. for 30 min and centrifugation at 12000×g (at 4° C.) for 30 min. The supernatants were collected and protein concentration determined with BCA protein assay kit (Thermofisher). 30-60m of protein was mixed with appropriate amount of reducing agent (10×) and LDS sample buffer (4×) and heated the samples at 95° C. for 5 min. 30 ul of samples was loaded in 4-12% PAGE gel at 100V for 2 hrs. The gel was transferred onto Immnobilon-P membranes for overnight at 25V. The membranes were blocked with 5% skim milk at room temperature for 30 min, probed with anti-CD247 or anti-CD3z, (BD Pharmingen™) and incubated at 4° C. for overnight. Subsequently, the membranes were incubated with diluted secondary antibodies at room temperature for 1 h. Detection of transferred proteins visualized by the SuperSignal™ West Pico PLUS Chemiluminescent Substrate kit (Thermofisher) according to the manufacturer's protocols.

CART CD19binders cell in vitro stress test. 2E6 CAR CD19 binder positive T cells were co-cultured with 8E6 IRF720⁺ Nalm6-wt or IRF720⁺ Nalm6-CD19 knockout (KO) cells for a 1:4 CAR⁺:Target ratio. After 3-4 days, 0.5 ml of the co-cultures were collected and stained to determine T cell number and phenotype by flow cytometry. CD19 binder T cell phenotypes were evaluated for viability using Live/dead violet fixable viability kit (Life Technologies) following the manufacturer's protocol and then stained with the following anti-human antibodies: BV605-CD45, PE-CD4, BV510-CD8, BV650-CD45RO, PerCP-CCR7, BV711-PD1, BV785-CD69, PE-Cy7-ICOS, and FITC-CD19 protein. CountBright™ Absolute Counting Beads (Invitrogen) were used as an internal standard to calculate absolute cell counts in cell suspensions. After calculations, CD19 binder T cells were seeded with fresh IRF720⁺ Nalm6-wt cells at a ratio 1:4 (CD45⁺:Nalm6-wt). This process was repeated every 3-4 days for 25 days, total 6 rounds. Flow cytometric data was acquired on an LSRII Fortessa™ Cytometer (BD Bioscience) and analyzed with FlowJo™ v10 software (FlowJo, LLC).

Mouse Experiments. NSG mice (NOD/scid/IL2rg) were purchased from Jackson Laboratory and bred in the animal facility at the University of Pennsylvania. 8-12 weeks old, male or female mice, were used in this study. For the Jeko-1 lymphoma mouse model, each mouse was tail vein injected with 1E6 Jekol-CBG-GFP cells and seven days later, with 1E5 human CAR CD19binder⁺ T cells. Mice were health monitored twice per week with tumor BLI and weight measured weekly. Mice were bled for T cells engraftment. Endpoint euthanization for study were disease progression (BLI>1E13 P/S), 20% weight loss and lethargic activity or hunched posture.

TruCount™ assay. The TruCount™ assay was performed to determine absolute numbers of huCD45⁺ cells circulating in mouse whole blood. Anti-human mAbs mix of CD45-BV605, CD4-BV711, CD8-V500, PE-conjugated huCD19 protein were added to TruCount™ tubes followed by 50 ul of anticoagulated whole blood. Samples were vortexed gently and incubated for 15 min in the dark at room temperature. Then 450 μL of BD FACS™ Lysing Solution was added to each tube, vortexed and incubated for 15 min in the dark at room temperature. Data was acquired within 1-3 hrs of staining on a LSRII Fortessa™ flow cytometer (BD Bioscience).

### Example 2: Generation of CD19 Binders

This example describes the identification of the novel CD19 binders disclosed herein.

Affinity tuning of CAR binding domains can reduce targeting of cells expressing lower levels of the targeted antigen. An affinity tuning platform to generate low affinity variants of CD19 binders was generated by comprehensively mutating heavy and light CDR3 regions in combination with high-throughput screening using a large yeast display human antibodies libraries and antibody characterization assays. See e.g., AvantGen Inc., avantgen.com/therapeutic-antibodies. Multiple single amino acid substitution variants were generated, with the goal of only maintaining the CD19 scFv binding specificity and key CAR properties, including high CAR T cell expansion and lack of tonic signaling, while also having a low affinity and a fast off-rate.

Identification of antibodies with reduced affinity represents a relatively uncommon objective in antibody discovery and poses unique challenges when developing appropriate screening approaches. However, the comprehensive mutagenesis approach taken enabled the identification of 12 binders showing various levels of reduced binding. Table 3, and FIGS. 2A-E.

### Example 3 Identification of Unique CD19-Binders

As shown in FIG. 1, six general steps were taken to identify the unique CD19-specific antibody clones used to generate the novel CD19 binders described herein. Specifically, human antibody library for CD19-specific antibody clones were screened and induced on phage display in approximately 100 million yeast cells (Step 1). In step 2, enrichment for clones that bound biotinylated CD19-Fc using a streptavidin microbead column (MACS technology) was performed twice (2×). FACS screen of 6 phage display libraries for enrichment for binding to CD19-Fc was performed. Multiple rounds of FACS enrichment with fluor-labeled CD19 to enrich for CD19-specific clones were conducted. From these screens, a panel of up to thirty (30) single-chain variable fragments (scFv) clones CD19-specific unique clones were identified. The sequences of these CD19 scFv clones were selected based on their specific binding to CD19 expressed on recombinant HEK 293F cells transfected with a human GFP-tagged CD19 (huCD19-GFP) plasmid.

These up to 30 scFv clones were further screened based on their ability to bind baculovirus or SIGLEC. Clones that bound biotinylated baculovirus or SIGLEC 7, 8 and 9 were removed from consideration. From this selection, clones A4 (clone 43 or 43), E4 (clone 44 or 44), and E7 (clone 45 or 45) were identified (Step 3).

To ensure that the up to (30) clones could recognize native CD19 expressed on tumor cells, the clones (scFv) were also screened against NALM6 tumor cell line positive (+) CD19 expression (NALM6 cell line+CD19). CD19 knockout Nalm6 cell line (NALM6 cell line⁻ CD1; or CD19KO Nalm6 cell line) were used as a negative control for the selection. CD19KO Nalm6 cell lines used for the screen were engineered CRISPR knocked-out (KO) Nalm6-CD19KO cells. Clones that bound CD19⁺ NALM6 tumor cells, but not CD19-NALM6 tumor cells (CD19K0) were selected (Step 4). From the CD19⁺ NALM6 tumor cells screen, the 12 CD19-specific antibody clones were identified. The clones, disclosed in Table 3 are called: A2 (clone 42; or 42); A4 (clone 43; or 43); E4 (clone 44 or 44); E7 (clone 45 or 45); 7 (clone 46 or 46); 10 (clone 47 or 47); 11 (clone 48 or 48); 14 (clone 49 or 49); 15 (clone 50 or 50); 16 (clone 51 or 51); 18 (clone 52 or 52); 23 (clone 53 or 53). FIG. 1 and Table 3. The nucleic acid sequences of these 12 novel CD19 binders are shown in the sequence alignment disclosed in FIGS. 2A-2E, and Table 1. The sequence similarities between these clones are shown in FIG. 2F.

Thus, the original up to 30 scFvs were reduced to twelve (12) novel CD19 scFvs that specifically bound to wild type NALM6-wt cells but not engineered CRISPR Nalm6-CD19KO cells. These twelve CD19 binders were designated 42, 43, 44, 45, 46, 47, 48, 49, 50, 51, 52 and 53 (FIG. 1; Table 3).

The 12 novel CD19 binders were used to engineer novel anti-CD19 chimeric antigen receptors (CD19) for further analysis. These selected scFvs were engineered to be functioning chimeric antigen receptors (CARs) designed to use structural components of the CD8 leader, hinge and transmembrane domain TM for membrane expression and signaling domains of 4-1BB and CD3zeta to direct activation responses. The novel anti-CD19 CARs comprised an antigen binding domain comprising an anti-CD19 antibody fragment or scFv selected from the group consisting of 42, 43, 44, 45, 46, 47, 48, 49, 50, 51, 52 and 53 or optimized variant thereof; a CD8 hinge domain; a CD8 transmembrane domain; the intracellular domain of a 4-1BB costimulatory molecule; and a CD3 zeta intracellular domain (anti-CD19 scFv-BBz CAR constructs).

Each of these CARs was cloned into a lentiviral expression plasmid pTRPE (pTRPE anti-CD19 scFv-BBz CAR constructs; FIG. 1). Each of the twelve CAR constructs was packaged and transduced in both Jurkat NFAT reporter cells and primary human T cells; and characterized as described herein.

Additional functional characterization of the 12 novel binders are described in the co-pending PCT application and U.S. application which claim priority to U.S. Provisional Application No. 63/417,220, filed on Oct. 18, 2022, and U.S. Provisional Application No. 63/426,967, filed on Nov. 21, 2022, the contents of which are hereby incorporated by reference in their entirety for all purposes.

### Example 4: Functional Characterization of the 12 CD19 Binders

CD19 CARs comprising the novel binders were initially transduced into Jurkat NFAT-GFP reporter cell line to evaluate CAR expression, detection, and their ability to activate NFAT transcription pathway and express GFP. These cells were transduced at high levels, for example greater than 70%, which allowed for greater sensitivity of ligand-independent activation of the NFAT reporter, known as tonic signaling.

Detectable surface expression of the CD19 CAR (CAR CD19binders) varied in levels among the 12 tested binders, with CARs 43 and 45 having the highest expression. The expression of a CAR comprising a clone 42 antigen binding domain was highly detected on the surface of transduced Jurkat-NFAT-GFP reporter cells (mean fluorescence intensity (mfi) of 74.6%), but the clone 42 CAR did not appear to induce tonic signaling (e.g., 1.5% mfi) in the transduced cells. Relative to a control CD19 CAR comprising a known binder (e.g., FMC63 binder), a CAR comprising a clone 42 antigen binding domain (CAR42, CAR CD19 42, or CAR CD19binder 24, CD19 CAR 42) produced a high amount of GFP expression, and no tonic signaling. A CAR comprising a clone 52 antigen binding domain was expressed on the surface of the transduced cells (medium mfi surface detection) and the clone 52 CAR appeared to induce some tonic signaling.

Together, these data demonstrated that CARs comprising the novel CD19 binders disclosed herein can activate the NFAT pathway without stimulation with CD19 ligand (tonic signaling or ligand independent activation). CD19-42 CARs showed the lowest level of CAR-induced tonic signaling. Other CARs appeared to induce tonic signaling even though the surface expression of these CARs was not readily detected on the CAR T cell surface. Generally, most CARs tested expressed on the cell surface and activated the NFAT pathway.

Further analyses of percent of detectable CAR transduced T cells and the MFI of their expression on both the CD4⁺ and CD8⁺ T cells at different times in expansion (e.g., D7 vs D11; data not shown) showed that CD19 CARs 44, 45, and 52 appeared to be the most stable in detectable transduction over time, but these CD19 CARs appeared to lose their MFI towards the end of expansion. Part of this loss was attributed to normal T cell size reduction. Similar results were observed with a control CD19 binder. While the CD19 control CAR and CD19 CARs comprising binders 44, 45, and 52 appeared to be the most stable in detectable transduction over time, all tested CAR T cells seemed to lose their MFI towards the end of expansion. Part of this loss could be a result of normal T cell size reduction.

Cytokine Production

The ability of CARs comprising the novel CD19 binders to produce cytokines after a 4 hr co-culture with Nalm6 cells was also evaluated by intracellular detection using flow cytometry. The cytokines tested were IL-2, TNFα, and/or IFNγ. Only CAR T cells expressing CD19 CARs comprising CD19 binder 42, 43, 44, 45, 46, or 52 induced significant and quantifiable levels of cytokine. CART cells expressing a positive CD19 CAR control also produced cytokines. As expected, CAR T cells did not produce cytokines in the absence of stimulation (e.g., before co-culture), all CAR T cells tested had equal potential to produce cytokines as shown with PMA/ionomycin stimulation.

All tested ND307 CD19 CAR T cells showed HLA-DR expression for both CD4⁺ and CD8⁺ T cells. These results indicated that the ND307 CD19 CAR T cells were still in a more activated state than untransduced (NTD T) cells. All tested ND307 CD19 CAR T cells also showed 4-1BB expression in both CD4⁺ and CD8⁺ T cells. 4-1BB (CD137) was used as an early activation surface marker and its expression or lack thereof indicated the resting state of the ND307 CD19 CAR T cells when compared to control CD19 CART cells. CAR T cells expressing CARs comprising a CD19 binder 42 had the lowest 4-1BB expression levels (0.70 and 0.57 mfi).

on Day 7, CD19 CAR T cells were still in a more activated state when compared to untransduced (NTD) T cells. However, the observed level of activity may not have been enough to produce cytokines in some CAR T cells. The CD19 CAR T cells were also tested to assess their activation level using an early activation surface marker 4-1BB (CD137). The early activation surface marker 4-1BB (CD137) was detected at Day 11 following transduction, which was an indication of their resting state.

Together, the expression of the CD19 CARs, HLA-DR and 4-1BB, and cytokine profiles analyses confirmed that CD19 CAR T cells expressing CARs comprising CD19 binders 42, 43, 44, 45, 46 and 52 produced cytokines in a CAR-dependent manner. CAR T cells expressing CARs comprising the CD19 binders 50 and 51 had a good detectable CAR surface expression, but a weaker CAR-induced cytokine production. As shown below, CARs comprising CD19 binders 42, 43, 44, 45, 46 50, 51, and 52 were further analyzed using additional primary T cells derived from two different donors.

### Example 5: Effective CD19 CAR T Cell Killing and Persistence

An effective CAR T cell is one that can kill and persist. To determine which of the novel CD19 binders would endow CAR T cells with these two properties (e.g., the best candidates matching this criterion) an activation stress test was performed. Thawed ND528 cells transduced with the optimized CD19 CARs were used for serial re-stimulation studies and for evaluating cell killing properties.

Re-stimulation stress test of ND528 CART cells expressing optimized CD19 binders was performed using optimized CD19 binders 42OP, 43OP, 44OP, 45OP, 46OP, 51OP, and 52OP. The killing target cells were Nalm6 cells. The low expression of CD19 binders 43OP, 46OP, and 51OP made testing difficult. At the end of each stimulation process, CD19 CAR T cells were stained and the numbers of live cells were determined by flow. New co-cultures were established and CAR T cells were evaluated using flow cytometry for T cell phenotypes (cytotoxicity).

In the activation stress test, CAR T cells expressing a novel CD19 binder (e.g., 42OP, 43OP, 44OP, 45OP, 46OP, 51OP, and 52OP) were co-cultured with wild type (wt) Nalm6 at a 4:1 ratio of Targets:CART cells on day 0 (D0). The cultures were evaluated every 3 to 4 days by flow cytometry and selected based on CD45 expression (CD45⁺). These co-cultures were re-stimulated (i.e., re-established co-cultures) at 4:1 for 6 rounds. Specifically, the cells were re-stimulated on Day 4, (Round 1, D4), Day 7 (Round 2, D7), Day 11 (Round 3, D11), Day 14, (D14, R4), Day 18 (Round 5, R5), and Day 21 (Round 6, R6).

For screening, the cells were serially gated to identify the correct population of CD19 CAR T cells. Initially, CAR T cells were gated for live cells and selected. Then, the live cells were gated for Nalm6 and Nalm6 negative (Nalm6⁻) cells were selected. These Nalm6⁻ cells were then gated for human CD45 (huCD45), and huCD45⁺ cells were selected. Lastly, huCD45⁺ cells were gated for CD8 (CD137, CCR7, and PD1) and CD4 (ICOS, CD45RO, and CD69).

Titrated Cell Killing

To determine the ability of each of the engineered CART cells to kill target cells (e.g., Nalm6) from the start, the thawed ND528 CAR T cells used for restimulation stress test, were evaluated for real time cell killing of one cycle at 3:1, 1:1, 1:3 and 1:10 CAR⁺:Nalm6 wt ratios.

The titrated cell killing results indicated that all CD19 CAR T cells tested killed target cells at relatively the same level. The initial count per image was about 1,000 for CD19-42OP CAR T cells. At 45 minutes, the count was less than 50 in 1:1 and 3:1 CAR⁺:Nalm6 wt ratios. However, in the 1:10 CAR⁺:Nalm6 wt ratio, the count was over 2500; and at 1:3 CAR⁺:Nalm6 wt ratio, the count was about 1500.

In the CD19-45OP CAR T cells, the count per image was about 900. At 45 minutes, the count was less than 50 in 1:1 and 3:1 CAR⁺:Nalm6 wt ratios. However, in the 1:10 CAR⁺:Nalm6 wt ratio, the count was over 2500; and at 1:3 CAR⁺:Nalm6 wt ratio, the count was about 500. In the CD19-44OP CAR T cells, the count per image was about 900. At 45 minutes, the count was about 0 in 3:1 CAR⁺:Nalm6 wt ratio. In 1:1 CAR⁺:Nalm6 wt ratio, the count was less than 50. However, in the 1:10 CAR⁺:Nalm6 wt ratio, the count was over 1100; and at 1:3 CAR⁺:Nalm6 wt ratio, the count was about 900. In the CD19-52OP CAR T cells, the count per image was about 900. At 45 minutes, the count was about 0 in 3:1 and 1:1 CAR⁺:Nalm6 wt ratios. However, in the 1:10 CAR⁺:Nalm6 wt ratio, the count was over 2000; and at 1:3 CAR⁺:Nalm6 wt ratio, the count was about 1000.

These results showed that the optimized CD19 CAR T cells killed target cells within about 45 minutes at 1:1 and 3:1 CAR⁺:Nalm6 wt ratios. At 1:10 and 1:3 ratios, there was an enhancement in the cell count rather than a decrease. The CAR T cells showed similar killing (e.g., cytotoxic) efficacy for CD19 42OP CAR T cells, CD19 44OP CAR T cells, CD19 45OP CAR T cells, and CD19 52OP CAR T cells.

CD8⁺ T Cells Expansion and CD4⁺ T Cells Collapse.

Concurrently, these same thawed ND528 CAR T cells were used to initiate the six rounds of restimulation stress tests to determine their ability to persist as effective therapeutics. In the first round, the CD19 CAR T cells were co-cultured with target cells, either Nalm6-wt or Nalm6-CD19KO. During the course of the six rounds of restimulation with Naplm6-wt, the percent increase in the population of CD8⁺ and CD4⁺CAR T cells was evaluated.

Expansion of ND528 CD4⁺ and CD8⁺CD19 CART cells over 6 re-stimulations was assessed. At the end of stimulation 1 with Nalm6-CD19KO, the population of CD4⁺ T cells in CART cells expressing 42OP, 44OP, 45OP, and 52OP were respectively, 53.6%, 40.1%, 47.6%, and 35.5%. A similar trend was observed in CAR T cells stimulated with Nalm6-WT. In particular, at the end of stimulation 1, the population of CD4⁺ T cells in CAR T cells expressing 42OP, 44OP, 45OP, and 52OP were respectively, 53.6%, 40.1%, 47.6%, and 35.5%.

Over the course of the six rounds of stimulation, the population of CD4⁺ T cells in ND528 CD19-42OP CART cells at the end of each round (R) was 53.6% (R1), 33.1% (R2), 15.7% (R3), 11.9% (R4), 9.47% (R5), and 8.89% (R6). The population of CD4⁺ T cells in ND528 CD19-44OP CART cells at the end of each round (R) was 40.1% (R1), 18.6% (R2), 11.2% (R3), 9.11% (R4), 9.73% (R5), and 11.9% (R6). The population of CD4⁺ T cells in ND528 CD19-45OP CART cells at the end of each round (R) was 47.6% (R1), 25.9% (R2), 14.8% (R3), 11.4% (R4), 9.84% (R5), and 9.68% (R6). The population of CD4⁺ T cells in ND528 CD19-45OP CART cells at the end of each round (R) was 35.5% (R1), 19.0% (R2), 10.9% (R3), 7.48% (R4), 9.84% (R5), and 6.11% (R6). Generally, CD19 42OP CAR T cells, CD19 44OP CAR T Cells, CD19 45OP CAR T cells, CD19 52OP CAR T cells and a control CAR T cell comprising anti-CD19 scFv showed a similar population decrease trend.

At the end of Stimulation 1 with Nalm6-CD19KO, the population of CD8⁺ T cells in CAR T cells expressing 42OP, 44OP, 45OP, and 52OP were respectively, 30.6%, 42.0%, 19.5%,54.3% and 48.0%. A similar trend was observed in CAR T cells stimulated with Nalm6-WT. In particular, at the end of stimulation 1, the population of CD8⁺ T cells in CAR T cells expressing 42OP, 44OP, 45OP, and 52OP were respectively, 30.6%, 42.0%, 19.5%,54.3% and 48.0%.

Over the course of the six rounds of stimulation, the population of CD8⁺ T cells in ND528 CD19-42OP CAR T cells at the end of each round (R) was 30.6% (R1), 41.2% (R2), 48% (R3), 54.3% (R4), 60.4% (R5), and 67.8% (R6). The population of CD8⁺ T cells in ND528 CD19-44OP CAR T cells at the end of each round (R) was 42.0% (R1), 54.3% (R2), 56.6% (R3), 62.1% (R4), 62.0% (R5), and 62.4% (R6). The population of CD8⁺ T cells in ND528 CD19-45OP CAR T cells at the end of each round (R) was 19.5% (R1), 27.7% (R2), 24.6% (R3), 33.0% (R4), 28.9% (R5), and 30.1% (R6). The population of CD8⁺ T cells in ND528 CD19-52OP CAR T cells at the end of each round (R) was 48.0% (R1), 54.3% (R2), 55.2% (R3), 62.4% (R4), 64.4% (R5), and 70.2% (R6).

Thus, in ND528, the manufacturing expansion led to different levels of CD8⁺ T cell population in control CD19 CAR, CD19 52OP CAR T cells, CD19 44OP CAR T cells, CD19 42OP CAR T cells, and CD19 45OPCAR T cells. For example, the CD8⁺ T cell population were respectively about 51.9%, 48%, 42%, 30.6% and 19.5%. CD19 42OP CART cells, CD19 44OP CAR T Cells, CD19 45OP CAR T cells, CD19 52OP CAR T cells and a control CAR T cell comprising anti-CD19 scFv showed a similar population increase trend.

The changes in CD8⁺ T cell population were respectively 55% for CD19 42OP CAR T cells (30.6 to 67.8); 33% for CD19 44OP CAR T Cells (42 to 62.4); 35% for CD19 45OP CAR T cells (19.5 to 30.1); 32% for CD19 52OP (48 to 70.2); and 35% for the control CAR (51.9 to 79.7).

Relatively similar expansion was observed in all tested ND528 CD19 CD4⁺ and CD8⁺CAR T cells over 6 re-stimulations. The percentages of CD8⁺ and CD4⁺ T cells at the beginning of the restimulation test (e.g., end of simulation 1) were respectively (a) 30.6% and 53.6% for CD19 42OP CART cells, (b) 42% and 40.1% for CD19 44OP CART cells; (c) 19.5% and 47.6% for CD19 45OP CAR T cells, and (d) 48% and 35.5% for CD19 52OP CART cells. The percentages of CD8⁺ and CD4⁺ T cells at the end of the restimulation test (e.g., end of simulation 6) were respectively (a) 67.8% and 8.9% for CD19 42OP CAR T cells, (b) 62.4% and 11.9% for CD19 44OP CAR T cells; (c) 30.1% and 9.7% for CD19 45OP CART cells; and (d) 70.2% and 6.1% for CD19 52OP CART cells. The loss of CD8⁺ T cells in CAR T cells expressing CD19 45OP was unexpected.

CD19 42OP CAR T cells were an exception to this trend because the increase in CD8⁺ T cell population was faster. For example, the initial CD8⁺ T cell population in CD19 42OP CAR T cells was about 30.6%, yet it reached substantially similar final CD8⁺ T cells levels as other tested CD19 CAR T cells (about 67.8%) within the same time frame. In contrast, an unusually high levels of CD4⁻CD8⁻ CAR T cells population were found in the CD19 45OP CAR T cells.

Tumor Clearance

The ability of the novel CD19 CAR T cells to maintain tumor clearance during the stress test was analyzed by flow cytometry. Dead cells were identified based on the IRFP720 fluorescence exclusion. Table 16 shows raw data from flow cytometry analysis of the long term Nalm6 cells killing activity of CD19 CART cells expressing CARs comprising CD19 binders 42OP, 44OP, 45OP, and 52OP. CD19 CAR T cells maintained long term cell killing activity over the course of the six restimulations. For the cytotoxicity assay, flow cytometry was gated for Nalm6 cells and viability was assessed based on the exclusion of IRFP720 fluorescence. IRFP720 negative (IRFP720⁻) cells were dead and IRFP720 positive (IRFP720⁺) cells were alive.

At the end of Stimulation 1 with Nalm6-CD19KO, the population of IRFP720⁺ cells exposed to CAR T cells expressing 42OP, 44OP, 45OP, and 52OP were respectively, 95.4%, 88.1%, 90.6% and 94.4%. While the population of IRFP720⁻ cells was respectively 4.34%, 11.9%, 9.33%, and 5.46%.

Specifically, over the course of the restimulation, the population of IRFP720⁺ Nalm6 cells exposed to CD19-42OP CART cells was 91.9% (R1), 5.92% (R2), 0.01% (R3), 4.53E-3% (R4), 5.69E-3% (R5), and 0% (R6). Conversely, the population of IRFP720⁻ Nalm6 cells (dead cells) exposed to CD19-42OP CAR T cells was 7.99% (R1), 93.0% (R2), 99.9% (R3), 99.9% (R4), 99.9% (R5), and 99.9% (R6). The population of IRFP720⁺ Nalm6 cells exposed to CD19-44OP CAR T cells was 22.0% (R1), 0.016% (R2),14.0% (R3), 0.02% (R4), 77.7% (R5), and 90.4% (R6). Conversely, the population of IRFP720⁻ Nalm6 cells (dead cells) exposed to CD19-44OP CART cells was 77.9% (R1), 99.6% (R2), 85.9% (R3), 99.8% (R4), 22.1% (R5), and 7.83% (R6). The population of IRFP720⁺ Nalm6 cells exposed to CD19-45OP CAR T cells was 71.5% (R1), 1.39% (R2), 1.79% (R3), 1.09% (R4), 73.3% (R5), and 82.6% (R6). Conversely, the population of IRFP720⁻ Nalm6 cells (dead cells) exposed to CD19-45OP CART cells was 28.4% (R1), 98.0% (R2), 98.1% (R3), 98.5% (R4), 26.3% (R5), and 16.7% (R6). The population of IRFP720⁺ Nalm6 cells exposed to CD19-52OP CART cells was 81.0% (R1), 0.04% (R2), 0.01% (R3), 0.01% (R4), 1.01% (R5), and 0.02% (R6). Conversely, the population of IRFP720⁻ Nalm6 cells (dead cells) exposed to CD19-52OP CART cells was 18.9% (R1), 98.1% (R2), 99.9% (R3), 99.8% (R4), 98.8% (R5), and 99.9% (R6).

These results showed that CD19 CAR T cells expressing a CAR comprising CD19 binder 42OP or 52OP maintained long term cell killing over all six stimulation tests. At the end of the 6 th stimulation 99.9% of Nalm6 cells remained dead in cells cocultured with CD19-42OP CAR T cells or CD19-52OP CAR T cells. CD19 binders 42OP, 52OP showed similar long-term ability in persistence and tumor clearance. CD19-44OP CAR T cells showed the greatest tumor clearance.

In contrast, at the end of the first four day rounds of re-stimulation between Nalm6-wt and Nalm6-CD19KO, 44OP and 45OP showed a greater number of dead Naml6 compared to 42OP, 52OP, but at the end of the 6 th stimulation, the population of Naml6 had recovered with each having 90.4% (44OP) and 82.6% (45OP) IRFP720⁺ Nalm6 cells. Yet, in the absence of CD19 activation by Nalm6 (e.g., Nalm6-CD19KO co-culture), 44OP had the highest level of cell death (11.9%) suggesting a higher level of basal activation. Nalm6 vs Nalm6-CD19KO was only done in first round. By the end of the fifth and sixth rounds of re-stimulation, 44OP and 45OP had failed to control the tumor and showed a decline of surviving T cells, suggesting a failure in long term therapeutic persistence.

Expression of Activation Markers CD137, PD1, CD69, and ICOS

To determine the specificity of CD19 CAR T cells for the CD19 antigen, the expression of early activation markers, CD137, PD1, CD69, and ICOS in CART cells after the first round of stimulation with either Nalm6-WT cells or Nalm6-CD19K0 cells was determined.

The expression of CD137, PD1, CD69 was enhanced in CD19 CART cells co-cultured with Nalm6-WT when compared to CD19 CAR T cells co-cultured with CD19 knock-out (KO) Nalm6. In CD19-42OP CART cells, CD139 expression was 51.0% compared to 0.31% (CD19K0); PD1 expression was 80% compared to 32.7% (CD19K0); CD69 expression was 76.3% compared to 20.4%; ICOS expression was 3.72% compared to 1.77% (CD19KO). In CD19-52OP CART cells, CD139 expression was 30.0% compared to 0.28% (CD19KO); PD1 expression was 74.8% compared to 45.1% (CD19KO); CD69 expression was 58.2% compared to 22.6%; ICOS expression was 4.19% compared to 14.5% (CD19KO). After one round of stimulation, these activation markers showed specificity to Nalm6-wt but not to Nalm6-CD19KO. In addition, co-cultures with targeted CD19 expressed on Nalm6-wt cells led to enhanced CD137, PD1 and CD69 expression after the first round of stimulation.

These data showed the specificity of CD19 CART cells for the CD19 antigen expressed on Nalm6-WT when compared to CD19 knock-out (KO) Nalm6 based on the activation of T cells markers (CD137, PD1, CD69 and ICOS). After one round of stimulation, these activation markers showed specificity to Nalm6-wt but not to Nalm6-CD19KO.

The expression of the activation markers were also assessed over the 6^{th} rounds of stimulation. The data from flow cytometry that assessed the expression of activation receptor, CD137, on CD4⁺ T cells over time showed a general trend toward a decrease in CD137 expression.

In particular, the expression of the CD137 on CD4⁺ T cells may have been downregulated over the six round of stimulation (e.g., high internalization or membrane turnover following activation). Alternatively, the cell surface or the epitope binding was blocked, for example after CD19 engagement or binding. This was because when T cells co-cultured with Nalm6 wt were compared to those co-cultured with Nalm6-CD19KO, the expression of CD19 CARs was no longer detectable on CAR T cells co-cultured with nalm6-wt. However, their surface expression remained detectable following Nalm6-CD19KO co-culture. Accordingly, it is most likely that internalization or high membrane turnover following CAR activation is a unique feature of the novel CD19 binders described herein because most antigen targeting CARs showed lower decrease in surface detection.

By the end of the third re-stimulation (R3), 4-1BB (CD137) expression was no longer observed in all CAR T cells tested (e.g., CD19CAR binder 42OP, 44OP, 45OP, and 52OP). 4-1BB (CD137) was used as an early activation marker, but its expression or lack thereof may not measure CART cells tumor control.

To determine whether CAR T cells were exhausted on both CD4⁺CAR T cells and CD8⁺CAR T, the expression of PD1, CD69, and ICOS was evaluated. The quantification of the expression of the CD19 CAR, the activation markers, PD1, CD69, and ICOS on CD4⁺ T cells at the end of the second and fifth re-stimulation showed that after five rounds of re-stimulation, the expression of PD1, CD69, and ICOS on the CD4⁺ T cell surface was downregulated. The quantification of the expression of the CD19 CAR, the activation markers, PD1, CD69, and ICOS on CD8⁺ T cells at the end of the second and fifth re-stimulation showed that after five rounds of re-stimulation, the expression of PD1, CD69, and ICOS on the CD8⁺ T cell surface was downregulated. No significant difference between 42OP, 44OP, 45OP, and 52OP CD19 CAR T cells was observed.

Furthermore, the expression of activation receptors, such as e.g., PD1, CD69, and ICOS, on CD4⁺ and CD8⁺ T cells decreased over time. These results suggested that CAR T cells expressing CARs comprising CD19 binders 42OP, 44OP, 45OP, and 52OP were not exhausted. Rather, CAR T cells expressing CAR comprising the tested novel CD19 binders were likely downregulated over time and/or that binding to CD19 protein was likely blocked. These data suggested that CD4⁺CAR T cells and CD8⁺CAR T cells expressing a CAR comprising a CD19 binder 42OP, 44OP, 45OP, or 52OP were not exhausted.

### Conclusion

The data indicated that CAR T cells expressing a CAR comprising CD19 binders 42, 42OP, 52 and 52OP had the best attributes of low tonic signaling, strong activation rate (e.g., NFAT), doublings during manufacturing expansion phase (e.g., expansion profiles), robust and stable surface expression (e.g., with good maintenance of mfi), enhanced killing, and long term persistence in therapeutic activity.

### Example 6: IL-18 Co-Expression does not Affect CD19 CAR Expression

Several immune modulators have been shown to increase the efficacy of engineered CAR T cells. These immune modulators enhance the efficacy of CAR T cells using different mechanisms. For example, the immune modulator can increase the recruitment of endogenous immune cells to the tumor site (e.g., NK cell infiltration), increase persistence, reduce T cell exhaustion; and/or enable resistance to checkpoint inhibitors. In addition, immune modulators, such as cytokines can enhance T cell priming, antigen presentation, and T cell infiltration in a solid tumor. To further enhance the efficacy of CAR T cells comprising the novel CD19 binders disclosed herein, the CD19 CAR were co-expressed in primary human T cells with a recombining IL-18 molecule. As shown herein, arming CD19 CAR T cells comprising an original or optimized CD19 binder 42 or 52 with significantly enhanced the anti-tumor activities of the CD19 CAR T cells while reducing side effects associated with CD19 CAR. The CD19 CARs exemplified herein comprised a novel CD19 binder (42original (42og); 42 optimized (42op); 52 original (52og); and 52 optimized (52op)), a 4-11BB costimulatory domain, and CD3 zeta intracellular domain, optionally a hinge domain.

Based on expression levels and stability of the novel CD19 CARs, lack of their tonic signaling, cytokine responses and their ability for long-term persistence and tumor control in in vitro stress test, CD19 binders 42 and 52 were chosen to further evaluate co-expressing IL-18. See e.g., co-pending PCT application and U.S. application, which claim priority to U.S. Provisional Application No. 63/417,220, filed on Oct. 18, 2022, and U.S. Provisional Application No. 63/426,967, filed on Nov. 21, 2022, the contents of which are hereby incorporated by reference in their entirety for all purposes.

To determine the effect of IL-18 co-expression on the CD19 CARs, a vector comprising a CD19 CAR fused to a recombinant IL-18 via a T2A peptide was transduced in human primary T cells. The expansions of the IL-18 armored CD19 CART cells were assessed for about 30 days. CD19-IL18 CART cells were transduced at similar levels as control CAR T cells (>60% transduction efficiency).

Tonic Released of IL-18 by Armored CD19 CAR T Cells

All transduced T cells transduced with CD19-IL-18 were able to tonically produce IL-18. CD19 CART cells expressing CD19CAR and IL-18 constitutively secreted IL-18 when compared to untransduced T cells. CD19-42-IL CAR T cells produced about 1×10⁻³ pg/T cell when compared to unstranduced T cells with about 1×10⁻⁶ pg/T cell in the presence of Nalm6 (E:T 1:1 ratio), K562-mesothelin, or no stimulation. These CAR T cells also proliferated after repeated exposure to Nalm6 and eliminated tumor cells. The decreasing values are intentional.

CD19 CAR Expression was Stable Over Time.

Initially, the expansions of ND609 CAR T cells expressing original and optimized versions of CD19-42 and CD19-52 CAR either alone (CD19-42, CD19-52, CD19-23OP, and CD19-52OP) or co-expressing IL-18 (CD19-42-IL-18, CD19-52-IL18, CD19-23OP-IL18, and CD19-52OP-IL18) were compared. As shown in FIG. 3A, ND609 expansions showed similar expansions up to about day 12. After 12 days, some CAR T cells had rested to around 350fL (FIG. 3B) and were frozen for in vivo evaluations. Two million cells (2×10⁶ cells) from each of the CAR T cells groups were maintained in culture from day 12 to 27 to determine their long-term persistence without further activation. The purpose of the continued culture without stimulation was to determine the effect of IL-18 co-expression on CAR T cells ability to rest down (i.e., contract).

In this assay, the inability to rest down reflected continued tonic signaling of the CAR. After day 12, CAR T cells comprising CD19 52op, 52op-IL-18, 52-IL18, and 42op-IL18 continued expanding even in the absence of further stimulation for up to 27 days. However, the expansion of CART cells comprising CD19 42og, CD19 42op, CD19 42og-IL-18, and CD19 52og decreased steadily over time (rested down).

The light lines with symbols showed that most CD19 CAR T cells lacking the recombinant IL-18 construct (all non-IL18 expressing constructs) contracted over time. The expansion of CD19 CART cells expressing CD19-52og, CD19-42og, and CD19-42op decreased steadily over time. However, CD19-52op CAR T cells continued to expand even in the absence of stimulation. In CD19 CART cells co-expressing IL-18, CART cells expressing CD19 binder 42 original decreased steadily over time. These results indicated that CD19 binder 42 original showed less tonic signaling when co-expressed IL-18. CD19-42og-IL-18 CAR T cells showed the least continuous expansion after activation. On the other hand, 52, 52OP and 42OP appeared to have a greater basal activity which worked with IL-18 to keep cells expanding.

Furthermore, the MFIs of CAR expression between the original and optimized version of 42 and 52 indicated that the co-expression of IL-18 did affect the CD19 CAR surface expression. In fact, the data showed that IL-18 co-expression enhanced the surface expression of CAR comprising the CD19 binder 42. The enhancement may be caused by higher transduction levels in that group.

### Example 7: CD19-IL-18 CAR T Cells Effectively Cleared Tumor

ND609 CD19 CAR T cells co-expressing IL-18 were further evaluated in the Jeko lymphoma model to determine their ability to clear tumors. Two experimental schemes were developed. A first timeline used to evaluate the in vivo cytotoxic effectiveness (e.g., killing) of the IL-18 armored CD19 CAR T cells using the experimental set up shown in Table 4. Specifically, seven days before CAR T cells administration, ND609 CAR T cells were transduced with original and optimized CD19 CARs (binders 42 and 52)-2A-IL18 constructs. 1×10⁵ CD19-IL-18 CAR T cells were then administered to Jeko NSG mice at Day 0. At days 7, 14, 21, 28, and 35, the Jeko NSG mice were evaluated for health, weight, and tumor BLI. Animals were bled during the 2nd and 3rd weeks to assess the peripheral blood levels of huCD45 and serum for IL-18. In addition, during weeks 2 to 3, depending on tumor clearance, cells were harvested from the femur and spleen in mixed group to evaluate their competitiveness.

TABLE 4
ND609 CD19binder in vivo study 2

| | | Mice CAR+ |
|---|---|---|
| CAR Groups | Mice | cells/mouse |
| 42op-IL18 | 8 | 1 × 10⁵ |
| 42original-IL18 | 8 | 1 × 10⁵ |
| 52original-IL18 | 8 | 1 × 10⁵ |
| Tumor alone | 7 | — |

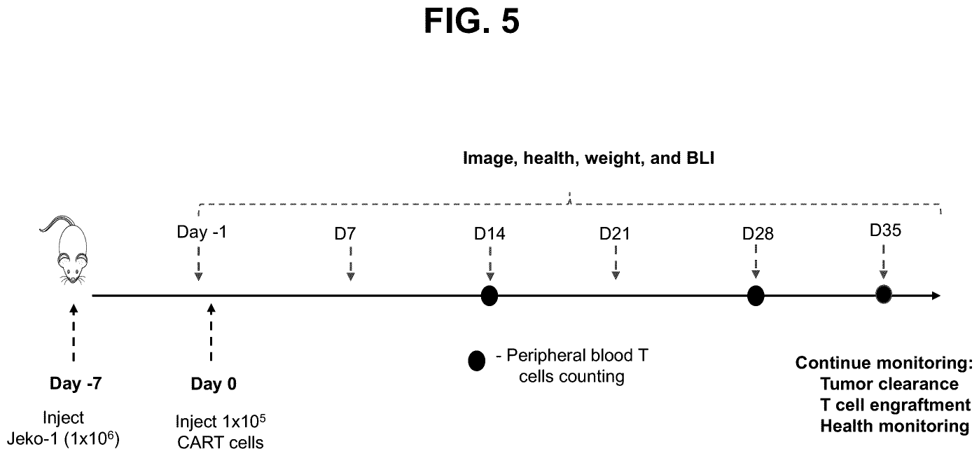

FIG. 5 shows another experimental set-up using a different manufacturing protocol and two new donors. In this scheme, a week before (D-7) CAR T cells administration, mice were injected with 1×10⁶ Jeko-1. A day before (D-1) CART cells administration, the animals were imaged, and the next day (DO), the animals were injected with 1×10⁵ CD19 CAR T cells expressing CD19-42op-IL-18; CD19-42og-IL-18; CD19-52op-IL-18; CD19-52og-IL-18; or untransduced T cells. At day 14 (D14), blood was withdrawn from the mice and peripheral blood T cells counted. The animals were imaged at day 7, 14, 21, 28, and later and monitored for tumor clearance, T cell engraftment, weight loss, BLI, and health. Results of these analyses are shown in FIGS. 6-12.

Anti-tumor activity of CD19 binders in Jekol NSG mouse model were tested using primary T cells from donor ND609 (FIGS. 6A-C), ND585 (FIGS. 6D-G), and ND608 (FIGS. 6H-K). ND609 CAR T cells expressing CD19 CAR comprising the 52OP binder did not survive the thaw in some experiments. These following data show that CAR T cells comprising CD19 binders 42og CAR armored with IL-18 cleared tumor faster than CAR T cells comprising a CD19 42op CAR, a control CD19 CAR, CD19 52og CAR and CD19 52op CAR.

Tumor Clearance

Each of the CD19 CART cells tested at 1×10⁵ CAR⁺/mouse was able to clear tumor without relapse based on tumor burden measurements (BLI/total flux) of surviving animals (FIG. 6A-K). FIGS. 6A-C show tumor clearance by ND609 CD19 CAR T cells from individual animals; and FIGS. 6D-K show tumor clearance in ND585 CAR T cells from individual animals.

Each of the CD19 42-IL18 and 52-IL18 CAR T cells showed an ability to clear tumor in mice. The CD19-42(original)-IL18 CAR T cells showed little more overall tightness in their time to initially clear tumor when compared to CD19-42(optimized)-IL18 CAR T cells. Similar tumor clearance efficacy was observed in mice injected with CD19-52 (original)-IL18 CAR T cells and CD19-52 (optimized)-IL18 CAR T cells. In all tested CAR T cells, tumor relapsed in at least one treated animal. However, the data suggested that the CD19-IL-18 CAR T cells had the ability to regain control of the relapsed tumors (FIGS. 6D-G). In contrast, CD19-42 CAR T cells that were not armored with IL-18 were not able to regain control of the relapsed tumors (FIGS. 6H-K). These data suggest that armoring CD19-42 and 52 original or optimized CART cells enhanced the efficacy of CD19 CART cells.

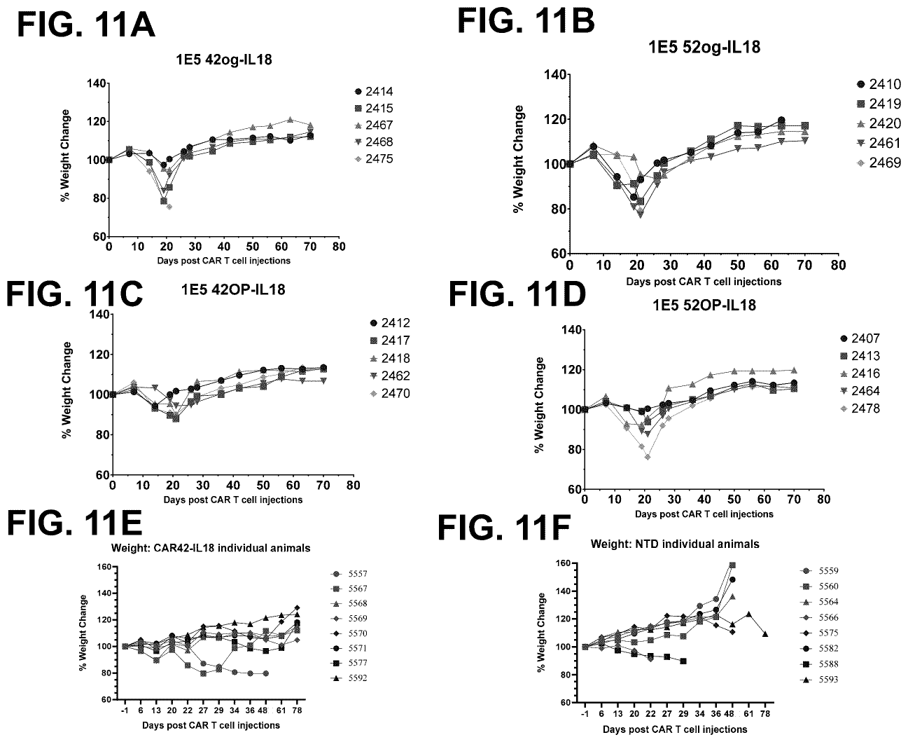

A donor-specific tumor clearance was observed during the assay. For example, tumor control by CAR T cells made from N585 donor (FIGS. 6D-F) was slower compared to CAR T cells made from N609 donor. All N585 CD19 CAR T cell took longer to clear tumor when compared to N609 CAR T cells (FIGS. 6A-C). Animals administered CD19-42(og)-IL18 CAR T cells, CD19-52(og)-IL18 CAR T cells, and CD19-52(op)-IL18 CAR T cells showed signs of critical weight loss around the time of tumor regression. However, the weights recovered with tumor clearance (FIGS. 11A-D). Generally, animals administered with optimized CD19-42 (op)-IL18 CAR T cells showed the least weight loss (FIG. 6E vs FIG. 11C). This reduced weigh loss correlated to a slower tumor clearance.

Significantly Improved CD19 CAR

Two mice injected with a well-known CD19 CAR relapsed. This was unexpected because that positive control CD19 CAR had been described to consistently clear tumor. Thus, its weakened response in this assay indicated that the novel CD19 binders disclosed herein are better, more efficient and less toxic than known CD19 binders when armored with IL-18. The positive control CD19 CAR T cells did not have the expected tumor control. Even though they showed signs of controlling tumor, the mice were euthanized because of excessive weight loss. The novel CD19 binders disclosed herein showed a stronger tumor clearance efficacy and less side effects when armored with IL-18 when compared to the positive control CAR.

T Cell Engraftment and Persistence

To determine the persistence of CD19-IL-18 CART cells injected mice, the percentages of human CD45⁺ cells in peripheral blood from treated mice were assessed by flow cytometry. As shown in FIGS. 7A-D, the concentration of huCD45⁺ T cells in the blood of administered animals decreased over time. Indeed, the huCD45⁺ T cells in murine peripheral blood linearly decreased started at day 20. This decreased correlated with the timing of tumor clearance shown in FIGS. 6A-C and G. These results further showed that CD19-42(og)-IL18 CAR T cells, CD19-42(op)-IL18 CAR T cells, CD19-52(og)-IL18 CAR T cells, and CD19-52(op)-IL18 CAR T cells contracted in numbers after tumor clearance. See FIG. 3 (compare IL-18 vs no IL-18).

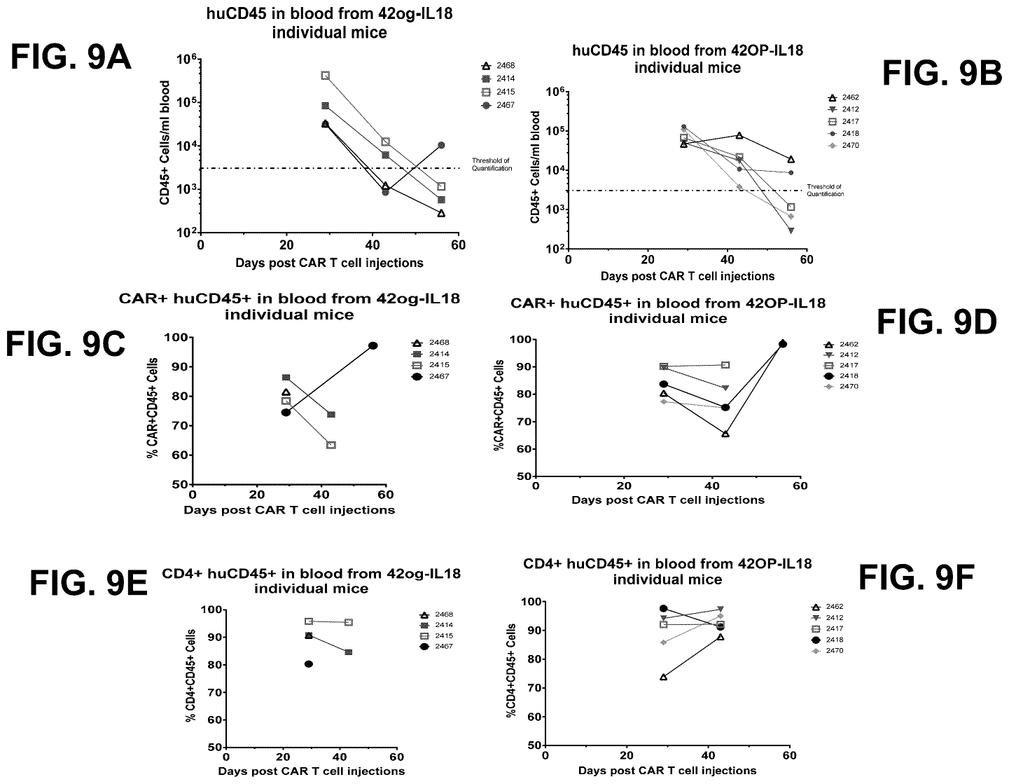

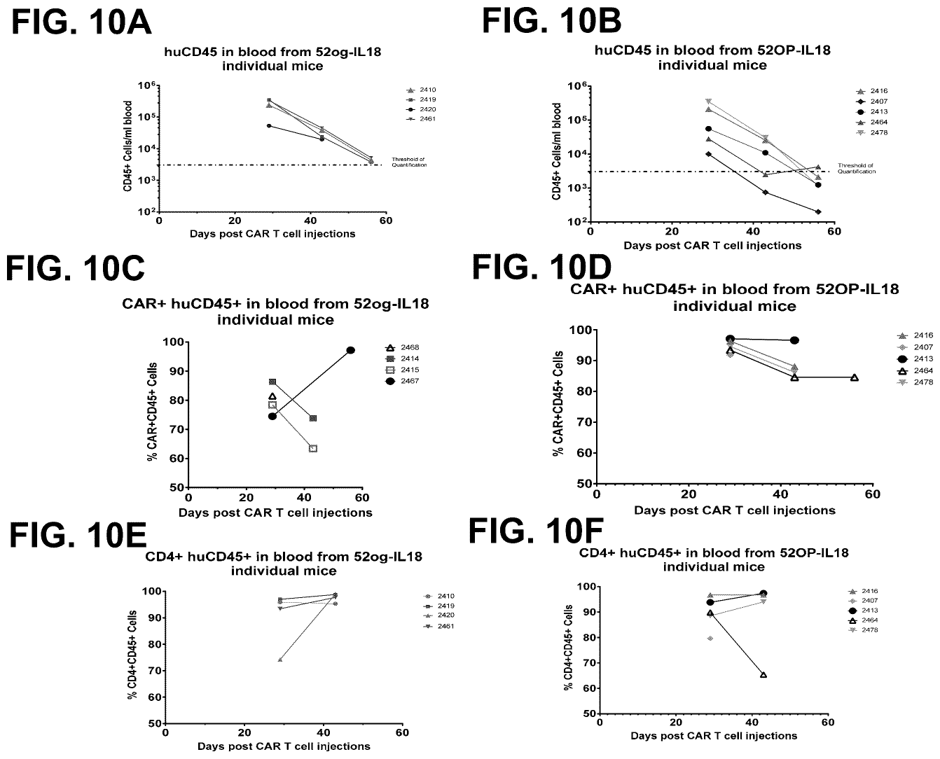

FIGS. 9A-F and 10A show that the engraftment of human CD45⁺ cells in peripheral blood contracted over time for all groups. In some cases, engraftments resulted in the expansion of huCD45⁺ T cells (FIG. 9A, on day 58 vs day 44). This was possibly a result of GVH response, which can be part of the animal model at longer end points. Peripheral blood from mouse 2467 administered with CD19-42(og)-IL18, mouse 2418 administered with CD19-42(op)-IL18, and mouse 2478 administered with CD19-52(op)-IL18 showed an expansion in huCD45⁺ cells/ml of blood on day 58 when compared to day 28. No humanCD45⁺ T cells were observed in the peripheral blood of these animals on day 15 for all groups tested. On day 58, these were the only mice administered with CD19-42(og)-IL18 and CD19-52(op)-IL18 that had detectable human CD45⁺ cells in their blood. Two of five mice administered with CD19-42(op)-IL18 and all mice administered with CD19-52(og)-IL18 had detectable numbers of human CD45⁺ cells. However, the percentage of human CD45⁺ cells in their peripheral blood were just above the threshold levels of quantification (dotted lines; FIGS. 9A-B and FIGS. 10A-B). FIGS. 9C-F and 10C-F show CAR⁺ cells in human CD45 cells in blood. FIGS. 7A-F show graphs demonstrating that the percentage of CAR positive and human CD45 positive (CD45⁺CAR T cells; FIGS. 8A-C) and the percentage of human CD45 positive and CD4 positive (huCD45⁺CD4⁺; FIGS. 8D-F)) cells in the peripheral blood of animals administered armored CAR T cells comprising the original or optimized CD19-42 CARs; and original CD19-52 CARs overtime. These figures (FIGS. 8A-F, 9C-F, and 10C-F) demonstrated that IL-18 co-expression enhanced or maintained a high percentage of CD4⁺ CD19 CAR T cells in treated animals.

In FIG. 7A, one mouse administered with CD19-42(og) CAR T cells CAR showed a linear expansion of huCD45⁺ T cells (mouse #534). Further analyses of this animal showed enhanced weight loss and was euthanized. While the positive control CD19 CAR showed similar behavior (e.g., peripheral T cells rose in numbers) in one animal, those CAR T cells decreased at a later time. The enhancement in peripheral T cells observed in mouse #534 was due to a GVHD response. FIGS. 8A and D show the zero response drove a CAR independent T cell expansion as the percent of CAR positive T cells was decreasing. Thus, the IL-18 co-expression drove or maintained a high percent of CD4⁺ T cells in the immunotherapeutic groups.

Kaplan Meier Survival Curve

The Kaplan-Meier survival curve shows the survival of animals administered with 1×10⁵ IL-18 armored ND585 CD19 CAR T cells expressing original or optimized CD19 binders 42 and 52 (FIG. 12). 100% of mice administered with CD19-42OP-IL18 CAR T cells and CD19-52OP-IL18 CAR T cells remained alive for the duration of the study (70 days). 80% of mice administered with CD19-42og-IL18 CART cells remained alive at the end of the study and one mouse died on Day 22 due to endpoint weight loss during tumor clearance. 60% of mice administered with CD19-52og-IL18 CAR T cells remained alive at the end of the study. In one mouse administered CD19-52og-IL18 CAR T cells died on Day 22 due to endpoint weight loss during tumor clearance; and a second mouse died on day 66 from spontaneous death. Some of these deaths could be have been caused by a GVH response, which is adherent in the mice model.

Mice expressing a positive control CD19 CAR T cells died at day 28 and mice administered with untransduced CAR T cells died at day 29 of excessive weigh loss. The results from the positive control CD19 CAR were unexpected, but consistent with the observations made with the tumor clearance assay above. These data showed that enhanced efficacy and tolerability of the novel CD19 binders described herein over existing CD19 binders (e.g., FMC63 binder).

### Example 8: CD19-IL-18 CAR T Cells Expression and Cytokine Production

Stable Expression Over Time

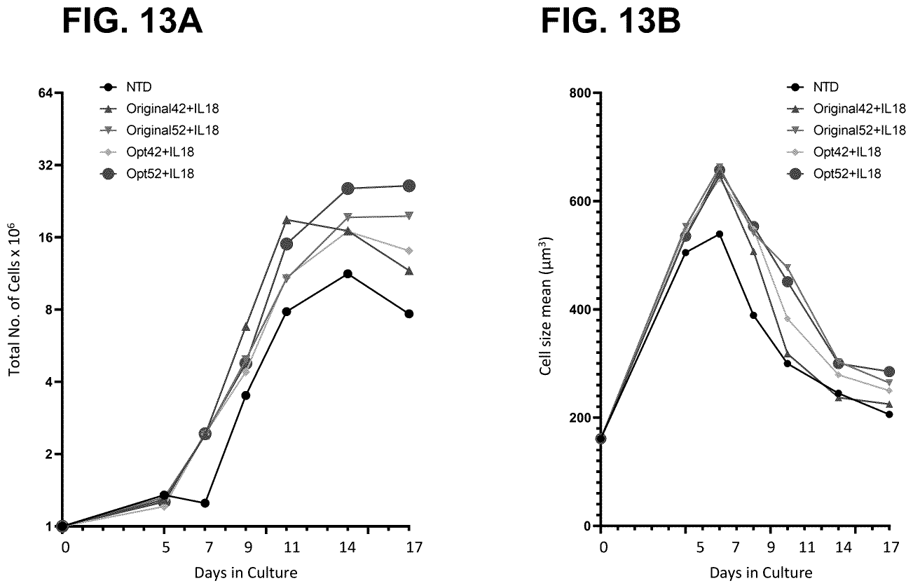

To further determine the effect of IL-18 co-expression on CD19 CAR surface expression, ND585 or ND307 T cells were transduced with CD19-42og-IL18; CD19-52og-IL18; CD19-42op-IL18; CD19-52op-IL18 or left untransduced. The resulting CD19 CAR T cells were assessed for growth (FIG. 13A) and cell size (FIG. 13B) over 17 days. All CAR T cells tested showed similar robust expansion and contraction. However, CD19-42og-IL18 CAR T cells expanded and contracted faster than CD19-52og-IL18 CAR T cells; CD19-42op-IL18 CAR T cells; or CD19-52op-IL18 CART cells. In addition, CD19-52og-lL18 CAR T cells and CD19-52op-IL18 CAR T cells contracted more slowly. These results are consistent with those obtained with ND609 CAR T cells (FIG. 3).

In addition, CD19 CAR expression profiled was assessed by flow cytometry. Table 5 shows the percentage of ND585 and ND307 T cells that expressed CD19 CAR on their cell surface. The expression of the CARs appeared to be stable in percent transduced over the 9-day manufacturing period.

TABLE 5
Expression of CD19 CAR-IL-18 transduced in ND585 T cells and
ND307 T cells (CD19 CAR Expression/FSC-H (% percentage))

| | ND585 T cells | | | ND307 T cells | |
|---|---|---|---|---|---|
| | Day 5 | Day 7 | Day 9 | Day 7 | Day 11 |
| Untransduced | 0.36 | 0.25 | 1.15 | 0.17 | 0.39 |
| 42og + IL-18 | 58.20 | 70.80 | 61.30 | 85.20 | 75.80 |
| 52og + IL-18 | 40.40 | 61.50 | 58.70 | 67.30 | 66.20 |
| 42OP + IL-18 | 66.60 | 76.70 | 69.60 | 89.00 | 82.00 |
| 52OP + IL-18 | 54.20 | 71.70 | 62.60 | 60.30 | 69.80 |

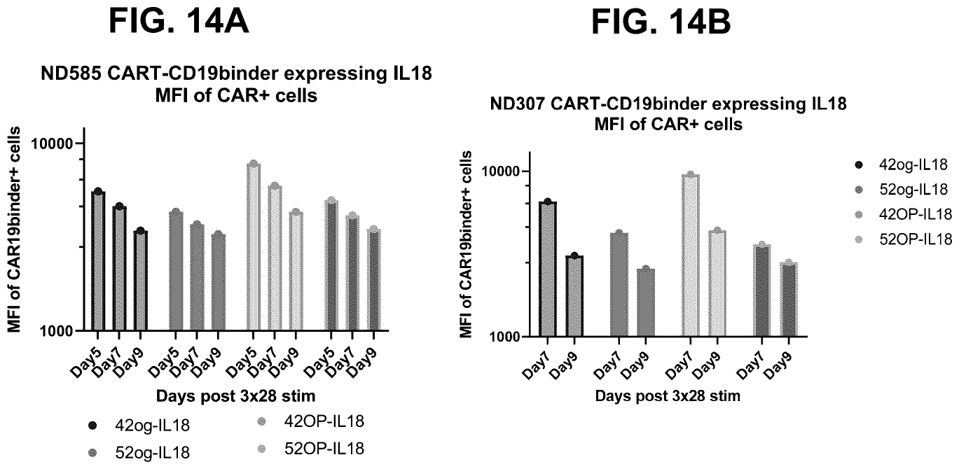

Original and optimized CD19 CART cells armored with IL-18 were further evaluate in two donors for in vitro and in vivo function as described herein. FIGS. 14A-B show the surface expression of CD19 CAR on CAR⁺ primary human T cells from ND585 donor (FIG. 14A) and ND307 donor (FIG. 14B). Expression of the CD19 CARs was stable over the time period tested in all groups tested. These cells were gated on CD4⁺ T cells. The figures demonstrate that expression of the CD19 CARs was stable over the time period tested in all groups tested. Gated on CD4⁺ T cells. In the long-term continuous cultures shown in FIG. 14A, CD19 CAR T cells co-expressing IL-18 and CD19-52og, CD19-52OP and CD19-42OP had a longer time expanding in culture without re-stimulation while CD19-42og rested down slower closer to the rate of a positive control CD19CAR-IL18 construct. The co-expression of IL-18 didn't negatively impact the MFI expression of these CD19 CARs. The overall surface expression levels of CD19-42 CARs were higher than the expression levels of CD19-52 CARs.

FIGS. 14A-B show that MFI of transduced cells decreased at a similar rate as would be expected as cell volume decreased. The overall MFIs of transduced T cells indicated that control CD19 CAR showed the most robust expression. In addition, the optimization of CD19-42 and CD19-52 may have enhanced the expression CD19-42 and 52 binders. In the other donor expansion ND307 (FIG. 14B), both the expressions of CD19-42op and CD19-42og were the closest to the control CD19 CAR expression (e.g., mfi). The trend to higher MFI with optimization was only observed with CD19-42. In ND307, CD19-52(og) and CD19-52(op) showed the lowest surface expression (MFI).

Cytokine Production

To further determine the functional characteristics of IL-18 armored CD19 CAR T cells, their cytokine production was assessed. In particular, the IL-2 and TNFα cytokine responses of CD4⁺ T cells expressing the CD19-IL-18 constructs was assessed. The T cells were from two donors-ND585 and ND307 and the CAR T cells were either stimulated with Jeko-1, or Nalm6 in co-cultures, or left unstimulated. Table 6 shows the flow cytometric analyses.

TABLE 6
IL-2 and TNF-α production from ND585 and ND307 CAR CD19
binders-IL18 CD4 T cells upon Nalm6 and Jeko-1 stimulation s

| | ND585 | | | ND307 | | |
|---|---|---|---|---|---|---|
| | IL-2 | TNF-α | IL-2 & TNF-α | IL-2 | TNF-α | IL-2 & TNF-α |
| | Jeko-1 Stimulation | | | | | |
| Untransduced | 0.130 | 0.510 | 0.850 | 0.280 | 0.081 | 1.450 |
| 42og + IL-18 | 1.850 | 25.800 | 17.000 | 6.870 | 18.600 | 20.900 |
| 52og + IL-18 | 1.900 | 24.300 | 22.300 | 8.150 | 15.500 | 17.100 |
| 42OP + IL-18 | 1.990 | 26.600 | 20.700 | 5.840 | 15.500 | 15.900 |
| 52OP + IL-18 | 1.650 | 26.700 | 22.900 | 7.050 | 16.400 | 14.100 |
| | Nalm6 Stimulation | | | | | |
| Untransduced | 0.410 | 0.260 | 2.660 | 0.260 | 0.000 | 2.550 |
| 42og + IL-18 | 3.490 | 23.900 | 39.500 | 8.240 | 19.300 | 28.700 |
| 52og + IL-18 | 2.900 | 24.700 | 35.200 | 7.260 | 17.500 | 19.000 |
| 42OP + IL-18 | 2.550 | 26.500 | 43.800 | 7.870 | 20.200 | 22.800 |
| 52OP + IL-18 | 2.170 | 25.400 | 42.900 | 7.220 | 18.100 | 16.800 |
| | No stimulation (Unstimulated) | | | | | |
| Untransduced | 0.720 | 0.160 | 1.240 | 0.630 | 0.027 | 1.330 |
| 42og + IL-18 | 0.210 | 0.500 | 0.500 | 0.500 | 0.041 | 0.500 |
| 52og + IL-18 | 0.820 | 3.990 | 2.020 | 0.460 | 0.300 | 0.570 |
| 42OP + IL-18 | 0.390 | 0.910 | 0.690 | 0.350 | 0.130 | 0.390 |
| 52OP + IL-18 | 0.660 | 4.180 | 1.730 | 0.870 | 0.420 | 0.390 |

These data showed that the production of IL-2 and TNF-α was substantially similar in original and optimized CD19 42 and 52 CAR T cells.

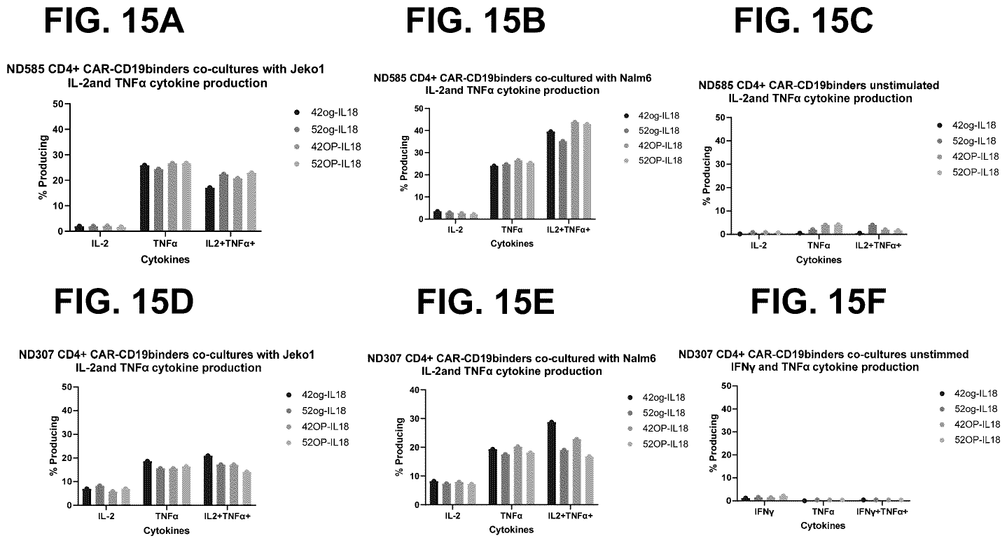

FIGS. 15A-F show bar graphs quantifying IL-2 and TNF-α production (IL-2 alone, TNF α alone, and combination of IL-2 and TNF-α) from ND585 (FIGS. 15A-C) or (ND307 (FIGS. 15D-F) CD4⁺CAR T cells expressing CD19-42og-IL-18, CD19-42op-IL-18, CD19-52og-IL-18, and CD19-52op-IL-18, upon Nalm6 (FIGS. 15B and E) and Jeko-1 (FIGS. 15A and D) stimulation on Day 9, or in the absence of any stimulation (FIGS. 15C and F).

The figures show that IL-2 and TNF-α production was substantially similar in all group tested (CD19 CAR comprising original and optimized CD19 binders 42 and 52). The mean fluorescence intensity (mfi) of CD19 surface expression in Nalm6 cells was about 13606 mfi and 6851 mfi in Jeko cells. Additional cytokine productions from CD19 CAR T cells from ND607, ND539, ND608, and ND518 are shown in Table 7.

TABLE 7
Summary of cytokine response from CAR CD19 binders expressed
from T cells from various donors upon Nalm6 stimulation

| | ND607 (Day 9) | | | ND539 (Day 8) | | |
|---|---|---|---|---|---|---|
| | IL-2 | TNF-α | IL-2 & TNF-α | IL-2 | TNF-α | IL-2 & TNF-α |
| 42og + IL-18 | 4.02 | 2.24 | 5.93 | 2.88 | 9.53 | 11.70 |
| 52og + IL-18 | 5.21 | 5.84 | 15.70 | 1.84 | 10.90 | 14.40 |
| 42OP + IL-18 | — | — | — | — | — | — |
| 52OP + IL-18 | — | — | — | — | — | — |
| | ND608 (Day 9) | | | ND518 (Day 9) | | |
| | IL-2 | TNF-α | IL-2 & TNF-α | IL-2 | TNF-α | IL-2 & TNF-α |
| 42og + IL-18 | 1.44 | 15.60 | 16.30 | — | — | — |
| 52og + IL-18 | 1.06 | 36.10 | 37.00 | — | — | — |
| 42OP + IL-18 | 1.30 | 27.10 | 35.20 | 1.27 | 6.98 | 5.99 |
| 52OP + IL-18 | 1.14 | 26.60 | 35.50 | 1.89 | 11.30 | 8.40 |

Table 8 shows the production of INF-y and TNF-α from CD8⁺ T cells. The production of IFN-γ and TNF-α from ND585 and ND307 CD8⁺CART cells expressing CD19 CARs and an IL18 armor was enhanced upon Nalm6 and Jeko-1 stimulation on Day 9. The cytokine production observed in these cells was higher than the production seen in CAR T cells expressing a positive control CD19 CAR. The cells were gated on CD8⁺ T cells.

TABLE 8
IFNγ and TNF-α production from ND585 and ND307CAR CD19 binders-
IL18 CD4 T cells upon Nalm6 and Jeko-1 stimulation on Day 9

| | ND585 | | | ND307 | | |
|---|---|---|---|---|---|---|
| | IFNγ | TNF-α | IFNγ & TNF-α | IFNγ | TNF-α | IFNγ & TNF-α |
| | Jeko-1 Stimulation | | | | | |
| Untransduced | 0.120 | 0.310 | 0.220 | 0.480 | 0.280 | 1.170 |
| 42op + IL-18 | 8.100 | 9.470 | 8.640 | 3.920 | 28.600 | 8.450 |
| 52op + IL-18 | 10.900 | 10.500 | 18.300 | 4.960 | 21.500 | 8.730 |
| 42 + IL-18 | 10.100 | 10.500 | 11.100 | 5.870 | 21.500 | 8.130 |
| 52 + IL-18 | 10.300 | 12.800 | 17.200 | 5.570 | 20.900 | 7.730 |
| | Nalm6 Stimulation | | | | | |
| Untransduced | 0.110 | 0.530 | 0.250 | 0.930 | 0.000 | 2.460 |
| 42op + IL-18 | 9.040 | 22.900 | 23.700 | 3.990 | 34.600 | 11.000 |
| 52op + IL-18 | 9.760 | 20.900 | 30.200 | 5.720 | 24.200 | 9.490 |
| 42 + IL-18 | 9.110 | 24.600 | 28.000 | 5.090 | 29.300 | 11.500 |
| 52 + IL-18 | 9.570 | 24.000 | 30.400 | 5.860 | 23.600 | 9.360 |
| | No stimulation (Unstimulated) | | | | | |
| Untransduced | 0.250 | 0.400 | 0.410 | 0.680 | 0.120 | 1.190 |
| 42op + IL-18 | 0.440 | 0.590 | 0.440 | 1.250 | 0.041 | 0.420 |
| 52op + IL-18 | 2.190 | 1.420 | 2.190 | 1.530 | 0.370 | 0.380 |
| 42 + IL-18 | 0.910 | 0.760 | 0.910 | 1.380 | 0.120 | 0.290 |
| 52 + IL-18 | 1.750 | 1.330 | 1.750 | 2.170 | 0.360 | 0.310 |

To determine the antigen-dependency of cytokine production by CD19 CAR T cells production, cytokine production was assessed following stimulation by cells (recombinant antigen presenting cells) expressing variant levels of the CD19 antigen. In particular, K562 cells were transfected with no CD19 antigen, low CD19 antigen (about 3% CD19 antigen expression), medium CD19 antigen (about 49% CD19 antigen expression); or high CD19 antigen (about 71% CD19 antigen expression). These recombinant K562 cells were co-cultured with CD19 CAR T cells and their cytokine production was assessed by flow cytometry.

TABLE 9
IL-2 and TNF-α production from ND307 CAR CD19 binders-
IL18 CD4 T cells as a function of surface antigen density

| | Gated on CD4⁺ T cells | | | Gated on CD8⁺ T cells | | |
|---|---|---|---|---|---|---|
| | IL-2 | TNF-α | IL-2 & TNF-α | IFNγ | TNF-α | IFNγ & TNF-α |
| | High Antigen density | | | | | |
| | (71% expression level in K562 cells | | | | | |
| | transfected with different | | | | | |
| | amount of truncated CD19 Antigen) | | | | | |
| Untransduced | 0.320 | 0.160 | 0.160 | 0.081 | 0.550 | 0.100 |
| 42og + IL-18 | 8.370 | 18.400 | 39.300 | 6.810 | 27.200 | 7.110 |
| 52og + IL-18 | 9.170 | 21.300 | 25.900 | 9.160 | 15.100 | 9.290 |
| 42OP + IL-18 | 8.820 | 20.700 | 34.900 | 8.860 | 21.300 | 8.100 |
| 52OP + IL-18 | 8.220 | 21.300 | 24.500 | 8.550 | 13.900 | 7.050 |
| | Medium Antigen density | | | | | |
| | (49% expression level in K562 cells | | | | | |
| | transfected with different | | | | | |
| | amount of truncated CD19 Antigen) | | | | | |
| Untransduced | 0.400 | 0.150 | 0.100 | 0.098 | 0.430 | 0.039 |
| 42og + IL-18 | 9.030 | 18.200 | 36.800 | 7.100 | 25.100 | 7.360 |
| 52og + IL-18 | 7.940 | 19.400 | 27.200 | 7.620 | 15.100 | 8.590 |
| 42OP + IL-18 | 7.590 | 21.500 | 33.000 | 8.580 | 19.500 | 8.160 |
| 52OP + IL-18 | 9.440 | 19.300 | 23.800 | 9.290 | 14.300 | 6.330 |
| | Low Antigen density | | | | | |
| | (3% expression level in K562 cells | | | | | |
| | transfected with different | | | | | |
| | amount of truncated CD19 Antigen) | | | | | |
| Untransduced | 0.400 | 0.000 | 0.400 | 0.059 | 0.690 | 0.120 |
| 42og + IL-18 | 8.700 | 18.400 | 29.100 | 6.760 | 18.800 | 6.140 |
| 52og + IL-18 | 8.350 | 14.300 | 16.000 | 7.130 | 10.700 | 4.740 |
| 42OP + IL-18 | 8.550 | 19.500 | 22.700 | 8.470 | 13.900 | 5.640 |
| 52OP + IL-18 | 6.180 | 15.900 | 15.600 | 6.840 | 11.600 | 3.900 |

No Antigen

| | | | | | | |
|---|---|---|---|---|---|---|
| Untransduced | 0.270 | 0.270 | 0.230 | 0.140 | 0.440 | 0.071 |
| 42og + IL-18 | 0.570 | 0.280 | 0.280 | 1.110 | 0.520 | 0.280 |
| 52og + IL-18 | 0.640 | 0.610 | 0.430 | 1.520 | 0.950 | 0.570 |
| 42OP + IL-18 | 0.700 | 0.370 | 0.370 | 1.940 | 0.930 | 0.410 |
| 52OP + IL-18 | 0.630 | 0.490 | 0.410 | 1.460 | 0.910 | 0.500 |

Unstimulated

| | | | | | | |
|---|---|---|---|---|---|---|
| Untransduced | 0.530 | 0.027 | 1.150 | 0.130 | 0.350 | 0.360 |
| 42og + IL-18 | 0.470 | 0.041 | 0.460 | 1.140 | 0.660 | 0.300 |
| 52og + IL-18 | 0.460 | 0.330 | 0.560 | 1.290 | 1.080 | 0.320 |
| 42OP + IL-18 | 0.320 | 0.130 | 0.350 | 1.350 | 0.800 | 0.270 |
| 52OP + IL-18 | 0.910 | 0.430 | 0.360 | 3.190 | 1.560 | 0.810 |

Table 9 shows flow cytometric results of cytokine production (IL-2, TNF-α, and IFN-γ) from ND307 CD4⁺ and CD8⁺CAR T cells expressing CD19-42og-IL-18, CD19-42op-IL-18, CD19-52og-IL-18, and CD19-52op-IL-18 upon stimulation with recombinant K562 cells transfected with different amount of RNA encoding a truncated CD19 antigen (CD19Ag RNA) on Day 9. The IL-2 and TNF-α production by the ND307 CD4⁺CAR T cells expressing the CD19 binders-IL18 was proportional to the CD19 antigen surface density on the transfected K562 cells. Cytokine production was enhanced even when the K562 cells expressed low levels of CD19 antigens (e.g., low antigen). Cells were gated for CD4⁺ T cells of IL-2 and TNF-α production. Similar trends were observed for IFN-γ and TNF-α production by CD8⁺ T cells.

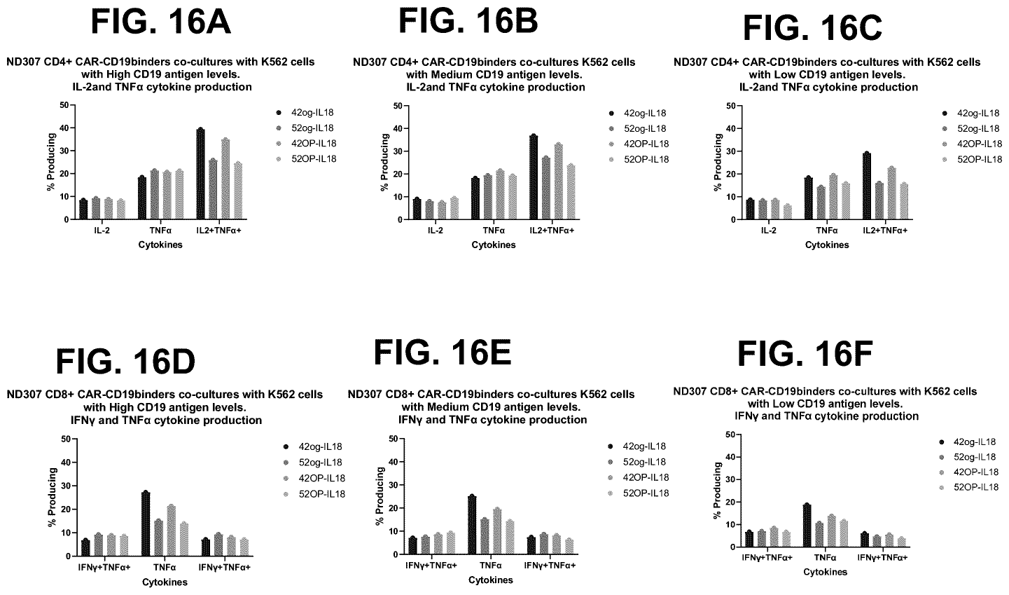

FIGS. 16A-F show bar graphs quantifying cytokine production (IL-2, TNF-α, and IFN-γ) by ND307 CD4⁺ and CD8⁺CAR T cells expressing CD19-42og-IL-18, CD19-42op-IL-18, CD19-52og-IL-18, and CD19-52op-IL-18 upon stimulation with recombinant K562 cells transfected with different amount of RNA encoding a truncated CD19 antigen (CD19Ag RNA). TNF-α production in ND307 CD4⁺CAR T cells was higher from CAR T cells expressing CD19-42og-IL-18. Plots of the percentages of single and polyproduction of IL-2 and TNFα on CD4⁺ T cells and IFNγ and TNFα on CD8⁺ T cells showed a greater cytokine production by CD19 42(og)-IL-18 CAR T cells. Cytokine production in CD19 42(og)-IL-18 CAR T cells was closer to cytokine production observed in the positive control CD19 CAR T cells. Generally, the activation of IL-2 and TNFα from CD4⁺ T cells was detected on CD19 CAR T cells at low CD19 antigen levels expressed on K562 cells.

### Conclusion

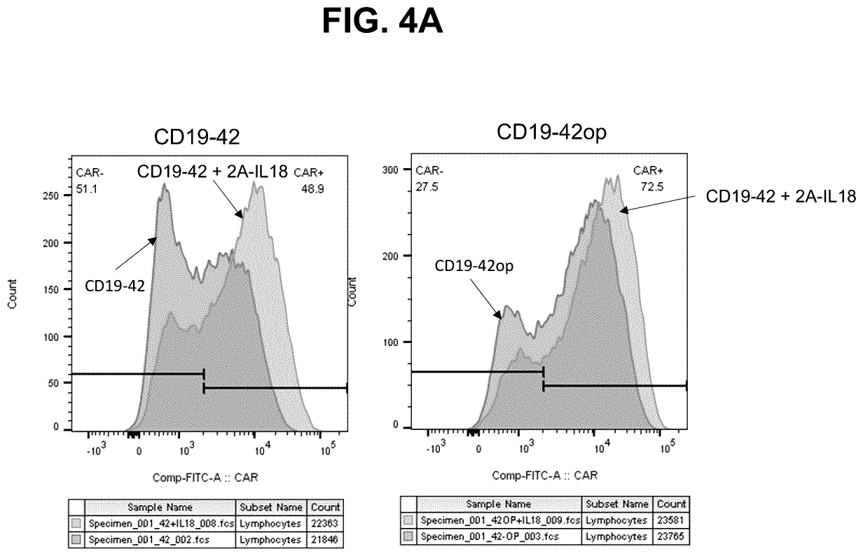

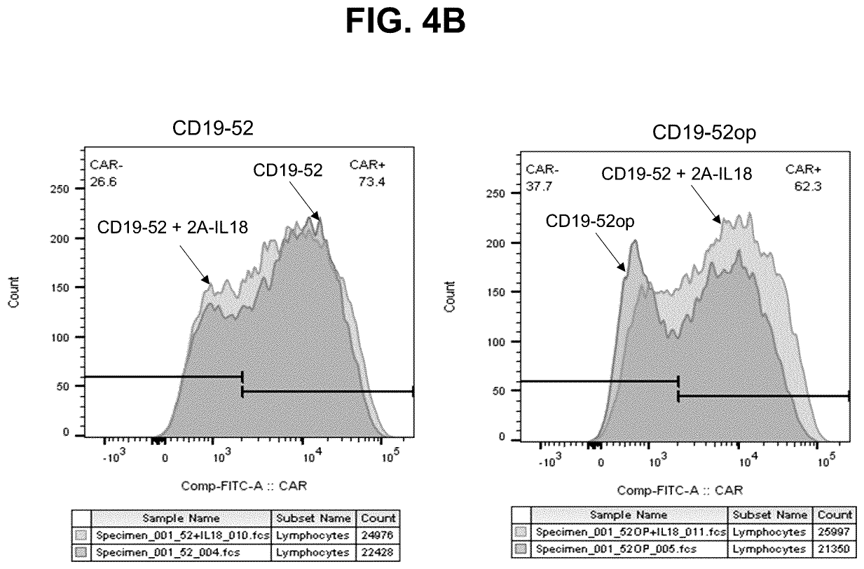

The stability and MFIs of CD19 CARs on CART cells co-expressing IL-18 was compared using T cells from donors ND585, ND307 and ND609. As shown herein, (FIGS. 4A-B), the cell surface expression of CD19 CAR (42og, 42op, 52og, and 52op) was not reduced when the CD19 CAR was co-expressed with a recombinant IL-18. Furthermore, the stability and MFI analyses of CD19 CAR surface expression (e.g., control CD19 binder, original and optimized novel binders 42 and 52) on both ND585 and ND307 T cells showed that overall, the expression of the control CD19 binder was higher and followed by the expression of CD19-42OP. The CD19-42og CART cells had similar levels or greater surface expression levels than CD19-52OP CAR T cells; the latter was greater than or equal to CD19-52og CAR T cells (Table 5). Overall, in MFI and stability surface expression profiles trend showed that control CD19CAR-IL18>CD19-42-IL18>CD19-52-IL18.

The comparison of the cytokine responses from the different donors consistently showed that the positive control CD19 CAR T cells were the least responsive in three out of four donors with CD19-42og CAR T cells being lower once (Table 6, and Table 7 and FIGS. 15A-B). With each of the donors where comparisons were possible, cytokine production in CD19-42og-IL-18 was consistently the lowest. In donors ND585 and ND307, the addition of IL-18 co-expression, there was no effective differences observed in the original and optimized versions of CD19-42 and CD19-52.

The comparison of in vivo antitumor activity from all donors showed that all CAR T cells tested effectively cleared tumor in mice. However, mice administered with CD19-42og-IL-18 CAR T cells consistently showed a quicker tighter clearance of tumor among the mice (FIGS. 6-9). Even with a wider spread of anti-tumor responses, each of the groups eventually controlled tumor.

Previous studies showed that the positive CD19 CAR used in these studied consistently cleared tumor. The present disclosure found that CART cells expressing this well-characterized binder showed a weaken tumor clearance response when compared to the novel CD19 binders disclosed herein. This weakened tumor clearance was unexpected, but it supports the uniqueness of the novel binders disclosed herein.

Following the tumor clearance, it was observed that peripheral huCD45 levels decreased with time to below quantifiable levels for each of the CD19-IL-18 CART cells. The cytokine response levels were similar for all CD19 CARs tested. Similarly, they tested CD19-CAR T cells showed good contractions in peripheral blood levels after clearing tumor. While all CAR novel CD19 binders disclosed herein were effective killers, the CD19-42og-IL18 consistently had quicker and tighter tumor clearance profile. Lastly, CD19-42-IL18 and CD19-52-IL18 in either original or optimize phase were effective therapeutics. CD19-42og-IL18 showed an effective overall response most closely compared and better than known CD19 binders in terms of tumor control, post tumor clearance, surface expression and stability, cytokine responses, and minimal tonic signaling.

### Example 9: Novel CD19 Binders Binding Affinity

To determine if the CD19 binder disclosed herein can result in lower altered affinities and rate constants, the binders can be biotinylated, purified and tested in vivo. The 12 CD19 binders may be subjected to biolayer interferometry (BLI) using streptavidin biosensors and recombinant CD19. It is expected that the affinities of the novel CD19 binders will range from about 175 nM to >10,000 nM, but lower than the affinity of the FMC63 binder. In particular, the FMC63 binder is disclosed to have a Kₒₙ=2.1×10⁵ M⁻¹ s⁻¹, K_{off}=6.8×10⁻⁵ s⁻¹, and a K_{D}=0.328 nM. In contrast, the present CD19 binders are expected to have between about 30-fold to about 60-fold lower affinity than the FMC63 binder. In particular, the novel CD19 binders may have Kori 2.1×10⁵ M⁻¹ s⁻¹, K_{off}=bout 1.0×10⁻³ s⁻¹ to about 5.0×10⁻³ s⁻¹, and a K_{D}=1 nM to about 175 nM, or a K_{D}>175 nM. In addition, a very close correlation between the novel CD19 binders' affinity (K_{D}) and TRF binding.

### Example 10: Generation and Expansion of CD19 CAR T Cells

The purpose of this example is to describe potential methods of making CAR-T cells described herein.

CAR T cells expressing the CD19 binders disclosed herein can be generated using any method known in the art for example as shown in WO 2014/153270. In particular, CD19 CAR disclosed herein can be expressed in a lentiviral vector. Primary human T lymphocytes can then be isolated and then stimulated with magnetic beads coated with anti-CD 3/anti-CD28 antibodies (Miltenyi Biotec) at cell to bead ratio of 1 to 2. Approximately 0, 5, 10, 12, or 24 hours after activation, T cells may be transfected with a lentiviral vector encoding the CD19 CAR. Transfected T cells can be used immediately or expanded for up to 3 days.

In one aspect of the disclosure, CAR T cells expressing the CD19 binders of the present disclosure are expected to exhibit enhanced expansion rates when compared to CD19 CAR known in the art.

### Example 11: Killing Assays

The purpose of this example is to describe exemplary killing assays for the CAR-Ts described herein.

To determine the effectiveness of CAR T cells expressing the CD19 binders of the present disclosure (P1-P13), target cells, such as K562, K562-CD19, and NALM6 can be tagged with GFP and/or luciferase (GL), and incubated with GFP expressed T cells or CD19 CAR T cells of the present disclosure, at the desired ratios in triplicate wells in U-bottomed 96-well plates. Viability of target cells can be tested about 18 h later by adding 100 dl/well substrate D-luciferin firefly (Life Sciences) at 150 μg/ml. Background luminescence may be negligible (<1% than the signal from the wells with only target cells). The viability percentage can be calculated as experimental signal/maximal signal×100%, and killing percentage was equal to 100−viability percentage.

It is expected that the CAR T cells comprising any one of P1-P13 binders and generated from PBMC of ALL patients will lyse more than 75% CD19-expressing NALM-6 cells. The results of killing assay with K562 cells will reveal that CD19 CAR T cells will specifically kill the K562-CD19 cells but not the K562 cells without CD19 expression. In particular, it is expected that CD19 CAR T cells comprising any one of P1-P13 binders shown in Table 3, or CDRs shown in Table 2 will show either equal or enhanced anti-tumor activity when compared to a CD19 CAR known in the art. It is further expected that CAR comprising any one of the P1-P13 binders will be selective for tumor cells over wild-type cells when compared to CD19 known in the art.

TABLE 1
All Sequences

| SEQ | | |
|---|---|---|
| ID | | |
| NO | Description | Sequences |
| 1 | 42og CDR-L1 | RASQTISNYLN |
| 2 | 42og CDR-L2 | AASSLQS |
| 3 | 42og CDR-L3 | QQSYSTPPT |
| 4 | 42og CDR-H1 | AASGFTFSNYAIS |
| 5 | 42og CDR-H2 | VSVITASGVDTYYADSV |
| 6 | 42og CDR-H3 | GGTPYFITTYDYYGFDV |
| 7 | CD19 42og V_{L} | DIQMTQSPSSLSASVGDRVTITCRASQTISNYLNW |
| | | YQQKPGKAPKLLIYAASSLQSGVPSRFSGSGSGT |
| | | DFTLTISSLQPEDFATYYCQQSYSTPPTFGQGTKV |
| | | EIK |
| 8 | CD19 42og V_{H} | EVQLLESGGGLVQPGGSLRLSCAASGFTFSNYAI |
| | | SWVRQAPGKGLEWVSVITASGVDTYYADSVKG |
| | | RFTISRDNSKNTLYLQMNSLRAEDTAVYYCARG |
| | | GTPYFITTYDYYGFDVWGQGTLVTVSS |
| 9 | scFv-CD19 42og | DIQMTQSPSSLSASVGDRVTITCRASQTISNYLNW |
| | human derived | YQQKPGKAPKLLIYAASSLQSGVPSRFSGSGSGT |
| | | DFTLTISSLQPEDFATYYCQQSYSTPPTFGQGTKV |
| | | EIKGGGGSGGGGSGGGGSEVQLLESGGGLVQPG |
| | | GSLRLSCAASGFTFSNYAISWVRQAPGKGLEWV |
| | | SVITASGVDTYYADSVKGRFTISRDNSKNTLYLQ |
| | | MNSLRAEDTAVYYCARGGTPYFITTYDYYGFDV |
| | | WGQGTLVTVSS |
| 10 | A4 CDR-L1 | RASQSVSSNYLA |
| 11 | A4 CDR-L2 | GASSRAT |
| 12 | A4 CDR-L3 | QQYESSPSWT |
| 13 | A4 CDR-H1 | KASGGTFSNYYIS |
| 14 | A4 CDR-H2 | MGGIIPLFGTTNYAQ |
| 15 | A4 CDR-H3 | GTWYAGDI |
| 16 | A4 V_{L} | EIVLTQSPGTLSLSPGERATLSCRASQSVSSNYLA |
| | | WYQQKPGQAPRLLIYGASSRATGIPDRFSGSGSG |
| | | TDFTLTISRLEPEDFAVYYCQQYESSPSWTFGQG |
| | | TKVEIK |
| 17 | A4 V_{H} | QVQLVQSGAEVKKPGSSVKVSCKASGGTFSNYY |
| | | ISWVRQAPGQGLEWMGGIIPLFGTTNYAQKFQG |
| | | RVTITADESTSTAYMELSSLRSEDTAVYYCARGT |
| | | WYAGDIWGQGTLVTVSS |
| 18 | scFv-CD19 A4 | EIVLTQSPGTLSLSPGERATLSCRASQSVSSNYLA |
| | human derived | WYQQKPGQAPRLLIYGASSRATGIPDRFSGSGSG |
| | | TDFTLTISRLEPEDFAVYYCQQYESSPSWTFGQG |
| | | TKVEIKGGGGSGGGGSGGGGSQVQLVQSGAEVK |
| | | KPGSSVKVSCKASGGTFSNYYISWVRQAPGQGL |
| | | EWMGGIIPLFGTTNYAQKFQGRVTITADESTSTA |
| | | YMELSSLRSEDTAVYYCARGTWYAGDIWGQGT |
| | | LVTVSS |
| 19 | Nucleic acid | gacatacaaatgacccagtctccttcttctctgagcgcatctgtcggcgataga |
| | sequence | gtgactattacatgtagggccagccagaccatctctaattatctgaattggtatc |
| | CD19 42og V_{L} | agcaaaaacccggcaaggctccaaaactgttgatctatgctgcttcaagcctg |
| | | caatccggagttccctcaagattttctggttccggctcaggaactgatttcacc |
| | | ctgactataagttctttgcagcctgaagactttgcaacatattactgtcagcaat |
| | | cttactctaccccaccaacattcgggcaaggtaccaaggtcgaaattaag |
| 20 | Nucleic acid | gaggttcagttgttagagagcggggggggtctggttcagcctggtggcagct |
| | sequence | taagactgagttgtgccgcttctggttttactttctctaattatgcaatatcctg |
| | CD19 42ogV_{H} | ggtgaggcaagcccccggtaaaggcctggaatgggtttcagttatcaccgcttct |
| | | ggtgttgatacctactacgccgatagcgtgaaaggtagattcactatttccagg |
| | | gacaattcaaagaacactttgtacttacagatgaactctttgagagctgaggac |
| | | accgcagtgtattactgtgctagaggtggtaccccatactttatcaccacctac |
| | | gattactacggttttgatgtttggggacaaggaactttggtcacagtgtcatct |
| 21 | Nucleic acid | gacatacaaatgacccagtctccttcttctctgagcgcatctgtcggcgataga |
| | sequence | gtgactattacatgtagggccagccagaccatctctaattatctgaattggtatc |
| | scFv-CD19 42og | agcaaaaacccggcaaggctccaaaactgttgatctatgctgcttcaagcctg |
| | | caatccggagttccctcaagattttctggttccggctcaggaactgatttcacc |
| | | ctgactataagttctttgcagcctgaagactttgcaacatattactgtcagcaat |
| | | cttactctaccccaccaacattcgggcaaggtaccaaggtcgaaattaagggt |
| | | ggtggtggttctggtggtggcggtagcggaggtggtggtagtgaggttcagt |
| | | tgttagagagcggggggggtctggttcagcctggtggcagcttaagactga |
| | | gttgtgccgcttctggttttactttctctaattatgcaatatcctgggtgaggcaa |
| | | gcccccggtaaaggcctggaatgggtttcagttatcaccgcttctggtgttgat |
| | | acctactacgccgatagcgtgaaaggtagattcactatttccagggacaattc |
| | | aaagaacactttgtacttacagatgaactctttgagagctgaggacaccgcag |
| | | tgtattactgtgctagaggtggtaccccatactttatcaccacctacgattacta |
| | | cggttttgatgtttggggacaaggaactttggtcacagtgtcatct |
| 22 | Nucleic acid | gaaatcgtcttgacccagtccccaggtactctgtctttaagtcccggggaaag |
| | sequence | agctactctgtcctgtagggcaagtcagtctgtttcttctaattatctggcctggt |
| | CD19 A4 V_{L} | atcagcaaaaacccggtcaagctccaagactgttgatctatggtgcaagcag |
| | | tagagccaccggaatccctgataggttttccggttcaggcagcggaactgac |
| | | ttcactctgacaatctcaagattggaacctgaggattttgccgtgtattactgtca |
| | | gcagtacgaatcttctccatcttggacattcgggcaaggtaccaaggtcgaaa |
| | | ttaag |
| 23 | Nucleic acid | caggtccagttagttcaatcaggtgccgaggtcaaaaagccaggttcttccgt |
| | sequence | caaagtgtcatgcaaggctagcggtggcaccttttctaattactacatctcttgg |
| | CD19 A4V_{H} | gtgagacaggcaccaggtcaaggactggaatggatgggaggtatcatccca |
| | | ctgtttggtaccaccaattatgcccagaagttccaaggcagggtgaccataac |
| | | tgctgatgagagtacatctaccgcatacatggaattaagttctctgagatccga |
| | | ggacaccgcagtgtattactgtgctagaggtacctggtacgctggtgatatct |
| | | ggggacaaggaactttggtcacagtgtcatct |
| 24 | Nucleic acid | gaaatcgtcttgacccagtccccaggtactctgtctttaagtcccggggaaag |
| | sequence | agctactctgtcctgtagggcaagtcagtctgtttcttctaattatctggcctggt |
| | scFv-CD19 A4 | atcagcaaaaacccggtcaagctccaagactgttgatctatggtgcaagcag |
| | | tagagccaccggaatccctgataggttttccggttcaggcagcggaactgac |
| | | ttcactctgacaatctcaagattggaacctgaggattttgccgtgtattactgtca |
| | | gcagtacgaatcttctccatcttggacattcgggcaaggtaccaaggtcgaaa |
| | | ttaagggtggtggtggttctggtggtggcggtagcggaggtggtggtagtca |
| | | ggtccagttagttcaatcaggtgccgaggtcaaaaagccaggttcttccgtca |
| | | aagtgtcatgcaaggctagcggtggcaccttttctaattactacatctcttgggt |
| | | gagacaggcaccaggtcaaggactggaatggatgggaggtatcatcccact |
| | | gtttggtaccaccaattatgcccagaagttccaaggcagggtgaccataactg |
| | | ctgatgagagtacatctaccgcatacatggaattaagttctctgagatccgag |
| | | gacaccgcagtgtattactgtgctagaggtacctggtacgctggtgatatctg |
| | | gggacaaggaactttggtcacagtgtcatct |
| 25 | CD8 Leader | MALPVTALLLPLALLLHAARP |
| 26 | CD8 Leader | atggccctgcctgtgacagccctgctgctgcctctggctctgctgctgcatgc |
| | (nucleic acid) | cgctagaccc |
| 27 | CD8 Hinge | TTTPAPRPPTPAPTIASQPLSLRPEACRPAAGGAV |
| | | HTRGLDFACD |
| 28 | CD8 Hinge nucleic | accacgacgccagcgccgcgaccaccaacaccggcgcccaccatcgcgt |
| | acid sequence | cgcagcccctgtccctgcgcccagaggcgtgccggccagcgggggggg |
| | | cgcagtgcacacgagggggctggacttcgcctgtgat |
| 29 | CD8 TM | IYIWAPLAGTCGVLLLSLVITLYC |
| 30 | CD8 TM nucleic | atctacatctgggcgcccttggccgggacttgtggggtccttctcctgtcactg |
| | acid sequence | gttatcaccctttactgc |
| 31 | CD28 | FWVLVVVGGVLACYSLLVTVAFIIFWV |
| | transmembrane | |
| | domain amino acid | |
| | sequence | |
| 32 | CD28 | ttttgggtgctggtggtggttggtggagtcctggcttgctatagcttgctagtaa |
| | transmembrane | cagtggcctttattattttctgggtg |
| | domain nucleic acid | |
| | sequence | |
| 33 | ICOS | FWLPIGCAAFVVVCILGCILICWL |
| | transmembrane | |
| | domain amino acid | |
| | sequence | |
| 34 | ICOS | ttctggttacccataggatgtgcagcctttgttgtagtct |
| | transmembrane | gcattttgggatgcatacttatttgttggctt |
| | domain nucleic acid | |
| | sequence | |
| 35 | IgG4 Hinge amino | ESKYGPPCPPCPAPEFLGGPSVFLFPPKPKDTLMIS |
| | acid sequence | RTPEVTCWVDVSQEDPEVQFNWYVDGVEVHNA |
| | | KTKPREEQFNSTYRVVSVLTVLHQDWLNGKEYK |
| | | CKVSNKGLPSSIEKTISKAKGQPREPQVYTLPPSQ |
| | | EEMTKNQVSLTCLVKGFYPSDIAVEWESNGQPE |
| | | NNYKTTPPVLDSDGSFFLYSRLTVDKSRWQEGN |
| | | VFSCSVMHEALHNHYTQKSLSLSLGKM |
| 36 | IgG4 Hinge nucleic | gagagcaagtacggccctccctgccccccttgccctgcccccgagttcctgg |
| | acid sequence | gcggacccagcgtgttcctgttcccccccaagcccaaggacaccctgatgat |
| | | cagccggacccccgaggtgacctgtgtggtggtggacgtgtcccaggagg |
| | | accccgaggtccagttcaactggtacgtggacggcgtggaggtgcacaacg |
| | | ccaagaccaagccccgggaggagcagttcaatagcacctaccgggtggtg |
| | | tccgtgctgaccgtgctgcaccaggactggctgaacggcaaggaatacaag |
| | | tgtaaggtgtccaacaagggcctgcccagcagcatcgagaaaaccatcagc |
| | | aaggccaagggccagcctcgggagccccaggtgtacaccctgccccctag |
| | | ccaagaggagatgaccaagaaccaggtgtccctgacctgcctggtgaagg |
| | | gcttctaccccagcgacatcgccgtggagtgggagagcaacggccagccc |
| | | gagaacaactacaagaccaccccccctgtgctggacagcgacggcagcttc |
| | | ttcctgtacagccggctgaccgtggacaagagccggtggcaggagggcaa |
| | | cgtctttagctgctccgtgatgcacgaggccctgcacaaccactacacccag |
| | | aagagcctgagcctgtccctgggcaagatg |
| 37 | 4-1BB domain | KRGRKKLLYIFKQPFMRPVQTTQEEDGCSCRFPE |
| | | EEEGGCEL |
| 38 | 4-1BB domain | aaacggggcagaaagaaactcctgtatatattcaaacaaccatttatgagacc |
| | nucleic acid | agtacaaactactcaagaggaagatggctgtagctgccgatttccagaagaa |
| | | gaagaaggaggatgtgaactg |
| 39 | CD28 intracellular | RSKRSRLLHSDYMNMTPRRPGPTRKHYQPYAPP |
| | domain amino acid | RDFAAYRS |
| | sequence | |
| 40 | CD28 intracellular | aggagtaagaggagcaggctcctgcacagtgactacatgaacatgactccc |
| | domain nucleic acid | cgccgccccgggcccacccgcaagcattaccagccctatgccccaccacg |
| | sequence | cgacttcgcagcctatcgctcc |
| 41 | CD28 intracellular | RSKRSRLLHSDYMFMTPRRPGPTRKHYQPYAPP |
| | domain variant | RDFAAYRS |
| | (YMFM) amino | |
| | acid sequence | |
| 42 | CD28 intracellular | AGGAGTAAGAGGAGCAGGCTCCTGCACAGTGA |
| | domain variant | CTACATGTTCATGACTCCCCGCCGCCCCGGGCC |
| | (YMFM) nucleic | CACCCGCAAGCATTACCAGCCCTATGCCCCAC |
| | acid sequence | CACGCGACTTCGCAGCCTATCGCTCC |
| 43 | ICOS costimulatory | TKKKYSSSVHDPNGEYMFMRAVNTAKKSRLTD |
| | domain amino acid | VTL |
| | sequence | |
| 44 | ICOS costimulatory | acaaaaaagaagtattcatccagtgtgcacgaccctaacggtgaatacatgt |
| | domain nucleic acid | tcatgagagcagtgaacacagccaaaaaatctagactcacagatgtgaccct |
| | sequence | a |
| 45 | ICOS-1 | acaaaaaagaagtattcatccagtgtgcacgaccctaacggtgaatacatgt |
| | costimulatory | tcatgagagcagtgaacacagccaaaaaatccagactcacagatgtgaccct |
| | domain nucleic acid | a |
| | sequence | |
| 46 | CD2 costimulatory | TKRKKQRSRRNDEELETRAHRVATEERGRKPHQ |
| | domain amino acid | IPASTPQNPATSQHPPPPPGHRSQAPSHRPPPPGH |
| | sequence | RVQHQPQKRPPAPSGTQVHQQKGPPLPRPRVQP |
| | | KPPHGAAENSLSPSSN |
| 47 | CD2 costimulatory | accaaaaggaaaaaacagaggagtcggagaaatgatgaggagctggaga |
| | domain nucleic acid | caagagcccacagagtagctactgaagaaaggggccggaagccccacca |
| | sequence | aattccagcttcaacccctcagaatccagcaacttcccaacatcctcctccacc |
| | | acctggtcatcgttcccaggcacctagtcatcgtcccccgcctcctggacacc |
| | | gtgttcagcaccagcctcagaagaggcctcctgctccgtcgggcacacaag |
| | | ttcaccagcagaaaggcccgcccctccccagacctcgagttcagccaaaac |
| | | ctccccatggggcagcagaaaactcattgtccccttcctctaat |
| 48 | CD27 intracellular | QRRKYRSNKGESPVEPAEPCRYSCPREEEGSTIPI |
| | domain amino acid | QEDYRKPEPACSP |
| | sequence | |
| 49 | CD27 intracellular | caacgaaggaaatatagatcaaacaaaggagaaagtcctgtggagcctgca |
| | domain nucleic acid | gagccttgtcgttacagctgccccagggaggaggagggcagcaccatccc |
| | sequence | catccaggaggattaccgaaaaccggagcctgcctgctccccc |
| 50 | OX40 intracellular | ALYLLRRDQRLPPDAHKPPGGGSFRTPIQEEQAD |
| | domain amino acid | AHSTLAKI |
| | sequence | |
| 51 | OX40 intracellular | gccctgtacctgctccgcagggaccagaggctgccccccgatgcccacaa |
| | domain nucleic acid | gccccctgggggaggcagtttcaggacccccatccaagaggagcaggcc |
| | sequence | gacgcccactccaccctggccaagatc |
| 52 | CD3 (Q14K) zeta | RVKFSRSADAPAYKQGQNQLYNELNLGRREEYD |
| | domain | VLDKRRGRDPEMGGKPRRKNPQEGLYNELQKD |
| | | KMAEAYSEIGMKGERRRGKGHDGLYQGLSTAT |
| | | KDTYDALHMQALPPR |
| 53 | CD3 (Q14K) zeta | agagtgaagttcagcaggagcgcagacgcccccgcgtacaagcagggcc |
| | domain nucleic acid | agaaccagctctataacgagctcaatctaggacgaagagaggagtacgatg |
| | | ttttggacaagagacgtggccgggaccctgagatggggggaaagccgaga |
| | | aggaagaaccctcaggaaggcctgtacaatgaactgcagaaagataagatg |
| | | gcggaggcctacagtgagattgggatgaaaggcgagcgccggaggggca |
| | | aggggcacgatggcctttaccagggtctcagtacagccaccaaggacacct |
| | | acgacgcccttcacatgcaggccctgccccctcgc |
| 54 | CD3 zeta domain | RVKFSRSADAPAYQQGQNQLYNELNLGRREEYD |
| | amino acid | VLDKRRGRDPEMGGKPRRKNPQEGLYNELQKD |
| | | KMAEAYSEIGMKGERRRGKGHDGLYQGLSTAT |
| | | KDTYDALHMQALPPR |
| 55 | CD3 zeta domain | agagtgaagttcagcaggagcgcagacgcccccgcgtaccagcagggcc |
| | nucleic acid | agaaccagctctataacgagctcaatctaggacgaagagaggagtacgatg |
| | | ttttggacaagagacgtggccgggaccctgagatggggggaaagccgaga |
| | | aggaagaaccctcaggaaggcctgtacaatgaactgcagaaagataagatg |
| | | gcggaggcctacagtgagattgggatgaaaggcgagcgccggaggggca |
| | | aggggcacgatggcctttaccagggtctcagtacagccaccaaggacacct |
| | | acgacgcccttcacatgcaggccctgccccctcgc |
| 92 | T2A amino acid | EGRGSLLTCGDVEENPGP |
| | sequence | |
| 93 | T2A nucleic acid | gagggcagaggaagtcttctaacatgcggtgacgtggaggagaatcccgg |
| | sequence | ccct |
| 94 | T2A spacer amino | SGRSGGG |
| | acid sequence | |
| 95 | T2A spacer nucleic | tccggaagatctggcggcgga |
| | acid sequence | |
| 96 | F2A amino acid | VKQTLNFDLLKLAGDVESNPGP |
| | sequence | |
| 97 | F2A nucleic acid | gtgaaacagactttgaattttgaccttctcaagttggcgggagacgtggagtc |
| | sequence | caacccagggccg |
| 98 | Furin-(G4S)2-T2A | cgtgcgaagaggggcggcgggggctccggcgggggaggcagtgaggg |
| | (F-GS2-T2A) linker | ccgcggctccctgctgacctgcggagatgtagaagagaacccaggcccc |
| | nucleic acid | |
| | sequence | |
| 99 | Furin-(G4S)2-T2A | RAKRGGGGSGGGGSEGRGSLLTCGDVEENPGP |
| | (F-GS2-T2A) linker | |
| | amino acid | |
| | sequence | |
| 100 | WPRE | INLWITKFVKD*LVFLTMLLLLRYVDTLL*CLCIM |
| | | LLLPVWLSFSPPCINPGCCLFMRSCGPLSGNVAW |
| | | CALCLLTQPPLVGALPPPVSSFPGLSLSPSLLPRRN |
| | | SSPPALPAAGQGLGCWALTIPWCCRGS*RPFLGC |
| | | SPVLPPGFCAGRPSATSLRPSIQRTFLPAACCRLC |
| | | GLFRVFAFALRRVGSPFGPPPRL |
| 101 | EF-1 alpha | cgtgaggctccggtgcccgtcagtgggcagagcgcacatcgcccacagtc |
| | promoter | cccgagaagttggggggaggggtcggcaattgaaccggtgcctagagaag |
| | | gtggcgcggggtaaactgggaaagtgatgtcgtgtactggctccgcctttttc |
| | | ccgaggggggggagaaccgtatataagtgcagtagtcgccgtgaacgttct |
| | | ttttcgcaacgggtttgccgccagaacacaggtaagtgccgtgtgtggttccc |
| | | gcgggcctggcctctttacgggttatggcccttgcgtgccttgaattacttcca |
| | | cctggctgcagtacgtgattcttgatcccgagcttcgggttggaagtgggtgg |
| | | gagagttcgaggccttgcgcttaaggagccccttcgcctcgtgcttgagttga |
| | | ggcctggcctgggcgctggggccgccgcgtgcgaatctggtggcaccttc |
| | | gcgcctgtctcgctgctttcgataagtctctagccatttaaaatttttgatgacct |
| | | gctgcgacgctttttttctggcaagatagtcttgtaaatgcgggccaagatctg |
| | | cacactggtatttcggtttttggggccgcgggcggcgacggggcccgtgcgt |
| | | cccagcgcacatgttcggcgaggcggggcctgcgagcgcggccaccgag |
| | | aatcggacgggggtagtctcaagctggccggcctgctctggtgcctggcctc |
| | | gcgccgccgtgtatcgccccgccctgggcggcaaggctggcccggtcggc |
| | | accagttgcgtgagcggaaagatggccgcttcccggccctgctgcaggga |
| | | gctcaaaatggaggacgcggcgctcgggagagcgggcgggtgagtcacc |
| | | cacacaaaggaaaagggcctttccgtcctcagccgtcgcttcatgtgactcca |
| | | cggagtaccgggcgccgtccaggcacctcgattagttctcgagcttttggagt |
| | | acgtcgtctttaggttggggggaggggttttatgcgatggagtttccccacact |
| | | gagtgggtggagactgaagttaggccagcttggcacttgatgtaattctccttg |
| | | gaatttgccctttttgagtttggatcttggttcattctcaagcctcagacagtggtt |
| | | caaagtttttttcttccatttcaggtgtcgtga. |
| 102 | Nucleic acid | gacatacaaatgacccagtctccttcttctctgagcgcatctgtcggcgataga |
| | sequence | gtgactattacatgtagggccagccagtctatctcttcttatctgaattggtatca |
| | scFv-CD19 E4 | gcaaaaacccggcaaggctccaaaactgttgatctatggtgcttcaagcctg |
| | P3 | caatccggagttccctcaagattttctggttccggctcaggaactgatttcacc |
| | | ctgactataagttctttgcagcctgaagactttgcaacatattactgtcagcaat |
| | | cttacagaaccccagttacattcgggcaaggtaccaaggtcgaaattaaggg |
| | | tggtggtggttctggtggtggcggtagcggaggtggtggtagtcaggtccag |
| | | ttagttcaatcaggtgccgaggtcaaaaagccaggttcttccgtcaaagtgtc |
| | | atgcaaggctagcggtggcaccttttctaattacgctatcaattgggtgagaca |
| | | ggcaccaggtcaaggactggaatggatgggaagaatcgttccactgctggg |
| | | tatcgctaattatgcccagaagttccaaggcagggtgaccataactgctgatg |
| | | agagtacatctaccgcatacatggaattaagttctctgagatccgaggacacc |
| | | gcagtgtattactgtgctagagaacatatcgcttacagaccaacctctgctggt |
| | | tactactactacatggatatctggggacaaggaactttggtcacagtgtcatct |
| 103 | Nucleic acid | gacatacaaatgacccagtctccttcttctctgagcgcatctgtcggcgataga |
| | sequence | gtgactattacatgtagggccagccaggatatcaccagatatctgaattggtat |
| | scFv-CD19 E7 | cagcaaaaacccggcaaggctccaaaactgttgatctatgctgcttcaagcct |
| | | gcaatccggagttccctcaagattttctggttccggctcaggaactgatttcac |
| | | cctgactataagttctttgcagcctgaagactttgcaacatattactgtcagcaa |
| | | tcttactcttacccaccaacattcgggcaaggtaccaaggtcgaaattaaggg |
| | | tggtggtggttctggtggtggcggtagcggaggtggtggtagtcaggttcag |
| | | ttagtcgagtctggtggcggtgtcgtccagcctggtagatccttaaggctgtca |
| | | tgtgccgctagcggatttacctttagagattacggtatgcattgggtgagacaa |
| | | gcccccggtaaaggcctggaatgggtcgctgtgataagttacgaaggttcta |
| | | acgaatactacgcagactccgttaagggtagattcactatttccagggataatt |
| | | caaagaacactttgtatctgcagatgaactcattgagagctgaggacaccgc |
| | | agtgtattactgtgctagagatagaggttttgctggttggtacgattacgcttttg |
| | | atccatggggacaaggaactttggtcacagtgtcatct |
| 104 | Nucleic acid | gacatacaaatgacccagtctccttcttctctgagcgcatctgtcggcgataga |
| | sequence | gtgactattacatgtagggccagccagtctatctctaaatatctgaattggtatc |
| | scFv-CD19-7 | agcaaaaacccggcaaggctccaaaactgttgatctatgatgcttcaagcctg |
| | | caatccggagttccctcaagattttctggttccggctcaggaactgatttcacc |
| | | ctgactataagttctttgcagcctgaagactttgcaacatattactgtcagcaat |
| | | cttacaccatcccactgacattcgggcaaggtaccaaggtcgaaattaaggg |
| | | tggtggtggttctggtggtggcggtagcggaggtggtggtagtcaggtccag |
| | | ttagttcaatcaggtgccgaggtcaaaaagccaggttcttccgtcaaagtgtc |
| | | atgcaaggctagcggtggcaccttttcttcttacgctttttcttgggtgagacag |
| | | gcaccaggtcaaggactggaatggatgggaggtatcgttccactgtttggtg |
| | | ctgttgaatatgcccagaagttccaaggcagggtgaccataactgctgatga |
| | | gagtacatctaccgcatacatggaattaagttctctgagatccgaggacaccg |
| | | cagtgtattactgtgctagagaaaaaggtttttacagatactttgatcattgggg |
| | | acaaggaactttggtcacagtgtcatct |
| 105 | hIL 18 | YFGKLESKLSVIRNLNDQVLFIDQGNRPLFEDMT |
| | (e.g., GenBank | DSDCRDNAPRTIFIISMYKDSQPRGMAVTISVKCE |
| | Acc. No. | KISTLSCENKIISFKEMNPPDNIKDTKSDIIFFQRSV |
| | AAK95950.1) | PGHDNKMQFESSSYEGYFLACEKERDLFKLILKK |
| | leaderless | EDELGDRSIMFTVQNED |
| 106 | CD8leader +hIL 18 | MALPVTALLLPLALLLHAARPYFGKLESKLSVIR |
| | | NLNDQVLFIDQGNRPLFEDMTDSDCRDNAPRTIF |
| | | IISMYKDSQPRGMAVTISVKCEKISTLSCENKIISF |
| | | KEMNPPDNIKDTKSDIIFFQRSVPGHDNKMQFES |
| | | SSYEGYFLACEKERDLFKLILKKEDELGDRSIMFT |
| | | VQNED |
| 107 | Human IL-18 | MAAEPVEDNCINFVAMKFIDNTLYFIAEDDENLE |
| | (GenBank Acc. No. | SDYFGKLESKLSVIRNLNDQVLFIDQGNRPLFED |
| | AAK95950.1) | MTDSDCRDNAPRTIFIISMYKDSQPRGMAVTISV |
| | | KCEKISTLSCENKIISFKEMNPPDNIKDTKSDIIFFQ |
| | | RSVPGHDNKMQFESSSYEGYFLACEKERDLFKLI |
| | | LKKEDELGDRSIMFTVQNED |
| 108 | Human IL-2 signal | MYRMQLLSCIALSLALVINS |
| | sequence | |
| 109 | Murine IL-2 signal | MYSMQLASCVTLTLVLLVNS |
| | sequence | |
| 110 | human kappa leader | METPAQLLFLLLLWLPDTTG |
| | sequence | |
| 111 | Murine kappa | METDTLLLWVLLLWVPGSTG |
| | leader sequence | |
| 112 | Human albumin | MKWVTFISLLFSSAYS |
| | signal sequence | |
| 113 | Human prolactin | MDSKGSSQKGSRLLLLLVVSNLLLCQGVVS |
| | signal sequence | |
| 114 | Nucleic acid | gacatacaaatgacccagtctccttcttctctgagcgcatctgtcggcgataga |
| | sequence | gtgactattacatgtagggccagccagtctatctctaattatctgaattggtatca |
| | scFv-CD19-14 | gcaaaaacccggcaaggctccaaaactgttgatctatgctgcttcaagcctgc |
| | | aatccggagttccctcaagattttctggttccggctcaggaactgatttcaccct |
| | | gactataagttctttgcagcctgaagactttgcaacatattactgtcagcaagct |
| | | tactctgctccaatcacattcgggcaaggtaccaaggtcgaaattaagggtgg |
| | | tggtggttctggtggtggcggtagcggaggtggtggtagtgaggttcagttgt |
| | | tagagagcggggggggtctggttcagcctggtggcagcttaagactgagtt |
| | | gtgccgcttctggttttactttcggtgattatgcaatgtcctgggtgaggcaagc |
| | | ccccggtaaaggcctggaatgggtttcagctatctctagaggtggtcatggta |
| | | cctactatgccgatagcgtgaaaggtagattcactatttccagggacaattcaa |
| | | agaacactttgtacttacagatgaactctttgagagctgaggacaccgcagtg |
| | | tattactgtgctagactggttggttacggtctggattactggggacaaggaact |
| | | ttggtcacagtgtcatct |
| 115 | Nucleic acid | caaatgacccagtctccttcttctctgagcgcatctgtcggcgatagagtgact |
| | sequence | attacatgtagggccagccagcctatcagaccttatctgaattggtatcagcaa |
| | scFv-CD19-15 | aaacccggcaaggctccaaaactgttgatctatgatgcttcaagcctgcaatc |
| | | cggagttccctcaagattttctggttccggctcaggaactgatttcaccctgact |
| | | ataagttctttgcagcctgaagactttgcaacatattactgtcagcaatcttactc |
| | | tgctccatacacattcgggcaaggtaccaaggtcgaaattaagggtggtggt |
| | | ggttctggtggtggcggtagcggaggtggtggtagtgaggttcagttgttaga |
| | | gagcggggggggtctggttcagcctggtggcagcttaagactgagttgtgc |
| | | cgcttctggttttactttctcttcttatgcaatgtcctgggtgaggcaagcccccg |
| | | gtaaaggcctggaatgggtttcagttatctctggtggtggtgctaatacctacta |
| | | cgccgatagcgtgaaaggtagattcactatttccagggacaattcaaagaac |
| | | actttgtacttacagatgaactctttgagagctgaggacaccgcagtgtattact |
| | | gtgctagagattggagatactttgatcattggggacaaggaactttggtcaca |
| | | gtgtcatct |
| 116 | Nucleic acid | cagtccgtgttgacccagcctccatcagtctcaggagccccaggccagaga |
| | sequence | gtgaccatttcttgtactggatcttcctcaaatatcggggccggttacgatgttc |
| | scFv-CD19-18 | attggtatcagcaactgcccggtacagctccaaaactgttgatctatggtacca |
| | | aaaacagacctagcggtgtgcccgacaggttctccggctcaaagagcggaa |
| | | caagtgcttctttagcaattaccggcctgcaggctgaagatgaggcagactat |
| | | tactgtcaatcctacgatgttagactgaaaggttgggtttttggtggcggtacca |
| | | aattgactgtcttaggtggtggtggttctggtggtggcggtagcggaggtggt |
| | | ggtagtcagttacagttacaggagagcggtccaggtttagtgaaaccatccg |
| | | aaactttatcactgacctgtacagtgtctggtggctccatcacctcttcttcttatt |
| | | actggggttggataagacagccacctggaaaagggctggagtggattggat |
| | | ctatctactacaccggtaccacctactacaatccttcattgaagagcagggtca |
| | | caataagcgtggataccagtaaaaaccagttttccttgaagctgagttctgtca |
| | | cagccgctgacaccgcagtgtattactgtgctagatacgttggtctgtctggtg |
| | | gttttgattactggggacaaggaactttggtcacagtgtcatct |
| 117 | Nucleic acid | gacatacaaatgacccagtctccttcttctctgagcgcatctgtcggcgataga |
| | sequence | gtgactattacatgtagggccagccagtctatctactcttatctgaattggtatca |
| | scFv-CD19-23 | gcaaaaacgcggcaaggctccaaaactgttgatctatgatgcttcaagcctg |
| | | caatccggagttccctcaagattttctggttccggctcaggaactgatttcacc |
| | | ctgactataagttctttgcagcctgaagactttgcaacatattactgtcagcaat |
| | | cttacaccgctccaccaacattcgggcaaggtaccaaggtcgaaattaaggg |
| | | tggtggtggttctggtggtggcggtagcggaggtggtggtagtgaggttcag |
| | | ttgttagagagcggggggggtctggttcagcctggtggcagcttaagactga |
| | | gttgtgccgcttctggttttactttctctaattatgcaatgtcctgggtgaggcaa |
| | | gcccccggtaaaggcctggaatgggtttcagctatctctgaatctggtcatgg |
| | | tacctactacgccgatagcgtgaaaggtagattcactatttccagggacaattc |
| | | aaagaacactttgtacttacagatgaactctttgagagctgaggacaccgcag |
| | | tgtattactgtgctagactggattgggctggttttgatgtttggggacaaggaac |
| | | tttggtcacagtgtcatct |
| 118 | Nucleic acid | gacatacaaatgacccagtctccttcttctctgagcgcatctgtcggcgataga |
| | sequence | gtgactattacatgtagggccagccagaccatctctagatatctgaattggtat |
| | scFv-CD19-#10 | cagcaaaaacccggcaaggctccaaaactgttgatctatgctgcttcaagcct |
| | | gcaatccggagttccctcaagattttctggttccggctcaggaactgatttcac |
| | | cctgactataagttctttgcagcctgaagactttgcaacatattactgtcagcaa |
| | | tcttacagaccaccactgacattcgggcaaggtaccaaggtcgaaattaagg |
| | | gtggtggtggttctggtggtggcggtagcggaggtggtggtagtgaggttca |
| | | gttgttagagagcggggggggtctggttcagcctggtggcagcttaagactg |
| | | agttgtgccgcttctggttttactttctcttcttatgcaatgtcctgggtgaggcaa |
| | | gcccccggtaaaggcctggaatgggtttcaaccatctctgctggtggtcatgg |
| | | tacctactacgccgatagcgtgaaaggtagattcactatttccagggacaattc |
| | | aaagaacactttgtacttacagatgaactctttgagagctgaggacaccgcag |
| | | tgtattactgtgctagaggtgctggttactttgattactggggacaaggaacttt |
| | | ggtcacagtgtcatct |
| 119 | Nucleic acid | gacatacaaatgacccagtctccttcttctctgagcgcatctgtcggcgataga |
| | sequence | gtgactattacatgtagggccagccagtctatctcttcttatctgaattggtatca |
| | scFv-CD19-#11 | gcaaaaacccggcaaggctccaaaactgttgatctatgctgcttcaagcctgc |
| | | aatccggagttccctcaagattttctggttccggctcaggaactgatttcaccct |
| | | gactataagttctttgcagcctgaagactttgcaacatattactgtcagcaaacc |
| | | ggtgctgttccatacacattcgggcaaggtaccaaggtcgaaattaagggtg |
| | | gtggtggttctggtggtggcggtagcggaggtggtggtagtgaggttcagtt |
| | | gttagagagcggggggggtctggttcagcctggtggcagcttaagactgag |
| | | ttgtgccgcttctggttttactttcagagattatgcaatgtcctgggtgaggcaa |
| | | gcccccggtaaaggcctggaatgggtttcagctatctctgaatctggtatcgat |
| | | acctactacgccgatagcgtgaaaggtagattcactatttccagggacaattc |
| | | aaagaacactttgtacttacagatgaactctttgagagctgaggacaccgcag |
| | | tgtattactgtgctagagttgctggttacgattctgattcttctacctactacgatt |
| | | acatggatgtttggggacaaggaactttggtcacagtgtcatct |
| 120 | Nucleic acid | cagtccgtgttgacccagcctccatcagtctcaggagccccaggccagaga |
| | sequence | gtgaccatttcttgtactggatcttcctcaaatatcggggccggttacgatgttc |
| | scFv-CD19-16 | attggtatcagcaactgcccggtacagctccaaaactgttgatctatggtaata |
| | | ataacagacctagcggtgtgcccgacaggttctccggctcaaagagcggaa |
| | | caagtgcttctttagcaattaccggcctgcaggctgaagatgaggcagactat |
| | | tactgtcaatcctacgatgtttctctgggtgtttgggtttttggtggcggtaccaa |
| | | attgactgtcttaggtggtggtggttctggtggtggcggtagcggaggtggtg |
| | | gtagtcagttacagttacaggagagcggtccaggtttagtgaaaccatccga |
| | | aactttatcactgacctgtacagtgtctggtggctccatctcttctccatcttatta |
| | | ctggggttggataagacagccacctggaaaagggctggagtggattggatc |
| | | tatctactacaccggtgctacctactacaatccttcattgaagagcagggtcac |
| | | aataagcgtggataccagtaaaaaccagttttccttgaagctgagttctgtcac |
| | | agccgctgacaccgcagtgtattactgtgctagatacggtccagctggtgttg |
| | | gttttgattactggggacaaggaactttggtcacagtgtcatct |
| 121 | linker | GGSG |
| 122 | linker | GGSGG |
| 123 | linker | GSGSG |
| 124 | linker | GSGGG |
| 125 | linker | GGGSG |
| 126 | linker | GSSSG |
| 127 | linker | GGGGS |
| 128 | linker | GGGGSGGGGSGGGGS |
| 129 | linker | GGTGGCGGTGGCTCGGGCGGTGGTGGGTCGGG |
| | | TGGCGGCGGATCT |
| 130 | hinge | DKTHT |
| 131 | hinge | CPPC |
| 132 | hinge | CPEPKSCDTPPPCPR |
| 133 | hinge | ELKTPLGDTTHT |
| 134 | hinge | KSCDKTHTCP |
| 135 | hinge | KCCVDCP |
| 136 | hinge | KYGPPCP |
| 137 | human IgG1 hinge | EPKSCDKTHTCPPCP |
| 138 | human IgG2 hinge | ERKCCVECPPCP |
| 139 | human IgG3 hinge | ELKTPLGDTTHTCPRCP |
| 140 | human IgG4 hinge | SPNMVPHAHHAQ |
| 56 | E4 CDR-L1 | RASQSISSYLN |
| 57 | E4 CDR-L2 | GASSLQS |
| 58 | E4 CDR-L3 | QQSYRTPVT |
| 59 | E4 CDR-H1 | KASGGTFSNYAIN |
| 60 | E4 CDR-H2 | MGRIVPLLGIANYAQ |
| 61 | E4 CDR-H3 | EHIAYRPTSAGYYYYMDI |
| 62 | CD19 E4 V_{L} | DIQMTQSPSSLSASVGDRVTITCRASQSISSYLNW |
| | | YQQKPGKAPKLLIYGASSLQSGVPSRFSGSGSGT |
| | | DFTLTISSLQPEDFATYYCQQSYRTPVTFGQGTK |
| | | VEIK |
| 63 | CD19 E4V_{H} | QVQLVQSGAEVKKPGSSVKVSCKASGGTFSNYA |
| | | INWVRQAPGQGLEWMGRIVPLLGIANYAQKFQG |
| | | RVTITADESTSTAYMELSSLRSEDTAVYYCAREH |
| | | IAYRPTSAGYYYYMDIWGQGTLVTVSS |
| 64 | scFv-CD19 E4 | DIQMTQSPSSLSASVGDRVTITCRASQSISSYLNW |
| | human derived | YQQKPGKAPKLLIYGASSLQSGVPSRFSGSGSGT |
| | | DFTLTISSLQPEDFATYYCQQSYRTPVTFGQGTK |
| | | VEIKGGGGSGGGGSGGGGSQVQLVQSGAEVKKP |
| | | GSSVKVSCKASGGTFSNYAINWVRQAPGQGLEW |
| | | MGRIVPLLGIANYAQKFQGRVTITADESTSTAYM |
| | | ELSSLRSEDTAVYYCAREHIAYRPTSAGYYYYM |
| | | DIWGQGTLVTVSS |
| 65 | Nucleic acid | gacatacaaatgacccagtctccttcttctctgagcgcatctgtcggcgataga |
| | sequence | gtgactattacatgtagggccagccagtctatctcttcttatctgaattggtatca |
| | CD19 E4 V_{L} | gcaaaaacccggcaaggctccaaaactgttgatctatggtgcttcaagcctg |
| | | caatccggagttccctcaagattttctggttccggctcaggaactgatttcacc |
| | | ctgactataagttctttgcagcctgaagactttgcaacatattactgtcagcaat |
| | | cttacagaaccccagttacattcgggcaaggtaccaaggtcgaaattaag |
| 66 | Nucleic acid | caggtccagttagttcaatcaggtgccgaggtcaaaaagccaggttcttccgt |
| | sequence | caaagtgtcatgcaaggctagcggtggcaccttttctaattacgctatcaattg |
| | CD19 E4V_{H} | ggtgagacaggcaccaggtcaaggactggaatggatgggaagaatcgttc |
| | | cactgctgggtatcgctaattatgcccagaagttccaaggcagggtgaccata |
| | | actgctgatgagagtacatctaccgcatacatggaattaagttctctgagatcc |
| | | gaggacaccgcagtgtattactgtgctagagaacatatcgcttacagaccaa |
| | | cctctgctggttactactactacatggatatctggggacaaggaactttggtca |
| | | cagtgtcatct |
| 67 | E7 CDR-L1 | RASQDITRYLN |
| 68 | E7 CDR-L2 | AASSLQS |
| 69 | E7 CDR-L3 | QQSYSYPPT |
| 70 | E7 CDR-H1 | AASGFTFRDYGMH |
| 71 | E7 CDR-H2 | VAVISYEGSNEYYADSV |
| 72 | E7 CDR-H3 | DRGFAGWYDYAFDP |
| 73 | CD19 E7 V_{L} | DIQMTQSPSSLSASVGDRVTITCRASQDITRYLN |
| | | WYQQKPGKAPKLLIYAASSLQSGVPSRFSGSGSG |
| | | TDFTLTISSLQPEDFATYYCQQSYSYPPTFGQGTK |
| | | VEIK |
| 74 | CD19 E7V_{H} | QVQLVESGGGVVQPGRSLRLSCAASGFTFRDYG |
| | | MHWVRQAPGKGLEWVAVISYEGSNEYYADSVK |
| | | GRFTISRDNSKNTLYLQMNSLRAEDTAVYYCAR |
| | | DRGFAGWYDYAFDPWGQGTLVTVSS |
| 75 | scFv-CD19 E7 | DIQMTQSPSSLSASVGDRVTITCRASQDITRYLN |
| | human derived | WYQQKPGKAPKLLIYAASSLQSGVPSRFSGSGSG |
| | | TDFTLTISSLQPEDFATYYCQQSYSYPPTFGQGTK |
| | | VEIKGGGGSGGGGSGGGGSQVQLVESGGGVVQP |
| | | GRSLRLSCAASGFTFRDYGMHWVRQAPGKGLE |
| | | WVAVISYEGSNEYYADSVKGRFTISRDNSKNTLY |
| | | LQMNSLRAEDTAVYYCARDRGFAGWYDYAFDP |
| | | WGQGTLVTVSS |
| 76 | Nucleic acid | gacatacaaatgacccagtctccttcttctctgagcgcatctgtcggcgataga |
| | sequence | gtgactattacatgtagggccagccaggatatcaccagatatctgaattggtat |
| | CD19 E7 V_{L} | cagcaaaaacccggcaaggctccaaaactgttgatctatgctgcttcaagcct |
| | | gcaatccggagttccctcaagattttctggttccggctcaggaactgatttcac |
| | | cctgactataagttctttgcagcctgaagactttgcaacatattactgtcagcaa |
| | | tcttactcttacccaccaacattcgggcaaggtaccaaggtcgaaattaag |
| 77 | Nucleic acid | caggttcagttagtcgagtctggtggcggtgtcgtccagcctggtagatcctta |
| | sequence | aggctgtcatgtgccgctagcggatttacctttagagattacggtatgcattgg |
| | CD19 E7V_{H} | gtgagacaagcccccggtaaaggcctggaatgggtcgctgtgataagttac |
| | | gaaggttctaacgaatactacgcagactccgttaagggtagattcactatttcc |
| | | agggataattcaaagaacactttgtatctgcagatgaactcattgagagctga |
| | | ggacaccgcagtgtattactgtgctagagatagaggttttgctggttggtacga |
| | | ttacgcttttgatccatggggacaaggaactttggtcacagtgtcatct |
| 78 | 7 CDR-L1 | RASQSISKYLN |
| 79 | 7 CDR-L2 | DASSLQS |
| 80 | 7 CDR-L3 | QQSYTIPLT |
| 81 | 7 CDR-H1 | KASGGTFSSYAFS |
| 82 | 7 CDR-H2 | MGGIVPLFGAVEYAQ |
| 83 | 7 CDR-H3 | EKGFYRYFDH |
| 84 | CD19-7 V_{L} | DIQMTQSPSSLSASVGDRVTITCRASQSISKYLNW |
| | | YQQKPGKAPKLLIYDASSLQSGVPSRFSGSGSGT |
| | | DFTLTISSLQPEDFATYYCQQSYTIPLTFGQGTKV |
| | | EIK |
| 85 | CD19-7V_{H} | QVQLVQSGAEVKKPGSSVKVSCKASGGTFSSYA |
| | | FSWVRQAPGQGLEWMGGIVPLFGAVEYAQKFQ |
| | | GRVTITADESTSTAYMELSSLRSEDTAVYYCARE |
| | | KGFYRYFDHWGQGTLVTVSS |
| 86 | scFv-CD19-7 | DIQMTQSPSSLSASVGDRVTITCRASQSISKYLNW |
| | human derived | YQQKPGKAPKLLIYDASSLQSGVPSRFSGSGSGT |
| | | DFTLTISSLQPEDFATYYCQQSYTIPLTFGQGTKV |
| | | EIKGGGGSGGGGSGGGGSQVQLVQSGAEVKKPG |
| | | SSVKVSCKASGGTFSSYAFSWVRQAPGQGLEWM |
| | | GGIVPLFGAVEYAQKFQGRVTITADESTSTAYME |
| | | LSSLRSEDTAVYYCAREKGFYRYFDHWGQGTLV |
| | | TVSS |
| 87 | Nucleic acid | gacatacaaatgacccagtctccttcttctctgagcgcatctgtcggcgataga |
| | sequence | gtgactattacatgtagggccagccagtctatctctaaatatctgaattggtatc |
| | CD19-7 V_{L} | agcaaaaacccggcaaggctccaaaactgttgatctatgatgcttcaagcctg |
| | | caatccggagttccctcaagattttctggttccggctcaggaactgatttcacc |
| | | ctgactataagttctttgcagcctgaagactttgcaacatattactgtcagcaat |
| | | cttacaccatcccactgacattcgggcaaggtaccaaggtcgaaattaag |
| 88 | Nucleic acid | caggtccagttagttcaatcaggtgccgaggtcaaaaagccaggttcttccgt |
| | sequence | caaagtgtcatgcaaggctagcggtggcaccttttcttcttacgctttttcttggg |
| | CD19-7V_{H} | tgagacaggcaccaggtcaaggactggaatggatgggaggtatcgttccac |
| | | tgtttggtgctgttgaatatgcccagaagttccaaggcagggtgaccataact |
| | | gctgatgagagtacatctaccgcatacatggaattaagttctctgagatccga |
| | | ggacaccgcagtgtattactgtgctagagaaaaaggtttttacagatactttgat |
| | | cattggggacaaggaactttggtcacagtgtcatct |
| 89 | 10 CDR-L1 | RASQTISRYLN |
| 90 | 10 CDR-L2 | AASSLQS |
| 91 | 10 CDR-L3 | QQSYRPPLT |
| 141 | 10 CDR-H1 | AASGFTFSSYAMS |
| 142 | 10 CDR-H2 | VSTISAGGHGTYYADSV |
| 143 | 10 CDR-H3 | GAGYFDY |
| 144 | CD19 10 V_{L} | DIQMTQSPSSLSASVGDRVTITCRASQTISRYLNW |
| | | YQQKPGKAPKLLIYAASSLQSGVPSRFSGSGSGT |
| | | DFTLTISSLQPEDFATYYCQQSYRPPLTFGQGTKV |
| | | EIK |
| 145 | CD19 10V_{H} | EVQLLESGGGLVQPGGSLRLSCAASGFTFSSYAM |
| | | SWVRQAPGKGLEWVSTISAGGHGTYYADSVKG |
| | | RFTISRDNSKNTLYLQMNSLRAEDTAVYYCARG |
| | | AGYFDYWGQGTLVTVSS |
| 146 | scFv-CD19 10 | DIQMTQSPSSLSASVGDRVTITCRASQTISRYLNW |
| | human derived | YQQKPGKAPKLLIYAASSLQSGVPSRFSGSGSGT |
| | | DFTLTISSLQPEDFATYYCQQSYRPPLTFGQGTKV |
| | | EIKGGGGSGGGGSGGGGSEVQLLESGGGLVQPG |
| | | GSLRLSCAASGFTFSSYAMSWVRQAPGKGLEWV |
| | | STISAGGHGTYYADSVKGRFTISRDNSKNTLYLQ |
| | | MNSLRAEDTAVYYCARGAGYFDYWGQGTLVTV |
| | | SS |
| 147 | Nucleic acid | gacatacaaatgacccagtctccttcttctctgagcgcatctgtcggcgataga |
| | sequence | gtgactattacatgtagggccagccagaccatctctagatatctgaattggtat |
| | CD19 10 V_{L} | cagcaaaaacccggcaaggctccaaaactgttgatctatgctgcttcaagcct |
| | | gcaatccggagttccctcaagattttctggttccggctcaggaactgatttcac |
| | | cctgactataagttctttgcagcctgaagactttgcaacatattactgtcagcaa |
| | | tcttacagaccaccactgacattcgggcaaggtaccaaggtcgaaattaag |
| 148 | Nucleic acid | gaggttcagttgttagagagcggggggggtctggttcagcctggtggcagct |
| | sequence | taagactgagttgtgccgcttctggttttactttctcttcttatgcaatgtcctgggt |
| | CD19 10V_{H} | gaggcaagcccccggtaaaggcctggaatgggtttcaaccatctctgctggt |
| | | ggtcatggtacctactacgccgatagcgtgaaaggtagattcactatttccag |
| | | ggacaattcaaagaacactttgtacttacagatgaactctttgagagctgagga |
| | | caccgcagtgtattactgtgctagaggtgctggttactttgattactggggaca |
| | | aggaactttggtcacagtgtcatct |
| 149 | 11 CDR-L1 | RASQSISSYLN |
| 150 | 11 CDR-L2 | AASSLQS |
| 151 | 11 CDR-L3 | QQTGAVPYTF |
| 152 | 11 CDR-H1 | AASGFTFRDYAMS |
| 153 | 11 CDR-H2 | VSAISESGIDTYYADSV |
| 154 | 11 CDR-H3 | VAGYDSDSSTYYDYMDV |
| 155 | CD19 11 V_{L} | DIQMTQSPSSLSASVGDRVTITCRASQSISSYLNW |
| | | YQQKPGKAPKLLIYAASSLQSGVPSRFSGSGSGT |
| | | DFTLTISSLQPEDFATYYCQQTGAVPYTFGQGTK |
| | | VEIK |
| 156 | CD19 11V_{H} | EVQLLESGGGLVQPGGSLRLSCAASGFTFRDYA |
| | | MSWVRQAPGKGLEWVSAISESGIDTYYADSVKG |
| | | RFTISRDNSKNTLYLQMNSLRAEDTAVYYCARV |
| | | AGYDSDSSTYYDYMDVWGQGTLVTVSS |
| 157 | scFv-CD19 11 | DIQMTQSPSSLSASVGDRVTITCRASQSISSYLNW |
| | human derived | YQQKPGKAPKLLIYAASSLQSGVPSRFSGSGSGT |
| | | DFTLTISSLQPEDFATYYCQQTGAVPYTFGQGTK |
| | | VEIKGGGGSGGGGSGGGGSEVQLLESGGGLVQP |
| | | GGSLRLSCAASGFTFRDYAMSWVRQAPGKGLE |
| | | WVSAISESGIDTYYADSVKGRFTISRDNSKNTLY |
| | | LQMNSLRAEDTAVYYCARVAGYDSDSSTYYDY |
| | | MDVWGQGTLVTVSS |
| 158 | Nucleic acid | gacatacaaatgacccagtctccttcttctctgagcgcatctgtcggcgataga |
| | sequence | gtgactattacatgtagggccagccagtctatctcttcttatctgaattggtatca |
| | CD19 11 V_{L} | gcaaaaacccggcaaggctccaaaactgttgatctatgctgcttcaagcctgc |
| | | aatccggagttccctcaagattttctggttccggctcaggaactgatttcaccct |
| | | gactataagttctttgcagcctgaagactttgcaacatattactgtcagcaaacc |
| | | ggtgctgttccatacacattcgggcaaggtaccaaggtcgaaattaag |
| 159 | Nucleic acid | gaggttcagttgttagagagcggggggggtctggttcagcctggtggcagct |
| | sequence | taagactgagttgtgccgcttctggttttactttcagagattatgcaatgtcctgg |
| | CD19 11V_{H} | gtgaggcaagcccccggtaaaggcctggaatgggtttcagctatctctgaat |
| | | ctggtatcgatacctactacgccgatagcgtgaaaggtagattcactatttcca |
| | | gggacaattcaaagaacactttgtacttacagatgaactctttgagagctgag |
| | | gacaccgcagtgtattactgtgctagagttgctggttacgattctgattcttctac |
| | | ctactacgattacatggatgtttggggacaaggaactttggtcacagtgtcatc |
| | | t |
| 160 | 14 CDR-L1 | RASQSISNYLN |
| 161 | 14 CDR-L2 | AASSLQS |
| 162 | 14 CDR-L3 | QQAYSAPIT |
| 163 | 14 CDR-H1 | AASGFTFGDYAMS |
| 164 | 14 CDR-H2 | VSAISRGGHGTYYADSV |
| 165 | 14 CDR-H3 | LVGYGLDY |
| 166 | CD19 14 V_{L} | DIQMTQSPSSLSASVGDRVTITCRASQSISNYLNW |
| | | YQQKPGKAPKLLIYAASSLQSGVPSRFSGSGSGT |
| | | DFTLTISSLQPEDFATYYCQQAYSAPITFGQGTKV |
| | | EIK |
| 167 | CD19 14V_{H} | EVQLLESGGGLVQPGGSLRLSCAASGFTFGDYA |
| | | MSWVRQAPGKGLEWVSAISRGGHGTYYADSVK |
| | | GRFTISRDNSKNTLYLQMNSLRAEDTAVYYCAR |
| | | LVGYGLDYWGQGTLVTVSS |
| 168 | scFv-CD19 14 | DIQMTQSPSSLSASVGDRVTITCRASQSISNYLNW |
| | human derived | YQQKPGKAPKLLIYAASSLQSGVPSRFSGSGSGT |
| | | DFTLTISSLQPEDFATYYCQQAYSAPITFGQGTKV |
| | | EIKGGGGSGGGGSGGGGSEVQLLESGGGLVQPG |
| | | GSLRLSCAASGFTFGDYAMSWVRQAPGKGLEW |
| | | VSAISRGGHGTYYADSVKGRFTISRDNSKNTLYL |
| | | QMNSLRAEDTAVYYCARLVGYGLDYWGQGTL |
| | | VTVSS |
| 169 | Nucleic acid | gacatacaaatgacccagtctccttcttctctgagcgcatctgtcggcgataga |
| | sequence | gtgactattacatgtagggccagccagtctatctctaattatctgaattggtatca |
| | CD19 14 V_{L} | gcaaaaacccggcaaggctccaaaactgttgatctatgctgcttcaagcctgc |
| | | aatccggagttccctcaagattttctggttccggctcaggaactgatttcaccct |
| | | gactataagttctttgcagcctgaagactttgcaacatattactgtcagcaagct |
| | | tactctgctccaatcacattcgggcaaggtaccaaggtcgaaattaag |
| 170 | Nucleic acid | gaggttcagttgttagagagcggggggggtctggttcagcctggtggcagct |
| | sequence | taagactgagttgtgccgcttctggttttactttcggtgattatgcaatgtcctgg |
| | CD19 14V_{H} | gtgaggcaagcccccggtaaaggcctggaatgggtttcagctatctctagag |
| | | gtggtcatggtacctactatgccgatagcgtgaaaggtagattcactatttcca |
| | | gggacaattcaaagaacactttgtacttacagatgaactctttgagagctgag |
| | | gacaccgcagtgtattactgtgctagactggttggttacggtctggattactgg |
| | | ggacaaggaactttggtcacagtgtcatct |
| 171 | 15 CDR-L1 | RASQPIRPYLN |
| 172 | 15 CDR-L2 | DASSLQS |
| 173 | 15 CDR-L3 | QQSYSAPYT |
| 174 | 15 CDR-H1 | AASGFTFSSYAMS |
| 175 | 15 CDR-H2 | VSVISGGGANTYYADSVK |
| 176 | 15 CDR-H3 | DWRYFDH |
| 177 | CD19 15 V_{L} | DIQMTQSPSSLSASVGDRVTITCRASQPIRPYLNW |
| | | YQQKPGKAPKLLIYDASSLQSGVPSRFSGSGSGT |
| | | DFTLTISSLQPEDFATYYCQQSYSAPYTFGQGTK |
| | | VEIK |
| 178 | CD19 15V_{H} | EVQLLESGGGLVQPGGSLRLSCAASGFTFSSYAM |
| | | SWVRQAPGKGLEWVSVISGGGANTYYADSVKG |
| | | RFTISRDNSKNTLYLQMNSLRAEDTAVYYCARD |
| | | WRYFDHWGQGTLVTVSS |
| 179 | scFv-CD19 15 | DIQMTQSPSSLSASVGDRVTITCRASQPIRPYLNW |
| | human derived | YQQKPGKAPKLLIYDASSLQSGVPSRFSGSGSGT |
| | | DFTLTISSLQPEDFATYYCQQSYSAPYTFGQGTK |
| | | VEIKGGGGSGGGGSGGGGSEVQLLESGGGLVQP |
| | | GGSLRLSCAASGFTFSSYAMSWVRQAPGKGLEW |
| | | VSVISGGGANTYYADSVKGRFTISRDNSKNTLYL |
| | | QMNSLRAEDTAVYYCARDWRYFDHWGQGTLV |
| | | TVSS |
| 180 | Nucleic acid | caaatgacccagtctccttcttctctgagcgcatctgtcggcgatagagtgact |
| | sequence | attacatgtagggccagccagcctatcagaccttatctgaattggtatcagcaa |
| | CD19 15 V_{L} | aaacccggcaaggctccaaaactgttgatctatgatgcttcaagcctgcaatc |
| | | cggagttccctcaagattttctggttccggctcaggaactgatttcaccctgact |
| | | ataagttctttgcagcctgaagactttgcaacatattactgtcagcaatcttactc |
| | | tgctccatacacattcgggcaaggtaccaaggtcgaaattaag |
| 181 | Nucleic acid | gaggttcagttgttagagagcggggggggtctggttcagcctggtggcagct |
| | sequence | taagactgagttgtgccgcttctggttttactttctcttcttatgcaatgtcctgggt |
| | CD19 15V_{H} | gaggcaagcccccggtaaaggcctggaatgggtttcagttatctctggtggt |
| | | ggtgctaatacctactacgccgatagcgtgaaaggtagattcactatttccag |
| | | ggacaattcaaagaacactttgtacttacagatgaactctttgagagctgagga |
| | | caccgcagtgtattactgtgctagagattggagatactttgatcattggggaca |
| | | aggaactttggtcacagtgtcatct |
| 182 | 16 CDR-L1 | TGSSSNIGAGYDVH |
| 183 | 16 CDR-L2 | GNNNRPS |
| 184 | 16 CDR-L3 | QSYDVSLGVWV |
| 185 | 16 CDR-H1 | TVSGGSISSPSYYWG |
| 186 | 16 CDR-H2 | IGSIYYTGATYYNPSL |
| 187 | 16 CDR-H3 | YGPAGVGFDY |
| 188 | CD19 16 V_{L} | QSVLTQPPSVSGAPGQRVTISCTGSSSNIGAGYDV |
| | | HWYQQLPGTAPKLLIYGNNNRPSGVPDRFSGSKS |
| | | GTSASLAITGLQAEDEADYYCQSYDVSLGVWVF |
| | | GGGTKLTVL |
| 189 | CD19 16V_{H} | QLQLQESGPGLVKPSETLSLTCTVSGGSISSPSYY |
| | | WGWIRQPPGKGLEWIGSIYYTGATYYNPSLKSR |
| | | VTISVDTSKNQFSLKLSSVTAADTAVYYCARYGP |
| | | AGVGFDYWGQGTLVTVSS |
| 190 | scFv-CD19 16 | QSVLTQPPSVSGAPGQRVTISCTGSSSNIGAGYDV |
| | human derived | HWYQQLPGTAPKLLIYGNNNRPSGVPDRFSGSKS |
| | | GTSASLAITGLQAEDEADYYCQSYDVSLGVWVF |
| | | GGGTKLTVLGGGGSGGGGSGGGGSQLQLQESGP |
| | | GLVKPSETLSLTCTVSGGSISSPSYYWGWIRQPPG |
| | | KGLEWIGSIYYTGATYYNPSLKSRVTISVDTSKN |
| | | QFSLKLSSVTAADTAVYYCARYGPAGVGFDYW |
| | | GQGTLVTVSS |
| 191 | Nucleic acid | cagtccgtgttgacccagcctccatcagtctcaggagccccaggccagaga |
| | sequence | gtgaccatttcttgtactggatcttcctcaaatatcggggccggttacgatgttc |
| | CD19 16 V_{L} | attggtatcagcaactgcccggtacagctccaaaactgttgatctatggtaata |
| | | ataacagacctagcggtgtgcccgacaggttctccggctcaaagagcggaa |
| | | caagtgcttctttagcaattaccggcctgcaggctgaagatgaggcagactat |
| | | tactgtcaatcctacgatgtttctctgggtgtttgggtttttggtggcggtaccaa |
| | | attgactgtctta |
| 192 | Nucleic acid | cagttacagttacaggagagcggtccaggtttagtgaaaccatccgaaacttt |
| | sequence | atcactgacctgtacagtgtctggtggctccatctcttctccatcttattactggg |
| | CD19 16V_{H} | gttggataagacagccacctggaaaagggctggagtggattggatctatcta |
| | | ctacaccggtgctacctactacaatccttcattgaagagcagggtcacaataa |
| | | gcgtggataccagtaaaaaccagttttccttgaagctgagttctgtcacagcc |
| | | gctgacaccgcagtgtattactgtgctagatacggtccagctggtgttggtttt |
| | | gattactggggacaaggaactttggtcacagtgtcatct |
| 193 | 18 CDR-L1 | TGSSSNIGAGYDVH |
| 194 | 18 CDR-L2 | GTKNRPS |
| 195 | 18 CDR-L3 | QSYDVRLKGWV |
| 196 | 18 CDR-H1 | TVSGGSITSSSYYWG |
| 197 | 18 CDR-H2 | IGSIYYTGTTYYNPSL |
| 198 | 18 CDR-H3 | YVGLSGGFDY |
| 199 | CD19 18 V_{L} | QSVLTQPPSVSGAPGQRVTISCTGSSSNIGAGYDV |
| | | HWYQQLPGTAPKLLIYGTKNRPSGVPDRFSGSKS |
| | | GTSASLAITGLQAEDEADYYCQSYDVRLKGWVF |
| | | GGGTKLTVL |
| 200 | CD19 18V_{H} | QLQLQESGPGLVKPSETLSLTCTVSGGSITSSSYY |
| | | WGWIRQPPGKGLEWIGSIYYTGTTYYNPSLKSRV |
| | | TISVDTSKNQFSLKLSSVTAADTAVYYCARYVGL |
| | | SGGFDYWGQGTLVTVSS |
| 201 | scFv-CD19 18 | QSVLTQPPSVSGAPGQRVTISCTGSSSNIGAGYDV |
| | human derived | HWYQQLPGTAPKLLIYGTKNRPSGVPDRFSGSKS |
| | | GTSASLAITGLQAEDEADYYCQSYDVRLKGWVF |
| | | GGGTKLTVLGGGGSGGGGSGGGGSQLQLQESGP |
| | | GLVKPSETLSLTCTVSGGSITSSSYYWGWIRQPPG |
| | | KGLEWIGSIYYTGTTYYNPSLKSRVTISVDTSKN |
| | | QFSLKLSSVTAADTAVYYCARYVGLSGGFDYW |
| | | GQGTLVTVSS |
| 202 | Nucleic acid | cagtccgtgttgacccagcctccatcagtctcaggagccccaggccagaga |
| | sequence | gtgaccatttcttgtactggatcttcctcaaatatcggggccggttacgatgttc |
| | CD19 18 V_{L} | attggtatcagcaactgcccggtacagctccaaaactgttgatctatggtacca |
| | | aaaacagacctagcggtgtgcccgacaggttctccggctcaaagagcggaa |
| | | caagtgcttctttagcaattaccggcctgcaggctgaagatgaggcagactat |
| | | tactgtcaatcctacgatgttagactgaaaggttgggtttttggtggcggtacca |
| | | aattgactgtctta |
| 203 | Nucleic acid | cagttacagttacaggagagcggtccaggtttagtgaaaccatccgaaacttt |
| | sequence | atcactgacctgtacagtgtctggtggctccatcacctcttcttcttattactggg |
| | CD19 18V_{H} | gttggataagacagccacctggaaaagggctggagtggattggatctatcta |
| | | ctacaccggtaccacctactacaatccttcattgaagagcagggtcacaataa |
| | | gcgtggataccagtaaaaaccagttttccttgaagctgagttctgtcacagcc |
| | | gctgacaccgcagtgtattactgtgctagatacgttggtctgtctggtggttttg |
| | | attactggggacaaggaactttggtcacagtgtcatct |
| 204 | 23 CDR-L1 | RASQSIYSYLN |
| 205 | 23 CDR-L2 | DASSLQS |
| 206 | 23 CDR-L3 | QQSYTAPPT |
| 207 | 23 CDR-H1 | AASGFTFSNYAMS |
| 208 | 23 CDR-H2 | VSAISESGHGTYYADSV |
| 209 | 23 CDR-H3 | LDWAGFDV |
| 210 | CD19 23 V_{L} | DIQMTQSPSSLSASVGDRVTITCRASQSIYSYLNW |
| | | YQQKRGKAPKLLIYDASSLQSGVPSRFSGSGSGT |
| | | DFTLTISSLQPEDFATYYCQQSYTAPPTFGQGTKV |
| | | EIK |
| 211 | CD19 23V_{H} | EVQLLESGGGLVQPGGSLRLSCAASGFTFSNYA |
| | | MSWVRQAPGKGLEWVSAISESGHGTYYADSVK |
| | | GRFTISRDNSKNTLYLQMNSLRAEDTAVYYCAR |
| | | LDWAGFDVWGQGTLVTVSS |
| 212 | scFv-CD19 23 | DIQMTQSPSSLSASVGDRVTITCRASQSIYSYLNW |
| | human derived | YQQKRGKAPKLLIYDASSLQSGVPSRFSGSGSGT |
| | | DFTLTISSLQPEDFATYYCQQSYTAPPTFGQGTKV |
| | | EIKGGGGSGGGGSGGGGSEVQLLESGGGLVQPG |
| | | GSLRLSCAASGFTFSNYAMSWVRQAPGKGLEW |
| | | VSAISESGHGTYYADSVKGRFTISRDNSKNTLYL |
| | | QMNSLRAEDTAVYYCARLDWAGFDVWGQGTL |
| | | VTVSS |
| 213 | Nucleic acid | gacatacaaatgacccagtctccttcttctctgagcgcatctgtcggcgataga |
| | sequence | gtgactattacatgtagggccagccagtctatctactcttatctgaattggtatca |
| | CD19 23 V_{L} | gcaaaaacgcggcaaggctccaaaactgttgatctatgatgcttcaagcctg |
| | | caatccggagttccctcaagattttctggttccggctcaggaactgatttcacc |
| | | ctgactataagttctttgcagcctgaagactttgcaacatattactgtcagcaat |
| | | cttacaccgctccaccaacattcgggcaaggtaccaaggtcgaaattaag |
| 214 | Nucleic acid | gaggttcagttgttagagagcggggggggtctggttcagcctggtggcagct |
| | sequence | taagactgagttgtgccgcttctggttttactttctctaattatgcaatgtcctggg |
| | CD19 23V_{H} | tgaggcaagcccccggtaaaggcctggaatgggtttcagctatctctgaatct |
| | | ggtcatggtacctactacgccgatagcgtgaaaggtagattcactatttccag |
| | | ggacaattcaaagaacactttgtacttacagatgaactctttgagagctgagga |
| | | caccgcagtgtattactgtgctagactggattgggctggttttgatgtttgggga |
| | | caaggaactttggtcacagtgtcatct |
| 215 | IL18(F170E) | YFGKLESKLSVIRNLNDQVLFIDQGNRPLFEDMT |
| | | DSDCRDNAPRTIFIISMYKDSQPRGMAVTISVKCE |
| | | KISTLSCENKIISFKEMNPPDNIKDTKSDIIFFQRSV |
| | | PGHDNKMQFESSSYEGYFLACEKERDLEKLILKK |
| | | EDELGDRSIMFTVQNED |
| 216 | Nucleic acid | cagtctgtgctgacacagcctccatctgtgtctggcgctccaggccagagag |
| | sequence | tgaccatcagctgtacaggcagcagcagcaatatcggagccggctatgacg |
| | CD19 52CO scFv | tgcactggtatcagcagctgcctggcacagcccctaaactgctgatctacgg |
| | | caccaagaacagacccagcggcgtgcccgatagattcagcggctctaagtc |
| | | tggcacaagcgccagcctggccattactggactgcaggccgaagatgagg |
| | | ccgactactactgccagagctacgacgtgcggctgaaaggctgggttttcgg |
| | | cggaggcacaaagctgacagtgcttggaggcggaggatctggcggaggtg |
| | | gaagtggcggaggcggatctcaactgcagctccaagaatctggccctggcc |
| | | tggtcaagcctagcgagacactgagcctgacctgtacagtgtccggcggca |
| | | gcatcacaagcagcagctattactggggctggatcagacagcctcctggca |
| | | aaggcctggaatggatcggctccatctactacaccggcaccacctactacaa |
| | | ccccagcctgaagtcccgcgtgaccatctctgtggacaccagcaagaacca |
| | | gttctccctgaagctgagcagcgtgacagccgccgatacagccgtgtactac |
| | | tgcgccagatacgtgggactgagcggcggctttgattattggggccagggc |
| | | acactggtcaccgtgtcatct |
| 217 | Full-length CD19 | MPPPRLLFFLLFLTPMEVRPEEPLVVKVEEGDNA |
| | (UniProtKB | VLQCLKGTSDGPTQQLTWSRESPLKPFLKLSLGL |
| | accession: | PGLGIHMRPLAIWLFIFNVSQQMGGFYLCQPGPP |
| | P15391-1) | SEKAWQPGWTVNVEGSGELFRWNVSDLGGLGC |
| | | GLKNRSSEGPSSPSGKLMSPKLYVWAKDRPEIWE |
| | | GEPPCLPPRDSLNQSLSQDLTMAPGSTLWLSCGV |
| | | PPDSVSRGPLSWTHVHPKGPKSLLSLELKDDRPA |
| | | RDMWVMETGLLLPRATAQDAGKYYCHRGNLT |
| | | MSFHLEITARPVLWHWLLRTGGWKVSAVTLAY |
| | | LIFCLCSLVGILHLQRALVLRRKRKRMTDPTRRFF |
| | | KVTPPPGSGPQNQYGNVLSLPTPTSGLGRAQRWAA |
| | | GLGGTAPSYGNPSSDVQADGALGSRSPPGVGPEEE |
| | | EGEGYEEPDSEEDSEFYENDSNLGQDQLSQDGSGY |
| | | ENPEDEPLGPEDEDSFSNAESYENEDEELTQPVART |
| | | MDFLSPHGSAWDPSREATSLGSQSYEDMRGILYAAP |
| | | QLRSIRGQPGPNHEEDADSYENMDNPDGPDPAWG |
| | | GGGRMGTWSTR |
| 218 | 7URV_1\|Chain | EEPLVVKVEEGDNAVLQCLKGTSDGPTQQLTWS |
| | A[auth C]\|B- | RESPLKPFLKLSLGLPGLGIHVSPLAIWLFISNVSQ |
| | lymphocyte antigen | QMGGFYLCQPGPPSEKAWQPGWTVNVEGSGEL |
| | CD19\|Homo | FRWNVSDLGGLGCGLKNRSSEGPSSPSGKLMSP |
| | sapiens (9606) | KLYVWAKDRPEIWEGEPPCLPPRDSLNQSLSQD |
| | | LTMAPGSTLWLSCGVPPDSVSRGPLSWTHVHPK |
| | | GPKSLLSLELKDDRPARDMWVMETGLLLPRATA |
| | | QDAGKYYCHRGNLTMSFHLEITAR |
| 219 | FMC63, 4G7, 3B10 | WAKDRPEIWEGEP |
| | epitope region 1 | |
| 220 | FMC63, 4G7, 3B10 | PKGPKSLLSLE |
| | epitope region 2 | |
| 221 | CD19 42og epitope | QPGPPSEKAWQP |
| | region 1 | |
| 222 | CD19 42og epitope | VPPDSVSRGPL |
| | region 2 | |
| 223 | CD19 42og epitope | QPGPPSEKA |
| | region 1 | |
| 224 | CD19 42og epitope | PPDSVSR |
| | region 2 | |
| 225 | Nucleic acid | GACATCCAGATGACACAGAGCCCTAGCAGCCT |
| | sequence | GTCTGCCAGCGTGGGAGACAGAGTGACCATCA |
| | CD19 42CO scFv | CCTGTAGAGCCAGCCAGACCATCAGCAACTAC |
| | (P14) | CTGAACTGGTATCAGCAGAAGCCCGGCAAGGC |
| | Codon optimized | CCCTAAGCTGCTGATCTATGCTGCCAGCTCTCT |
| | version of P1 (42 | GCAGTCTGGCGTGCCCAGCAGATTTTCTGGCA |
| | opt) | GCGGCTCTGGCACCGACTTCACCCTGACCATAT |
| | 42 scFv Codon | CTAGCCTGCAGCCTGAGGACTTCGCCACCTACT |
| | Optimized- | ACTGCCAGCAGAGCTACAGCACCCCTCCTACA |
| | nucleotides | TTTGGCCAGGGCACCAAGGTGGAAATCAAAGG |
| | | CGGCGGAGGATCTGGCGGAGGTGGAAGTGGC |
| | | GGAGGCGGATCTGAAGTTCAGCTGCTTGAATC |
| | | TGGCGGCGGACTGGTTCAACCTGGCGGATCTC |
| | | TGAGACTGAGCTGTGCCGCCAGCGGCTTCACC |
| | | TTCAGCAATTACGCCATCAGCTGGGTCCGACA |
| | | GGCCCCTGGAAAAGGCCTTGAATGGGTGTCCG |
| | | TGATCACAGCCAGCGGCGTGGACACCTATTAC |
| | | GCCGATTCTGTGAAGGGCAGATTCACCATCAG |
| | | CCGGGACAACAGCAAGAACACCCTGTACCTGC |
| | | AGATGAACAGCCTGAGAGCCGAGGACACCGCC |
| | | GTGTACTATTGTGCCAGAGGCGGCACCCCTTA |
| | | CTTCATCACCACCTACGACTACTACGGCTTCGA |
| | | CGTGTGGGGCCAGGGAACACTGGTTACCGTTA |
| | | GCTCT |
| 226 | amino acid | DIQMTQSPSSLSASVGDRVTITCRASQTISNYLNW |
| | sequence | YQQKPGKAPKLLIYAASSLQSGVPSRFSGSGSGT |
| | CD19 42CO scFv | DFTLTISSLQPEDFATYYCQQSYSTPPTFGQGTKV |
| | (P14) | EIKGGGGSGGGGSGGGGSEVQLLESGGGLVQPG |
| | Codon optimized | GSLRLSCAASGFTFSNYAISWVRQAPGKGLEWV |
| | version of P1 (42 | SVITASGVDTYYADSVKGRFTISRDNSKNTLYLQ |
| | opt) | MNSLRAEDTAVYYCARGGTPYFITTYDYYGFDV |
| | 42 scFv Codon | WGQGTLVTVSS |
| | Optimized-amino | |
| | acids | |

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

What is claimed is:

1. A vector comprising:
(a) a first polynucleotide comprising a constitutive promoter operably linked to a nucleic acid encoding a chimeric antigen receptor (CAR), wherein the CAR comprises a single chain antibody or a single chain antibody fragment comprising an anti-CD19 binding domain, a transmembrane domain, a costimulatory, and an intracellular signaling domain; and
(b) a second polynucleotide comprising a nucleic acid encoding a polypeptide that enhances an immune cell function, or a functional derivative thereof;
wherein the first polynucleotide is operably linked to the second polypeptide via a linker peptide
wherein the anti-CD19 binding domain comprises:
(a) a light chain variable domain including a light chain complementary determining region 1 (LC CDR1) of SEQ ID NO: 1, a light chain complementary determining region 2 (LC CDR2) of SEQ ID NO: 2, and a light chain complementary determining region 3 (LC CDR3) of SEQ ID NO: 3; and
a heavy chain variable domain including a heavy chain complementary determining region 1 (HC CDR1) of SEQ ID NO: 4, a heavy chain complementary determining region 2 (HC CDR2) of SEQ ID NO: 5, and a heavy chain complementary determining region 3 (HC CDR3) of SEQ ID NO: 6; or
(b) a light chain variable domain including a light chain complementary determining region 1 (LC CDR1) of SEQ ID NO: 193, a light chain complementary determining region 2 (LC CDR2) of SEQ ID NO: 194, and a light chain complementary determining region 3 (LC CDR3) of SEQ ID NO: 195; and
a heavy chain variable domain including a heavy chain complementary determining region 1 (HC CDR1) of SEQ ID NO: 196, a heavy chain complementary determining region 2 (HC CDR2) of SEQ ID NO: 197, and a heavy chain complementary determining region 3 (HC CDR3) of SEQ ID NO: 198; or
(c) a light chain variable domain comprising a light chain complementary determining region 1 (LC CDR1), a light chain complementary determining region 2 (LC CDR2), and a light chain complementary determining region 3 (LC CDR3) disclosed in Table 2; and
a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2.

2. The vector of claim 1, wherein the polypeptide that enhances an immune cell function, or a functional derivative thereof is selected from the group consisting of a cytokine, an interferon, a chemokine, an antibody or antibody fragment, a checkpoint inhibitor antagonist, a dominant negative receptor, a switch receptor, and a combination thereof.

3. The vector of claim 1, wherein the polypeptide that enhances an immune cell function, or a functional derivative thereof is:
(a) a chemokine, a chemokine receptor, a cytokine, a cytokine receptor, and a combination thereof;
(b) a cytokine selected from the group consisting of Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), Interleukin-18 (IL-18), Interleukin-18 receptor (IL-18R), Interleukin-21 (IL-21), granulocyte macrophage colony stimulating factor, alpha, beta or gamma interferon, erythropoietin, and a combination thereof; or
(c) a chemokine selected from CCL21, CCL19, or a combination thereof.

4. The vector of claim 1, wherein the polypeptide that enhances an immune cell function, or a functional derivative thereof further comprises:
(a) a leader sequence selected from the group consisting of an IL-2 signal sequence, an IL-12 signal sequence, a kappa leader sequence, a CD8 leader sequence, or any equivalent thereof; or
(b) the amino acid sequence of SEQ ID NO: 25 or an amino acid sequence having about 90%, about 91%, about 92%, about 93%, about 94%, about 95%, about 96%, about 97%, about 98%, or about 99% identity to SEQ ID NO: 25.

5. The vector of claim 1, wherein the polypeptide that enhances an immune cell function, or a functional derivative thereof comprises:
(a) an IL-18 polypeptide or a polypeptide having the amino acid sequence of SEQ ID NO: 105, SEQ ID NO: 215, SEQ ID NO: 106, SEQ ID NO: 107 or an amino acid sequence having about 90%, about 91%, about 92%, about 93%, about 94%, about 95%, about 96%, about 97%, about 98%, or about 99% identity to SEQ ID NO: 105, SEQ ID NO: 215, SEQ ID NO: 106, or SEQ ID NO: 107; and
(b) further comprises a CD8 leader sequence having the amino acid sequence of SEQ ID NO: 25 or an amino acid sequence having about 90%, about 91%, about 92%, about 93%, about 94%, about 95%, about 96%, about 97%, about 98%, or about 99% identity to SEQ ID NO: 25.

6. The vector of claim 5, wherein the IL-18 polypeptide comprises the amino acid sequence of SEQ ID NO: 105, SEQ ID NO: 215, SEQ ID NO: 106, SEQ ID NO: 107 and the amino acid sequence of SEQ ID NO: 25.

7. The vector of claim 5, wherein the IL-18 polypeptide comprises:
(a) a mutation at a position selected from the group consisting of position 42, 74, 85, 87, 89, 104, 112, 10, 132, 143, 149, 163, and 189 of SEQ ID NO: 107; or
(b) E42A; E42K; K89A; E42A and K89A; E42K and K89A; E42A and C74S; E42A, C74S, and K89A; C74S, and K89A; C74S, C112S, and C112S; E42A, C74S, C112S, and C112S; E42A, K89A, C74S, C112S, and C112S in SEQ ID NO: 107.

8. The vector of claim 7, wherein the mutated IL-18 polypeptide:
(a) exhibits at least an about 2-fold increased activity when compared to WT IL-18;
(b) is resistant to IL18BP inhibition when compared to WT IL-18; and/or
(c) requires at least an about 4 fold concentration of IL-18BP for neutralization when compared to WT IL-18.

9. The vector of claim 1, wherein the vector:
(a) is selected from the group consisting of a DNA, a RNA, a plasmid, a lentiviral vector, an adenoviral vector, or a retroviral vector; or
(b) is a lentiviral vector; or
(c) is an in vitro transcribed vector;
(d) further comprises a rev response element (RRE), a poly(A) tail, a 3′ UTR, a woodchuck hepatitis virus posttranscriptional regulatory element (WPRE), and/or a cPPT sequence;
(e) comprises a WPRE sequence of SEQ ID NO: 100.

10. The vector of claim 1, wherein the constitutive promoter comprises:
(a) a promoter selected from the group consisting of an EF-1alpha promoter, a PGK-1 promoter, a truncated PGK-1 promoter, an UBC promoter, a CMV promoter, a CAGG promoter, and an SV40 promoter; or
(b) the sequence of SEQ ID NO: 101.

11. The vector of claim 1, wherein the anti-CD19 binding domain comprises:
a light chain variable domain including a light chain complementary determining region 1 (LC CDR1) of SEQ ID NO: 1, a light chain complementary determining region 2 (LC CDR2) of SEQ ID NO: 2, and a light chain complementary determining region 3 (LC CDR3) of SEQ ID NO: 3; and
a heavy chain variable domain including a heavy chain complementary determining region 1 (HC CDR1) of SEQ ID NO: 4, a heavy chain complementary determining region 2 (HC CDR2) of SEQ ID NO: 5, and a heavy chain complementary determining region 3 (HC CDR3) of SEQ ID NO: 6.

12. The vector of claim 1, wherein:
(a) the light chain variable region comprises the amino acid sequence of SEQ ID NO: 7, or an amino acid sequence having at least about 90%, about 91%, about 92%, about 93%, about 94%, about 95%, about 96%, about 97%, about 98%, or about 99% identity to SEQ ID NO: 7; and
(b) the heavy chain variable region comprises the amino acid sequence of SEQ ID NO: 8, or an amino acid sequence having at least about 90%, about 91%, about 92%, about 93%, about 94%, about 95%, about 96%, about 97%, about 98%, or about 99% identity to SEQ ID NO: 8.

13. The vector of claim 1, wherein:
(a) the light chain variable region comprises the amino acid sequence of SEQ ID NO: 199, or an amino acid sequence having at least about 90%, about 91%, about 92%, about 93%, about 94%, about 95%, about 96%, about 97%, about 98%, or about 99% identity to SEQ ID NO: 199; and
(b) the heavy chain variable region comprises the amino acid sequence of SEQ ID NO: 200, or an amino acid sequence having at least about 90%, about 91%, about 92%, about 93%, about 94%, about 95%, about 96%, about 97%, about 98%, or about 99% identity to SEQ ID NO: 200.

14. The vector of claim 1, wherein:
(a) the light chain variable region comprises the amino acid sequence of SEQ ID NO: 7 and the heavy chain variable region comprises the amino acid sequence of SEQ ID NO: 8; or
(b) the light chain variable region comprises the amino acid sequence of SEQ ID NO: 199 and the heavy chain variable region comprises the amino acid sequence of SEQ ID NO: 200.

15. The vector of claim 1, wherein the CD19 binding domain:
(a) is a scFv;
(b) comprises an amino acid sequence selected from the group consisting of SEQ ID NO: 9, 18, 64, 75, 86, 190, 157, 212, 201, 226, 179, 168, and 146, or a sequence having 90%, about 91%, about 92%, about 93%, about 94%, about 95%, about 96%, about 97%, about 98%, or about 99% identity to SEQ ID NO: 9, 18, 64, 75, 86, 190, 157, 212, 201, 226, 179, 168, and 146; or
(c) is encoded by:
(i) a nucleic acid sequence selected from the group consisting of SEQ ID NO: 21, SEQ ID NO: 24, SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, and SEQ ID NO: 216; or
(ii) a sequence having 90%, about 91%, about 92%, about 93%, about 94%, about 95%, about 96%, about 97%, about 98%, or about 99% identity to SEQ ID NO: 21, SEQ ID NO: 24 SEQ ID NO: 102, SEQ ID NO: 103, SEQ ID NO: 104, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 225, or SEQ ID NO: 216.

16. A vector comprising:
(a) a first polynucleotide comprising a constitutive promoter operably linked to a nucleic acid encoding an anti-CD19 chimeric antigen receptor (CAR), wherein the CAR comprises:
(i) an anti-CD19 binding domain comprising:
(1) LC CDR1 of SEQ ID NO: 1, LC CDR2 of SEQ ID NO: 2, and LC CDR3, HC CDR1 of SEQ ID NO: 4, HC CDR2 of SEQ ID NO: 5, and HC CDR3 of SEQ ID NO: 6; or
(2) LC CDR1 of SEQ ID NO: 10, LC CDR2 of SEQ ID NO: 11, LC CDR3 of SEQ ID NO: 12; HC CDR1 of SEQ ID NO: 13, HC CDR2 of SEQ ID NO: 14, and HC CDR3 of SEQ ID NO: 15; or
(3) a light chain variable domain comprising a light chain complementary determining region 1 (LC CDR1), a light chain complementary determining region 2 (LC CDR2), and a light chain complementary determining region 3 (LC CDR3) disclosed in Table 2; and a heavy chain variable domain comprising a heavy chain complementary determining region 1 (HC CDR1), a heavy chain complementary determining region 2 (HC CDR2), and a heavy chain complementary determining region 3 (HC CDR3) disclosed in Table 2;
(ii) a transmembrane domain selected from CD28 or CD8 transmembrane domain;
(iii) a costimulatory domain comprising an intracellular signaling domain of a protein selected from the group consisting of OX40, CD27, CD2, CD28, ICOS, and 4-1BB; and
(iv) an intracellular signaling domain comprising of CD3-zeta; and
(b) a second polynucleotide comprising a nucleic acid encoding Interleukin-18 (IL-18), and/or Interleukin-18 receptor (IL-18R);
wherein the first polynucleotide is operably linked to the second polypeptide via a linker peptide selected from the group consisting of F2A, E2A, P2A, T2A, and Furin-(G4S)2-T2A.

17. A modified cell comprising a vector of claim 1.

18. The modified cell of claim 17, further comprising:
(a) a switch receptor comprising a first polypeptide that comprises at least a portion of an inhibitory molecule selected from the group consisting of PD1, TGFβR, TIM-2 and BTLA, conjugated to a second polypeptide that comprises an intracellular signaling domain of a molecule selected from the group consisting of OX40, CD27, CD28, IL-12R, ICOS, and 4-1BB;
(b) a dominant negative receptor comprising a truncated variant of a receptor selected from the group consisting of PD1, TGFβR, TIM-2 and BTLA; and/or
(c) a polypeptide that enhances an immune cell function, or a functional derivative thereof selected from the group consisting of a chemokine, a chemokine receptor, a cytokine, a cytokine receptor, Interleukin-7 (IL-7), Interleukin-7 receptor (IL-7R), Interleukin-15 (IL-15), Interleukin-15 receptor (IL-15R), Interleukin-21 (IL-21), CCL21, CCL19, and a combination thereof.

19. A composition comprising a modified cell or a population of modified cells of claim 17.

20. A method of treating a mammal having a disease associated with expression of CD19 comprising administering to the mammal an effective amount of a modified cell of claim 17.


SEQUENCE LISTING (229 sequences)

SEQ ID NO: 1 - protein, 11 aa, synthetic construct
  source 1..11: note = 42og CDR-L1
RASQTISNYLN

SEQ ID NO: 2 - protein, 7 aa, synthetic construct
  source 1..7: note = 42og CDR-L2
AASSLQS

SEQ ID NO: 3 - protein, 9 aa, synthetic construct
  source 1..9: note = 42og CDR-L3
QQSYSTPPT

SEQ ID NO: 4 - protein, 13 aa, synthetic construct
  source 1..13: note = 42og CDR-H1
AASGFTFSNYAIS

SEQ ID NO: 5 - protein, 17 aa, synthetic construct
  source 1..17: note = 42og CDR-H2
VSVITASGVDTYYADSV

SEQ ID NO: 6 - protein, 17 aa, synthetic construct
  source 1..17: note = 42og CDR-H3
GGTPYFITTYDYYGFDV

SEQ ID NO: 7 - protein, 107 aa, synthetic construct
  source 1..107: note = CD19 42og VL
DIQMTQSPSSLSASVGDRVTITCRASQTISNYLNWYQQKPGKAPKLLIYAASSLQSGVPSRFSGSGSGTDFTLTISSLQPEDFATYYCQQSYSTPPTFGQGTKVEIK

SEQ ID NO: 8 - protein, 126 aa, synthetic construct
  source 1..126: note = CD19 42ogVH
EVQLLESGGGLVQPGGSLRLSCAASGFTFSNYAISWVRQAPGKGLEWVSVITASGVDTYYADSVKGRFTISRDNSKNTLYLQMNSLRAEDTAVYYCARGGTPYFITTYDYYGFDVWGQGTLVTVSS

SEQ ID NO: 9 - protein, 248 aa, Homo sapiens
  source 1..248: note = scFv-CD19 42og human derived
DIQMTQSPSSLSASVGDRVTITCRASQTISNYLNWYQQKPGKAPKLLIYAASSLQSGVPSRFSGSGSGTDFTLTISSLQPEDFATYYCQQSYSTPPTFGQGTKVEIKGGGGSGGGGSGGGGSEVQLLESGGGLVQPGGSLRLSCAASGFTFSNYAISWVRQAPGKGLEWVSVITASGVDTYYADSVKGRFTISRDNSKNTLYLQMNSLRAEDTAVYYCARGGTPYFITTYDYYGFDVWGQGTLVTVSS

SEQ ID NO: 10 - protein, 12 aa, synthetic construct
  source 1..12: note = A4 CDR-L1
RASQSVSSNYLA

SEQ ID NO: 11 - protein, 7 aa, synthetic construct
  source 1..7: note = A4 CDR-L2
GASSRAT

SEQ ID NO: 12 - protein, 10 aa, synthetic construct
  source 1..10: note = A4 CDR-L3
QQYESSPSWT

SEQ ID NO: 13 - protein, 13 aa, synthetic construct
  source 1..13: note = A4 CDR-H1
KASGGTFSNYYIS

SEQ ID NO: 14 - protein, 15 aa, synthetic construct
  source 1..15: note = A4 CDR-H2
MGGIIPLFGTTNYAQ

SEQ ID NO: 15 - protein, 8 aa, synthetic construct
  source 1..8: note = A4 CDR-H3
GTWYAGDI

SEQ ID NO: 16 - protein, 109 aa, synthetic construct
  source 1..109: note = A4 VL
EIVLTQSPGTLSLSPGERATLSCRASQSVSSNYLAWYQQKPGQAPRLLIYGASSRATGIPDRFSGSGSGTDFTLTISRLEPEDFAVYYCQQYESSPSWTFGQGTKVEIK

SEQ ID NO: 17 - protein, 117 aa, synthetic construct
  source 1..117: note = A4 VH
QVQLVQSGAEVKKPGSSVKVSCKASGGTFSNYYISWVRQAPGQGLEWMGGIIPLFGTTNYAQKFQGRVTITADESTSTAYMELSSLRSEDTAVYYCARGTWYAGDIWGQGTLVTVSS

SEQ ID NO: 18 - protein, 241 aa, Homo sapiens
  source 1..241: note = scFv-CD19 A4 human derived
EIVLTQSPGTLSLSPGERATLSCRASQSVSSNYLAWYQQKPGQAPRLLIYGASSRATGIPDRFSGSGSGTDFTLTISRLEPEDFAVYYCQQYESSPSWTFGQGTKVEIKGGGGSGGGGSGGGGSQVQLVQSGAEVKKPGSSVKVSCKASGGTFSNYYISWVRQAPGQGLEWMGGIIPLFGTTNYAQKFQGRVTITADESTSTAYMELSSLRSEDTAVYYCARGTWYAGDIWGQGTLVTVSS

SEQ ID NO: 19 - DNA, 321 bp, synthetic construct
  source 1..321: mol_type = other DNA; note = Nucleic acid sequence CD19 42og VL
gacatacaaatgacccagtctccttcttctctgagcgcatctgtcggcgatagagtgactattacatgtagggccagccagaccatctctaattatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaatcttactctaccccaccaacattcgggcaaggtaccaaggtcgaaattaag

SEQ ID NO: 20 - DNA, 378 bp, synthetic construct
  source 1..378: mol_type = other DNA; note = Nucleic acid sequence CD19 42ogVH
gaggttcagttgttagagagcggggggggtctggttcagcctggtggcagcttaagactgagttgtgccgcttctggttttactttctctaattatgcaatatcctgggtgaggcaagcccccggtaaaggcctggaatgggtttcagttatcaccgcttctggtgttgatacctactacgccgatagcgtgaaaggtagattcactatttccagggacaattcaaagaacactttgtacttacagatgaactctttgagagctgaggacaccgcagtgtattactgtgctagaggtggtaccccatactttatcaccacctacgattactacggttttgatgtttggggacaaggaactttggtcacagtgtcatct

SEQ ID NO: 21 - DNA, 744 bp, synthetic construct
  source 1..744: mol_type = other DNA; note = Nucleic acid sequence scFv-CD19 42og
gacatacaaatgacccagtctccttcttctctgagcgcatctgtcggcgatagagtgactattacatgtagggccagccagaccatctctaattatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaatcttactctaccccaccaacattcgggcaaggtaccaaggtcgaaattaagggtggtggtggttctggtggtggcggtagcggaggtggtggtagtgaggttcagttgttagagagcggggggggtctggttcagcctggtggcagcttaagactgagttgtgccgcttctggttttactttctctaattatgcaatatcctgggtgaggcaagcccccggtaaaggcctggaatgggtttcagttatcaccgcttctggtgttgatacctactacgccgatagcgtgaaaggtagattcactatttccagggacaattcaaagaacactttgtacttacagatgaactctttgagagctgaggacaccgcagtgtattactgtgctagaggtggtaccccatactttatcaccacctacgattactacggttttgatgtttggggacaaggaactttggtcacagtgtcatct

SEQ ID NO: 22 - DNA, 327 bp, synthetic construct
  source 1..327: mol_type = other DNA; note = Nucleic acid sequence CD19 A4 VL
gaaatcgtcttgacccagtccccaggtactctgtctttaagtcccggggaaagagctactctgtcctgtagggcaagtcagtctgtttcttctaattatctggcctggtatcagcaaaaacccggtcaagctccaagactgttgatctatggtgcaagcagtagagccaccggaatccctgataggttttccggttcaggcagcggaactgacttcactctgacaatctcaagattggaacctgaggattttgccgtgtattactgtcagcagtacgaatcttctccatcttggacattcgggcaaggtaccaaggtcgaaattaag

SEQ ID NO: 23 - DNA, 351 bp, synthetic construct
  source 1..351: mol_type = other DNA; note = Nucleic acid sequence CD19 A4VH
caggtccagttagttcaatcaggtgccgaggtcaaaaagccaggttcttccgtcaaagtgtcatgcaaggctagcggtggcaccttttctaattactacatctcttgggtgagacaggcaccaggtcaaggactggaatggatgggaggtatcatcccactgtttggtaccaccaattatgcccagaagttccaaggcagggtgaccataactgctgatgagagtacatctaccgcatacatggaattaagttctctgagatccgaggacaccgcagtgtattactgtgctagaggtacctggtacgctggtgatatctggggacaaggaactttggtcacagtgtcatct

SEQ ID NO: 24 - DNA, 723 bp, synthetic construct
  source 1..723: mol_type = other DNA; note = Nucleic acid sequence scFv-CD19 A4
gaaatcgtcttgacccagtccccaggtactctgtctttaagtcccggggaaagagctactctgtcctgtagggcaagtcagtctgtttcttctaattatctggcctggtatcagcaaaaacccggtcaagctccaagactgttgatctatggtgcaagcagtagagccaccggaatccctgataggttttccggttcaggcagcggaactgacttcactctgacaatctcaagattggaacctgaggattttgccgtgtattactgtcagcagtacgaatcttctccatcttggacattcgggcaaggtaccaaggtcgaaattaagggtggtggtggttctggtggtggcggtagcggaggtggtggtagtcaggtccagttagttcaatcaggtgccgaggtcaaaaagccaggttcttccgtcaaagtgtcatgcaaggctagcggtggcaccttttctaattactacatctcttgggtgagacaggcaccaggtcaaggactggaatggatgggaggtatcatcccactgtttggtaccaccaattatgcccagaagttccaaggcagggtgaccataactgctgatgagagtacatctaccgcatacatggaattaagttctctgagatccgaggacaccgcagtgtattactgtgctagaggtacctggtacgctggtgatatctggggacaaggaactttggtcacagtgtcatct

SEQ ID NO: 25 - protein, 21 aa, synthetic construct
  source 1..21: note = CD8 Leader
MALPVTALLLPLALLLHAARP

SEQ ID NO: 26 - DNA, 63 bp, synthetic construct
  source 1..63: mol_type = other DNA; note = CD8 Leader (nucleic acid)
atggccctgcctgtgacagccctgctgctgcctctggctctgctgctgcatgccgctagaccc

SEQ ID NO: 27 - protein, 45 aa, synthetic construct
  source 1..45: note = CD8 Hinge
TTTPAPRPPTPAPTIASQPLSLRPEACRPAAGGAVHTRGLDFACD

SEQ ID NO: 28 - DNA, 135 bp, synthetic construct
  source 1..135: mol_type = other DNA; note = CD8 Hinge nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 29 - protein, 24 aa, synthetic construct
  source 1..24: note = CD8 TM
IYIWAPLAGTCGVLLLSLVITLYC

SEQ ID NO: 30 - DNA, 72 bp, synthetic construct
  source 1..72: mol_type = other DNA; note = CD8 TM nucleic acid sequence
atctacatctgggcgcccttggccgggacttgtggggtccttctcctgtcactggttatcaccctttactgc

SEQ ID NO: 31 - protein, 27 aa, synthetic construct
  source 1..27: note = CD28 transmembrane domain amino acid sequence
FWVLVVVGGVLACYSLLVTVAFIIFWV

SEQ ID NO: 32 - DNA, 81 bp, synthetic construct
  source 1..81: mol_type = other DNA; note = CD28 transmembrane domain nucleic acid sequence
ttttgggtgctggtggtggttggtggagtcctggcttgctatagcttgctagtaacagtggcctttattattttctgggtg

SEQ ID NO: 33 - protein, 24 aa, synthetic construct
  source 1..24: note = ICOS transmembrane domain amino acid sequence
FWLPIGCAAFVVVCILGCILICWL

SEQ ID NO: 34 - DNA, 72 bp, synthetic construct
  source 1..72: mol_type = other DNA; note = ICOS transmembrane domain nucleic acid sequence
ttctggttacccataggatgtgcagcctttgttgtagtctgcattttgggatgcatacttatttgttggctt

SEQ ID NO: 35 - protein, 229 aa, synthetic construct
  source 1..229: note = IgG4 Hinge amino acid sequence
ESKYGPPCPPCPAPEFLGGPSVFLFPPKPKDTLMISRTPEVTCWVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 36 - DNA, 690 bp, synthetic construct
  source 1..690: mol_type = other DNA; note = IgG4 Hinge nucleic acid sequence
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 37 - protein, 42 aa, synthetic construct
  source 1..42: note = 4-1BB domain
KRGRKKLLYIFKQPFMRPVQTTQEEDGCSCRFPEEEEGGCEL

SEQ ID NO: 38 - DNA, 126 bp, synthetic construct
  source 1..126: mol_type = other DNA; note = 4-1BB domain nucleic acid
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 39 - protein, 41 aa, synthetic construct
  source 1..41: note = CD28 intracellular domain amino acid sequence
RSKRSRLLHSDYMNMTPRRPGPTRKHYQPYAPPRDFAAYRS

SEQ ID NO: 40 - DNA, 123 bp, synthetic construct
  source 1..123: mol_type = other DNA; note = CD28 intracellular domain nucleic acid sequence
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 41 - protein, 41 aa, synthetic construct
  source 1..41: note = CD28 intracellular domain variant (YMFM) amino acid sequence
RSKRSRLLHSDYMFMTPRRPGPTRKHYQPYAPPRDFAAYRS

SEQ ID NO: 42 - DNA, 123 bp, synthetic construct
  source 1..123: mol_type = other DNA; note = CD28 intracellular domain variant (YMFM) nucleic acid sequence
aggagtaagaggagcaggctcctgcacagtgactacatgttcatgactccccgccgccccgggcccacccgcaagcattaccagccctatgccccaccacgcgacttcgcagcctatcgctcc

SEQ ID NO: 43 - protein, 35 aa, synthetic construct
  source 1..35: note = ICOS costimulatory domain amino acid sequence
TKKKYSSSVHDPNGEYMFMRAVNTAKKSRLTDVTL

SEQ ID NO: 44 - DNA, 105 bp, synthetic construct
  source 1..105: mol_type = other DNA; note = ICOS costimulatory domain nucleic acid sequence
acaaaaaagaagtattcatccagtgtgcacgaccctaacggtgaatacatgttcatgagagcagtgaacacagccaaaaaatctagactcacagatgtgacccta

SEQ ID NO: 45 - DNA, 105 bp, synthetic construct
  source 1..105: mol_type = other DNA; note = ICOS-1 costimulatory domain nucleic acid sequence
acaaaaaagaagtattcatccagtgtgcacgaccctaacggtgaatacatgttcatgagagcagtgaacacagccaaaaaatccagactcacagatgtgacccta

SEQ ID NO: 46 - protein, 117 aa, synthetic construct
  source 1..117: note = CD2 costimulatory domain amino acid sequence
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 47 - DNA, 351 bp, synthetic construct
  source 1..351: mol_type = other DNA; note = CD2 costimulatory domain nucleic acid sequence
accaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaat

SEQ ID NO: 48 - protein, 48 aa, synthetic construct
  source 1..48: note = CD27 intracellular domain amino acid sequence
QRRKYRSNKGESPVEPAEPCRYSCPREEEGSTIPIQEDYRKPEPACSP

SEQ ID NO: 49 - DNA, 144 bp, synthetic construct
  source 1..144: mol_type = other DNA; note = CD27 intracellular domain nucleic acid sequence
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 50 - protein, 42 aa, synthetic construct
  source 1..42: note = OX40 intracellular domain amino acid sequence
ALYLLRRDQRLPPDAHKPPGGGSFRTPIQEEQADAHSTLAKI

SEQ ID NO: 51 - DNA, 126 bp, synthetic construct
  source 1..126: mol_type = other DNA; note = OX40 intracellular domain nucleic acid sequence
gccctgtacctgctccgcagggaccagaggctgccccccgatgcccacaagccccctgggggaggcagtttcaggacccccatccaagaggagcaggccgacgcccactccaccctggccaagatc

SEQ ID NO: 52 - protein, 112 aa, synthetic construct
  source 1..112: note = CD3 (Q14K) zeta domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 336 bp, synthetic construct
  source 1..336: mol_type = other DNA; note = CD3 (Q14K) zeta domain nucleic acid
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 54 - protein, 112 aa, synthetic construct
  source 1..112: note = CD3 zeta domain amino acid
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - DNA, 336 bp, synthetic construct
  source 1..336: mol_type = other DNA; note = CD3 zeta domain nucleic acid
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 56 - protein, 11 aa, synthetic construct
  source 1..11: note = E4 CDR-L1
RASQSISSYLN

SEQ ID NO: 57 - protein, 7 aa, synthetic construct
  source 1..7: note = E4 CDR-L2
GASSLQS

SEQ ID NO: 58 - protein, 9 aa, synthetic construct
  source 1..9: note = E4 CDR-L3
QQSYRTPVT

SEQ ID NO: 59 - protein, 13 aa, synthetic construct
  source 1..13: note = E4 CDR-H1
KASGGTFSNYAIN

SEQ ID NO: 60 - protein, 15 aa, synthetic construct
  source 1..15: note = E4 CDR-H2
MGRIVPLLGIANYAQ

SEQ ID NO: 61 - protein, 18 aa, synthetic construct
  source 1..18: note = E4 CDR-H3
EHIAYRPTSAGYYYYMDI

SEQ ID NO: 62 - protein, 107 aa, synthetic construct
  source 1..107: note = CD19 E4 VL
DIQMTQSPSSLSASVGDRVTITCRASQSISSYLNWYQQKPGKAPKLLIYGASSLQSGVPSRFSGSGSGTDFTLTISSLQPEDFATYYCQQSYRTPVTFGQGTKVEIK

SEQ ID NO: 63 - protein, 127 aa, synthetic construct
  source 1..127: note = CD19 E4VH
QVQLVQSGAEVKKPGSSVKVSCKASGGTFSNYAINWVRQAPGQGLEWMGRIVPLLGIANYAQKFQGRVTITADESTSTAYMELSSLRSEDTAVYYCAREHIAYRPTSAGYYYYMDIWGQGTLVTVSS

SEQ ID NO: 64 - protein, 249 aa, Homo sapiens
  source 1..249: note = scFv-CD19 E4 human derived
DIQMTQSPSSLSASVGDRVTITCRASQSISSYLNWYQQKPGKAPKLLIYGASSLQSGVPSRFSGSGSGTDFTLTISSLQPEDFATYYCQQSYRTPVTFGQGTKVEIKGGGGSGGGGSGGGGSQVQLVQSGAEVKKPGSSVKVSCKASGGTFSNYAINWVRQAPGQGLEWMGRIVPLLGIANYAQKFQGRVTITADESTSTAYMELSSLRSEDTAVYYCAREHIAYRPTSAGYYYYMDIWGQGTLVTVSS

SEQ ID NO: 65 - DNA, 321 bp, synthetic construct
  source 1..321: mol_type = other DNA; note = Nucleic acid sequence CD19 E4 VL
gacatacaaatgacccagtctccttcttctctgagcgcatctgtcggcgatagagtgactattacatgtagggccagccagtctatctcttcttatctgaattggtatcagcaaaaacccggcaaggctccaaaactgttgatctatggtgcttcaagcctgcaatccggagttccctcaagattttctggttccggctcaggaactgatttcaccctgactataagttctttgcagcctgaagactttgcaacatattactgtcagcaatcttacagaaccccagttacattcgggcaaggtaccaaggtcgaaattaag

SEQ ID NO: 66 - DNA, 381 bp, synthetic construct
  source 1..381: mol_type = other DNA; note = Nucleic acid sequence CD19 E4VH
caggtccagttagttcaatcaggtgccgaggtcaaaaagccaggttcttccgtcaaagtgtcatgcaaggctagcggtggcaccttttctaattacgctatcaattgggtgagacaggcaccaggtcaaggactggaatggatgggaagaatcgttccactgctgggtatcgctaattatgcccagaagttccaaggcagggtgaccataactgctgatgagagtacatctaccgcatacatggaattaagttctctgagatccgaggacaccgcagtgtattactgtgctagagaacatatcgcttacagaccaacctctgctggttactactactacatggatatctggggacaaggaactttggtcacagtgtcatct

SEQ ID NO: 67 - protein, 11 aa, synthetic construct
  source 1..11: note = E7 CDR-L1
RASQDITRYLN

SEQ ID NO: 68 - protein, 7 aa, synthetic construct
  source 1..7: note = E7 CDR-L2
AASSLQS

SEQ ID NO: 69 - protein, 9 aa, synthetic construct
  source 1..9: note = E7 CDR-L3
QQSYSYPPT

SEQ ID NO: 70 - protein, 13 aa, synthetic construct
  source 1..13: note = E7 CDR-H1
AASGFTFRDYGMH

SEQ ID NO: 71 - protein, 17 aa, synthetic construct
  source 1..17: note = E7 CDR-H2
VAVISYEGSNEYYADSV

SEQ ID NO: 72 - protein, 14 aa, synthetic construct
  source 1..14: note = E7 CDR-H3
DRGFAGWYDYAFDP

SEQ ID NO: 73 - protein, 107 aa, synthetic construct
  source 1..107: note = CD19 E7 VL
DIQMTQSPSSLSASVGDRVTITCRASQDITRYLNWYQQKPGKAPKLLIYAASSLQSGVPSRFSGSGSGTDFTLTISSLQPEDFATYYCQQSYSYPPTFGQGTKVEIK

SEQ ID NO: 74 - protein, 123 aa, synthetic construct
  source 1..123: note = CD19 E7VH
QVQLVESGGGVVQPGRSLRLSCAASGFTFRDYGMHWVRQAPGKGLEWVAVISYEGSNEYYADSVKGRFTISRDNSKNTLYLQMNSLRAEDTAVYYCARDRGFAGWYDYAFDPWGQGTLVTVSS

SEQ ID NO: 75 - protein, 245 aa, Homo sapiens
  source 1..245: note = scFv-CD19 E7 human derived
DIQMTQSPSSLSASVGDRVTITCRASQDITRYLNWYQQKPGKAPKLLIYAASSLQSGVPSRFSGSGSGTDFTLTISSLQPEDFATYYCQQSYSYPPTFGQGTKVEIKGGGGSGGGGSGGGGSQVQLVESGGGVVQPGRSLRLSCAASGFTFRDYGMHWVRQAPGKGLEWVAVISYEGSNEYYADSVKGRFTISRDNSKNTLYLQMNSLRAEDTAVYYCARDRGFAGWYDYAFDPWGQGTLVTVSS

SEQ ID NO: 76 - DNA, 321 bp, synthetic construct
  source 1..321: mol_type = other DNA; note = Nucleic acid sequence CD19 E7 VL
gacatacaaatgacccagtctccttcttctctgagcgcatctgtcggcgatagagtgactattacatgtagggccagccaggatatcaccagatatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaatcttactcttacccaccaacattcgggcaaggtaccaaggtcgaaattaag

SEQ ID NO: 77 - DNA, 369 bp, synthetic construct
  source 1..369: mol_type = other DNA; note = Nucleic acid sequence CD19 E7VH
caggttcagttagtcgagtctggtggcggtgtcgtccagcctggtagatccttaaggctgtcatgtgccgctagcggatttacctttagagattacggtatgcattgggtgagacaagcccccggtaaaggcctggaatgggtcgctgtgataagttacgaaggttctaacgaatactacgcagactccgttaagggtagattcactatttccagggataattcaaagaacactttgtatctgcagatgaactcattgagagctgaggacaccgcagtgtattactgtgctagagatagaggttttgctggttggtacgattacgcttttgatccatggggacaaggaactttggtcacagtgtcatct

SEQ ID NO: 78 - protein, 11 aa, synthetic construct
  source 1..11: note = 7 CDR-L1
RASQSISKYLN

SEQ ID NO: 79 - protein, 7 aa, synthetic construct
  source 1..7: note = 7 CDR-L2
DASSLQS

SEQ ID NO: 80 - protein, 9 aa, synthetic construct
  source 1..9: note = 7 CDR-L3
QQSYTIPLT

SEQ ID NO: 81 - protein, 13 aa, synthetic construct
  source 1..13: note = 7 CDR-H1
KASGGTFSSYAFS

SEQ ID NO: 82 - protein, 15 aa, synthetic construct
  source 1..15: note = 7 CDR-H2
MGGIVPLFGAVEYAQ

SEQ ID NO: 83 - protein, 10 aa, synthetic construct
  source 1..10: note = 7 CDR-H3
EKGFYRYFDH

SEQ ID NO: 84 - protein, 107 aa, synthetic construct
  source 1..107: note = CD19-7 VL
DIQMTQSPSSLSASVGDRVTITCRASQSISKYLNWYQQKPGKAPKLLIYDASSLQSGVPSRFSGSGSGTDFTLTISSLQPEDFATYYCQQSYTIPLTFGQGTKVEIK

SEQ ID NO: 85 - protein, 119 aa, synthetic construct
  source 1..119: note = CD19-7VH
QVQLVQSGAEVKKPGSSVKVSCKASGGTFSSYAFSWVRQAPGQGLEWMGGIVPLFGAVEYAQKFQGRVTITADESTSTAYMELSSLRSEDTAVYYCAREKGFYRYFDHWGQGTLVTVSS

SEQ ID NO: 86 - protein, 241 aa, Homo sapiens
  source 1..241: note = scFv-CD19-7 human derived
DIQMTQSPSSLSASVGDRVTITCRASQSISKYLNWYQQKPGKAPKLLIYDASSLQSGVPSRFSGSGSGTDFTLTISSLQPEDFATYYCQQSYTIPLTFGQGTKVEIKGGGGSGGGGSGGGGSQVQLVQSGAEVKKPGSSVKVSCKASGGTFSSYAFSWVRQAPGQGLEWMGGIVPLFGAVEYAQKFQGRVTITADESTSTAYMELSSLRSEDTAVYYCAREKGFYRYFDHWGQGTLVTVSS

SEQ ID NO: 87 - DNA, 321 bp, synthetic construct
  source 1..321: mol_type = other DNA; note = Nucleic acid sequence CD19-7 VL
gacatacaaatgacccagtctccttcttctctgagcgcatctgtcggcgatagagtgactattacatgtagggccagccagtctatctctaaatatctgaattggtatcagcaaaaacccggcaaggctccaaaactgttgatctatgatgcttcaagcctgcaatccggagttccctcaagattttctggttccggctcaggaactgatttcaccctgactataagttctttgcagcctgaagactttgcaacatattactgtcagcaatcttacaccatcccactgacattcgggcaaggtaccaaggtcgaaattaag

SEQ ID NO: 88 - DNA, 357 bp, synthetic construct
  source 1..357: mol_type = other DNA; note = Nucleic acid sequence CD19-7VH
caggtccagttagttcaatcaggtgccgaggtcaaaaagccaggttcttccgtcaaagtgtcatgcaaggctagcggtggcaccttttcttcttacgctttttcttgggtgagacaggcaccaggtcaaggactggaatggatgggaggtatcgttccactgtttggtgctgttgaatatgcccagaagttccaaggcagggtgaccataactgctgatgagagtacatctaccgcatacatggaattaagttctctgagatccgaggacaccgcagtgtattactgtgctagagaaaaaggtttttacagatactttgatcattggggacaaggaactttggtcacagtgtcatct

SEQ ID NO: 89 - protein, 11 aa, synthetic construct
  source 1..11: note = 10 CDR-L1
RASQTISRYLN

SEQ ID NO: 90 - protein, 7 aa, synthetic construct
  source 1..7: note = 10 CDR-L2
AASSLQS

SEQ ID NO: 91 - protein, 9 aa, synthetic construct
  source 1..9: note = 10 CDR-L3
QQSYRPPLT

SEQ ID NO: 92 - protein, 18 aa, synthetic construct
  source 1..18: note = T2A amino acid sequence
EGRGSLLTCGDVEENPGP

SEQ ID NO: 93 - DNA, 54 bp, synthetic construct
  source 1..54: mol_type = other DNA; note = T2A nucleic acid sequence
gagggcagaggaagtcttctaacatgcggtgacgtggaggagaatcccggccct

SEQ ID NO: 94 - protein, 7 aa, synthetic construct
  source 1..7: note = T2A spacer amino acid sequence
SGRSGGG

SEQ ID NO: 95 - DNA, 21 bp, synthetic construct
  source 1..21: mol_type = other DNA; note = T2A spacer nucleic acid sequence
tccggaagatctggcggcgga

SEQ ID NO: 96 - protein, 22 aa, synthetic construct
  source 1..22: note = F2A amino acid sequence
VKQTLNFDLLKLAGDVESNPGP

SEQ ID NO: 97 - DNA, 66 bp, synthetic construct
  source 1..66: mol_type = other DNA; note = F2A nucleic acid sequence
gtgaaacagactttgaattttgaccttctcaagttggcgggagacgtggagtccaacccagggccg

SEQ ID NO: 98 - DNA, 96 bp, synthetic construct
  source 1..96: mol_type = other DNA; note = Furin-(G4S)2-T2A (F-GS2-T2A) linker nucleic acid sequence
cgtgcgaagaggggcggcgggggctccggcgggggaggcagtgagggccgcggctccctgctgacctgcggagatgtagaagagaacccaggcccc

SEQ ID NO: 99 - protein, 32 aa, synthetic construct
  source 1..32: note = Furin-(G4S)2-T2A (F-GS2-T2A) linker amino acid sequence
RAKRGGGGSGGGGSEGRGSLLTCGDVEENPGP

SEQ ID NO: 100 - protein, 194 aa, Woodchuck hepatitis virus
  source 1..194: note = WPRE
INLWITKFVKDLVFLTMLLLLRYVDTLLCLCIMLLLPVWLSFSPPCINPGCCLFMRSCGPLSGNVAWCALCLLTQPPLVGALPPPVSSFPGLSLSPSLLPRRNSSPPALPAAGQGLGCWALTIPWCCRGSRPFLGCSPVLPPGFCAGRPSATSLRPSIQRTFLPAACCRLCGLFRVFAFALRRVGSPFGPPPRL

SEQ ID NO: 101 - DNA, 1184 bp, synthetic construct
  source 1..1184: mol_type = other DNA; note = EF-1 alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 102 - DNA, 747 bp, synthetic construct
  source 1..747: mol_type = other DNA; note = Nucleic acid sequence scFv-CD19 E4 P3
gacatacaaatgacccagtctccttcttctctgagcgcatctgtcggcgatagagtgactattacatgtagggccagccagtctatctcttcttatctgaattggtatcagcaaaaacccggcaaggctccaaaactgttgatctatggtgcttcaagcctgcaatccggagttccctcaagattttctggttccggctcaggaactgatttcaccctgactataagttctttgcagcctgaagactttgcaacatattactgtcagcaatcttacagaaccccagttacattcgggcaaggtaccaaggtcgaaattaagggtggtggtggttctggtggtggcggtagcggaggtggtggtagtcaggtccagttagttcaatcaggtgccgaggtcaaaaagccaggttcttccgtcaaagtgtcatgcaaggctagcggtggcaccttttctaattacgctatcaattgggtgagacaggcaccaggtcaaggactggaatggatgggaagaatcgttccactgctgggtatcgctaattatgcccagaagttccaaggcagggtgaccataactgctgatgagagtacatctaccgcatacatggaattaagttctctgagatccgaggacaccgcagtgtattactgtgctagagaacatatcgcttacagaccaacctctgctggttactactactacatggatatctggggacaaggaactttggtcacagtgtcatct

SEQ ID NO: 103 - DNA, 735 bp, synthetic construct
  source 1..735: mol_type = other DNA; note = Nucleic acid sequence scFv-CD19 E7
gacatacaaatgacccagtctccttcttctctgagcgcatctgtcggcgatagagtgactattacatgtagggccagccaggatatcaccagatatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaatcttactcttacccaccaacattcgggcaaggtaccaaggtcgaaattaagggtggtggtggttctggtggtggcggtagcggaggtggtggtagtcaggttcagttagtcgagtctggtggcggtgtcgtccagcctggtagatccttaaggctgtcatgtgccgctagcggatttacctttagagattacggtatgcattgggtgagacaagcccccggtaaaggcctggaatgggtcgctgtgataagttacgaaggttctaacgaatactacgcagactccgttaagggtagattcactatttccagggataattcaaagaacactttgtatctgcagatgaactcattgagagctgaggacaccgcagtgtattactgtgctagagatagaggttttgctggttggtacgattacgcttttgatccatggggacaaggaactttggtcacagtgtcatct

SEQ ID NO: 104 - DNA, 723 bp, synthetic construct
  source 1..723: mol_type = other DNA; note = Nucleic acid sequence scFv-CD19-7
gacatacaaatgacccagtctccttcttctctgagcgcatctgtcggcgatagagtgactattacatgtagggccagccagtctatctctaaatatctgaattggtatcagcaaaaacccggcaaggctccaaaactgttgatctatgatgcttcaagcctgcaatccggagttccctcaagattttctggttccggctcaggaactgatttcaccctgactataagttctttgcagcctgaagactttgcaacatattactgtcagcaatcttacaccatcccactgacattcgggcaaggtaccaaggtcgaaattaagggtggtggtggttctggtggtggcggtagcggaggtggtggtagtcaggtccagttagttcaatcaggtgccgaggtcaaaaagccaggttcttccgtcaaagtgtcatgcaaggctagcggtggcaccttttcttcttacgctttttcttgggtgagacaggcaccaggtcaaggactggaatggatgggaggtatcgttccactgtttggtgctgttgaatatgcccagaagttccaaggcagggtgaccataactgctgatgagagtacatctaccgcatacatggaattaagttctctgagatccgaggacaccgcagtgtattactgtgctagagaaaaaggtttttacagatactttgatcattggggacaaggaactttggtcacagtgtcatct

SEQ ID NO: 105 - protein, 157 aa, synthetic construct
  source 1..157: note = hIL18 (e.g., GenBank Acc. No. AAK95950.1) leaderless
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 106 - protein, 178 aa, synthetic construct
  source 1..178: note = CD8leader +hIL18
MALPVTALLLPLALLLHAARPYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 107 - protein, 193 aa, Homo sapiens
  source 1..193: note = Human IL-18 (GenBank Acc. No. AAK95950.1)
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 108 - protein, 20 aa, Homo sapiens
  source 1..20: note = Human IL-2 signal sequence
MYRMQLLSCIALSLALVTNS

SEQ ID NO: 109 - protein, 20 aa, Murine leukemia virus
  source 1..20: note = Murine IL-2 signal sequence
MYSMOLASCVTLTLVLLVNS

SEQ ID NO: 110 - protein, 20 aa, Homo sapiens
  source 1..20: note = human kappa leader sequence
METPAQLLFLLLLWLPDTTG

SEQ ID NO: 111 - protein, 20 aa, Murine leukemia virus
  source 1..20: note = Murine kappa leader sequence
METDTLLLWVLLLWVPGSTG

SEQ ID NO: 112 - protein, 16 aa, Homo sapiens
  source 1..16: note = Human albumin signal sequence
MKWVTFISLLFSSAYS

SEQ ID NO: 113 - protein, 30 aa, Homo sapiens
  source 1..30: note = Human prolactin signal sequence
MDSKGSSQKGSRLLLLLVVSNLLLCQGVVS

SEQ ID NO: 114 - DNA, 717 bp, synthetic construct
  source 1..717: mol_type = other DNA; note = Nucleic acid sequence scFv-CD19 -14
gacatacaaatgacccagtctccttcttctctgagcgcatctgtcggcgatagagtgactattacatgtagggccagccagtctatctctaattatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaagcttactctgctccaatcacattcgggcaaggtaccaaggtcgaaattaagggtggtggtggttctggtggtggcggtagcggaggtggtggtagtgaggttcagttgttagagagcggggggggtctggttcagcctggtggcagcttaagactgagttgtgccgcttctggttttactttcggtgattatgcaatgtcctgggtgaggcaagcccccggtaaaggcctggaatgggtttcagctatctctagaggtggtcatggtacctactatgccgatagcgtgaaaggtagattcactatttccagggacaattcaaagaacactttgtacttacagatgaactctttgagagctgaggacaccgcagtgtattactgtgctagactggttggttacggtctggattactggggacaaggaactttggtcacagtgtcatct

SEQ ID NO: 115 - DNA, 708 bp, synthetic construct
  source 1..708: mol_type = other DNA; note = Nucleic acid sequence scFv-CD19 -15
caaatgacccagtctccttcttctctgagcgcatctgtcggcgatagagtgactattacatgtagggccagccagcctatcagaccttatctgaattggtatcagcaaaaacccggcaaggctccaaaactgttgatctatgatgcttcaagcctgcaatccggagttccctcaagattttctggttccggctcaggaactgatttcaccctgactataagttctttgcagcctgaagactttgcaacatattactgtcagcaatcttactctgctccatacacattcgggcaaggtaccaaggtcgaaattaagggtggtggtggttctggtggtggcggtagcggaggtggtggtagtgaggttcagttgttagagagcggggggggtctggttcagcctggtggcagcttaagactgagttgtgccgcttctggttttactttctcttcttatgcaatgtcctgggtgaggcaagcccccggtaaaggcctggaatgggtttcagttatctctggtggtggtgctaatacctactacgccgatagcgtgaaaggtagattcactatttccagggacaattcaaagaacactttgtacttacagatgaactctttgagagctgaggacaccgcagtgtattactgtgctagagattggagatactttgatcattggggacaaggaactttggtcacagtgtcatct

SEQ ID NO: 116 - DNA, 738 bp, synthetic construct
  source 1..738: mol_type = other DNA; note = Nucleic acid sequence scFv-CD19 -18
cagtccgtgttgacccagcctccatcagtctcaggagccccaggccagagagtgaccatttcttgtactggatcttcctcaaatatcggggccggttacgatgttcattggtatcagcaactgcccggtacagctccaaaactgttgatctatggtaccaaaaacagacctagcggtgtgcccgacaggttctccggctcaaagagcggaacaagtgcttctttagcaattaccggcctgcaggctgaagatgaggcagactattactgtcaatcctacgatgttagactgaaaggttgggtttttggtggcggtaccaaattgactgtcttaggtggtggtggttctggtggtggcggtagcggaggtggtggtagtcagttacagttacaggagagcggtccaggtttagtgaaaccatccgaaactttatcactgacctgtacagtgtctggtggctccatcacctcttcttcttattactggggttggataagacagccacctggaaaagggctggagtggattggatctatctactacaccggtaccacctactacaatccttcattgaagagcagggtcacaataagcgtggataccagtaaaaaccagttttccttgaagctgagttctgtcacagccgctgacaccgcagtgtattactgtgctagatacgttggtctgtctggtggttttgattactggggacaaggaactttggtcacagtgtcatct

SEQ ID NO: 117 - DNA, 717 bp, synthetic construct
  source 1..717: mol_type = other DNA; note = Nucleic acid sequence scFv-CD19 -23
gacatacaaatgacccagtctccttcttctctgagcgcatctgtcggcgatagagtgactattacatgtagggccagccagtctatctactcttatctgaattggtatcagcaaaaacgcggcaaggctccaaaactgttgatctatgatgcttcaagcctgcaatccggagttccctcaagattttctggttccggctcaggaactgatttcaccctgactataagttctttgcagcctgaagactttgcaacatattactgtcagcaatcttacaccgctccaccaacattcgggcaaggtaccaaggtcgaaattaagggtggtggtggttctggtggtggcggtagcggaggtggtggtagtgaggttcagttgttagagagcggggggggtctggttcagcctggtggcagcttaagactgagttgtgccgcttctggttttactttctctaattatgcaatgtcctgggtgaggcaagcccccggtaaaggcctggaatgggtttcagctatctctgaatctggtcatggtacctactacgccgatagcgtgaaaggtagattcactatttccagggacaattcaaagaacactttgtacttacagatgaactctttgagagctgaggacaccgcagtgtattactgtgctagactggattgggctggttttgatgtttggggacaaggaactttggtcacagtgtcatct

SEQ ID NO: 118 - DNA, 714 bp, synthetic construct
  source 1..714: mol_type = other DNA; note = Nucleic acid sequence scFv-CD19 -#10
gacatacaaatgacccagtctccttcttctctgagcgcatctgtcggcgatagagtgactattacatgtagggccagccagaccatctctagatatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaatcttacagaccaccactgacattcgggcaaggtaccaaggtcgaaattaagggtggtggtggttctggtggtggcggtagcggaggtggtggtagtgaggttcagttgttagagagcggggggggtctggttcagcctggtggcagcttaagactgagttgtgccgcttctggttttactttctcttcttatgcaatgtcctgggtgaggcaagcccccggtaaaggcctggaatgggtttcaaccatctctgctggtggtcatggtacctactacgccgatagcgtgaaaggtagattcactatttccagggacaattcaaagaacactttgtacttacagatgaactctttgagagctgaggacaccgcagtgtattactgtgctagaggtgctggttactttgattactggggacaaggaactttggtcacagtgtcatct

SEQ ID NO: 119 - DNA, 744 bp, synthetic construct
  source 1..744: mol_type = other DNA; note = Nucleic acid sequence scFv-CD19 -#11
gacatacaaatgacccagtctccttcttctctgagcgcatctgtcggcgatagagtgactattacatgtagggccagccagtctatctcttcttatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaaaccggtgctgttccatacacattcgggcaaggtaccaaggtcgaaattaagggtggtggtggttctggtggtggcggtagcggaggtggtggtagtgaggttcagttgttagagagcggggggggtctggttcagcctggtggcagcttaagactgagttgtgccgcttctggttttactttcagagattatgcaatgtcctgggtgaggcaagcccccggtaaaggcctggaatgggtttcagctatctctgaatctggtatcgatacctactacgccgatagcgtgaaaggtagattcactatttccagggacaattcaaagaacactttgtacttacagatgaactctttgagagctgaggacaccgcagtgtattactgtgctagagttgctggttacgattctgattcttctacctactacgattacatggatgtttggggacaaggaactttggtcacagtgtcatct

SEQ ID NO: 120 - DNA, 738 bp, synthetic construct
  source 1..738: mol_type = other DNA; note = Nucleic acid sequence scFv-CD19 -16
cagtccgtgttgacccagcctccatcagtctcaggagccccaggccagagagtgaccatttcttgtactggatcttcctcaaatatcggggccggttacgatgttcattggtatcagcaactgcccggtacagctccaaaactgttgatctatggtaataataacagacctagcggtgtgcccgacaggttctccggctcaaagagcggaacaagtgcttctttagcaattaccggcctgcaggctgaagatgaggcagactattactgtcaatcctacgatgtttctctgggtgtttgggtttttggtggcggtaccaaattgactgtcttaggtggtggtggttctggtggtggcggtagcggaggtggtggtagtcagttacagttacaggagagcggtccaggtttagtgaaaccatccgaaactttatcactgacctgtacagtgtctggtggctccatctcttctccatcttattactggggttggataagacagccacctggaaaagggctggagtggattggatctatctactacaccggtgctacctactacaatccttcattgaagagcagggtcacaataagcgtggataccagtaaaaaccagttttccttgaagctgagttctgtcacagccgctgacaccgcagtgtattactgtgctagatacggtccagctggtgttggttttgattactggggacaaggaactttggtcacagtgtcatct

SEQ ID NO: 121 - protein, 4 aa, synthetic construct
  source 1..4: note = linker
GGSG

SEQ ID NO: 122 - protein, 5 aa, synthetic construct
  source 1..5: note = linker
GGSGG

SEQ ID NO: 123 - protein, 5 aa, synthetic construct
  source 1..5: note = linker
GSGSG

SEQ ID NO: 124 - protein, 5 aa, synthetic construct
  source 1..5: note = linker
GSGGG

SEQ ID NO: 125 - protein, 5 aa, synthetic construct
  source 1..5: note = linker
GGGSG

SEQ ID NO: 126 - protein, 5 aa, synthetic construct
  source 1..5: note = linker
GSSSG

SEQ ID NO: 127 - protein, 5 aa, synthetic construct
  source 1..5: note = linker
GGGGS

SEQ ID NO: 128 - protein, 15 aa, synthetic construct
  source 1..15: note = linker
GGGGSGGGGSGGGGS

SEQ ID NO: 129 - protein, 45 aa, synthetic construct
  source 1..45: note = linker
GGTGGCGGTGGCTCGGGCGGTGGTGGGTCGGGTGGCGGCGGATCT

SEQ ID NO: 130 - protein, 5 aa, synthetic construct
  source 1..5: note = hinge
DKTHT

SEQ ID NO: 131 - protein, 4 aa, synthetic construct
  source 1..4: note = hinge
CPPC

SEQ ID NO: 132 - protein, 15 aa, synthetic construct
  source 1..15: note = hinge
CPEPKSCDTPPPCPR

SEQ ID NO: 133 - protein, 12 aa, synthetic construct
  source 1..12: note = hinge
ELKTPLGDTTHT

SEQ ID NO: 134 - protein, 10 aa, synthetic construct
  source 1..10: note = hinge
KSCDKTHTCP

SEQ ID NO: 135 - protein, 7 aa, synthetic construct
  source 1..7: note = hinge
KCCVDCP

SEQ ID NO: 136 - protein, 7 aa, synthetic construct
  source 1..7: note = hinge
KYGPPCP

SEQ ID NO: 137 - protein, 15 aa, Homo sapiens
  source 1..15: note = human IgG1 hinge
EPKSCDKTHTCPPCP

SEQ ID NO: 138 - protein, 12 aa, Homo sapiens
  source 1..12: note = human IgG2 hinge
ERKCCVECPPCP

SEQ ID NO: 139 - protein, 17 aa, Homo sapiens
  source 1..17: note = human IgG3 hinge
ELKTPLGDTTHTCPRCP

SEQ ID NO: 140 - protein, 12 aa, Homo sapiens
  source 1..12: note = human IgG4 hinge
SPNMVPHAHHAQ

SEQ ID NO: 141 - protein, 13 aa, synthetic construct
  source 1..13: note = 10 CDR-H1
AASGFTFSSYAMS

SEQ ID NO: 142 - protein, 17 aa, synthetic construct
  source 1..17: note = 10 CDR-H2
VSTISAGGHGTYYADSV

SEQ ID NO: 143 - protein, 7 aa, synthetic construct
  source 1..7: note = 10 CDR-H3
GAGYFDY

SEQ ID NO: 144 - protein, 107 aa, synthetic construct
  source 1..107: note = CD19 10 VL
DIQMTQSPSSLSASVGDRVTITCRASQTISRYLNWYQQKPGKAPKLLIYAASSLQSGVPSRFSGSGSGTDFTLTISSLQPEDFATYYCQQSYRPPLTFGQGTKVEIK

SEQ ID NO: 145 - protein, 116 aa, synthetic construct
  source 1..116: note = CD19 10VH
EVQLLESGGGLVQPGGSLRLSCAASGFTFSSYAMSWVRQAPGKGLEWVSTISAGGHGTYYADSVKGRFTISRDNSKNTLYLQMNSLRAEDTAVYYCARGAGYFDYWGQGTLVTVSS

SEQ ID NO: 146 - protein, 238 aa, Homo sapiens
  source 1..238: note = scFv-CD19 10 human derived
DIQMTQSPSSLSASVGDRVTITCRASQTISRYLNWYQQKPGKAPKLLIYAASSLQSGVPSRFSGSGSGTDFTLTISSLQPEDFATYYCQQSYRPPLTFGQGTKVEIKGGGGSGGGGSGGGGSEVQLLESGGGLVQPGGSLRLSCAASGFTFSSYAMSWVRQAPGKGLEWVSTISAGGHGTYYADSVKGRFTISRDNSKNTLYLQMNSLRAEDTAVYYCARGAGYFDYWGQGTLVTVSS

SEQ ID NO: 147 - protein, 321 aa, synthetic construct
  source 1..321: note = Nucleic acid sequence CD19 10 VL
GACATACAAATGACCCAGTCTCCTTCTTCTCTGAGCGCATCTGTCGGCGATAGAGTGACTATTACATGTAGGGCCAGCCAGACCATCTCTAGATATCTGAATTGGTATCAGCAAAAACCCGGCAAGGCTCCAAAACTGTTGATCTATGCTGCTTCAAGCCTGCAATCCGGAGTTCCCTCAAGATTTTCTGGTTCCGGCTCAGGAACTGATTTCACCCTGACTATAAGTTCTTTGCAGCCTGAAGACTTTGCAACATATTACTGTCAGCAATCTTACAGACCACCACTGACATTCGGGCAAGGTACCAAGGTCGAAATTAAG

SEQ ID NO: 148 - protein, 348 aa, synthetic construct
  source 1..348: note = Nucleic acid sequence CD19 10VH
GAGGTTCAGTTGTTAGAGAGCGGGGGGGGTCTGGTTCAGCCTGGTGGCAGCTTAAGACTGAGTTGTGCCGCTTCTGGTTTTACTTTCTCTTCTTATGCAATGTCCTGGGTGAGGCAAGCCCCCGGTAAAGGCCTGGAATGGGTTTCAACCATCTCTGCTGGTGGTCATGGTACCTACTACGCCGATAGCGTGAAAGGTAGATTCACTATTTCCAGGGACAATTCAAAGAACACTTTGTACTTACAGATGAACTCTTTGAGAGCTGAGGACACCGCAGTGTATTACTGTGCTAGAGGTGCTGGTTACTTTGATTACTGGGGACAAGGAACTTTGGTCACAGTGTCATCT

SEQ ID NO: 149 - protein, 11 aa, synthetic construct
  source 1..11: note = 11 CDR-L1
RASQSISSYLN

SEQ ID NO: 150 - protein, 7 aa, synthetic construct
  source 1..7: note = 11 CDR-L2
AASSLQS

SEQ ID NO: 151 - protein, 10 aa, synthetic construct
  source 1..10: note = 11 CDR-L3
QQTGAVPYTF

SEQ ID NO: 152 - protein, 13 aa, synthetic construct
  source 1..13: note = 11 CDR-H1
AASGFTFRDYAMS

SEQ ID NO: 153 - protein, 17 aa, synthetic construct
  source 1..17: note = 11 CDR-H2
VSAISESGIDTYYADSV

SEQ ID NO: 154 - protein, 17 aa, synthetic construct
  source 1..17: note = 11 CDR-H3
VAGYDSDSSTYYDYMDV

SEQ ID NO: 155 - protein, 107 aa, synthetic construct
  source 1..107: note = CD19 11 VL
DIQMTQSPSSLSASVGDRVTITCRASQSISSYLNWYQQKPGKAPKLLIYAASSLQSGVPSRFSGSGSGTDFTLTISSLQPEDFATYYCQQTGAVPYTFGQGTKVEIK

SEQ ID NO: 156 - protein, 126 aa, synthetic construct
  source 1..126: note = CD19 11VH
EVQLLESGGGLVQPGGSLRLSCAASGFTFRDYAMSWVRQAPGKGLEWVSAISESGIDTYYADSVKGRFTISRDNSKNTLYLQMNSLRAEDTAVYYCARVAGYDSDSSTYYDYMDVWGQGTLVTVSS

SEQ ID NO: 157 - protein, 248 aa, Homo sapiens
  source 1..248: note = scFv-CD19 11 human derived
DIQMTQSPSSLSASVGDRVTITCRASQSISSYLNWYQQKPGKAPKLLIYAASSLQSGVPSRFSGSGSGTDFTLTISSLQPEDFATYYCQQTGAVPYTFGQGTKVEIKGGGGSGGGGSGGGGSEVQLLESGGGLVQPGGSLRLSCAASGFTFRDYAMSWVRQAPGKGLEWVSAISESGIDTYYADSVKGRFTISRDNSKNTLYLQMNSLRAEDTAVYYCARVAGYDSDSSTYYDYMDVWGQGTLVTVSS

SEQ ID NO: 158 - DNA, 321 bp, synthetic construct
  source 1..321: mol_type = other DNA; note = Nucleic acid sequence CD19 11 VL
gacatacaaatgacccagtctccttcttctctgagcgcatctgtcggcgatagagtgactattacatgtagggccagccagtctatctcttcttatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaaaccggtgctgttccatacacattcgggcaaggtaccaaggtcgaaattaag

SEQ ID NO: 159 - DNA, 378 bp, synthetic construct
  source 1..378: mol_type = other DNA; note = Nucleic acid sequence CD19 11VH
gaggttcagttgttagagagcggggggggtctggttcagcctggtggcagcttaagactgagttgtgccgcttctggttttactttcagagattatgcaatgtcctgggtgaggcaagcccccggtaaaggcctggaatgggtttcagctatctctgaatctggtatcgatacctactacgccgatagcgtgaaaggtagattcactatttccagggacaattcaaagaacactttgtacttacagatgaactctttgagagctgaggacaccgcagtgtattactgtgctagagttgctggttacgattctgattcttctacctactacgattacatggatgtttggggacaaggaactttggtcacagtgtcatct

SEQ ID NO: 160 - protein, 11 aa, synthetic construct
  source 1..11: note = 14 CDR-L1
RASQSISNYLN

SEQ ID NO: 161 - protein, 7 aa, synthetic construct
  source 1..7: note = 14 CDR-L2
AASSLQS

SEQ ID NO: 162 - protein, 9 aa, synthetic construct
  source 1..9: note = 14 CDR-L3
QQAYSAPIT

SEQ ID NO: 163 - protein, 13 aa, synthetic construct
  source 1..13: note = 14 CDR-H1
AASGFTFGDYAMS

SEQ ID NO: 164 - protein, 17 aa, synthetic construct
  source 1..17: note = 14 CDR-H2
VSAISRGGHGTYYADSV

SEQ ID NO: 165 - protein, 8 aa, synthetic construct
  source 1..8: note = 14 CDR-H3
LVGYGLDY

SEQ ID NO: 166 - protein, 107 aa, synthetic construct
  source 1..107: note = CD19 14 VL
DIQMTQSPSSLSASVGDRVTITCRASQSISNYLNWYQQKPGKAPKLLIYAASSLQSGVPSRFSGSGSGTDFTLTISSLQPEDFATYYCQQAYSAPITFGQGTKVEIK

SEQ ID NO: 167 - protein, 117 aa, synthetic construct
  source 1..117: note = CD19 14VH
EVQLLESGGGLVQPGGSLRLSCAASGFTFGDYAMSWVRQAPGKGLEWVSAISRGGHGTYYADSVKGRFTISRDNSKNTLYLQMNSLRAEDTAVYYCARLVGYGLDYWGQGTLVTVSS

SEQ ID NO: 168 - protein, 239 aa, Homo sapiens
  source 1..239: note = scFv-CD19 14 human derived
DIQMTQSPSSLSASVGDRVTITCRASQSISNYLNWYQQKPGKAPKLLIYAASSLQSGVPSRFSGSGSGTDFTLTISSLQPEDFATYYCQQAYSAPITFGQGTKVEIKGGGGSGGGGSGGGGSEVQLLESGGGLVQPGGSLRLSCAASGFTFGDYAMSWVRQAPGKGLEWVSAISRGGHGTYYADSVKGRFTISRDNSKNTLYLQMNSLRAEDTAVYYCARLVGYGLDYWGQGTLVTVSS

SEQ ID NO: 169 - DNA, 321 bp, synthetic construct
  source 1..321: mol_type = other DNA; note = Nucleic acid sequence CD19 14 VL
gacatacaaatgacccagtctccttcttctctgagcgcatctgtcggcgatagagtgactattacatgtagggccagccagtctatctctaattatctgaattggtatcagcaaaaacccggcaaggctccaaaactgttgatctatgctgcttcaagcctgcaatccggagttccctcaagattttctggttccggctcaggaactgatttcaccctgactataagttctttgcagcctgaagactttgcaacatattactgtcagcaagcttactctgctccaatcacattcgggcaaggtaccaaggtcgaaattaag

SEQ ID NO: 170 - DNA, 351 bp, synthetic construct
  source 1..351: mol_type = other DNA; note = Nucleic acid sequence CD19 14VH
gaggttcagttgttagagagcggggggggtctggttcagcctggtggcagcttaagactgagttgtgccgcttctggttttactttcggtgattatgcaatgtcctgggtgaggcaagcccccggtaaaggcctggaatgggtttcagctatctctagaggtggtcatggtacctactatgccgatagcgtgaaaggtagattcactatttccagggacaattcaaagaacactttgtacttacagatgaactctttgagagctgaggacaccgcagtgtattactgtgctagactggttggttacggtctggattactggggacaaggaactttggtcacagtgtcatct

SEQ ID NO: 171 - protein, 11 aa, synthetic construct
  source 1..11: note = 15 CDR-L1
RASQPIRPYLN

SEQ ID NO: 172 - protein, 7 aa, synthetic construct
  source 1..7: note = 15 CDR-L2
DASSLQS

SEQ ID NO: 173 - protein, 9 aa, synthetic construct
  source 1..9: note = 15 CDR-L3
QQSYSAPYT

SEQ ID NO: 174 - protein, 13 aa, synthetic construct
  source 1..13: note = 15 CDR-H1
AASGFTFSSYAMS

SEQ ID NO: 175 - protein, 18 aa, synthetic construct
  source 1..18: note = 15 CDR-H2
VSVISGGGANTYYADSVK

SEQ ID NO: 176 - protein, 7 aa, synthetic construct
  source 1..7: note = 15 CDR-H3
DWRYFDH

SEQ ID NO: 177 - protein, 107 aa, synthetic construct
  source 1..107: note = CD19 15 VL
DIQMTQSPSSLSASVGDRVTITCRASQPIRPYLNWYQQKPGKAPKLLIYDASSLQSGVPSRFSGSGSGTDFTLTISSLQPEDFATYYCQQSYSAPYTFGQGTKVEIK

SEQ ID NO: 178 - protein, 116 aa, synthetic construct
  source 1..116: note = CD19 15VH
EVQLLESGGGLVQPGGSLRLSCAASGFTFSSYAMSWVRQAPGKGLEWVSVISGGGANTYYADSVKGRFTISRDNSKNTLYLQMNSLRAEDTAVYYCARDWRYFDHWGQGTLVTVSS

SEQ ID NO: 179 - protein, 238 aa, Homo sapiens
  source 1..238: note = scFv-CD19 15 human derived
DIQMTQSPSSLSASVGDRVTITCRASQPIRPYLNWYQQKPGKAPKLLIYDASSLQSGVPSRFSGSGSGTDFTLTISSLQPEDFATYYCQQSYSAPYTFGQGTKVEIKGGGGSGGGGSGGGGSEVQLLESGGGLVQPGGSLRLSCAASGFTFSSYAMSWVRQAPGKGLEWVSVISGGGANTYYADSVKGRFTISRDNSKNTLYLQMNSLRAEDTAVYYCARDWRYFDHWGQGTLVTVSS

SEQ ID NO: 180 - DNA, 315 bp, synthetic construct
  source 1..315: mol_type = other DNA; note = Nucleic acid sequence CD19 15 VL
caaatgacccagtctccttcttctctgagcgcatctgtcggcgatagagtgactattacatgtagggccagccagcctatcagaccttatctgaattggtatcagcaaaaacccggcaaggctccaaaactgttgatctatgatgcttcaagcctgcaatccggagttccctcaagattttctggttccggctcaggaactgatttcaccctgactataagttctttgcagcctgaagactttgcaacatattactgtcagcaatcttactctgctccatacacattcgggcaaggtaccaaggtcgaaattaag

SEQ ID NO: 181 - DNA, 348 bp, synthetic construct
  source 1..348: mol_type = other DNA; note = Nucleic acid sequence CD19 15VH
gaggttcagttgttagagagcggggggggtctggttcagcctggtggcagcttaagactgagttgtgccgcttctggttttactttctcttcttatgcaatgtcctgggtgaggcaagcccccggtaaaggcctggaatgggtttcagttatctctggtggtggtgctaatacctactacgccgatagcgtgaaaggtagattcactatttccagggacaattcaaagaacactttgtacttacagatgaactctttgagagctgaggacaccgcagtgtattactgtgctagagattggagatactttgatcattggggacaaggaactttggtcacagtgtcatct

SEQ ID NO: 182 - protein, 14 aa, synthetic construct
  source 1..14: note = 16 CDR-L1
TGSSSNIGAGYDVH

SEQ ID NO: 183 - protein, 7 aa, synthetic construct
  source 1..7: note = 16 CDR-L2
GNNNRPS

SEQ ID NO: 184 - protein, 11 aa, synthetic construct
  source 1..11: note = 16 CDR-L3
QSYDVSLGVWV

SEQ ID NO: 185 - protein, 15 aa, synthetic construct
  source 1..15: note = 16 CDR-H1
TVSGGSISSPSYYWG

SEQ ID NO: 186 - protein, 16 aa, synthetic construct
  source 1..16: note = 16 CDR-H2
IGSIYYTGATYYNPSL

SEQ ID NO: 187 - protein, 10 aa, synthetic construct
  source 1..10: note = 16 CDR-H3
YGPAGVGFDY

SEQ ID NO: 188 - protein, 111 aa, synthetic construct
  source 1..111: note = CD19 16 VL
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDVSLGVWVFGGGTKLTVL

SEQ ID NO: 189 - protein, 120 aa, synthetic construct
  source 1..120: note = CD19 16VH
QLQLQESGPGLVKPSETLSLTCTVSGGSISSPSYYWGWIRQPPGKGLEWIGSIYYTGATYYNPSLKSRVTISVDTSKNQFSLKLSSVTAADTAVYYCARYGPAGVGFDYWGQGTLVTVSS

SEQ ID NO: 190 - protein, 246 aa, Homo sapiens
  source 1..246: note = scFv-CD19 16 human derived
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDVSLGVWVFGGGTKLTVLGGGGSGGGGSGGGGSQLQLQESGPGLVKPSETLSLTCTVSGGSISSPSYYWGWIRQPPGKGLEWIGSIYYTGATYYNPSLKSRVTISVDTSKNQFSLKLSSVTAADTAVYYCARYGPAGVGFDYWGQGTLVTVSS

SEQ ID NO: 191 - DNA, 333 bp, synthetic construct
  source 1..333: mol_type = other DNA; note = Nucleic acid sequence CD19 16 VL
cagtccgtgttgacccagcctccatcagtctcaggagccccaggccagagagtgaccatttcttgtactggatcttcctcaaatatcggggccggttacgatgttcattggtatcagcaactgcccggtacagctccaaaactgttgatctatggtaataataacagacctagcggtgtgcccgacaggttctccggctcaaagagcggaacaagtgcttctttagcaattaccggcctgcaggctgaagatgaggcagactattactgtcaatcctacgatgtttctctgggtgtttgggtttttggtggcggtaccaaattgactgtctta

SEQ ID NO: 192 - DNA, 360 bp, synthetic construct
  source 1..360: mol_type = other DNA; note = Nucleic acid sequence CD19 16VH
cagttacagttacaggagagcggtccaggtttagtgaaaccatccgaaactttatcactgacctgtacagtgtctggtggctccatctcttctccatcttattactggggttggataagacagccacctggaaaagggctggagtggattggatctatctactacaccggtgctacctactacaatccttcattgaagagcagggtcacaataagcgtggataccagtaaaaaccagttttccttgaagctgagttctgtcacagccgctgacaccgcagtgtattactgtgctagatacggtccagctggtgttggttttgattactggggacaaggaactttggtcacagtgtcatct

SEQ ID NO: 193 - protein, 14 aa, synthetic construct
  source 1..14: note = 18 CDR-L1
TGSSSNIGAGYDVH

SEQ ID NO: 194 - protein, 7 aa, synthetic construct
  source 1..7: note = 18 CDR-L2
GTKNRPS

SEQ ID NO: 195 - protein, 11 aa, synthetic construct
  source 1..11: note = 18 CDR-L3
QSYDVRLKGWV

SEQ ID NO: 196 - protein, 15 aa, synthetic construct
  source 1..15: note = 18 CDR-H1
TVSGGSITSSSYYWG

SEQ ID NO: 197 - protein, 16 aa, synthetic construct
  source 1..16: note = 18 CDR-H2
IGSIYYTGTTYYNPSL

SEQ ID NO: 198 - protein, 10 aa, synthetic construct
  source 1..10: note = 18 CDR-H3
YVGLSGGFDY

SEQ ID NO: 199 - protein, 111 aa, synthetic construct
  source 1..111: note = CD19 18 VL
QSVLTQPPSVSGAPGQRVTISCTGSSSNIGAGYDVHWYQQLPGTAPKLLIYGTKNRPSGVPDRFSGSKSGTSASLAITGLQAEDEADYYCQSYDVRLKGWVFGGGTKLTVL

SEQ ID NO: 200 - protein, 120 aa, synthetic construct
  source 1..120: note = CD19 18VH
QLQLQESGPGLVKPSETLSLTCTVSGGSITSSSYYWGWIRQPPGKGLEWIGSIYYTGTTYYNPSLKSRVTISVDTSKNQFSLKLSSVTAADTAVYYCARYVGLSGGFDYWGQGTLVTVSS

SEQ ID NO: 201 - protein, 246 aa, Homo sapiens
  source 1..246: note = scFv-CD19 18 human derived
QSVLTQPPSVSGAPGQRVTISCTGSSSNIGAGYDVHWYQQLPGTAPKLLIYGTKNRPSGVPDRFSGSKSGTSASLAITGLQAEDEADYYCQSYDVRLKGWVFGGGTKLTVLGGGGSGGGGSGGGGSQLQLQESGPGLVKPSETLSLTCTVSGGSITSSSYYWGWIRQPPGKGLEWIGSIYYTGTTYYNPSLKSRVTISVDTSKNQFSLKLSSVTAADTAVYYCARYVGLSGGFDYWGQGTLVTVSS

SEQ ID NO: 202 - DNA, 333 bp, synthetic construct
  source 1..333: mol_type = other DNA; note = Nucleic acid sequence CD19 18 VL
cagtccgtgttgacccagcctccatcagtctcaggagccccaggccagagagtgaccatttcttgtactggatcttcctcaaatatcggggccggttacgatgttcattggtatcagcaactgcccggtacagctccaaaactgttgatctatggtaccaaaaacagacctagcggtgtgcccgacaggttctccggctcaaagagcggaacaagtgcttctttagcaattaccggcctgcaggctgaagatgaggcagactattactgtcaatcctacgatgttagactgaaaggttgggtttttggtggcggtaccaaattgactgtctta

SEQ ID NO: 203 - DNA, 360 bp, synthetic construct
  source 1..360: mol_type = other DNA; note = Nucleic acid sequence CD19 18VH
cagttacagttacaggagagcggtccaggtttagtgaaaccatccgaaactttatcactgacctgtacagtgtctggtggctccatcacctcttcttcttattactggggttggataagacagccacctggaaaagggctggagtggattggatctatctactacaccggtaccacctactacaatccttcattgaagagcagggtcacaataagcgtggataccagtaaaaaccagttttccttgaagctgagttctgtcacagccgctgacaccgcagtgtattactgtgctagatacgttggtctgtctggtggttttgattactggggacaaggaactttggtcacagtgtcatct

SEQ ID NO: 204 - protein, 11 aa, synthetic construct
  source 1..11: note = 23 CDR-L1
RASQSIYSYLN

SEQ ID NO: 205 - protein, 7 aa, synthetic construct
  source 1..7: note = 23 CDR-L2
DASSLQS

SEQ ID NO: 206 - protein, 9 aa, synthetic construct
  source 1..9: note = 23 CDR-L3
QQSYTAPPT

SEQ ID NO: 207 - protein, 13 aa, synthetic construct
  source 1..13: note = 23 CDR-H1
AASGFTFSNYAMS

SEQ ID NO: 208 - protein, 17 aa, synthetic construct
  source 1..17: note = 23 CDR-H2
VSAISESGHGTYYADSV

SEQ ID NO: 209 - protein, 8 aa, synthetic construct
  source 1..8: note = 23 CDR-H3
LDWAGFDV

SEQ ID NO: 210 - protein, 107 aa, synthetic construct
  source 1..107: note = CD19 23 VL
DIQMTQSPSSLSASVGDRVTITCRASQSIYSYLNWYQQKRGKAPKLLIYDASSLQSGVPSRFSGSGSGTDFTLTISSLQPEDFATYYCQQSYTAPPTFGQGTKVEIK

SEQ ID NO: 211 - protein, 117 aa, synthetic construct
  source 1..117: note = CD19 23VH
EVQLLESGGGLVQPGGSLRLSCAASGFTFSNYAMSWVRQAPGKGLEWVSAISESGHGTYYADSVKGRFTISRDNSKNTLYLQMNSLRAEDTAVYYCARLDWAGFDVWGQGTLVTVSS

SEQ ID NO: 212 - protein, 239 aa, Homo sapiens
  source 1..239: note = scFv-CD19 23 human derived
DIQMTQSPSSLSASVGDRVTITCRASQSIYSYLNWYQQKRGKAPKLLIYDASSLQSGVPSRFSGSGSGTDFTLTISSLQPEDFATYYCQQSYTAPPTFGQGTKVEIKGGGGSGGGGSGGGGSEVQLLESGGGLVQPGGSLRLSCAASGFTFSNYAMSWVRQAPGKGLEWVSAISESGHGTYYADSVKGRFTISRDNSKNTLYLQMNSLRAEDTAVYYCARLDWAGFDVWGQGTLVTVSS

SEQ ID NO: 213 - DNA, 321 bp, synthetic construct
  source 1..321: mol_type = other DNA; note = Nucleic acid sequence CD19 23 VL
gacatacaaatgacccagtctccttcttctctgagcgcatctgtcggcgatagagtgactattacatgtagggccagccagtctatctactcttatctgaattggtatcagcaaaaacgcggcaaggctccaaaactgttgatctatgatgcttcaagcctgcaatccggagttccctcaagattttctggttccggctcaggaactgatttcaccctgactataagttctttgcagcctgaagactttgcaacatattactgtcagcaatcttacaccgctccaccaacattcgggcaaggtaccaaggtcgaaattaag

SEQ ID NO: 214 - DNA, 351 bp, synthetic construct
  source 1..351: mol_type = other DNA; note = Nucleic acid sequence CD19 23VH
gaggttcagttgttagagagcggggggggtctggttcagcctggtggcagcttaagactgagttgtgccgcttctggttttactttctctaattatgcaatgtcctgggtgaggcaagcccccggtaaaggcctggaatgggtttcagctatctctgaatctggtcatggtacctactacgccgatagcgtgaaaggtagattcactatttccagggacaattcaaagaacactttgtacttacagatgaactctttgagagctgaggacaccgcagtgtattactgtgctagactggattgggctggttttgatgtttggggacaaggaactttggtcacagtgtcatct

SEQ ID NO: 215 - protein, 157 aa, synthetic construct
  source 1..157: note = IL18(F170E)
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLEKLILKKEDELGDRSIMFTVQNED

SEQ ID NO: 216 - DNA, 738 bp, synthetic construct
  source 1..738: mol_type = other DNA; note = Nucleic acid sequence CD19 52CO scFv
cagtctgtgctgacacagcctccatctgtgtctggcgctccaggccagagagtgaccatcagctgtacaggcagcagcagcaatatcggagccggctatgacgtgcactggtatcagcagctgcctggcacagcccctaaactgctgatctacggcaccaagaacagacccagcggcgtgcccgatagattcagcggctctaagtctggcacaagcgccagcctggccattactggactgcaggccgaagatgaggccgactactactgccagagctacgacgtgcggctgaaaggctgggttttcggcggaggcacaaagctgacagtgcttggaggcggaggatctggcggaggtggaagtggcggaggcggatctcaactgcagctccaagaatctggccctggcctggtcaagcctagcgagacactgagcctgacctgtacagtgtccggcggcagcatcacaagcagcagctattactggggctggatcagacagcctcctggcaaaggcctggaatggatcggctccatctactacaccggcaccacctactacaaccccagcctgaagtcccgcgtgaccatctctgtggacaccagcaagaaccagttctccctgaagctgagcagcgtgacagccgccgatacagccgtgtactactgcgccagatacgtgggactgagcggcggctttgattattggggccagggcacactggtcaccgtgtcatct

SEQ ID NO: 217 - protein, 556 aa, synthetic construct [sequence omitted]

SEQ ID NO: 218 - protein, 257 aa, Homo sapiens
  source 1..257: note = 7URV_1|Chain A[auth C]|B-lymphocyte antigen CD19|Homo sapiens (9606)
EEPLVVKVEEGDNAVLQCLKGTSDGPTQQLTWSRESPLKPFLKLSLGLPGLGIHVSPLAIWLFISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITAR

SEQ ID NO: 219 - protein, 13 aa, synthetic construct
  source 1..13: note = FMC63, 4G7, 3B10 epitope region 1
WAKDRPEIWEGEP

SEQ ID NO: 220 - protein, 11 aa, synthetic construct
  source 1..11: note = FMC63, 4G7, 3B10 epitope region 2
PKGPKSLLSLE

SEQ ID NO: 221 - protein, 12 aa, synthetic construct
  source 1..12: note = CD19 42og epitope region 1
QPGPPSEKAWQP

SEQ ID NO: 222 - protein, 11 aa, synthetic construct
  source 1..11: note = CD19 42og epitope region 2
VPPDSVSRGPL

SEQ ID NO: 223 - protein, 9 aa, synthetic construct
  source 1..9: note = CD19 42og epitope region 1
QPGPPSEKA

SEQ ID NO: 224 - protein, 7 aa, synthetic construct
  source 1..7: note = CD19 42og epitope region 2
PPDSVSR

SEQ ID NO: 225 - DNA, 744 bp, synthetic construct
  source 1..744: mol_type = other DNA; note = Nucleic acid sequence CD19 42CO scFv (P14) Codon optimized version of P1 (42 opt) 42 scFv Codon Optimized-nucleotides
gacatccagatgacacagagccctagcagcctgtctgccagcgtgggagacagagtgaccatcacctgtagagccagccagaccatcagcaactacctgaactggtatcagcagaagcccggcaaggcccctaagctgctgatctatgctgccagctctctgcagtctggcgtgcccagcagattttctggcagcggctctggcaccgacttcaccctgaccatatctagcctgcagcctgaggacttcgccacctactactgccagcagagctacagcacccctcctacatttggccagggcaccaaggtggaaatcaaaggcggcggaggatctggcggaggtggaagtggcggaggcggatctgaagttcagctgcttgaatctggcggcggactggttcaacctggcggatctctgagactgagctgtgccgccagcggcttcaccttcagcaattacgccatcagctgggtccgacaggcccctggaaaaggccttgaatgggtgtccgtgatcacagccagcggcgtggacacctattacgccgattctgtgaagggcagattcaccatcagccgggacaacagcaagaacaccctgtacctgcagatgaacagcctgagagccgaggacaccgccgtgtactattgtgccagaggcggcaccccttacttcatcaccacctacgactactacggcttcgacgtgtggggccagggaacactggttaccgttagctct

SEQ ID NO: 226 - protein, 248 aa, synthetic construct; identical to SEQ ID NO: 9
  source 1..248: note = amino acid sequence CD19 42CO scFv (P14) Codon optimized version of P1 (42 opt) 42 scFv Codon Optimized-amino acids

SEQ ID NO: 227 - protein, 15 aa, Homo sapiens
  source 1..15: note = human IgG1 hinge
EPKSCDKTYTCPPCP

SEQ ID NO: 228 - protein, 5 aa, synthetic construct
  source 1..5: note = linker
GSGGS

SEQ ID NO: 229 - protein, 4 aa, synthetic construct
  source 1..4: note = linker
GGGS